(19) United States
(12) Patent Application Publication
(10) Pub. No.: US 2026/0061043 A1
(43) Pub. Date: Mar. 5, 2026

(54) IMMUNOGENIC COMPOSITIONS COMPRISING CONJUGATED CAPSULAR SACCHARIDE ANTIGENS, KITS COMPRISING THE SAME AND USES THEREOF

(71) Applicant: Pfizer Inc., New York, NY (US)

(72) Inventors: Annaliesa Sybil Anderson, Upper Saddle River, NJ (US); Isis Kanevsky, New York, NY (US); Farid Latif Khan, King of Prussia, PA (US); Charles Harold Jones, Monroe, NY (US); John Michael McLaughlin, Wayne, PA (US)

(21) Appl. No.: 19/021,372

(22) Filed: Jan. 15, 2025

Related U.S. Application Data
(63) Continuation of application No. 17/601,949, now abandoned.
(60) Provisional application No. 62/832,245, filed on Apr. 10, 2019.

(51) Int. Cl. A61K 39/09; A61K 39/00

(52) U.S. Cl. CPC A61K 39/092; A61K 2039/55561; A61K 2039/6037; A61K 2039/70

(57) ABSTRACT

The present invention relates to new immunogenic compositions comprising conjugated Streptococcus pneumoniae capsular saccharide antigens (glycoconjugates), kits comprising said immunogenic compositions and uses thereof. Immunogenic compositions of the present invention will typically comprise at least one glycoconjugate from a S. pneumoniae serotype not found in PREVNAR®, SYNFLORIX® and/or PREVNAR 13®. The invention also relates to vaccination of human subjects, in particular infants and elderly, against pneumococcal infections using said novel immunogenic compositions.

### CROSS REFERENCE TO RELATED APPLICATIONS

This application is a continuation of U.S. application Ser. No. 17/601,949, filed Oct. 7, 2025, which is a national stage application under 35 U.S.C. 371 of PCT/IB2020/053280, filed Apr. 6, 2020, which claims the benefit of priority of U.S. Provisional Application Ser. No. 62/832,245, filed on Apr. 10, 2019, the contents of each of which is hereby incorporated by reference in their entirety.

### REFERENCE TO SEQUENCE LISTING

This application includes an electronically submitted Sequence Listing XML file named “PC072506B_SeqListing_ST26.xml” created on Jan. 15, 2025 and having a size of 52,050 bytes. The material in the Sequence Listing XML file “PC072506B_SeqListing_ST26.xml” is part of the specification and is incorporated by reference herein in its entirety.

### FIELD OF THE INVENTION

The present invention relates to new immunogenic compositions comprising conjugated capsular saccharide antigens (glycoconjugates), kits comprising the immunogenic compositions, and uses thereof. Immunogenic compositions of the present invention typically comprise glycoconjugates, wherein the saccharides are derived from serotypes of Streptococcus pneumoniae. The invention also relates to vaccination of human subjects, in particular infants and elderly subjects, against pneumococcal infections using the novel immunogenic compositions and kits.

### BACKGROUND OF THE INVENTION

Infections caused by pneumococci are a major cause of morbidity and mortality throughout the world. Pneumonia, febrile bacteraemia and meningitis are the most common manifestations of invasive pneumococcal disease, whereas bacterial spread within the respiratory tract may result in middle-ear infection, sinusitis or recurrent bronchitis. Compared with invasive disease, the non-invasive manifestations are usually less severe, but considerably more common.

In Europe and the United States, pneumococcal pneumonia is the most common community-acquired bacterial pneumonia, estimated to affect approximately 100 per 100,000 adults each year. The corresponding figures for febrile bacteraemia and meningitis are 15-19 per 100 000 and 1-2 per 100,000, respectively. The risk for one or more of these manifestations is much higher in infants and elderly people, as well as immune compromised persons of any age. Even in economically developed regions, invasive pneumococcal disease carries high mortality; for adults with pneumococcal pneumonia the mortality rate averages 10%-20%, while it may exceed 50% in the high-risk groups. Pneumonia is by far the most common cause of pneumococcal death worldwide.

The etiological agent of pneumococcal diseases, Streptococcus pneumoniae (pneumococcus), is a Gram-positive encapsulated coccus, surrounded by a polysaccharide capsule. Differences in the composition of this capsule permit serological differentiation between about 91 capsular types, some of which are frequently associated with pneumococcal disease, others rarely. Invasive pneumococcal infections include pneumonia, meningitis and febrile bacteremia; among the common non-invasive manifestations are otitis media, sinusitis and bronchitis.

Pneumococcal conjugate vaccines (PCVs) are pneumococcal vaccines used to protect against disease caused by S. pneumoniae (pneumococcus). There are currently three PCV vaccines available on the global market: PREVNAR® (PREVENAR® in some countries) (heptavalent vaccine), SYNFLORIX® (a decavalent vaccine) and PREVNAR 13® (PREVENAR 13® in some countries) (tridecavalent vaccine).

The recent development of widespread microbial resistance to essential antibiotics and the increasing number of immunocompromised persons underline the need for pneumococcal vaccines with even broader protection.

In particular, there is a need to address remaining unmet medical need for coverage of pneumococcal disease due to serotypes not found in PREVNAR 13® and potential for emergence of non PREVNAR 13® serotypes. The specific serotypes causing disease beyond the 13 in PREVNAR 13® vary by region, population, and may change over time due to acquisition of antibiotic resistance, pneumococcal vaccine introduction and secular trends of unknown origin. There is a need for immunogenic compositions that can be used to induce an immune response against additional Streptococcus pneumoniae serotypes in humans and in particular in children less than 2 years old.

An object of the new immunogenic compositions of the present invention is to provide for appropriate protection against S. pneumoniae serotypes not found in PREVNAR 13®. In one aspect, an object of the immunogenic compositions of the present invention is to provide for appropriate protection against S. pneumoniae serotypes not found in PREVNAR® (heptavalent vaccine), SYNFLORIX® and/or PREVNAR 13® while maintaining an immune response against serotypes currently covered by said vaccines.

### SUMMARY OF THE INVENTION

To meet these and other needs, the present invention relates to novel immunogenic compositions, kits comprising the same and uses thereof. The following clauses describe some aspects and embodiments of the invention.
1. An immunogenic composition comprising at least one glycoconjugate selected from the group consisting of S. pneumoniae serotype 6C, 7C, 9N, 15A, 15B, 15C, 16F, 17F, 20, 23A, 23B, 31, 34, 35B, 35F, and 38, wherein said composition is a 1, 2, 3, 4, 5, 6, 7, 8, 9, 10, 11, 12, 13, 14, 15, or 16-valent pneumococcal conjugate composition.
2. The immunogenic composition of claim 1, wherein said composition comprises at least one glycoconjugate from S. pneumoniae serotype 6C.
3. The immunogenic composition of claim 1, wherein said composition comprises at least one glycoconjugate from S. pneumoniae serotype 7C.
4. the immunogenic composition of claim 1, wherein said composition comprises at least one glycoconjugate from S. pneumoniae serotype 9N.
5. The immunogenic composition of claim 1, wherein said composition comprises at least one glycoconjugate from S. pneumoniae serotype 15A.
6. The immunogenic composition of claim 1, wherein said composition comprises at least one glycoconjugate from S. pneumoniae serotype 15B.
7. The immunogenic composition of claim 1, wherein said composition comprises at least one glycoconjugate from S. pneumoniae serotype 15C.
8. The immunogenic composition of claim 1, wherein said composition comprises at least one glycoconjugate from S. pneumoniae serotype 16F.
9. The immunogenic composition of claim 1, wherein said composition comprises at least one glycoconjugate from S. pneumoniae serotype 17F.
10. The immunogenic composition of claim 1, wherein said composition comprises at least one glycoconjugate from S. pneumoniae serotype 20.
11. The immunogenic composition of claim 1, wherein said composition comprises at least one glycoconjugate from S. pneumoniae serotype 23A.
12. The immunogenic composition of claim 1, wherein said composition comprises at least one glycoconjugate from S. pneumoniae serotype 23B.
13. The immunogenic composition of claim 1, wherein said composition comprises at least one glycoconjugate from S. pneumoniae serotype 31.
14. The immunogenic composition of claim 1, wherein said composition comprises at least one glycoconjugate from S. pneumoniae serotype 34.
15. The immunogenic composition of claim 1, wherein said composition comprises at least one glycoconjugate from S. pneumoniae serotype 35B.
16. The immunogenic composition of claim 1, wherein said composition comprises at least one glycoconjugate from S. pneumoniae serotype 35F.
17. The immunogenic composition of claim 1, wherein said composition comprises at least one glycoconjugate from S. pneumoniae serotype 38.
18. The immunogenic composition of any one of claims 1-17, wherein said composition comprises a glycoconjugate from S. pneumoniae serotype 6C, a glycoconjugate from S. pneumoniae serotype 7C, glycoconjugate from S. pneumoniae serotype 9N, a glycoconjugate from S. pneumoniae serotype 15A, a glycoconjugate from S. pneumoniae serotype 15B, a glycoconjugate from S. pneumoniae serotype 15C, a glycoconjugate from S. pneumoniae serotype 16F, a glycoconjugate from S. pneumoniae serotype 17F, a glycoconjugate from S. pneumoniae serotype 20, a glycoconjugate from S. pneumoniae serotype 23A, a glycoconjugate from S. pneumoniae serotype 23B, a glycoconjugate from S. pneumoniae serotype 31, a glycoconjugate from S. pneumoniae serotype 34, a glycoconjugate from S. pneumoniae serotype 35B, a glycoconjugate from S. pneumoniae serotype 35F, and a glycoconjugate from S. pneumoniae serotype 38, wherein said composition is a 16-valent pneumococcal conjugate composition.
19. The immunogenic composition of any one of claims 1-18, wherein said glycoconjugates are individually conjugated to CRM₁₉₇.
20. The immunogenic composition of any one of claims 1-18, wherein said glycoconjugates are individually conjugated to PD.
21. The immunogenic composition of any one of claims 1-18, wherein said glycoconjugates are individually conjugated to TT.
22. The immunogenic composition of any one of claims 1-18, wherein said glycoconjugates are individually conjugated to DT.
23. The immunogenic composition of any one of claims 1-18, wherein at least one of said serotype 6C, 7C, 9N, 15A, 15B, 15C, 16F, 17F, 20, 23A, 23B, 31, 34, 35B, 35F, or 38 glycoconjugate has a molecular weight of between 1,000 kDa and 20,000 kDa.
24. The immunogenic composition of any one of claims 1-18, wherein at least one of said serotype 6C, 7C, 9N, 15A, 15B, 15C, 16F, 17F, 20, 23A, 23B, 31, 34, 35B, 35F, or 38 glycoconjugate comprises less than about 50% of free serotype 6C, 7C, 9N, 15A, 15B, 15C, 16F, 17F, 20, 23A, 23B, 31, 34, 35B, 35F, or 38 capsular polysaccharide compared to the total amount of serotype 6C, 7C, 9N, 15A, 15B, 15C, 16F, 17F, 20, 23A, 23B, 31, 34, 35B, 35F, or 38 capsular polysaccharide.
25 The immunogenic composition of any one of claims 1-18, wherein at least 40% of the serotype 6C, 7C, 9N, 15A, 15B, 15C, 16F, 17F, 20, 23A, 23B, 31, 34, 35B, 35F, or 38 glycoconjugates have a K_{d} below or equal to 0.3 in a CL-4B column.
26. The immunogenic composition of any one of claims 1-18, wherein the degree of conjugation of at least one of said serotype 6C, 7C, 9N, 15A, 15B, 15C, 16F, 17F, 20, 23A, 23B, 31, 34, 35B, 35F, or 38 glycoconjugate is between 2 and 15.
27. The immunogenic composition of any one of claims 1-22, wherein the carrier protein of at least one of said serotype 6C, 7C, 9N, 15A, 15B, 15C, 16F, 17F, 20, 23A, 23B, 31, 34, 35B, 35F, or 38 glycoconjugate is CRM₁₉₇.
28. The immunogenic composition of any one of claims 1-22, wherein at least one of said serotype 6C, 7C, 9N, 15A, 15B, 15C, 16F, 17F, 20, 23A, 23B, 31, 34, 35B, 35F, or 38 glycoconjugate is prepared using reductive amination.
29. The immunogenic composition of any one preceding claim, wherein at least one of said serotype 6C, 7C, 9N, 15A, 15B, 15C, 16F, 17F, 20, 23A, 23B, 31, 34, 35B, 35F, or 38 glycoconjugate has a molecular weight of between 400 kDa and 15,000 kDa.
30. The immunogenic composition of any preceding claim, wherein at least one of said serotype 6C, 7C, 9N, 15A, 15B, 15C, 16F, 17F, 20, 23A, 23B, 31, 34, 35B, 35F, or 38 glycoconjugate has a molecular weight of between 1,000 kDa and 8,000 kDa.
31. The immunogenic composition of any preceding claim, wherein at least one of said serotype 6C, 7C, 9N, 15A, 15B, 15C, 16F, 17F, 20, 23A, 23B, 31, 34, 35B, 35F, or 38 glycoconjugate is prepared using reductive amination.
32. The immunogenic composition of any preceding claim, wherein each dose of said immunogenic composition comprises 0.1 μg to 100 μg of polysaccharide of each serotype.
33. The immunogenic composition of any preceding claim, wherein each dose of said immunogenic composition comprises 1.0 μg to 10 μg of polysaccharide of each serotype.
34. The immunogenic composition of any preceding claim, wherein each dose of said immunogenic composition comprises about 1.0 μg, about 1.2 μg, about 1.4 μg, about 1.6 μg, about 1.8 μg, 2.0 μg, about 2.2 μg, about 2.4 μg, about 2.6 μg, about 2.8 μg, about 3.0 μg, about 3.2 μg, about 3.4 μg, about 3.6 μg, about 3.8 μg, about 4.0 μg, about 4.2 μg, about 4.4 μg, about 4.6 μg, about 4.8 μg, about 5.0 μg, about 5.2 μg, about 5.4 μg, about 5.6 μg, about 5.8 μg or about 6.0 μg of polysaccharide for each serotype glycoconjugate.
35. The immunogenic composition of any preceding claim, wherein each dose of said immunogenic composition comprises about 1.5 μg to about 3.0 μg of polysaccharide for each glycoconjugate from S. pneumoniae serotype 6C, 7C, 9N, 15A, 15B, 15C, 16F, 17F, 20, 23A, 23B, 31, 34, 35B, 35F, and/or 38, if present.
36. The immunogenic composition of any preceding claim, wherein each dose of said immunogenic composition comprises 10 μg to 150 μg of carrier protein.
37. The immunogenic composition of any preceding claim, wherein each dose of said immunogenic composition comprises about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg or about 75 μg of carrier protein.
38. The immunogenic composition of any preceding claim, wherein said immunogenic composition further comprises at least one antigen from other pathogens.
39. The immunogenic composition of any preceding claim, wherein said immunogenic composition further comprises at least one antigen selected from the group consisting of a diphtheria toxoid (D), a tetanus toxoid (T), a pertussis antigen (P), an acellular pertussis antigen (Pa), a hepatitis B virus (HBV) surface antigen (HBsAg), a hepatitis A virus (HAV) antigen, a conjugated Haemophilus influenzae type b capsular saccharide (Hib), and inactivated poliovirus vaccine (IPV).
40. The immunogenic composition of any preceding claim, wherein said immunogenic composition further comprises D, T and Pa.
41. The immunogenic composition of any preceding claim, wherein said immunogenic composition further comprises D, T, Pa and Hib.
42. The immunogenic composition of any preceding claim, wherein said immunogenic composition further comprises D, T, Pa and IPV.
43. The immunogenic composition of any preceding claim, wherein said immunogenic composition further comprises D, T, Pa and HBsAg.
44. The immunogenic composition of any preceding claim, wherein said immunogenic composition further comprises D, T, Pa, HBsAg and IPV.
45. The immunogenic composition of any preceding claim, wherein said immunogenic composition further comprises D, T, Pa, HBsAg and Hib.
46. The immunogenic composition of any preceding claim, wherein said immunogenic composition further comprises D, T, Pa, HBsAg, IPV and Hib.
47. The immunogenic composition of any preceding claim, wherein said immunogenic composition further comprises a conjugated N. meningitidis serogroup Y capsular saccharide (MenY).
48. The immunogenic composition of any preceding claim, wherein said immunogenic composition further comprises a conjugated N. meningitidis serogroup C capsular saccharide (MenC).
49. The immunogenic composition of any preceding claim, wherein said immunogenic composition further comprises a conjugated N. meningitidis serogroup A capsular saccharide (MenA).
50. The immunogenic composition of any preceding claim, wherein said immunogenic composition further comprises a conjugated N. meningitidis serogroup W135 capsular saccharide (MenW135).
51. The immunogenic composition of any preceding claim, wherein said immunogenic composition further comprises a conjugated N. meningitidis serogroup Y capsular saccharide (MenY) and a conjugated N. meningitidis serogroup C capsular saccharide (MenC).
52. The immunogenic composition of any preceding claim, wherein said immunogenic composition further comprises a conjugated N. meningitidis serogroup W135 capsular saccharide (MenW135), a conjugated N. meningitidis serogroup Y capsular saccharide (MenY), and/or a conjugated N. meningitidis serogroup C capsular saccharide (MenC).
53. The immunogenic composition of any preceding claim, wherein said immunogenic composition further comprises a conjugated N. meningitidis serogroup A capsular saccharide (MenA), a conjugated N. meningitidis serogroup W135 capsular saccharide (MenW135), a conjugated N. meningitidis serogroup Y capsular saccharide (MenY), and/or a conjugated N. meningitidis serogroup C capsular saccharide (MenC).
54. The immunogenic composition of any preceding claim, wherein said immunogenic composition further comprises at least one adjuvant.
55. The immunogenic composition of any preceding claim, wherein said immunogenic composition further comprises at least one adjuvant selected from the group consisting of aluminum phosphate, aluminum sulfate or aluminum hydroxide, calcium phosphate, liposomes, an oil-in-water emulsion, MF59 (4.3% w/v squalene, 0.5% w/v polysorbate 80, 0.5% w/v sorbitan trioleate), a water-in-oil emulsion, MONTANIDE™, poly(D,L-lactide-co-glycolide) (PLG) microparticles and poly(D,L-lactide-co-glycolide) (PLG) nanoparticles.
56. The immunogenic composition of any preceding claim wherein said immunogenic composition further comprise at least one adjuvant selected from the group consisting of aluminum phosphate, aluminum sulfate and aluminum hydroxide.
57. The immunogenic composition of any preceding claim wherein said immunogenic composition further comprise aluminum phosphate as adjuvant.
58. The immunogenic composition of any preceding claim wherein said immunogenic composition further comprise aluminum sulfate as adjuvant.
50. The immunogenic composition of any preceding claim wherein said immunogenic composition further comprise aluminum hydroxide as adjuvant.
60. The immunogenic composition of any preceding claim wherein said immunogenic composition comprise from 0.1 mg/mL to 1 mg/ml of elemental aluminum in the form of aluminum phosphate as adjuvant.
61. The immunogenic composition of any preceding claim wherein said immunogenic composition comprise from 0.2 mg/mL to 0.3 mg/ml of elemental aluminum in the form of aluminum phosphate as adjuvant.
62. The immunogenic composition of any preceding claim wherein said immunogenic composition comprise about 0.25 mg/ml of elemental aluminum in the form of aluminum phosphate as adjuvant.
63. The immunogenic composition of any preceding claim, wherein said immunogenic composition further comprises a CpG Oligonucleotide.
64. The immunogenic composition of any preceding claim, wherein said immunogenic composition is formulated in a liquid form.
65. The immunogenic composition of any preceding claim, wherein said immunogenic composition is formulated in a lyophilized form.
66. The immunogenic composition of any preceding claim, wherein said immunogenic composition is formulated in an aqueous liquid form.
67. The immunogenic composition of any preceding claim, wherein said immunogenic composition comprises one or more of a buffer, a salt, a divalent cation, a non-ionic detergent, a cryoprotectant such as a sugar, and an anti-oxidant such as a free radical scavenger or chelating agent, or any combinations thereof.
68. The immunogenic composition of any preceding claim, wherein said immunogenic composition comprises a buffer.
69. The immunogenic composition of any preceding claim, wherein said buffer has a pKa of about 3.5 to about 7.5.
70. The immunogenic composition of any preceding claim, wherein said buffer is phosphate, succinate, histidine or citrate.
71. The immunogenic composition of any preceding claim, wherein said buffer is succinate at a final concentration of 1.0 mM to 10 mM.
72. The immunogenic composition of any preceding claim, wherein said buffer is succinate at a final concentration of about 5.0 mM.
73. The immunogenic composition of any preceding claim, wherein the immunogenic composition comprises a salt.
74. The immunogenic composition of any preceding claim, wherein said salt is selected from the group consisting of magnesium chloride, potassium chloride, sodium chloride and a combination thereof.
75. The immunogenic composition of any preceding claim, wherein said salt is sodium chloride.
76. The immunogenic composition of any preceding claim, wherein said salt is sodium chloride at a concentration of about 150 mM.
77. The immunogenic composition of any preceding claim, wherein the immunogenic composition comprises a surfactant.
78. The immunogenic composition of any preceding claim, wherein said surfactant is selected from the group consisting of polysorbate 20, polysorbate 40, polysorbate 60, polysorbate 65, polysorbate 80, polysorbate 85, Triton N-101, Triton X-100, oxtoxynol 40, nonoxynol-9, triethanolamine, triethanolamine polypeptide oleate, polyoxyethylene-660 hydroxystearate, polyoxyethylene-35-ricinoleate, soy lecithin and a poloxamer.
79. The immunogenic composition of any preceding claim, wherein said surfactant is selected from the group polysorbate 20, polysorbate 40, polysorbate 60, polysorbate 65, polysorbate 80, polysorbate 85 and a poloxamer.
80. The immunogenic composition of any preceding claim, wherein said surfactant is polysorbate 80.
81. The immunogenic composition of any preceding claim, wherein the surfactant is polysorbate 80 at a final concentration of at least 0.0001% to 10% weight to weight (w/w).
82. The immunogenic composition of any preceding claim, wherein the surfactant is polysorbate 80 at a final concentration of at least 0.001% to 1% weight to weight (w/w).
83. The immunogenic composition of any preceding claim, wherein the surfactant is polysorbate 80 at a final concentration of at least 0.01% to 1% weight to weight (w/w).
84. The immunogenic composition of any preceding claim, wherein the surfactant is polysorbate 80 at a final concentration of 0.01%, 0.02%, 0.03%, 0.04%, 0.05%, 0.06%, 0.07%, 0.08%, 0.09% or 0.1% weight to weight (w/w).
85. The immunogenic composition of any preceding claim, wherein said immunogenic composition has a pH of 5.5 to 7.5.
86. The immunogenic composition of any preceding claim, wherein said immunogenic composition has a pH of 5.6 to 7.0.
87. The immunogenic composition of any preceding claim, wherein said immunogenic composition has a pH of 5.8 to 6.0.
88. A kit comprising: (a) a first immunogenic composition comprising said immunogenic composition of any one of claims 1-165; and (b) a second immunogenic composition comprising at least one glycoconjugate from a Streptococcus pneumoniae serotype selected from the group consisting of serotypes 1, 3, 4, 5, 6A, 6B, 7F, 9V, 14, 18C, 19A, 19F, 23F, 22F and 33F.
89. The kit of claim 88, wherein said second immunogenic composition comprises glycoconjugates from S. pneumoniae serotypes 4, 6B, 9V, 14, 18C, 19F and 23F.
90. The kit of claim 88, wherein said second immunogenic composition comprises glycoconjugates from S. pneumoniae serotypes 1, 4, 5, 6B, 7F, 9V, 14, 18C, 19F and 23F.
91. The kit of claim 88, wherein said second immunogenic composition comprises glycoconjugates from S. pneumoniae serotypes 1, 4, 5, 6A, 6B, 7F, 9V, 14, 18C, 19A, 19F and 23F.
92. The kit of claim 88, wherein said second immunogenic composition comprises glycoconjugates from S. pneumoniae serotypes 1, 3, 4, 5, 6A, 6B, 7F, 9V, 14, 18C, 19A, 19F and 23F.
93. The kit of claim 88, wherein said second immunogenic composition comprises glycoconjugates from S. pneumoniae serotypes 1, 4, 5, 6B, 7F, 9V, 14, 18C, 19F, 23F and 22F.
94. The kit of claim 88 wherein said second immunogenic composition comprises glycoconjugates from S. pneumoniae serotypes 1, 4, 5, 6B, 7F, 9V, 14, 18C, 19F, 23F and 33F.
95. The kit of claim 88, wherein said second immunogenic composition comprises glycoconjugates from S. pneumoniae serotypes 1, 4, 5, 6B, 7F, 9V, 14, 18C, 19F, 23F, 22F and 33F.
96. The kit of claim 88, wherein said second immunogenic composition comprises glycoconjugates from S. pneumoniae serotypes 1, 3, 4, 5, 6A, 6B, 7F, 9V, 14, 18C, 19A, 19F, 23F and 22F.
97. The kit of claim 88, wherein said second immunogenic composition comprises glycoconjugates from S. pneumoniae serotypes 1, 3, 4, 5, 6A, 6B, 7F, 9V, 14, 18C, 19A, 19F, 23F and 33F.
98. The kit of claim 88, wherein said second immunogenic composition comprises glycoconjugates from S. pneumoniae serotypes 1, 3, 4, 5, 6A, 6B, 7F, 9V, 14, 18C, 19A, 19F, 23F, 22F and 33F.
99. The kit of any preceding claim, wherein said glycoconjugates from S. pneumoniae serotypes 4, 6B, 9V, 14, 18C, 19F and 23F are conjugated to CRM₁₉₇.
100. The kit of any preceding claim, wherein said glycoconjugates from S. pneumoniae serotypes 1, 5 and 7F are conjugated to CRM₁₉₇.
101. The kit of any preceding claim, wherein said glycoconjugates from S. pneumoniae serotypes 6A and 19A are conjugated to CRM₁₉₇.
102. The kit of any preceding claim, wherein said glycoconjugate from S. pneumoniae serotypes 3 is conjugated to CRM₁₉₇.
103. The kit of any preceding claim, wherein said glycoconjugate from S. pneumoniae serotypes 22F is conjugated to CRM₁₉₇.
104. The kit of any preceding claim, wherein said glycoconjugate from S. pneumoniae serotypes 33F is conjugated to CRM₁₉₇.
105. The kit of any one of claims 166-182, wherein said glycoconjugates are all individually conjugated to CRM₁₉₇.
106. The kit of any preceding claim, wherein said glycoconjugates from S. pneumoniae serotypes 1, 4, 5, 6B, 7F, 9V, 14 and 23F are individually conjugated to PD.
107. The kit of any preceding claim, wherein said glycoconjugate from S. pneumoniae serotype 18C is conjugated to TT.
108. The kit of any preceding claim, wherein said glycoconjugate from S. pneumoniae serotype 19F is conjugated to DT.
109. The kit of any preceding claim, wherein said glycoconjugates from S. pneumoniae serotypes 1, 4, 5, 6B, 7F, 9V, 14 and/or 23F are individually conjugated to PD, said glycoconjugate from S. pneumoniae serotype 18C is conjugated to TT and said glycoconjugate from S. pneumoniae serotype 19F is conjugated to DT.
110. The kit of any preceding claim, wherein said glycoconjugate from S. pneumoniae serotypes 22F is conjugated to CRM₁₉₇.
111. The kit of any preceding claim, wherein said glycoconjugate from S. pneumoniae serotypes 33F is conjugated to CRM₁₉₇
112. The kit of any preceding claim, wherein said second immunogenic composition is a 7, 8, 9, 10, 11, 12, 13, 14 or 15-valent pneumococcal conjugate composition.
113. The kit of any preceding claim, wherein said second immunogenic composition is a 10, 11, 12, 13, 14 or 15-valent pneumococcal conjugate composition.
114. The kit of any preceding claim, wherein said second immunogenic composition is a 13-valent pneumococcal conjugate composition.
115. The kit of any preceding claim, wherein said second immunogenic composition is an 11-valent pneumococcal conjugate composition wherein said 11 conjugates consists of glycoconjugates from S. pneumoniae serotypes 1, 4, 5, 6B, 7F, 9V, 14 and 23F individually conjugated to PD, glycoconjugate from S. pneumoniae serotype 18C conjugated to TT, glycoconjugate from S. pneumoniae serotype 19F conjugated to DT and glycoconjugate from S. pneumoniae serotype 22F conjugated to CRM₁₉₇.
116. The kit of any preceding claim, wherein said second immunogenic composition is an 11-valent pneumococcal conjugate composition wherein said 11 conjugates consists of glycoconjugates from S. pneumoniae serotypes 1, 4, 5, 6B, 7F, 9V, 14 and 23F individually conjugated to PD, glycoconjugate from S. pneumoniae serotype 18C conjugated to TT, glycoconjugate from S. pneumoniae serotype 19F conjugated to DT and glycoconjugate from S. pneumoniae serotype 33F conjugated to CRM₁₉₇.
117. The kit of any preceding claim, wherein said second immunogenic composition is a 12-valent pneumococcal conjugate composition wherein said 12 conjugates consists of glycoconjugates from S. pneumoniae serotypes 1, 4, 5, 6B, 7F, 9V, 14 and 23F individually conjugated to PD, glycoconjugate from S. pneumoniae serotype 18C conjugated to TT, glycoconjugate from S. pneumoniae serotype 19F conjugated to DT, glycoconjugate from S. pneumoniae serotype 22F conjugated to CRM₁₉₇ and glycoconjugate from S. pneumoniae serotype 33F conjugated to CRM₁₉₇.
118. The kit of any preceding claim, wherein said second immunogenic composition is a 13-valent pneumococcal conjugate composition wherein said 13 conjugates consists of glycoconjugates from S. pneumoniae serotypes 1, 3, 4, 5, 6A, 6B, 7F, 9V, 14, 18C, 19A, 19F and 23F individually conjugated to CRM₁₉₇.
119. The kit of any preceding claim, wherein said second immunogenic composition is a 14-valent pneumococcal conjugate composition wherein said 14 conjugates consists of glycoconjugates from S. pneumoniae serotypes 1, 3, 4, 5, 6A, 6B, 7F, 9V, 14, 18C, 19A, 19F, 23F and 22F individually conjugated to CRM₁₉₇.
120. The kit of any preceding claim, wherein said second immunogenic composition is a 14-valent pneumococcal conjugate composition wherein said 14 conjugates consists of glycoconjugates from S. pneumoniae serotypes 1, 3, 4, 5, 6A, 6B, 7F, 9V, 14, 18C, 19A, 19F, 23F and 33F individually conjugated to CRM₁₉₇.
121. The kit of any preceding claim, wherein said second immunogenic composition is a 15-valent pneumococcal conjugate composition wherein said 15 conjugates consists of glycoconjugates from S. pneumoniae serotypes 1, 3, 4, 5, 6A, 6B, 7F, 9V, 14, 18C, 19A, 19F, 23F, 22F and 33F individually conjugated to CRM₁₉₇.
122. The kit of any preceding claim, wherein said glycoconjugates of the second immunogenic composition are all conjugated to the carrier protein by reductive amination.
123. The kit of any preceding claim, wherein each dose of said second immunogenic composition comprises 1.0 μg to 10 μg of polysaccharide of each serotype.
124. The kit of any preceding claim, wherein each dose of said second immunogenic composition comprises 10 μg to 150 μg of carrier protein.
125. The kit of any preceding claim, wherein each dose of said second immunogenic composition comprises about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg or about 50 μg of carrier protein.
126. The kit of any preceding claim, wherein said second immunogenic composition further comprises at least one antigen from other pathogens.
127. The kit of any preceding claim, wherein said second immunogenic composition further comprises at least one adjuvant.
128. The kit of any preceding claim, wherein said second immunogenic composition further comprises at least one adjuvant selected from the group consisting of aluminum phosphate, aluminum sulfate and aluminum hydroxide.
129. The kit of any preceding claim, wherein said second immunogenic composition further comprises aluminum phosphate as adjuvant.
130. The kit of any preceding claim, wherein said second immunogenic composition further comprises from 0.2 mg/mL to 0.3 mg/mL of elemental aluminum in the form of aluminum phosphate as adjuvant.
131. The kit of any preceding claim, wherein said second immunogenic composition further comprises about 0.25 mg/ml of elemental aluminum in the form of aluminum phosphate as adjuvant.
132. The kit of any preceding claim, wherein said second immunogenic composition further comprises a buffer.
133. The kit of any preceding claim, wherein said buffer has a pKa of about 3.5 to about 7.5.
134. The kit of any preceding claim, wherein said buffer is phosphate, succinate, histidine or citrate.
135. The kit of any preceding claim, wherein said buffer is succinate at a final concentration of about 5.0 mM.
136. The kit of any preceding claim, wherein said second immunogenic composition further comprises a salt.
137. The kit of any preceding claim, wherein said salt is selected from the group consisting of magnesium chloride, potassium chloride, sodium chloride and a combination thereof.
138. The kit of any preceding claim, wherein said second immunogenic composition comprises sodium chloride at a final concentration of 150 mM.
139. The kit of any preceding claim, wherein said second immunogenic composition further comprises a surfactant.
140. The kit of any preceding claim, wherein said surfactant is polysorbate 80.
141. The kit of any preceding claim, wherein the final concentration of polysorbate 80 is 0.01%, 0.02%, 0.03%, 0.04%, 0.05%, 0.06%, 0.07%, 0.08%, 0.09% or 0.1% (w/w).
142. The kit of any preceding claim, wherein said second immunogenic composition has a pH of 5.8 to 6.0.
143. The kit of any preceding claim, wherein said first immunogenic composition and said second immunogenic composition are in separate containers.
144. The kit of any preceding claim, wherein said first and second immunogenic compositions are formulated in a liquid form.
145. The kit of any preceding claim, wherein said first and second immunogenic compositions are formulated in a lyophilized form.
146. The kit of any preceding claim, wherein said first immunogenic composition is in a liquid form and said second immunogenic composition is in a lyophilized form.
147. The kit of any preceding claim, wherein said first immunogenic composition is in lyophilized form and said second immunogenic composition is in liquid form.
148. The immunogenic composition of any preceding claim, wherein said immunogenic composition is simultaneously, concurrently, concomitantly or sequentially administered with a second immunogenic composition.
149. The immunogenic composition of any preceding claim, for simultaneous, concurrent, concomitant or sequential administration with a second immunogenic composition.
150. The immunogenic composition of any preceding claim for simultaneous, concurrent, concomitant or sequential administration with any of the immunogenic compositions disclosed at section 3 above.
151. The immunogenic composition of any preceding claim, wherein said second immunogenic composition comprises at least one glycoconjugate from a Streptococcus pneumoniae serotype selected from the group consisting of serotypes 1, 3, 4, 5, 6A, 6B, 7F, 9V, 14, 18C, 19A, 19F, 23F, 22F and 33F.
152. The immunogenic composition of any preceding claim, wherein said second immunogenic composition comprises glycoconjugates from S. pneumoniae serotypes 4, 6B, 9V, 14, 18C, 19F and 23F.
153. The immunogenic composition of any preceding claim, wherein said second immunogenic composition comprises glycoconjugates from S. pneumoniae serotypes 1, 4, 5, 6B, 7F, 9V, 14, 18C, 19F and 23F.
154. The immunogenic composition of any preceding claim, wherein said second immunogenic composition comprises glycoconjugates from S. pneumoniae serotypes 1, 4, 5, 6A, 6B, 7F, 9V, 14, 18C, 19A, 19F and 23F.
155. The immunogenic composition of any preceding claim, wherein said second immunogenic composition comprises glycoconjugates from S. pneumoniae serotypes 1, 3, 4, 5, 6A, 6B, 7F, 9V, 14, 18C, 19A, 19F and 23F.
156. The immunogenic composition of any preceding claim, wherein said second immunogenic composition comprises glycoconjugates from S. pneumoniae serotypes 1, 4, 5, 6B, 7F, 9V, 14, 18C, 19F, 23F and 22F.
157. The immunogenic composition of any preceding claim, wherein said second immunogenic composition comprises glycoconjugates from S. pneumoniae serotypes 1, 4, 5, 6B, 7F, 9V, 14, 18C, 19F, 23F and 33F.
158. The immunogenic composition of any preceding claim, wherein said second immunogenic composition comprises glycoconjugates from S. pneumoniae serotypes 1, 4, 5, 6B, 7F, 9V, 14, 18C, 19F, 23F, 22F and 33F.
159. The immunogenic composition of any preceding claim, wherein said second immunogenic composition comprises glycoconjugates from S. pneumoniae serotypes 1, 3, 4, 5, 6A, 6B, 7F, 9V, 14, 18C, 19A, 19F, 23F and 22F.
160. The immunogenic composition of any preceding claim, wherein said second immunogenic composition comprises glycoconjugates from S. pneumoniae serotypes 1, 3, 4, 5, 6A, 6B, 7F, 9V, 14, 18C, 19A, 19F, 23F and 33F.
161. The immunogenic composition of any preceding claim, wherein said second immunogenic composition comprises glycoconjugates from S. pneumoniae serotypes 1, 3, 4, 5, 6A, 6B, 7F, 9V, 14, 18C, 19A, 19F, 23F, 22F and 33F.
162. The immunogenic composition of any preceding claim, wherein said glycoconjugates from S. pneumoniae serotypes 4, 6B, 9V, 14, 18C, 19F and 23F are conjugated to CRM₁₉₇.
163. The immunogenic composition of any preceding claim, wherein said glycoconjugates from S. pneumoniae serotypes 1, 5 and 7F are conjugated to CRM₁₉₇.
164. The immunogenic composition of any preceding claim, wherein said glycoconjugates from S. pneumoniae serotypes 6A and 19A are conjugated to CRM₁₉₇.
165. The immunogenic composition of any preceding claim, wherein said glycoconjugate from S. pneumoniae serotypes 3 is conjugated to CRM₁₉₇.
166. The immunogenic composition of any preceding claim, wherein said glycoconjugate from S. pneumoniae serotypes 22F is conjugated to CRM₁₉₇.
167. The immunogenic composition of any preceding claim, wherein said glycoconjugate from S. pneumoniae serotypes 33F is conjugated to CRM₁₉₇.
168. The immunogenic composition of any preceding claim, wherein said glycoconjugates are all individually conjugated to CRM₁₉₇.
169. The immunogenic composition of any preceding claim, wherein said glycoconjugates from S. pneumoniae serotypes 1, 4, 5, 6B, 7F, 9V, 14 and 23F are individually conjugated to PD.
170. The immunogenic composition of any preceding claim, wherein said glycoconjugate from S. pneumoniae serotype 18C is conjugated to TT.
171. The immunogenic composition of any preceding claim, wherein said glycoconjugate from S. pneumoniae serotype 19F is conjugated to DT.
172. The immunogenic composition of any preceding claim, wherein said glycoconjugates from S. pneumoniae serotypes 1, 4, 5, 6B, 7F, 9V, 14 and/or 23F are individually conjugated to PD, said glycoconjugate from S. pneumoniae serotype 18C is conjugated to TT and said glycoconjugate from S. pneumoniae serotype 19F is conjugated to DT.
173. The immunogenic composition of any preceding claim, wherein said glycoconjugate from S. pneumoniae serotype 22F is conjugated to CRM₁₉₇.
174. The immunogenic composition of any preceding claim, wherein said glycoconjugate from S. pneumoniae serotype 33F is conjugated to CRM₁₉₇.
175. The immunogenic composition of any preceding claim, wherein said second immunogenic composition is a 7, 8, 9, 10, 11, 12, 13, 14 or 15-valent pneumococcal conjugate composition.
176. The immunogenic composition of any preceding claim, wherein said second immunogenic composition is a 10, 11, 12, 13, 14 or 15-valent pneumococcal conjugate composition.
177. The immunogenic composition of any preceding claim, wherein said second immunogenic composition is a 13, 14 or 15-valent pneumococcal conjugate composition.
178. The immunogenic composition of any preceding claim, wherein said second immunogenic composition is a 13-valent pneumococcal conjugate composition.
179. The immunogenic composition of any preceding claim, wherein said second immunogenic composition is an 11-valent pneumococcal conjugate composition wherein said 11 conjugates consists of glycoconjugates from S. pneumoniae serotypes 1, 4, 5, 6B, 7F, 9V, 14 and 23F individually conjugated to PD, glycoconjugate from S. pneumoniae serotype 18C conjugated to TT, glycoconjugate from S. pneumoniae serotype 19F conjugated to DT and glycoconjugate from S. pneumoniae serotype 22F conjugated to CRM₁₉₇.
180. The immunogenic composition of any preceding claim, wherein said second immunogenic composition is an 11-valent pneumococcal conjugate composition wherein said 11 conjugates consists of glycoconjugates from S. pneumoniae serotypes 1, 4, 5, 6B, 7F, 9V, 14 and 23F individually conjugated to PD, glycoconjugate from S. pneumoniae serotype 18C conjugated to TT, glycoconjugate from S. pneumoniae serotype 19F conjugated to DT and glycoconjugate from S. pneumoniae serotype 33F conjugated to CRM₁₉₇.
181. The immunogenic composition of any preceding claim, wherein said second immunogenic composition is a 12-valent pneumococcal conjugate composition wherein said 12 conjugates consists of glycoconjugates from S. pneumoniae serotypes 1, 4, 5, 6B, 7F, 9V, 14 and 23F individually conjugated to PD, glycoconjugate from S. pneumoniae serotype 18C conjugated to TT, glycoconjugate from S. pneumoniae serotype 19F conjugated to DT, glycoconjugate from S. pneumoniae serotype 22F conjugated to CRM₁₉₇ and glycoconjugate from S. pneumoniae serotype 33F conjugated to CRM₁₉₇.
182. The immunogenic composition of any preceding claim, wherein said second immunogenic composition is a 13-valent pneumococcal conjugate composition wherein said 13 conjugates consists of glycoconjugates from S. pneumoniae serotypes 1, 3, 4, 5, 6A, 6B, 7F, 9V, 14, 18C, 19A, 19F and 23F individually conjugated to CRM₁₉₇.
183. The immunogenic composition of any preceding claim, wherein said second immunogenic composition is a 14-valent pneumococcal conjugate composition wherein said 14 conjugates consists of glycoconjugates from S. pneumoniae serotypes 1, 3, 4, 5, 6A, 6B, 7F, 9V, 14, 18C, 19A, 19F, 23F and 22F individually conjugated to CRM₁₉₇.
184. The immunogenic composition of any preceding claim, wherein said second immunogenic composition is a 14-valent pneumococcal conjugate composition wherein said 14 conjugates consists of glycoconjugates from S. pneumoniae serotypes 1, 3, 4, 5, 6A, 6B, 7F, 9V, 14, 18C, 19A, 19F, 23F and 33F individually conjugated to CRM₁₉₇.
185. The immunogenic composition of any preceding claim, wherein said second immunogenic composition is a 15-valent pneumococcal conjugate composition wherein said 15 conjugates consists of glycoconjugates from S. pneumoniae serotypes 1, 3, 4, 5, 6A, 6B, 7F, 9V, 14, 18C, 19A, 19F, 23F, 22F and 33F individually conjugated to CRM₁₉₇.
186. The immunogenic composition of any preceding claim, wherein said glycoconjugates of the second immunogenic composition are all conjugated to the carrier protein by reductive amination.
187. The immunogenic composition of any preceding claim, wherein each dose of said second immunogenic composition comprises 1 to 10 μg of polysaccharide of each serotype.
188. The immunogenic composition of any preceding claim, wherein each dose of said second immunogenic composition comprises 10 μg to 150 μg of carrier protein.
189. The immunogenic composition of any preceding claim, wherein each dose of said second immunogenic composition comprises about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg or about 50 μg of carrier protein.
190. The immunogenic composition of any preceding claim, wherein said second immunogenic composition further comprise antigens from other pathogens.
191. The immunogenic composition of any preceding claim, wherein said second immunogenic composition further comprises at least one adjuvant.
192. The immunogenic composition of any preceding claim, wherein said second immunogenic composition further comprises at least one adjuvant selected from the group consisting of aluminum phosphate, aluminum sulfate and aluminum hydroxide.
193. The immunogenic composition of any preceding claim, wherein said second immunogenic composition further comprises aluminum phosphate as adjuvant.
194. The immunogenic composition of any preceding claim, wherein said second immunogenic composition further comprises from 0.2 mg/mL to 0.3 mg/mL of elemental aluminum in the form of aluminum phosphate as adjuvant.
195. The immunogenic composition of any preceding claim, wherein said second immunogenic composition further comprises about 0.25 mg/mL of elemental aluminum in the form of aluminum phosphate as adjuvant.
196. The immunogenic composition of any preceding claim, wherein said second immunogenic composition further comprises a buffer.
197. The immunogenic composition of any preceding claim, wherein said second immunogenic composition comprises a buffer having a pKa of about 3.5 to about 7.5.
198. The immunogenic composition of any preceding claim, wherein said buffer of said second immunogenic composition is phosphate, succinate, histidine or citrate.
199. The immunogenic composition of any preceding claim, wherein said buffer of said second immunogenic composition is succinate at a final concentration of about 5.0 mM.
200. The immunogenic composition of any preceding claim, wherein said second immunogenic composition further comprises a salt.
201. The immunogenic composition of any preceding claim wherein said salt of said second immunogenic composition is selected from the group consisting of magnesium chloride, potassium chloride, sodium chloride and a combination thereof.
202. The immunogenic composition of any preceding claim, wherein said second immunogenic composition comprises sodium chloride at a final concentration of 150 mM.
203. The immunogenic composition of any preceding claim, wherein said second immunogenic composition further comprises a surfactant.
204. The immunogenic composition of any preceding claim, wherein said surfactant of said second immunogenic composition is polysorbate 80.
205. The immunogenic composition of any preceding claim, wherein the final concentration of polysorbate 80 in said second immunogenic composition is 0.01%, 0.02%, 0.03%, 0.04%, 0.05%, 0.06%, 0.07%, 0.08%, 0.09% or 0.1% (w/w).
206. The immunogenic composition of any preceding claim, wherein said second immunogenic composition has a pH of 5.8 to 6.0.
207. The immunogenic composition of any preceding claim for use in vaccination wherein the vaccination schedule is a single dose schedule.
208. The immunogenic composition of any preceding claim for use in vaccination wherein the vaccination schedule is a multiple dose schedule.
209. The immunogenic composition of any preceding claim for use in vaccination wherein the vaccination schedule consists of a series of 2 doses separated by an interval of about 1 month to about 12 months.
210. The immunogenic composition of any preceding claim for use in vaccination wherein the vaccination schedule consists of a series of 2 doses separated by an interval of about 1 month to about 6 months.
211. The immunogenic composition of any preceding claim for use in vaccination wherein the vaccination schedule consists of a series of 2 doses separated by an interval of about 1 month to about 2 months 212. The immunogenic composition of any preceding claim for use in vaccination wherein the vaccination schedule consists of a series of 3 doses separated by an interval of about 1 month to about 12 months.
213. The immunogenic composition of any preceding claim for use in vaccination wherein the vaccination schedule consists of a series of 3 doses separated by an interval of about 1 month to about 6 months.
214. The immunogenic composition of any preceding claim for use in vaccination wherein the vaccination schedule consists of a series of 3 doses separated by an interval of about 1 month to about 2 months.
215. The immunogenic composition of any preceding claim for use in vaccination wherein the vaccination schedule consists of a series of 3 doses separated by an interval of about 1 month to about 4 months followed by a fourth dose about 10 months to about 13 months after the first dose.
216. The immunogenic composition of any preceding claim for use in vaccination wherein the vaccination schedule consists of a series of 3 doses separated by an interval of about 1 month to about 2 months followed by a fourth dose about 10 months to about 13 months after the first dose.
217. The immunogenic composition of any preceding claim for use in vaccination wherein the vaccination schedule consists of a series of 2 or 3 doses separated by an interval of about 1 month to about 2 months, starting at 2 months of age, followed by a toddler dose at 12-18 months of age.
218. The immunogenic composition of any preceding claim for use in vaccination wherein the vaccination schedule consists of a series of 2 doses separated by an interval of about 2 months, starting at 2 months of age, followed by a toddler dose at 12-18 months of age.
219. The immunogenic composition of any preceding claim for use in vaccination wherein the vaccination schedule consists of 4 doses of vaccine administered at 2, 4, 6, and 12-15 months of age.
220. The immunogenic composition of any preceding claim for use in vaccination wherein the vaccination schedule consists of a prime dose given at day 0 and one or more booster doses given at intervals that range from about 2 to about 24 weeks.
221. The kit of any preceding claim for simultaneous, concurrent, concomitant or sequential administration of the first and second immunogenic compositions.
222. The immunogenic composition or kit of any preceding claim for use in a method of simultaneous administration of the first and second immunogenic compositions.
223. The immunogenic composition or kit of any preceding claim wherein the schedule of vaccination of said simultaneous administration is a single dose.
224. The immunogenic composition or kit of any preceding claim wherein the schedule of vaccination of said simultaneous administration is a multiple dose schedule.
225. The immunogenic composition or kit of any preceding claim wherein said multiple dose schedule consists of a series of 2 doses separated by an interval of about 1 month to about 12 months.
226. The immunogenic composition or kit of any preceding claim wherein said multiple dose schedule consists of a series of 2 doses separated by an interval of about 1 month to about 2 months.
227. The immunogenic composition or kit of any preceding claim wherein said multiple dose schedule consists of a series of 3 doses separated by an interval of about 1 month to about 12 months.
228. The immunogenic composition or kit of any preceding claim wherein said multiple dose schedule consists of a series of 3 doses separated by an interval of about 1 month to about 2 months.
229. The immunogenic composition or kit of any preceding claim wherein said multiple dose schedule consists of a series of 3 doses separated by an interval of about 1 month to about 2 months followed by a fourth dose about 10 months to about 13 months after the first dose.
230. The immunogenic composition or kit of any preceding claim wherein said multiple dose schedule consists of a series of 3 doses wherein each dose is separated by an interval of about 1, 2, 3 or 4 months followed by a fourth dose about 10 months to about 13 months after the first dose.
231. The immunogenic composition or kit of any preceding claim wherein said multiple dose schedule consists of at least one dose (e.g., 1, 2 or 3 doses) in the first year of age followed by at least one toddler dose.
232. The immunogenic composition or kit of any preceding claim wherein said multiple dose schedule consists of a series of 2 or 3 doses separated by an interval of about 1 month to about 2 months (for example 28-56 days between doses), starting at 2 months of age, followed by a toddler dose at 12-18 months of age.
233. The immunogenic composition or kit of any preceding claim wherein said multiple dose schedule consists of a 4 dose series of vaccine administered at 2, 4, 6, and 12-15 months of age.
234. The immunogenic composition or kit of any preceding claim wherein said multiple dose schedule consists of a prime dose given at day 0 and one or more booster doses given at intervals that range from about 2 to about 24 weeks, preferably with a dosing interval of 4-8 weeks.
235. The immunogenic composition or kit of any preceding claim wherein said multiple dose schedule consists of a prime dose given at day 0 and a booster dose given about 3 months later.
236. The immunogenic composition or kit of any preceding claim for use in a method of concomitant administration of the first and second immunogenic compositions.
237. The immunogenic composition or kit of any preceding claim wherein the schedule of vaccination of said concomitant administration is a single dose.
238. The immunogenic composition or kit of any preceding claim wherein the schedule of vaccination of said concomitant administration is a multiple dose schedule.
239. The immunogenic composition of any one of claims 226-284 or the kit of claim 299 for use in a method of concurrent administration of the first and second immunogenic compositions.
240. The immunogenic composition or kit of any preceding claim wherein the schedule of vaccination of said concurrent administration is a single dose.
241. The immunogenic composition or kit of any preceding claim wherein the schedule of vaccination of said concurrent administration is a multiple dose schedule.
242. The immunogenic composition or kit of any preceding claim wherein said multiple dose schedule consists of a series of 2 doses separated by an interval of about 1 month to about 12 months.
243. The immunogenic composition or kit of any preceding claim wherein said multiple dose schedule consists of a series of 2 doses separated by an interval of about 1 month to about 2 months.
244. The immunogenic composition or kit of any preceding claim wherein said multiple dose schedule consists of a series of 3 doses separated by an interval of about 1 month to about 12 months.
245. The immunogenic composition or kit of any preceding claim wherein said multiple dose schedule consists of a series of 3 doses separated by an interval of about 1 month to about 2 months.
246. The immunogenic composition or kit of any preceding claim wherein said multiple dose schedule consists of a series of 3 doses separated by an interval of about 1 month to about 4 months followed by a fourth dose about 10 months to about 13 months after the first dose.
247. The immunogenic composition or kit of any preceding claim wherein said multiple dose schedule consists of a series of 3 doses separated by an interval of about 1 month to about 2 months followed by a fourth dose about 10 months to about 13 months after the first dose.
248. The immunogenic composition or kit of any preceding claim wherein said multiple dose schedule consists of at least one dose (e.g., 1, 2 or 3 doses) in the first year of age followed by at least one toddler dose.
249. The immunogenic composition or kit of any preceding claim wherein said multiple dose schedule consists of a series of 2 or 3 doses separated by an interval of about 1 month to about 2 months (for example 28-56 days between doses), starting at 2 months of age, followed by a toddler dose at 12-18 months of age.
250. The immunogenic composition or kit of any preceding claim wherein said multiple dose schedule consists of a 4-dose series of vaccine administered at 2, 4, 6, and 12-15 months of age.
251. The immunogenic composition or kit of any preceding claim wherein said multiple dose schedule consists of a prime dose given at day 0 and one or more booster doses given at intervals that range from about 2 to about 24 weeks, preferably with a dosing interval of 4-8 weeks.
252. The immunogenic composition or kit of any preceding claim wherein said multiple dose schedule consists of a prime dose given at day 0 and a booster dose given about 3 months later.
253. The immunogenic composition or the kit of any preceding claim for use in a method of sequential administration of the first and second immunogenic compositions.
254. The immunogenic composition or kit of any preceding claim wherein the schedule of vaccination of said sequential administration consists of a series of 2, 3, 4, 5, 6, 7 or 8 doses.
255. The immunogenic composition or kit of any preceding claim wherein the schedule of vaccination of said sequential administration consists of a series of 2, 3 or 4 doses.
256. The immunogenic composition or kit of any preceding claim wherein the first immunogenic composition is administered first and the second immunogenic composition is administered second.
257. The immunogenic composition or kit of any preceding claim wherein the second immunogenic composition is administered first and the first immunogenic composition is administered second.
258. The immunogenic composition or kit of any preceding claim wherein the schedule of vaccination consists of a series of 2 doses separated by an interval of about 1 month to about 12 months.
259. The immunogenic composition or kit of any preceding claim wherein the schedule of vaccination consists of a series of 2 doses separated by an interval of about 1 month to about 2 months.
260. The immunogenic composition or kit of any preceding claim wherein the first and second doses are administered in the first year of age.
261. The immunogenic composition or kit of any preceding claim wherein the first dose is administered in the first year of age and the second dose is a toddler dose.
262. The immunogenic composition or kit of any preceding claim wherein said toddler dose is administered at 12-18 months of age.
263. The immunogenic composition or kit of any preceding claim wherein the schedule of vaccination of said sequential administration consists of a series of 3 doses.
264. The immunogenic composition or kit of any preceding claim wherein said schedule consists of a series of 3 doses wherein each dose is separated by an interval of about 1 month to about 12 months.
265. The immunogenic composition or kit of any preceding claim wherein said schedule consists of a series of 3 doses wherein each dose is separated by an interval of about 1 month to about 2 months.
266. The immunogenic composition or kit of any preceding claim wherein the first and second doses are administered in the first year of age and the third dose is a toddler dose.
267. The immunogenic composition or kit of any preceding claim wherein the first and second doses are separated by an interval of about 1 month to about 2 months (for example 28-56 days between doses), starting at 2 months of age, and the third dose is a toddler dose at 12-18 months of age.
268. The immunogenic composition or kit of any preceding claim wherein the first immunogenic composition is administered as the first and second doses and the second immunogenic composition is administered as the third dose.
269. The immunogenic composition or kit of any preceding claim wherein the second immunogenic composition is administered as the first and second doses and the first immunogenic composition is administered as the third dose.
270. The immunogenic composition or kit of any preceding claim wherein the first immunogenic composition is administered as the first dose, the second immunogenic composition is administered as the second dose and the first immunogenic composition is administered as the third dose.
271. The immunogenic composition or kit of any preceding claim wherein the second immunogenic composition is administered as the first dose, the first immunogenic composition is administered as the second dose and the second immunogenic composition is administered as the third dose.
272. The immunogenic composition or kit of any preceding claim wherein the first immunogenic composition is administered as the first dose and the second immunogenic composition is administered as the second and third doses.
273. The immunogenic composition or kit of any preceding claim wherein the second immunogenic composition is administered as the first dose and the first immunogenic composition is administered as the second and third doses.
274. The immunogenic composition or kit of any preceding claim wherein the schedule of vaccination of said sequential administration consists of a series of 4 doses.
275. The immunogenic composition or kit of any preceding claim wherein the first, second and third doses are separated by an interval of about 1 month to about 4 months followed by the fourth dose about 10 months to about 13 months after the first dose.
276. The immunogenic composition or kit of any preceding claim wherein the first, second and third doses are separated by an interval of about 1 month to about 2 months followed by the fourth dose about 10 months to about 13 months after the first dose.
277. The immunogenic composition or kit of any preceding claim wherein the first, second and third doses are administered in the first year of age and the fourth dose is a toddler dose.
278. The immunogenic composition or kit of any preceding claim wherein the first, second and third doses are separated by an interval of about 1 month to about 2 months (for example 28-56 days between doses), starting at 2 months of age, and the fourth dose is a toddler dose at 12-18 months of age.
279. The immunogenic composition or kit of any preceding claim wherein the first immunogenic composition is administered as the first, second and third doses and the second immunogenic composition is administered as the fourth dose.
280. The immunogenic composition or kit of any preceding claim wherein the second immunogenic composition is administered as the first, second and third doses and the first immunogenic composition is administered as the fourth dose.
281. The immunogenic composition or kit of any preceding claim wherein the first immunogenic composition is administered as the first and second doses and the second immunogenic composition is administered as the third and fourth doses.
282. The immunogenic composition or kit of any preceding claim wherein the second immunogenic composition is administered as the first and second doses and the first immunogenic composition is administered as the third and fourth doses.
283. The immunogenic composition or kit of any preceding claim wherein the first immunogenic composition is administered as the first and second doses, the second immunogenic composition is administered as the third dose and the first immunogenic composition is administered as the fourth dose.
284. The immunogenic composition or kit of any preceding claim wherein the second immunogenic composition is administered as the first and second doses, the first immunogenic composition is administered as the third dose and the second immunogenic composition is administered as the fourth dose.
285. The immunogenic composition or kit of any preceding claim wherein the first immunogenic composition is administered as the first dose and the second immunogenic composition is administered as the second, third and fourth doses.
286. The immunogenic composition or kit of any preceding claim wherein the second immunogenic composition is administered as the first dose and the first immunogenic composition is administered as the second, third and fourth doses.
287. The immunogenic composition or kit of any preceding claim wherein the first immunogenic composition is administered as the first dose, the second immunogenic composition is administered as the second dose, the first immunogenic composition is administered as the third dose and the second immunogenic composition is administered as the fourth dose.
288. The immunogenic composition or kit of any preceding claim wherein the second immunogenic composition is administered as the first dose, the first immunogenic composition is administered as the second dose, the second immunogenic composition is administered as the third dose and the first immunogenic composition is administered as the fourth dose.
289. The immunogenic composition or kit of any preceding claim wherein the first immunogenic composition is administered as the first dose, the second immunogenic composition is administered as the second dose and the first immunogenic composition is administered as the third and fourth doses.
290. The immunogenic composition or kit of any preceding claim wherein the second immunogenic composition is administered as the first dose, the first immunogenic composition is administered as the second dose and the second immunogenic composition is administered as the third and fourth doses.
291. The immunogenic composition or kit of any preceding claim wherein the first immunogenic composition is administered as the first dose, the second immunogenic composition is administered as the second and third doses and the first immunogenic composition is administered as the fourth dose.
292. The immunogenic composition or kit of any preceding claim wherein the second immunogenic composition is administered as the first dose, the first immunogenic composition is administered as the second and third doses and the second immunogenic composition is administered as the fourth dose.
293. The immunogenic composition or kit of any preceding claim wherein the schedule of vaccination of said sequential administration consists of a series of 5 doses.
294. The immunogenic composition or kit of any preceding claim wherein the schedule of vaccination consists of a series of 4 doses separated by an interval of about 1 month to about 3 months followed by a fifth dose about 10 months to about 13 months after the first dose.
295. The immunogenic composition or kit of any preceding claim wherein the first, second, third and fourth doses are administered in the first year of age and the fifth dose is a toddler dose.
296. The immunogenic composition or kit of any preceding claim wherein the first immunogenic composition (1^{st} IC) and the second immunogenic composition (2^{nd} IC) are administered according to any of the following schedules:

Dose

| 1 | 2 | 3 | 4 | 5 |
|---|---|---|---|---|
| 2^{nd} IC | 2^{nd} IC | 2^{nd} IC | 2^{nd} IC | 1^{st} IC |
| 2^{nd} IC | 2^{nd} IC | 2^{nd} IC | 1^{st} IC | 2^{nd} IC |
| 2^{nd} IC | 2^{nd} IC | 2^{nd} IC | 1^{st} IC | 1^{st} IC |
| 2^{nd} IC | 2^{nd} IC | 1^{st} IC | 2^{nd} IC | 2^{nd} IC |
| 2^{nd} IC | 2^{nd} IC | 1^{st} IC | 1^{st} IC | 2^{nd} IC |
| 2^{nd} IC | 2^{nd} IC | 1^{st} IC | 1^{st} IC | 1^{st} IC |
| 2^{nd} IC | 2^{nd} IC | 1^{st} IC | 2^{nd} IC | 1^{st} IC |
| 2^{nd} IC | 1^{st} IC | 2^{nd} IC | 2^{nd} IC | 2^{nd} IC |
| 2^{nd} IC | 1^{st} IC | 2^{nd} IC | 2^{nd} IC | 1^{st} IC |
| 2^{nd} IC | 1^{st} IC | 2^{nd} IC | 1^{st} IC | 2^{nd} IC |
| 2^{nd} IC | 1^{st} IC | 2^{nd} IC | 1^{st} IC | 1^{st} IC |
| 2^{nd} IC | 1^{st} IC | 1^{st} IC | 2^{nd} IC | 2^{nd} IC |
| 2^{nd} IC | 1^{st} IC | 1^{st} IC | 2^{nd} IC | 1^{st} IC |
| 2^{nd} IC | 1^{st} IC | 1^{st} IC | 1^{st} IC | 2^{nd} IC |
| 1^{st} IC | 2^{nd} IC | 2^{nd} IC | 2^{nd} IC | 2^{nd} IC |
| 1^{st} IC | 2^{nd} IC | 2^{nd} IC | 2^{nd} IC | 1^{st} IC |
| 1^{st} IC | 2^{nd} IC | 2^{nd} IC | 1^{st} IC | 1^{st} IC |
| 1^{st} IC | 2^{nd} IC | 2^{nd} IC | 1^{st} IC | 1^{st} IC |
| 1^{st} IC | 2^{nd} IC | 1^{st} IC | 2^{nd} IC | 2^{nd} IC |
| 1^{st} IC | 2^{nd} IC | 1^{st} IC | 2^{nd} IC | 1^{st} IC |
| 1^{st} IC | 2^{nd} IC | 1^{st} IC | 1^{st} IC | 2^{nd} IC |
| 1^{st} IC | 2^{nd} IC | 1^{st} IC | 1^{st} IC | 1^{st} IC |
| 1^{st} IC | 1^{st} IC | 2^{nd} IC | 2^{nd} IC | 2^{nd} IC |
| 1^{st} IC | 1^{st} IC | 2^{nd} IC | 2^{nd} IC | 1^{st} IC |
| 1^{st} IC | 1^{st} IC | 2^{nd} IC | 1^{st} IC | 2^{nd} IC |
| 1^{st} IC | 1^{st} IC | 2^{nd} IC | 1^{st} IC | 1^{st} IC |
| 1^{st} IC | 1^{st} IC | 1^{st} IC | 2^{nd} IC | 2^{nd} IC |
| 1^{st} IC | 1^{st} IC | 1^{st} IC | 2^{nd} IC | 1^{st} IC |
| 1^{st} IC | 1^{st} IC | 1^{st} IC | 1^{st} IC | 2^{nd} IC |

297. The immunogenic composition or kit of any preceding claim wherein the schedule of vaccination of said sequential administration consists of a series of 6 doses.
298. The immunogenic composition or kit of any preceding claim wherein the schedule of vaccination consists of a series of 5 doses separated by an interval of about 1 month to about 2 months followed by a sixth dose about 10 months to about 13 months after the first dose.
298. The immunogenic composition or kit of any preceding claim wherein the first, second, third, fourth and fifth doses are administered in the first year of age and the sixth dose is a toddler dose.
299. The immunogenic composition or kit of any preceding claim wherein the first immunogenic composition and the second immunogenic composition are administered according to any of the schedules of claim 374 followed by a sixth dose.
300. The immunogenic composition or kit of any preceding claim wherein the first immunogenic composition according to the invention is administered as the sixth dose.
301. The immunogenic composition or kit of any preceding claim wherein the second immunogenic composition according to the invention is administered as the sixth dose.
302. The immunogenic composition or kit of any preceding claim wherein the schedule of vaccination of said sequential administration consists of a series of 7 doses.
303. The immunogenic composition or kit of any preceding claim wherein the schedule of vaccination consists of a series of 6 doses separated by an interval of about 1 month followed by a seventh dose about 10 months to about 13 months after the first dose.
304. The immunogenic composition or kit of any preceding claim wherein the first, second, third, fourth, fifth and sixth doses are administered in the first year of age and the seventh dose is a toddler dose.
305. The immunogenic composition or kit of any preceding claim wherein the first immunogenic composition and the second immunogenic composition are administered according to any of the schedules of claim 379 or 380 followed by a seventh dose.
306. The immunogenic composition or kit of any preceding claim wherein the first immunogenic composition according to the invention is administered as the seventh dose.
307. The immunogenic composition or kit of any preceding claim wherein the second immunogenic composition according to the invention is administered as the seventh dose.
308. The immunogenic composition or kit of any preceding claim wherein the schedule of vaccination of said sequential administration consists of a series of 8 doses.
309. The immunogenic composition or kit of any preceding claim wherein the schedule of vaccination consists of a series of 7 doses separated by an interval of about 1 month followed by an eighth dose about 10 months to about 13 months after the first dose.
310. The immunogenic composition or kit of any preceding claim wherein the first, second, third, fourth, fifth, sixth and seventh doses are administered in the first year of age and the seventh dose is a toddler dose.
311. The immunogenic composition or kit of any preceding claim wherein the first immunogenic composition and the second immunogenic composition are administered according to any of the schedules of claim 385 or 386 followed by a eighth dose.
312. The immunogenic composition or kit of any preceding claim wherein the first immunogenic composition according to the invention is administered as the eighth dose.
313. The immunogenic composition or kit of any preceding claim wherein the second immunogenic composition according to the invention is administered as the eighth dose.
314. The immunogenic composition or kit of any preceding claim wherein the schedule of vaccination consists of the sequential administration of:
   the first immunogenic composition and
   the concomitant or concurrent administration of the first immunogenic
   composition with the second immunogenic composition.
315. The immunogenic composition or kit of any preceding claim wherein the schedule of vaccination consists of a series of 2 administrations.
316. The immunogenic composition or kit of any preceding claim wherein the schedule of vaccination consists of a series of 2 administrations separated by an interval of about 1 month to about 12 months.
317. The immunogenic composition or kit of any preceding claim wherein the first immunogenic composition is administered first and the concomitant or concurrent administration is administered second.
318. The immunogenic composition or kit of any preceding claim wherein the concomitant or concurrent administration is administered first and the first immunogenic composition is administered second.
319. The immunogenic composition or kit of any preceding claim wherein the first and second administrations are administered in the first year of age.
310. The immunogenic composition or kit of any preceding claim wherein the first administration is administered in the first year of age and the second administration is a toddler administration.
311. The immunogenic composition or kit of any preceding claim wherein said toddler administration is administered at 12-18 months of age.
312. The immunogenic composition or kit of any preceding claim wherein the schedule of vaccination consists of a series of 3 administrations.
313. The immunogenic composition or kit of any preceding claim wherein said schedule consists of a series of 3 administrations separated by an interval of about 1 month to about 12 months.
314. The immunogenic composition or kit of any preceding claim wherein the first and second administrations are administered in the first year of age and the third administration is a toddler administration.
315. The immunogenic composition or kit of any preceding claim wherein the first and second administrations are separated by an interval of about 1 month to about 2 months (for example 28-56 days between administrations), starting at 2 months of age, and the third administration is a toddler administration at 12-18 months of age.
316. The immunogenic composition or kit of any preceding claim wherein the first immunogenic composition is administered at the first and second administrations and the concomitant or concurrent administration is administered at the third administration.
317. The immunogenic composition or kit of any preceding claim wherein the concomitant or concurrent administration is administered at the first and second administrations and the first immunogenic composition is administered at the third administration.
318. The immunogenic composition or kit of any preceding claim wherein the first immunogenic composition is administered at the first administration, the concomitant or concurrent administration is administered at the second administration and the first immunogenic composition is administered at the third administration.
319. The immunogenic composition or kit of any preceding claim wherein the concomitant or concurrent administration is administered at the first administration, the first immunogenic composition is administered at the second administration and the concomitant or concurrent is administered at the third administration.
320. The immunogenic composition or kit of any preceding claim wherein the first immunogenic composition is administered at the first administration and the concomitant or concurrent administration is administered at the second and third administrations.
321. The immunogenic composition or kit of any preceding claim wherein the concomitant or concurrent administration is administered at the first administration and the first immunogenic composition is administered at the second and third administrations.
322. The immunogenic composition or kit of any preceding claim wherein the schedule of vaccination consists of a series of 4 administrations.
323. The immunogenic composition or kit of any preceding claim wherein the first, second and third administrations are separated by an interval of about 1 month to about 4 months followed by the fourth administration about 10 months to about 13 months after the first administration.
324. The immunogenic composition or kit of any preceding claim wherein the first, second and third administrations are separated by an interval of about 1 month to about 2 months followed by the fourth administration about 10 months to about 13 months after the first administration.
325. The immunogenic composition or kit of any preceding claim wherein the first, second and third administrations are administered in the first year of age and the fourth administration is a toddler administration.
326. The immunogenic composition or kit of any preceding claim wherein the first, second and third administrations are separated by an interval of about 1 month to about 2 months (for example 28-56 days between administrations), starting at 2 months of age, and the fourth administration is a toddler administration at 12-18 months of age.
327. The immunogenic composition or kit of any preceding claim wherein the first immunogenic composition is administered at the first, second and third administrations and the concomitant or concurrent administration is administered at the fourth administration.
328. The immunogenic composition or kit of any preceding claim wherein, the concomitant or concurrent administration is administered at the first, second, and third administrations and the first immunogenic composition is administered at the fourth administration.
329. The immunogenic composition or kit of any preceding claim wherein the first immunogenic composition is administered at the first and second administrations and the concomitant or concurrent administration is administered at the third and fourth administrations.
330. The immunogenic composition or kit of any preceding claim wherein the concomitant or concurrent administration is administered at the first and second administrations and the first immunogenic composition is administered at the third and fourth administrations.
331. The immunogenic composition or kit of any preceding claim wherein the first immunogenic composition is administered at the first and second administrations, the concomitant or concurrent administration is administered at the third administration and the first immunogenic composition is administered at the fourth administration.
332. The immunogenic composition or kit of any preceding claim wherein the concomitant or concurrent administration is administered at the first and second administrations, the first immunogenic composition is administered at the third administration and the concomitant or concurrent administration is administered at the fourth administration.
333. The immunogenic composition or kit of any preceding claim wherein, the first immunogenic composition is administered at the first administration and the concomitant or concurrent administration is administered at the second, third and fourth administrations.
334. The immunogenic composition or kit of any preceding claim wherein the concomitant or concurrent administration is administered at the first administration and the first immunogenic composition is administered at the second, third and fourth administrations.
335. The immunogenic composition or kit of any preceding claim wherein the first immunogenic composition is administered at the first administration, the concomitant or concurrent administration is administered at the second administration, the first immunogenic composition is administered at the third administration and the concomitant or concurrent administration is administered at the fourth administration.
336. The immunogenic composition or kit of any preceding claim wherein the concomitant or concurrent administration is administered at the first administration, the first immunogenic composition is administered at the second administration, the concomitant or concurrent administration is administered at the third administration and the first immunogenic composition is administered at the fourth administration.
337. The immunogenic composition or kit of any preceding claim wherein the first immunogenic composition is administered at the first administration, the concomitant or concurrent administration is administered at the second administration and the first immunogenic composition is administered at the third and fourth administrations.
338. The immunogenic composition or kit of any preceding claim wherein the concomitant or concurrent administration is administered at the first administration, the first immunogenic composition is administered at the second administration and the concomitant or concurrent administration is administered at the third and fourth administrations.
339. The immunogenic composition or kit of any preceding claim wherein the first immunogenic composition is administered at the first administration, the concomitant or concurrent administration is administered at the second and third administrations and the first immunogenic composition is administered at the fourth administration.
340. The immunogenic composition or kit of any preceding claim wherein the concomitant or concurrent administration is administered at the first administration, the first immunogenic composition is administered at the second and third administrations and the concomitant or concurrent administration is administered at the fourth administration.
341. The immunogenic composition or kit of any preceding claim wherein the schedule of vaccination consists of a series of 5 administrations.
342. The immunogenic composition or kit of any preceding claim wherein the schedule consists of a series of 4 administrations wherein each dose is separated by an interval of about 1 month to about 3 months followed by a fifth administration about 10 months to about 13 months after the first administration.
343. The immunogenic composition or kit of any preceding claim wherein, the first, second, third and fourth administrations are administered in the first year of age and the fifth administration is a toddler dose.
344. The immunogenic composition or kit of any preceding claim wherein, the first immunogenic composition (1^{st} IC) and the concomitant or concurrent administration of the first immunogenic composition with the second immunogenic composition (1^{st} IC/2^{nd} IC) are administered according to any of the following schedules:

Dose

| 1 | 2 | 3 | 4 | 5 |
|---|---|---|---|---|
| 1st IC/2nd IC | 1st IC/2nd IC | 1st IC/2nd IC | 1st IC/2nd IC | 1^{st} IC |
| 1st IC/2nd IC | 1st IC/2nd IC | 1st IC/2nd IC | 1^{st} IC | 1st IC/2nd IC |
| 1st IC/2nd IC | 1st IC/2nd IC | 1st IC/2nd IC | 1^{st} IC | 1^{st} IC |
| 1st IC/2nd IC | 1st IC/2nd IC | 1^{st} IC | 1st IC/2nd IC | 1st IC/2nd IC |
| 1st IC/2nd IC | 1st IC/2nd IC | 1^{st} IC | 1^{st} IC | 1st IC/2nd IC |
| 1st IC/2nd IC | 1st IC/2nd IC | 1^{st} IC | 1^{st} IC | 1^{st} IC |
| 1st IC/2nd IC | 1st IC/2nd IC | 1^{st} IC | 1st IC/2nd IC | 1^{st} IC |
| 1st IC/2nd IC | 1^{st} IC | 1st IC/2nd IC | 1st IC/2nd IC | 1st IC/2nd IC |
| 1st IC/2nd IC | 1^{st} IC | 1st IC/2nd IC | 1st IC/2nd IC | 1^{st} IC |
| 1st IC/2nd IC | 1^{st} IC | 1st IC/2nd IC | 1^{st} IC | 1st IC/2nd IC |
| 1st IC/2nd IC | 1^{st} IC | 1st IC/2nd IC | 1^{st} IC | 1^{st} IC |
| 1st IC/2nd IC | 1^{st} IC | 1^{st} IC | 1st IC/2nd IC | 1st IC/2nd IC |
| 1st IC/2nd IC | 1^{st} IC | 1^{st} IC | 1st IC/2nd IC | 1^{st} IC |
| 1st IC/2nd IC | 1^{st} IC | 1^{st} IC | 1^{st} IC | 1st IC/2nd IC |
| 1st IC/2nd IC | 1^{st} IC | 1^{st} IC | 1^{st} IC | 1^{st} IC |
| 1^{st} IC | 1st IC/2nd IC | 1st IC/2nd IC | 1st IC/2nd IC | 1st IC/2nd IC |
| 1^{st} IC | 1st IC/2nd IC | 1st IC/2nd IC | 1st IC/2nd IC | 1^{st} IC |
| 1^{st} IC | 1st IC/2nd IC | 1st IC/2nd IC | 1^{st} IC | 1st IC/2nd IC |
| 1^{st} IC | 1st IC/2nd IC | 1st IC/2nd IC | 1^{st} IC | 1^{st} IC |
| 1^{st} IC | 1st IC/2nd IC | 1^{st} IC | 1st IC/2nd IC | 1st IC/2nd IC |
| 1^{st} IC | 1st IC/2nd IC | 1^{st} IC | 1st IC/2nd IC | 1^{st} IC |
| 1^{st} IC | 1st IC/2nd IC | 1^{st} IC | 1^{st} IC | 1st IC/2nd IC |
| 1^{st} IC | 1st IC/2nd IC | 1^{st} IC | 1^{st} IC | 1^{st} IC |
| 1^{st} IC | 1^{st} IC | 1st IC/2nd IC | 1st IC/2nd IC | 1st IC/2nd IC |
| 1^{st} IC | 1^{st} IC | 1st IC/2nd IC | 1st IC/2nd IC | 1^{st} IC |
| 1^{st} IC | 1^{st} IC | 1st IC/2nd IC | 1^{st} IC | 1st IC/2nd IC |
| 1^{st} IC | 1^{st} IC | 1st IC/2nd IC | 1^{st} IC | 1^{st} IC |
| 1^{st} IC | 1^{st} IC | 1^{st} IC | 1st IC/2nd IC | 1st IC/2nd IC |
| 1^{st} IC | 1^{st} IC | 1^{st} IC | 1st IC/2nd IC | 1^{st} IC |
| 1^{st} IC | 1^{st} IC | 1^{st} IC | 1^{st} IC | 1st IC/2nd IC |

345. The immunogenic composition or kit of any preceding claim wherein the schedule of vaccination consists of a series of 6 administrations.
3346. The immunogenic composition or kit of any preceding claim wherein the schedule consists of a series of 5 administrations wherein each administration is separated by an interval of about 1 month to about 2 months followed by a sixth administration about 10 months to about 13 months after the first administration.
347. The immunogenic composition or kit of any preceding claim wherein the first, second, third, fourth and fifth administrations are administered in the first year of age and the sixth administration is a toddler administration.
348. The immunogenic composition or kit any preceding claim wherein the first immunogenic composition and the concomitant or concurrent administration of the first immunogenic composition with the second immunogenic composition are administered according to any of the schedules of claim 433 followed by a sixth administration.
349. The immunogenic composition or the kit of any preceding claim wherein the first immunogenic composition is administered as the sixth administration.
350. The immunogenic composition or the kit of any preceding claim wherein the concomitant or concurrent administration of the first immunogenic composition with the second immunogenic composition is administered at the sixth administration.
351. The immunogenic composition or the kit of any preceding claim wherein the schedule of vaccination consists of a series of 7 administrations.
352. The immunogenic composition or the kit of any preceding claim wherein the schedule of vaccination consists of a series of 6 administrations wherein each administration is separated by an interval of about 1 month followed by a seventh administration about 10 months to about 13 months after the first administration.
353. The immunogenic composition or the kit of any preceding claim wherein, the first, second, third, fourth, fifth and sixth administrations are administered in the first year of age and the seventh administration is a toddler administration.
354. The immunogenic composition or the kit of any preceding claim wherein the first immunogenic composition and the concomitant administration of the first immunogenic composition with the second immunogenic composition are administered according to the schedule of any preceding claim followed by a seventh administration.
355. The immunogenic composition or kit of any preceding claim wherein the first immunogenic composition is administered as the seventh administration.
356. The immunogenic composition or the kit of any preceding claim wherein the concomitant or concurrent administration of the first immunogenic composition with the second immunogenic composition is administered as the seventh administration.
357. The immunogenic composition or the kit of any preceding claim wherein the schedule of vaccination consists of a series of 8 administrations.
358. The immunogenic composition or the kit of any preceding claim wherein the schedule of vaccination consists of a series of 7 administrations wherein each administration is separated by an interval of about 1 month followed by an eighth administration about 10 months to about 13 months after the first administration.
359. The immunogenic composition or the kit of any preceding claim wherein, the first, second, third, fourth, fifth, sixth and seventh administrations are administered in the first year of age and the seventh administration is a toddler administration.
360. The immunogenic composition or the kit of any preceding claim wherein the first immunogenic composition and the concomitant or concurrent administration of the first immunogenic composition with the second immunogenic composition are administered according to any of the schedule of any preceding claim followed by an eighth administration.
361. The immunogenic composition or the kit of any preceding claim wherein the first immunogenic composition is administered as the eighth administration.
362. The immunogenic composition or the kit of any preceding claim wherein the concomitant or concurrent administration of the first immunogenic composition with the second immunogenic composition is administered as the eighth administration.
363. The immunogenic composition or the kit of any preceding claim wherein the schedule of vaccination consists of the sequential administration of:
   the second immunogenic composition and
   the concomitant or concurrent administration of the first immunogenic
   composition with the second immunogenic composition
364. The immunogenic composition or the kit of any preceding claim wherein said schedule is any one of the schedule according to claims 394-451 wherein administration of said second immunogenic composition of (a) replaces administration of the first immunogenic composition of (a) in said claims.
365. The immunogenic composition or the kit of any preceding claim for use as a medicament.
366. The immunogenic composition or the kit of any preceding claim for use as a vaccine.
367. The immunogenic composition or the kit of any preceding claim for use in a method for preventing, treating or ameliorating a bacterial infection, disease or condition in a subject.
368. The immunogenic composition or the kit of any preceding claim for use in a method for preventing a bacterial infection, disease or condition in a subject.
369. The immunogenic composition or the kit of any preceding claim for use in a method to protect or treat a human susceptible to pneumococcal infection, by means of administering said immunogenic compositions via a systemic or mucosal route.
370. The immunogenic composition or the kit of any preceding claim wherein said immunogenic composition(s) is/are administered by intramuscular, intraperitoneal, intradermal or subcutaneous routes.
371. The immunogenic composition or the kit of any preceding claim for use as a vaccine, wherein the subject to be vaccinated is human being less than 1 year of age.
372. The immunogenic composition or the kit of any preceding claim for use as a vaccine, wherein the subject to be vaccinated is a human being less than 2 year of age.
373. The immunogenic composition or the kit of any preceding claim for use as a vaccine, wherein the subject to be vaccinated is a human adult 50 years of age or older.
374. The immunogenic composition or the kit of any preceding claim for use as a vaccine, wherein the subject to be vaccinated is an immunocompromised human.
375. The immunogenic composition or the kit of any preceding claim for use in a single dose schedule.
376. The immunogenic composition or the kit of any preceding claim for use in a multiple dose schedule.
377. The immunogenic composition or kit of any preceding claim wherein said multiple dose schedule consists of a series of 2 doses separated by an interval of about 1 month to about 2 months.
378. The immunogenic composition or kit of any preceding claim wherein said multiple dose schedule consists of a series of 3 doses separated by an interval of about 1 month to about 2 months.
379. The immunogenic composition or kit of any preceding claim wherein said multiple dose schedule consists of a series of 3 doses separated by an interval of about 1 month to about 2 months followed by a fourth dose about 10 months to about 13 months after the first dose.
380. The immunogenic composition or kit of any preceding claim wherein said multiple dose schedule consists of at least one dose in the first year of age followed by at least one toddler dose.
381. The immunogenic composition or the kit of any preceding claim wherein said multiple dose schedule consists of a series of 2 or 3 doses separated by an interval of about 1 month to about 2 months, starting at 2 months of age, and followed by a toddler dose at 12-18 months of age.
382. The immunogenic composition or the kit of any preceding claim wherein said multiple dose schedule consists of 4 doses series of vaccine administered at 2, 4, 6, and 12-15 months of age.

### 1. Glycoconjugates of the Invention

Immunogenic compositions of the present invention typically comprise conjugated capsular saccharide antigens (also referred to as glycoconjugates), wherein the saccharides are derived from serotypes of S. pneumoniae.

If the protein carrier is the same for 2 or more saccharides in the composition, the saccharides may be conjugated to the same molecule of the protein carrier (carrier molecules having 2 or more different saccharides conjugated to it) (see, for example, WO2004/083251).

In an embodiment, the saccharides are each individually conjugated to different molecules of the protein carrier (each molecule of protein carrier only having one type of saccharide conjugated to it). In this embodiment, the capsular saccharides are said to be individually conjugated to the carrier protein.

For the purposes of the invention, the term ‘glycoconjugate’ indicates a capsular saccharide linked covalently to a carrier protein. In one embodiment, a capsular saccharide is linked directly to a carrier protein. In another embodiment, a bacterial saccharide is linked to a protein through a spacer/linker.

### 1.1 Carrier Protein of the Invention

A component of the glycoconjugate of the invention is a carrier protein to which the saccharide is conjugated. The terms “protein carrier” or “carrier protein” or “carrier” may be used interchangeably herein. Carrier proteins should be amenable to standard conjugation procedures.

In an embodiment, the carrier protein of the glycoconjugates is selected from: DT (Diphtheria toxin), TT (tetanus toxid) or fragment C of TT, CRM₁₉₇ (a nontoxic but antigenically identical variant of diphtheria toxin), other DT mutants (such as CRM176, CRM228, CRM45 (Uchida et al. (1973) J. Biol. Chem. 218:3838-3844), CRM9, CRM102, CRM103 or CRM107; and other mutations described by Nicholls and Youle in Genetically Engineered Toxins, Ed: Frankel, Maecel Dekker Inc. (1992); deletion or mutation of Glu-148 to Asp, Gln or Ser and/or Ala 158 to Gly and other mutations disclosed in U.S. Pat. Nos. 4,709,017 and 4,950,740; mutation of at least one or more residues Lys 516, Lys 526, Phe 530 and/or Lys 534 and other mutations disclosed in U.S. Pat. Nos. 5,917,017 and 6,455,673; or fragment disclosed in U.S. Pat. No. 5,843,711, pneumococcal pneumolysin (ply) (Kuo et al. (1995) Infect Immun 63:2706-2713) including ply detoxified in some fashion, for example dPLY-GMBS (WO 2004/081515, WO 2006/032499) or dPLY-formol, PhtX, including PhtA, PhtB, PhtD, PhtE (sequences of PhtA, PhtB, PhtD or PhtE are disclosed in WO 00/37105 and WO 00/39299) and fusions of Pht proteins, for example PhtDE fusions, PhtBE fusions, Pht A-E (WO 01/98334, WO 03/054007, WO 2009/000826), OMPC (meningococcal outer membrane protein), which is usually extracted from Neisseria meningitidis serogroup B (EP0372501), PorB (from N. meningitidis), PD (Haemophilus influenzae protein D; see, e.g., EP0594610 B), or immunologically functional equivalents thereof, synthetic peptides (EP0378881, EP0427347), heat shock proteins (WO93/17712, WO94/03208), pertussis proteins (WO98/58668, EP0471177), cytokines, lymphokines, growth factors or hormones (WO91/01146), artificial proteins comprising multiple human CD4+ T cell epitopes from various pathogen derived antigens (Falugi et al. (2001) Eur J Immunol 31:3816-3824) such as N19 protein (Baraldoi et al. (2004) Infect Immun 72:4884-4887) pneumococcal surface protein PspA (WO02/091998), iron uptake proteins (WO01/72337), toxin A or B of Clostridium difficile (WO00/61761), transferrin binding proteins, pneumococcal adhesion protein (PsaA), recombinant Pseudomonas aeruginosa exotoxin A (in particular non-toxic mutants thereof (such as exotoxin A bearing a substitution at glutamic acid 553 (Douglas et al. (1987) J. Bacteriol. 169 (11): 4967-4971)). Other proteins, such as ovalbumin, keyhole limpet hemocyanin (KLH), bovine serum albumin (BSA) or purified protein derivative of tuberculin (PPD) also can be used as carrier proteins. Other suitable carrier proteins include inactivated bacterial toxins such as cholera toxoid (e.g., as described in WO2004/083251), Escherichia coli LT, E. coli ST, and exotoxin A from P. aeruginosa.

In an embodiment, the carrier protein of the glycoconjugates is selected from TT, DT, DT mutants (such as CRM₁₉₇), H. influenzae protein D, PhtX, PhtD, PhtDE fusions (particularly those described in WO01/98334 and WO03/054007), detoxified pneumolysin, PorB, N19 protein, PspA, OMPC, toxin A or B of C. difficile and PsaA.

In an embodiment, the carrier protein of the glycoconjugates of the invention is DT (Diphtheria toxoid). In another embodiment, the carrier protein of the glycoconjugates of the invention is TT (tetanus toxid).

In another embodiment, the carrier protein of the glycoconjugates of the invention is PD (H. influenzae protein D; see, e.g., EP0594610 B).

In an embodiment, the capsular saccharides of the invention are conjugated to CRM₁₉₇ protein. The CRM₁₉₇ protein is a nontoxic form of diphtheria toxin but is immunologically indistinguishable from the diphtheria toxin. CRM₁₉₇ is produced by Corynebacterium diphtheriae infected by the nontoxigenic phage β197^{tox-} created by nitrosoguanidine mutagenesis of the toxigenic corynephage beta (Uchida et al. (1971) Nature New Biology 233:8-11). The CRM₁₉₇ protein has the same molecular weight as the diphtheria toxin but differs therefrom by a single base change (guanine to adenine) in the structural gene. This single base change causes an amino acid substitution (glutamic acid for glycine) in the mature protein and eliminates the toxic properties of diphtheria toxin. The CRM₁₉₇ protein is a safe and effective T-cell dependent carrier for saccharides. Further details about CRM₁₉₇ and production thereof can be found, e.g., in U.S. Pat. No. 5,614,382.

In an embodiment, the capsular saccharides of the invention are conjugated to CRM₁₉₇ protein or the A chain of CRM₁₉₇ (see CN103495161). In an embodiment, the capsular saccharides of the invention are conjugated the to A chain of CRM₁₉₇ obtained via expression by genetically recombinant E. coli (see CN103495161). In an embodiment, the capsular saccharides of the invention are all conjugated to CRM₁₉₇. In an embodiment, the capsular saccharides of the invention are all conjugated to the A chain of CRM₁₉₇.

Accordingly, in one or more embodiments, the glycoconjugates of the invention comprise CRM₁₉₇ as the carrier protein, wherein the capsular polysaccharide is covalently linked to CRM₁₉₇.

Also, in one or more embodiments, the glycoconjugates of the invention comprise TT as the carrier protein, wherein the capsular polysaccharide is covalently linked to TT.

### 1.2 Capsular Saccharide of the Invention

The term “saccharide” throughout this specification may indicate a polysaccharide or oligosaccharide, and includes both polysaccharide and oligonucleosaccharide. In one or more embodiments, the saccharide is a polysaccharide, in particular a S. pneumoniae capsular polysaccharide.

Capsular polysaccharides are prepared by standard techniques known to those of ordinary skill in the art.

In the present invention, capsular polysaccharides may be prepared, e.g., from serotypes 6C, 7C, 9N, 15A, 15B, 15C, 16F, 17F, 20, 23A, 23B, 31, 34, 35B, 35F, and 38 of S. pneumoniae. Typically, capsular polysaccharides are produced by growing each S. pneumoniae serotype in a medium (e.g. in a soy-based medium), the polysaccharides are then prepared from the bacteria culture. Bacterial strains of S. pneumoniae used to make the respective polysaccharides that are used in the glycoconjugates of the invention may be obtained from established culture collections or clinical specimens.

The population of the organism (each S. pneumoniae serotype) is often scaled up from a seed vial to seed bottles and passaged through one or more seed fermentors of increasing volume until production scale fermentation volumes are reached. At the end of the growth cycle the cells are lysed and the lysate broth is then harvested for downstream (purification) processing (see, for example, WO2006/110381, WO2008/118752, and U.S. Patent App. Pub. Nos. 2006/0228380, 2006/0228381, 2008/0102498 and 2008/0286838).

The individual polysaccharides are typically purified through centrifugation, precipitation, ultra-filtration, and/or column chromatography (see, for example, WO2006/110352 and WO2008/118752).

Purified polysaccharides may be activated (e.g., chemically activated) to make them capable of reacting (e.g., with the eTEC spacer) and then incorporated into glycoconjugates of the invention, as further described herein.

S. pneumoniae capsular polysaccharides comprise repeating oligosaccharide units which may contain up to 8 sugar residues.

In an embodiment, capsular saccharide of the invention may be one oligosaccharide unit or a shorter than native length saccharide chain of repeating oligosaccharide units. In an embodiment, capsular saccharide of the invention is one repeating oligosaccharide unit of the relevant serotype.

In an embodiment, capsular saccharide of the invention may be oligosaccharides. Oligosaccharides have a low number of repeat units (typically 5-15 repeat units) and are typically derived synthetically or by hydrolysis of polysaccharides.

In one or more embodiments, all of the capsular saccharides of the present invention and in the immunogenic compositions of the present invention are polysaccharides. High molecular weight capsular polysaccharides are able to induce certain antibody immune responses due to the epitopes present on the antigenic surface. The isolation and purification of high molecular weight capsular polysaccharides is also used in the conjugates, compositions and methods of the present invention.

In one or more embodiments, the purified polysaccharides before conjugation have a molecular weight of between 10 kDa and 4,000 kDa. In one or more embodiments, the polysaccharide has a molecular weight of between 50 kDa and 4,000 kDa. In further such embodiments, the polysaccharide has a molecular weight of between 50 kDa and 3,500 kDa; between 50 kDa and 3,000 kDa; between 50 kDa and 2,500 kDa; between 50 kDa and 2,000 kDa; between 50 kDa and 1,750 kDa; between 50 kDa and 1,500 kDa; between 50 kDa and 1,250 kDa; between 50 kDa and 1,000 kDa; between 50 kDa and 750 kDa; between 50 kDa and 500 kDa; between 100 kDa and 4,000 kDa; between 100 kDa and 3,500 kDa; 100 kDa and 3,000 kDa; 100 kDa and 2,500 kDa; 100 kDa and 2,250 kDa; between 100 kDa and 2,000 kDa; between 100 kDa and 1,750 kDa; between 100 kDa and 1,500 kDa; between 100 kDa and 1,250 kDa; between 100 kDa and 1,000 kDa; between 100 kDa and 750 kDa; between 100 kDa and 500 kDa; between 200 kDa and 4,000 kDa; between 200 kDa and 3,500 kDa; between 200 kDa and 3,000 kDa; between 200 kDa and 2,500 kDa; between 200 kDa and 2,250 kDa; between 200 kDa and 2,000 kDa; between 200 kDa and 1,750 kDa; between 200 kDa and 1,500 kDa; between 200 kDa and 1,250 kDa; between 200 kDa and 1,000 kDa; between 200 kDa and 750 kDa; or between 200 kDa and 500 kDa. Any whole number integer within any of the above ranges is contemplated as an embodiment of the disclosure.

A polysaccharide can become slightly reduced in size during normal purification procedures. Additionally, as described herein, the polysaccharides may be subjected to sizing techniques before conjugation. Mechanical or chemical sizing may also be employed. Chemical hydrolysis may be conducted using acetic acid. Mechanical sizing may be conducted using High-pressure Homogenization Shearing. The molecular weight ranges mentioned above refer to purified polysaccharides before conjugation (e.g., before activation).

In an embodiment the purified polysaccharides are capsular polysaccharides from serotypes 6C, 7C, 9N, 15A, 15B, 15C, 16F, 17F, 20, 23A, 23B, 31, 34, 35B, 35F, or 38 of S. pneumoniae, wherein the capsular polysaccharide has a molecular weight falling within one of the molecular weight ranges as described herein.

As used herein, the term “molecular weight” of polysaccharide or of carrier protein-polysaccharide conjugate refers to molecular weight calculated by size exclusion chromatography (SEC) combined with multiangle laser light scattering detector (MALLS).

In one or more embodiments, at least one of the pneumococcal saccharides from serotypes 6C, 7C, 9N, 15A, 15B, 15C, 16F, 17F, 20, 23A, 23B, 31, 34, 35B, 35F, and 38 of S. pneumoniae is O-acetylated. In one or more embodiments, two or more of the pneumococcal saccharides from serotypes 6C, 7C, 9N, 15A, 15B, 15C, 16F, 17F, 20, 23A, 23B, 31, 34, 35B, 35F, and/or 38 of S. pneumoniae are O-acetylated.

In one or more embodiments, at least one of the pneumococcal saccharides from serotypes 6C, 7C, 9N, 15A, 15B, 15C, 16F, 17F, 20, 23A, 23B, 31, 34, 35B, 35F, and 38 of S. pneumoniae is de-O-acetylated. In one or more embodiments, two or more of the pneumococcal saccharides from serotypes 6C, 7C, 9N, 15A, 15B, 15C, 16F, 17F, 20, 23A, 23B, 31, 34, 35B, 35F, and/or 38 of S. pneumoniae are de-O-acetylated.

The purified polysaccharides described herein are chemically activated to make the saccharides capable of reacting with the carrier protein. These pneumococcal conjugates are prepared by separate processes and formulated into a single dosage formulation as described below.

1.2.1 Pneumococcal Polysaccharide from S. pneumoniae Serotypes 6C, 7C, 9N, 15A, 15B, 15C, 16F, 17F, 20, 23A, 23B, 31, 34, 35B, 35F, and 38

Capsular saccharides from S. pneumoniae serotypes 6C, 7C, 9N, 15A, 15B, 15C, 16F, 17F, 20, 23A, 23B, 31, 34, 35B, 35F, and 38 may be prepared by standard techniques known to those of ordinary skill in the art (see for example WO 2006/110381). Capsular polysaccharides can be produced by growing each S. pneumoniae serotype in a medium; at the end of the growth cycle the cells are lysed and the lysate broth is then harvested for downstream (purification) processing. The individual polysaccharides are typically purified through centrifugation, precipitation, ultra-filtration, and/or column chromatography (see for example WO 2006/110352 and WO 2008/118752). Purified polysaccharides may be further processed as further described herein to prepare glycoconjugates of the invention.

In one or more embodiments, the purified polysaccharides from S. pneumoniae serotypes 6C, 7C, 9N, 15A, 15B, 15C, 16F, 17F, 20, 23A, 23B, 31, 34, 35B, 35F, and/or 38 before conjugation have a molecular weight of between 10 kDa and 4,000 kDa. In other such embodiments, the polysaccharide has a molecular weight of between 50 kDa and 4,000 kDa; between 50 kDa and 3,000 kDa or between 50 kDa and 2,000 kDa. In further such embodiments, the polysaccharide has a molecular weight of between between 50 kDa and 3,500 kDa; between 50 kDa and 3,000 kDa; between 50 kDa and 2,500 kDa; between 50 kDa and 2,000 kDa; 50 kDa and 1,750 kDa; between 50 kDa and 1,500 kDa; between 50 kDa and 1,250 kDa; between 50 kDa and 1,000 kDa; between 50 kDa and 750 kDa; between 50 kDa and 500 kDa; between 100 kDa and 4,000 kDa; between 100 kDa and 3,500 kDa; between 100 kDa and 3,000 kDa; between 100 kDa and 2,500 kDa; between 100 kDa and 2,000 kDa; between 100 kDa and 1,750 kDa; between 100 kDa and 1,500 kDa; between 100 kDa and 1,250 kDa; between 100 kDa and 1,000 kDa; between 100 kDa and 750 kDa; between 100 kDa and 500 kDa; between 200 kDa and 4,000 kDa; between 200 kDa and 3,500 kDa; between 200 kDa and 3,000 kDa; between 200 kDa and 2,500 kDa; between 200 kDa and 2,000 kDa; between 200 kDa and 1,750 kDa; between 200 kDa and 1,500 kDa; between 200 kDa and 1,250 kDa; between 200 kDa and 1,000 kDa; between 200 kDa and 750 kDa; or between 200 kDa and 500 kDa. Any whole number integer within any of the above ranges is contemplated as an embodiment of the disclosure.

A polysaccharide can become slightly reduced in size during normal purification procedures. Additionally, as described herein, polysaccharide can be subjected to sizing techniques before conjugation. The molecular weight ranges mentioned above refer to purified polysaccharides before conjugation (e.g., before activation) after an eventual sizing step.

### 1.2.2 Pneumococcal Polysaccharide Serotype 6C

Serotype 6C saccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO 2008/118752). In addition, they can be produced using synthetic protocols.

Serotype 6C S. pneumoniae strains may be obtained from established culture collections (such as for example the Streptococcal Reference Laboratory (Centers for Disease Control and Prevention, Atlanta, GA)) or clinical specimens.

In one or more embodiments, the purified polysaccharides from S. pneumoniae serotype 6C before conjugation have a molecular weight of between 10 kDa and 2,000 kDa. In one embodiment, the capsular polysaccharide has a molecular weight of between 50 kDa and 1,000 kDa. In another embodiment, the capsular polysaccharide has a molecular weight of between 70 kDa and 900 kDa. In another embodiment, the capsular polysaccharide has a molecular weight of between 100 kDa and 800 kDa.

In one or more further embodiments, the capsular polysaccharide has a molecular weight of 100 kDa to 600 kDa; 100 kDa to 500 kDa; 100 kDa to 400 kDa; 150 kDa to 600 kDa; 150 kDa to 500 kDa; 150 kDa to 400 kDa; 200 kDa to 600 kDa; 200 kDa to 500 kDa; 200 kDa to 400 kDa; 250 kDa to 600; 250 kDa to 500 kDa; 250 kDa to 400 kDa; 250 kDa to 350 kDa; 300 kDa to 600 kDa; 300 kDa to 500 kDa; 300 kDa to 400 kDa; 400 kDa to 600 kDa; 500 kDa to 600 kDa; and similar desired molecular weight ranges. Any whole number integer within any of the above ranges is contemplated as an embodiment of the disclosure.

A polysaccharide can become slightly reduced in size during normal purification procedures. Additionally, as described herein, polysaccharide can be subjected to sizing techniques before conjugation. The molecular weight ranges mentioned above refer to purified polysaccharides before conjugation (e.g., before activation) after an eventual sizing step.

### 1.2.3 Pneumococcal Polysaccharide Serotype 7C

Serotype 7C saccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO 2008/118752). In addition, they can be produced using synthetic protocols.

Serotype 7C S. pneumoniae strains may be obtained from established culture collections (such as for example the Streptococcal Reference Laboratory (Centers for Disease Control and Prevention, Atlanta, GA)) or clinical specimens.

In one or more embodiments, the purified polysaccharides from S. pneumoniae serotype 7C before conjugation have a molecular weight of between 10 kDa and 2,000 kDa. In one embodiment, the capsular polysaccharide has a molecular weight of between 50 kDa and 1,000 kDa. In another embodiment, the capsular polysaccharide has a molecular weight of between 70 kDa and 900 kDa. In another embodiment, the capsular polysaccharide has a molecular weight of between 100 kDa and 800 kDa.

In one or more further embodiments, the capsular polysaccharide has a molecular weight of 100 kDa to 600 kDa; 100 kDa to 500 kDa; 100 kDa to 400 kDa; 150 kDa to 600 kDa; 150 kDa to 500 kDa; 150 kDa to 400 kDa; 200 kDa to 600 kDa; 200 kDa to 500 kDa; 200 kDa to 400 kDa; 250 kDa to 600 kDa; 250 kDa to 500 kDa; 250 kDa to 400 kDa; 250 kDa to 350 kDa; 300 kDa to 600 kDa; 300 kDa to 500 kDa; 300 kDa to 400 kDa; 400 kDa to 600 kDa; 500 kDa to 600 kDa; and similar desired molecular weight ranges. Any whole number integer within any of the above ranges is contemplated as an embodiment of the disclosure.

A polysaccharide can become slightly reduced in size during normal purification procedures. Additionally, as described herein, polysaccharide can be subjected to sizing techniques before conjugation. The molecular weight ranges mentioned above refer to purified polysaccharides before conjugation (e.g., before activation) after an eventual sizing step.

### 1.2.4 Pneumococcal Polysaccharide Serotype 9N

Serotype 9N saccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO2008/118752). In addition, they can be produced using synthetic protocols.

Serotype 9N S. pneumoniae strains may be obtained from established culture collections (such as for example the Streptococcal Reference Laboratory (Centers for Disease Control and Prevention, Atlanta, GA)) or clinical specimens.

The isolated serotype 9N capsular polysaccharide obtained by purification of serotype 9N polysaccharide from the S. pneumoniae lysate and optionally sizing of the purified polysaccharide may be characterized by different attributes including, for example, the molecular weight (MW) and the mM of acetate per mM of said serotype 9N capsular polysaccharide.

In one or more embodiments, the purified polysaccharides from S. pneumoniae serotype 9N before conjugation have a molecular weight of between 10 kDa and 2,000 kDa. In one embodiment, the capsular polysaccharide has a molecular weight of between 50 kDa and 1,000 kDa. In another embodiment, the capsular polysaccharide has a molecular weight of between 70 kDa and 900 kDa. In another embodiment, the capsular polysaccharide has a molecular weight of between 100 kDa and 800 kDa.

In further embodiments, the capsular polysaccharide has a molecular weight of 100 kDa to 600 kDa; 100 kDa to 500 kDa; 100 kDa to 400 kDa; 100 kDa to 300 kDa; 100 kDa to 200 kDa; 150 kDa to 600 kDa; 150 kDa to 500 kDa; 150 kDa to 400 kDa; 150 kDa to 300 kDa; 150 kDa to 200 kDa; 200 kDa to 600 kDa; 200 kDa to 500 kDa; 200 kDa to 400 kDa; 250 kDa to 600 kDa; 250 kDa to 500 kDa; 250 kDa to 400 kDa; 250 kDa to 350 kDa; 300 kDa to 600 kDa; 300 kDa to 500 kDa; 300 kDa to 400 kDa; 400 kDa to 600 kDa; 500 kDa to 600 kDa; and similar desired molecular weight ranges. Any whole number integer within any of the above ranges is contemplated as an embodiment of the disclosure.

A polysaccharide can become slightly reduced in size during normal purification procedures. Additionally, as described herein, polysaccharide can be subjected to sizing techniques before conjugation. The molecular weight ranges mentioned above refer to purified polysaccharides before conjugation (e.g., before activation) after an eventual sizing step.

In an embodiment, the size of the purified serotype 9N polysaccharide is reduced by high-pressure homogenization. High-pressure homogenization achieves high shear rates by pumping the process stream through a flow path with sufficiently small dimensions. The shear rate is increased by using a larger applied homogenization pressure, and exposure time can be increased by recirculating the feed stream through the homogenizer.

The high-pressure homogenization process is particularly appropriate for reducing the size of the purified serotype 9N polysaccharide while preserving the structural features of the polysaccharide, such as the presence of O-acetyl groups.

The presence of O-acetyl in a purified, isolated or activated serotype 9N capsular polysaccharide or in a serotype 9N polysaccharide-carrier protein conjugate is expressed as the number of mM of acetate per mM of said polysaccharide or as the number of O-acetyl group per polysaccharide repeating unit.

In an embodiment, the purified polysaccharides from S. pneumoniae serotype 9N has at least 0.2, 0.4, 0.6, 0.8, 1.0, 1.2, 1.4 or 1.6 μmol acetate per μmol of said serotype 9N capsular polysaccharide.

### 1.2.5 Pneumococcal Polysaccharide Serotype 15A

Serotype 15A Streptococcus pneumoniae strains may be obtained from established culture collections (such as for example the Streptococcal Reference Laboratory (Centers for Disease Control and Prevention, Atlanta, GA)) or clinical specimens.

Capsular saccharides from S. pneumoniae serotype 15A are prepared by standard techniques known to those of ordinary skill in the art. Typically capsular polysaccharides are produced by growing each S. pneumoniae serotype in a medium (e.g., in a soy-based medium), the polysaccharides are then prepared from the bacteria culture. The population of the organism (S. pneumoniae serotype 15A) is often scaled up from a seed vial to seed bottles and passaged through one or more seed fermentors of increasing volume until production scale fermentation volumes are reached. At the end of the growth cycle, the cells are lysed and the lysate broth is then harvested for downstream (purification) processing (see for example WO 2006/110381 and WO 2008/118752, U.S. Patent App. Pub. Nos. 2006/0228380, 2006/0228381, 2008/0102498 and US2008/0286838). The polysaccharides are typically purified through centrifugation, precipitation, ultra-filtration, and/or column chromatography (see for example WO 2006/110352 and WO 2008/118752).

Purified polysaccharides from serotype 15A may be activated (e.g., chemically activated) to make them capable of reacting and then incorporated into glycoconjugates of the invention, as further described herein.

In some embodiments, the purified polysaccharides from S. pneumoniae serotype 15A before conjugation have a molecular weight of between 10 kDa and 2,000 kDa. In one embodiment, the capsular polysaccharide has a molecular weight of between 50 kDa and 1,000 kDa. In another embodiment, the capsular polysaccharide has a molecular weight of between 50 kDa and 300 kDa. In another embodiment, the capsular polysaccharide has a molecular weight of between 70 kDa and 300 kDa. In further embodiments, the capsular polysaccharide has a molecular weight of 90 kDa to 250 kDa; 90 kDa to 150 kDa; 90 kDa to 120 kDa; 80 kDa to 120 kDa; 70 kDa to 100 kDa; 70 kDa to 110 kDa; 70 kDa to 120 kDa; 70 kDa to 130 kDa; 70 kDa to 140 kDa; 70 kDa to 150 kDa; 70 kDa to 160 kDa; 80 kDa to 110 kDa; 80 kDa to 120 kDa; 80 kDa to 130 kDa; 80 kDa to 140 kDa; 80 kDa to 150 kDa; 80 kDa to 160 kDa; 90 kDa to 110 kDa; 90 kDa to 120 kDa; 90 kDa to 130 kDa; 90 kDa to 140 kDa; 90 kDa to 150 kDa; 90 kDa to 160 kDa; 100 kDa to 120 kDa; 100 kDa to 130 kDa; 100 kDa to 140 kDa; 100 kDa to 150 kDa; 100 kDa to 160 kDa; and similar desired molecular weight ranges. Any whole number integer within any of the above ranges is contemplated as an embodiment of the disclosure.

A polysaccharide can become slightly reduced in size during normal purification procedures. Additionally, as described herein, polysaccharide can be subjected to sizing techniques before conjugation. The molecular weight ranges mentioned above refer to purified polysaccharides before conjugation (e.g., before activation) after an eventual sizing step.

### 1.2.6 Pneumococcal Polysaccharide Serotype 15B

Serotype 15B polysaccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO 2008/118752). The 15B polysaccharides can also be produced using synthetic protocols known to one skilled in the art.

Serotype 15B S. pneumoniae strains may be obtained from established culture collections (such as for example the American Type Culture Collection (ATCC, Manassas, VA USA) (e.g., deposit strain No. ATCC10354) or the Streptococcal Reference Laboratory (Centers for Disease Control and Prevention, Atlanta, GA USA)) or from clinical specimens.

The bacterial cells are grown in a medium, preferably in a soy based medium. Following fermentation of bacterial cells that produce S. pneumoniae serotype 15B capsular polysaccharides, the bacterial cells are lysed to produce a cell lysate. The serotype 15B polysaccharide may then be isolated from the cell lysate using purification techniques known in the art, including the use of centrifugation, depth filtration, precipitation, ultra-filtration, treatment with activate carbon, diafiltration and/or column chromatography (see, for example, U.S. Patent App. Pub. Nos. 2006/0228380, 2006/0228381, 2007/0184071, 2007/0184072, 2007/0231340, and 2008/0102498 and WO2008/118752). The purified serotype 15B capsular polysaccharide can then be used for the preparation of immunogenic conjugates.

The isolated serotype 15B capsular polysaccharide obtained by purification of serotype 15B polysaccharide from the S. pneumoniae lysate and optionally sizing of the purified polysaccharide can be characterized by different parameters including, for example, the molecular weight (MW), the mM of acetate per mM of said serotype 15B capsular polysaccharide and the mM of glycerol per mM of said serotype 15B capsular polysaccharide.

Preferably, in order to generate 15B conjugates with advantageous filterability characteristics and/or yields, sizing of the polysaccharide to a target molecular weight range is performed prior to the conjugation to a carrier protein. Advantageously, the size of the purified serotype 15B polysaccharide is reduced while preserving critical features of the structure of the polysaccharide such as for example the presence of O-acetyl groups. Preferably, the size of the purified serotype 15B polysaccharide is reduced by mechanical homogenization.

In an embodiment, the size of the purified serotype 15B polysaccharide is reduced by high-pressure homogenization. High-pressure homogenization achieves high shear rates by pumping the process stream through a flow path with sufficiently small dimensions. The shear rate is increased by using a larger applied homogenization pressure, and exposure time can be increased by recirculating the feed stream through the homogenizer.

The high-pressure homogenization process is particularly appropriate for reducing the size of the purified serotype 15B polysaccharide while preserving the structural features of the polysaccharide, such as the presence of O-acetyl groups.

In an embodiment, the isolated serotype 15B capsular polysaccharide has a molecular weight between 5 kDa and 500 kDa, between 50 kDa and 500 kDa, between 50 kDa and 450 kDa, between 100 kDa and 400 kDa, and between 100 kDa and 350 kDa. In an embodiment, the isolated serotype 15B capsular polysaccharide has a molecular weight between 100 kDa and 350 kDa. In an embodiment, the isolated serotype 15B capsular polysaccharide has a molecular weight between 100 kDa and 300 kDa. In an embodiment, the isolated serotype 15B capsular polysaccharide has a molecular weight between 150 kDa and 300 kDa. In an embodiment, the isolated serotype 15B capsular polysaccharide has a molecular weight between 150 kDa and 350 kDa. In further embodiments, the capsular polysaccharide has a molecular weight of 100 kDa to 500 kDa; 100 kDa to 400 kDa; 100 kDa to 300 kDa; 100 kDa to 200 kDa; 150 kDa to 500 kDa; 150 kDa to 400 kDa; 150 kDa to 300 kDa; 150 kDa to 200 kDa; 200 kDa to 500 kDa; 200 kDa to 400 kDa; 250 kDa to 500 kDa; 250 kDa to 400 kDa; 250 kDa to 350 kDa; 300 kDa to 500 kDa; 300 kDa to 400 kDa; and similar desired molecular weight ranges. Any whole number integer within any of the above ranges is contemplated as an embodiment of the disclosure.

Serotype 15B polysaccharide is O-acetylated and the total amount of O-acetylation is approximately 0.8-0.9 O-acetyl groups per polysaccharide repeating unit. The degree of O-acetylation of the polysaccharide can be determined by any method known in the art, for example, by proton NMR (see for example Lemercinier et al. (1996) Carbohydrate Research 296:83-96; Jones et al. (2002) J. Pharmaceutical and Biomedical Analysis 30:1233-1247; WO2005/033148 and WO00/56357). Another commonly used method is described in Hestrin, S. (1949) J. Biol. Chem. 180:249-261. Preferably, the presence of O-acetyl groups is determined by ion-HPLC analysis.

The presence of O-acetyl in a purified, isolated or activated serotype 15B capsular polysaccharide or in a serotype 15B polysaccharide-carrier protein conjugate is expressed as the number of mM of acetate per mM of said polysaccharide or as the number of O-acetyl group per polysaccharide repeating unit.

In an embodiment, the isolated serotype 15B capsular polysaccharide comprises at least 0.1, 0.2, 0.3, 0.4, 0.5, 0.6, 0.7 or 0.8 mM acetate per mM of said serotype 15B capsular polysaccharide. In another embodiment, the isolated serotype 15B capsular polysaccharide comprises at least 0.5, 0.6 or 0.7 mM acetate per mM of said serotype 15B capsular polysaccharide. In still another embodiment, the isolated serotype 15B capsular polysaccharide comprises at least 0.6 mM acetate per mM of said serotype 15B capsular polysaccharide. In yet another embodiment, the isolated serotype 15B capsular polysaccharide comprises at least 0.7 mM acetate per mM of said serotype 15B capsular polysaccharide.

The presence of glycerolphosphate side chains is determined by measurement of glycerol using high performance anion exchange chromatography with pulsed amperometric detection (HPAEC-PAD) after its release by treatment of the polysaccharide with hydrofluoric acid (HF). The presence of glycerol in a purified, isolated or activated serotype 15B polysaccharide or in a serotype 15B polysaccharide-carrier protein conjugate is expressed as the number of mM of glycerol per mM of serotype 15B polysaccharide.

In an embodiment, the isolated serotype 15B capsular polysaccharide comprises at least 0.1, 0.2, 0.3, 0.4, 0.5, 0.6, 0.7 or 0.8 mM glycerol per mM of said serotype 15B capsular polysaccharide. In another embodiment, the isolated serotype 15B capsular polysaccharide comprises at least 0.5, 0.6 or 0.7 mM glycerol per mM of said serotype 15B capsular polysaccharide. In still another embodiment, the isolated serotype 15B capsular polysaccharide comprises at least 0.6 mM glycerol per mM of said serotype 15B capsular polysaccharide. In yet another embodiment, the isolated serotype 15B capsular polysaccharide comprises at least 0.7 mM glycerol per mM of said serotype 15B capsular polysaccharide.

In an embodiment, the isolated serotype 15B capsular polysaccharide has a molecular weight between 100 kDa and 350 kDa and comprises at least 0.6 mM acetate per mM of said serotype 15B capsular polysaccharide.

In an embodiment, the isolated serotype 15B capsular polysaccharide has a molecular weight between 100 kDa and 350 kDa and comprises at least 0.6 mM glycerol per mM of said serotype 15B capsular polysaccharide.

In an embodiment, the isolated serotype 15B capsular polysaccharide has a molecular weight between 150 kDa and 300 kDa and comprises at least 0.6 mM acetate per mM of said serotype 15B capsular polysaccharide.

In an embodiment, the isolated serotype 15B capsular polysaccharide has a molecular weight between 150 kDa and 300 kDa and comprises at least 0.6 mM glycerol per mM of said serotype 15B capsular polysaccharide.

In an embodiment, the isolated serotype 15B capsular polysaccharide has a molecular weight between 150 kDa and 350 kDa and comprises at least 0.6 mM acetate per mM of said serotype 15B capsular polysaccharide.

In an embodiment, the isolated serotype 15B capsular polysaccharide has a molecular weight between 150 kDa and 350 kDa and comprises at least 0.6 mM glycerol per mM of said serotype 15B capsular polysaccharide.

In an embodiment, the isolated serotype 15B capsular polysaccharide comprises at least 0.6 mM acetate per mM of said serotype 15B capsular polysaccharide and at least 0.6 mM glycerol per mM of said serotype 15B capsular polysaccharide.

In an embodiment, the isolated serotype 15B capsular polysaccharide has a molecular weight between 100 kDa and 350 kDa and comprises at least 0.6 mM acetate per mM of said serotype 15B capsular polysaccharide and at least 0.6 mM glycerol per mM of said serotype 15B capsular polysaccharide.

In an embodiment, the isolated serotype 15B capsular polysaccharide has a molecular weight between 150 kDa and 300 kDa and comprises at least 0.6 mM acetate per mM of said serotype 15B capsular polysaccharide and at least 0.6 mM glycerol per mM of said serotype 15B capsular polysaccharide.

In an embodiment, the isolated serotype 15B capsular polysaccharide has a molecular weight between 150 kDa and 350 kDa and comprises at least 0.6 mM acetate per mM of said serotype 15B capsular polysaccharide and at least 0.6 mM glycerol per mM of said serotype 15B capsular polysaccharide.

### 1.2.7 Pneumococcal Polysaccharide Serotype 15C

Serotype 15C polysaccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO 2008/118752). The 15C polysaccharides can also be produced using synthetic protocols known to one skilled in the art.

Serotype 15C S. pneumoniae strains may be obtained from established culture collections (such as for example the American Type Culture Collection (ATCC, Manassas, VA USA) (e.g., deposit strain No. ATCC10354) or the Streptococcal Reference Laboratory (Centers for Disease Control and Prevention, Atlanta, GA USA)) or from clinical specimens.

The bacterial cells are grown in a medium, preferably in a soy based medium. Following fermentation of bacterial cells that produce S. pneumoniae serotype 15C capsular polysaccharides, the bacterial cells are lysed to produce a cell lysate. The serotype 15C polysaccharide may then be isolated from the cell lysate using purification techniques known in the art, including the use of centrifugation, depth filtration, precipitation, ultra-filtration, treatment with activate carbon, diafiltration and/or column chromatography (see, for example, U.S. Patent App. Pub. Nos. 2006/0228380, 2006/0228381, 2007/0184071, 2007/0184072, 2007/0231340, and 2008/0102498 and WO2008/118752). The purified serotype 15B capsular polysaccharide can then be used for the preparation of immunogenic conjugates.

The isolated serotype 15C capsular polysaccharide obtained by purification of serotype 15C polysaccharide from the S. pneumoniae lysate and optionally sizing of the purified polysaccharide can be characterized by different parameters including, for example, the molecular weight (MW), the mM of acetate per mM of said serotype 15C capsular polysaccharide and the mM of glycerol per mM of said serotype 15C capsular polysaccharide.

Preferably, in order to generate 15C conjugates with advantageous filterability characteristics and/or yields, sizing of the polysaccharide to a target molecular weight range is performed prior to the conjugation to a carrier protein. Advantageously, the size of the purified serotype 15C polysaccharide is reduced while preserving critical features of the structure of the polysaccharide such as for example the presence of O-acetyl groups. Preferably, the size of the purified serotype 15C polysaccharide is reduced by mechanical homogenization.

In an embodiment, the size of the purified serotype 15C polysaccharide is reduced by high-pressure homogenization. High-pressure homogenization achieves high shear rates by pumping the process stream through a flow path with sufficiently small dimensions. The shear rate is increased by using a larger applied homogenization pressure, and exposure time can be increased by recirculating the feed stream through the homogenizer.

The high-pressure homogenization process is particularly appropriate for reducing the size of the purified serotype 15C polysaccharide while preserving the structural features of the polysaccharide, such as the presence of O-acetyl groups.

In an embodiment, the isolated serotype 15C capsular polysaccharide has a molecular weight between 5 kDa and 500 kDa, between 50 kDa and 500 kDa, between 50 kDa and 450 kDa, between 100 kDa and 400 kDa, and between 100 kDa and 350 kDa. In an embodiment, the isolated serotype 15C capsular polysaccharide has a molecular weight between 100 kDa and 350 kDa. In an embodiment, the isolated serotype 15C capsular polysaccharide has a molecular weight between 100 kDa and 300 kDa. In an embodiment, the isolated serotype 15C capsular polysaccharide has a molecular weight between 150 kDa and 300 kDa. In an embodiment, the isolated serotype 15C capsular polysaccharide has a molecular weight between 150 kDa and 350 kDa. In further embodiments, the capsular polysaccharide has a molecular weight of 100 kDa to 500 kDa; 100 kDa to 400 kDa; 100 kDa to 300 kDa; 100 kDa to 200 kDa; 150 kDa to 500 kDa; 150 kDa to 400 kDa; 150 kDa to 300 kDa; 150 kDa to 200 kDa; 200 kDa to 500 kDa; 200 kDa to 400 kDa; 250 kDa to 500 kDa; 250 kDa to 400 kDa; 250 kDa to 350 kDa; 300 kDa to 500 kDa; 300 kDa to 400 kDa; and similar desired molecular weight ranges. Any whole number integer within any of the above ranges is contemplated as an embodiment of the disclosure.

Serotype 15C polysaccharide is O-acetylated and the total amount of O-acetylation is approximately 0.8-0.9 O-acetyl groups per polysaccharide repeating unit. The degree of O-acetylation of the polysaccharide can be determined by any method known in the art, for example, by proton NMR (see for example Lemercinier et al. (1996) Carbohydrate Research 296:83-96; Jones et al. (2002) J. Pharmaceutical and Biomedical Analysis 30:1233-1247; WO2005/033148 and WO00/56357). Another commonly used method is described in Hestrin, S. (1949) J. Biol. Chem. 180:249-261. Preferably, the presence of O-acetyl groups is determined by ion-HPLC analysis.

The presence of O-acetyl in a purified, isolated or activated serotype 15C capsular polysaccharide or in a serotype 15C polysaccharide-carrier protein conjugate is expressed as the number of mM of acetate per mM of said polysaccharide or as the number of O-acetyl group per polysaccharide repeating unit.

In an embodiment, the isolated serotype 15C capsular polysaccharide comprises at least 0.1, 0.2, 0.3, 0.4, 0.5, 0.6, 0.7 or 0.8 mM acetate per mM of said serotype 15C capsular polysaccharide. In another embodiment, the isolated serotype 15C capsular polysaccharide comprises at least 0.5, 0.6 or 0.7 mM acetate per mM of said serotype 15C capsular polysaccharide. In still another embodiment, the isolated serotype 15C capsular polysaccharide comprises at least 0.6 mM acetate per mM of said serotype 15C capsular polysaccharide. In yet another embodiment, the isolated serotype 15C capsular polysaccharide comprises at least 0.7 mM acetate per mM of said serotype 15C capsular polysaccharide.

The presence of glycerolphosphate side chains is determined by measurement of glycerol using high performance anion exchange chromatography with pulsed amperometric detection (HPAEC-PAD) after its release by treatment of the polysaccharide with hydrofluoric acid (HF). The presence of glycerol in a purified, isolated or activated serotype 15C polysaccharide or in a serotype 15C polysaccharide-carrier protein conjugate is expressed as the number of mM of glycerol per mM of serotype 15C polysaccharide.

In an embodiment, the isolated serotype 15C capsular polysaccharide comprises at least 0.1, 0.2, 0.3, 0.4, 0.5, 0.6, 0.7 or 0.8 mM glycerol per mM of said serotype 15C capsular polysaccharide. In another embodiment, the isolated serotype 15C capsular polysaccharide comprises at least 0.5, 0.6 or 0.7 mM glycerol per mM of said serotype 15C capsular polysaccharide. In still another embodiment, the isolated serotype 15C capsular polysaccharide comprises at least 0.6 mM glycerol per mM of said serotype 15C capsular polysaccharide. In yet another embodiment, the isolated serotype 15C capsular polysaccharide comprises at least 0.7 mM glycerol per mM of said serotype 15C capsular polysaccharide.

In an embodiment, the isolated serotype 15C capsular polysaccharide has a molecular weight between 100 kDa and 350 kDa and comprises at least 0.6 mM acetate per mM of said serotype 15C capsular polysaccharide.

In an embodiment, the isolated serotype 15C capsular polysaccharide has a molecular weight between 100 kDa and 350 kDa and comprises at least 0.6 mM glycerol per mM of said serotype 15C capsular polysaccharide.

In an embodiment, the isolated serotype 15C capsular polysaccharide has a molecular weight between 150 kDa and 300 kDa and comprises at least 0.6 mM acetate per mM of said serotype 15C capsular polysaccharide.

In an embodiment, the isolated serotype 15C capsular polysaccharide has a molecular weight between 150 kDa and 300 kDa and comprises at least 0.6 mM glycerol per mM of said serotype 15C capsular polysaccharide.

In an embodiment, the isolated serotype 15C capsular polysaccharide has a molecular weight between 150 kDa and 350 kDa and comprises at least 0.6 mM acetate per mM of said serotype 15C capsular polysaccharide.

In an embodiment, the isolated serotype 15C capsular polysaccharide has a molecular weight between 150 kDa and 350 kDa and comprises at least 0.6 mM glycerol per mM of said serotype 15C capsular polysaccharide.

In an embodiment, the isolated serotype 15C capsular polysaccharide comprises at least 0.6 mM acetate per mM of said serotype 15C capsular polysaccharide and at least 0.6 mM glycerol per mM of said serotype 15C capsular polysaccharide.

In an embodiment, the isolated serotype 15C capsular polysaccharide has a molecular weight between 100 kDa and 350 kDa and comprises at least 0.6 mM acetate per mM of said serotype 15C capsular polysaccharide and at least 0.6 mM glycerol per mM of said serotype 15C capsular polysaccharide.

In an embodiment, the isolated serotype 15C capsular polysaccharide has a molecular weight between 150 kDa and 300 kDa and comprises at least 0.6 mM acetate per mM of said serotype 15C capsular polysaccharide and at least 0.6 mM glycerol per mM of said serotype 15C capsular polysaccharide.

In an embodiment, the isolated serotype 15C capsular polysaccharide has a molecular weight between 150 kDa and 350 kDa and comprises at least 0.6 mM acetate per mM of said serotype 15C capsular polysaccharide and at least 0.6 mM glycerol per mM of said serotype 15C capsular polysaccharide.

### 1.2.8 Pneumococcal Polysaccharide Serotype 16F

Serotype 16F polysaccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO2008/118752). In addition, they can be produced using synthetic protocols.

Serotype 16F S. pneumoniae strains may be obtained from established culture collections (such as for example the Streptococcal Reference Laboratory (Centers for Disease Control and Prevention, Atlanta, GA)) or clinical specimens.

Purified polysaccharides from serotype 16F may be activated (e.g., chemically activated) to make them capable of reacting and then incorporated into glycoconjugates of the invention, as further described herein.

The isolated serotype 16F capsular polysaccharide obtained by purification of serotype 16F polysaccharide from the S. pneumoniae lysate and optionally sizing of the purified polysaccharide can be characterized by different parameters including, for example, the molecular weight and the mM of acetate per mM of said serotype 16F capsular polysaccharide.

In some embodiments, the purified polysaccharides from S. pneumoniae serotype 16F before conjugation have a molecular weight of between between 10 kDa and 2,000 kDa. In other such embodiments, the saccharide has a molecular weight of between 50 kDa and 2,000 kDa. In further such embodiments, the saccharide has a molecular weight of between 50 kDa and 1,750 kDa; between 50 kDa and 1,500 kDa; between 50 kDa and 1,250 kDa; between 50 kDa and 1,000 kDa; between 50 kDa and 750 kDa; between 50 kDa and 500 kDa; between 100 kDa and 2,000 kDa; between 100 kDa and 1,750 kDa; between 100 kDa and 1,500 kDa; between 100 kDa and 1,250 kDa; between 100 kDa and 1,000 kDa; between 100 kDa and 750 kDa; between 100 kDa and 500 kDa; between 200 kDa and 2,000 kDa; between 200 kDa and 1,750 kDa; between 200 kDa and 1,500 kDa; between 200 kDa and 1,250 kDa; between 200 kDa and 1,000 kDa; between 200 kDa and 750 kDa; or between 200 kDa and 500 kDa. Any whole number integer within any of the above ranges is contemplated as an embodiment of the disclosure.

A polysaccharide can become slightly reduced in size during normal purification procedures. Additionally, as described herein, polysaccharide can be subjected to sizing techniques before conjugation. The molecular weight ranges mentioned above refer to purified polysaccharides before conjugation (e.g., before activation) after an eventual sizing step.

The presence of O-acetyl in a purified, isolated or activated serotype 16F capsular polysaccharide or in a serotype 16F polysaccharide-carrier protein conjugate is expressed as the number of mM of acetate per mM of said polysaccharide or as the number of O-acetyl group per polysaccharide repeating unit.

In an embodiment, the purified polysaccharides from S. pneumoniae serotype 16F has at least 0.2, 0.4, 0.6, 0.8, 1.0, 1.2, 1.4 or 1.6 μmol acetate per μmol of said serotype 16F capsular polysaccharide.

### 1.2.9 Pneumococcal Polysaccharide Serotype 17F

Serotype 17F polysaccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO2008/118752). In addition, they can be produced using synthetic protocols.

Serotype 17F S. pneumoniae strains may be obtained from established culture collections (such as for example the Streptococcal Reference Laboratory (Centers for Disease Control and Prevention, Atlanta, GA)) or clinical specimens.

Purified polysaccharides from serotype 17F may be activated (e.g., chemically activated) to make them capable of reacting and then incorporated into glycoconjugates of the invention, as further described herein.

The isolated serotype 17F capsular polysaccharide obtained by purification of serotype 16F polysaccharide from the S. pneumoniae lysate and optionally sizing of the purified polysaccharide can be characterized by different parameters including, for example, the molecular weight and the mM of acetate per mM of said serotype 16F capsular polysaccharide.

In some embodiments, the purified polysaccharides from S. pneumoniae serotype 17F before conjugation have a molecular weight of between 10 kDa and 2,000 kDa. In other such embodiments, the saccharide has a molecular weight of between 50 kDa and 2,000 kDa. In further such embodiments, the saccharide has a molecular weight of between 50 kDa and 1,750 kDa; between 50 kDa and 1,500 kDa; between 50 kDa and 1,250 kDa; between 50 kDa and 1,000 kDa; between 50 kDa and 750 kDa; between 50 kDa and 500 kDa; between 100 kDa and 2,000 kDa; between 100 kDa and 1,750 kDa; between 100 kDa and 1,500 kDa; between 100 kDa and 1,250 kDa; between 100 kDa and 1,000 kDa; between 100 kDa and 750 kDa; between 100 kDa and 500 kDa; between 200 kDa and 2,000 kDa; between 200 kDa and 1,750 kDa; between 200 kDa and 1,500 kDa; between 200 kDa and 1,250 kDa; between 200 kDa and 1,000 kDa; between 200 kDa and 750 kDa; or between 200 kDa and 500 kDa. Any whole number integer within any of the above ranges is contemplated as an embodiment of the disclosure.

A polysaccharide can become slightly reduced in size during normal purification procedures. Additionally, as described herein, polysaccharide can be subjected to sizing techniques before conjugation. The molecular weight ranges mentioned above refer to purified polysaccharides before conjugation (e.g., before activation) after an eventual sizing step.

The presence of O-acetyl in a purified, isolated or activated serotype 17F capsular polysaccharide or in a serotype 17F polysaccharide-carrier protein conjugate is expressed as the number of mM of acetate per mM of said polysaccharide or as the number of O-acetyl group per polysaccharide repeating unit.

In an embodiment, the purified polysaccharides from S. pneumoniae serotype 17F has at least 0.2, 0.4, 0.6, 0.8, 1.0, 1.2, 1.4 or 1.6 μmol acetate per μmol of said serotype 17F capsular polysaccharide.

### 1.2.10 Pneumococcal Polysaccharide Serotype 20

Serotype 20 polysaccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO2008/118752). In addition, they can be produced using synthetic protocols.

Serotype 20 S. pneumoniae strains may be obtained from established culture collections (such as for example the Streptococcal Reference Laboratory (Centers for Disease Control and Prevention, Atlanta, GA)) or clinical specimens.

Purified polysaccharides from serotype 20 may be activated (e.g., chemically activated) to make them capable of reacting and then incorporated into glycoconjugates of the invention, as further described herein.

The isolated serotype 20 capsular polysaccharide obtained by purification of serotype 20 polysaccharide from the S. pneumoniae lysate and optionally sizing of the purified polysaccharide can be characterized by different parameters including, for example, the molecular weight and the mM of acetate per mM of said serotype 20 capsular polysaccharide.

In some embodiments, the purified polysaccharides from S. pneumoniae serotype 20 before conjugation have a molecular weight of between 10 kDa and 2,000 kDa. In other such embodiments, the saccharide has a molecular weight of between 50 kDa and 2,000 kDa. In further such embodiments, the saccharide has a molecular weight of between 50 kDa and 1,750 kDa; between 50 kDa and 1,500 kDa; between 50 kDa and 1,250 kDa; between 50 kDa and 1,000 kDa; between 50 kDa and 750 kDa; between 50 kDa and 500 kDa; between 100 kDa and 2,000 kDa; between 100 kDa and 1,750 kDa; between 100 kDa and 1,500 kDa; between 100 kDa and 1,250 kDa; between 100 kDa and 1,000 kDa; between 100 kDa and 750 kDa; between 100 kDa and 500 kDa; between 200 kDa and 2,000 kDa; between 200 kDa and 1,750 kDa; between 200 kDa and 1,500 kDa; between 200 kDa and 1,250 kDa; between 200 kDa and 1,000 kDa; between 200 kDa and 750 kDa; or between 200 kDa and 500 kDa. Any whole number integer within any of the above ranges is contemplated as an embodiment of the disclosure.

A polysaccharide can become slightly reduced in size during normal purification procedures. Additionally, as described herein, polysaccharide can be subjected to sizing techniques before conjugation. The molecular weight ranges mentioned above refer to purified polysaccharides before conjugation (e.g., before activation) after an eventual sizing step.

The presence of O-acetyl in a purified, isolated or activated serotype 20 capsular polysaccharide or in a serotype 20 polysaccharide-carrier protein conjugate is expressed as the number of mM of acetate per mM of said polysaccharide or as the number of O-acetyl group per polysaccharide repeating unit.

In an embodiment, the purified polysaccharides from S. pneumoniae serotype 20 has at least 0.2, 0.4, 0.6, 0.8, 1.0, 1.2, 1.4 or 1.6 μmol acetate per μmol of said serotype 20 capsular polysaccharide.

### 1.2.11 Pneumococcal Polysaccharide Serotype 23A

Serotype 23A polysaccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO2008/118752). In addition, they can be produced using synthetic protocols.

Serotype 23A S. pneumoniae strains may be obtained from established culture collections (such as for example the Streptococcal Reference Laboratory (Centers for Disease Control and Prevention, Atlanta, GA)) or clinical specimens.

Purified polysaccharides from serotype 23A may be activated (e.g., chemically activated) to make them capable of reacting and then incorporated into glycoconjugates of the invention, as further described herein.

The isolated serotype 23A capsular polysaccharide obtained by purification of serotype 23A polysaccharide from the S. pneumoniae lysate and optionally sizing of the purified polysaccharide can be characterized by different parameters including, for example, the molecular weight and the mM of acetate per mM of said serotype 23A capsular polysaccharide.

In some embodiments, the purified polysaccharides from S. pneumoniae serotype 23A before conjugation have a molecular weight of between 10 kDa and 2,000 kDa. In other such embodiments, the saccharide has a molecular weight of between 50 kDa and 2,000 kDa. In further such embodiments, the saccharide has a molecular weight of between 50 kDa and 1,750 kDa; between 50 kDa and 1,500 kDa; between 50 kDa and 1,250 kDa; between 50 kDa and 1,000 kDa; between 50 kDa and 750 kDa; between 50 kDa and 500 kDa; between 100 kDa and 2,000 kDa; between 100 kDa and 1,750 kDa; between 100 kDa and 1,500 kDa; between 100 kDa and 1,250 kDa; between 100 kDa and 1,000 kDa; between 100 kDa and 750 kDa; between 100 kDa and 500 kDa; between 200 kDa and 2,000 kDa; between 200 kDa and 1,750 kDa; between 200 kDa and 1,500 kDa; between 200 kDa and 1,250 kDa; between 200 kDa and 1,000 kDa; between 200 kDa and 750 kDa; or between 200 kDa and 500 kDa. Any whole number integer within any of the above ranges is contemplated as an embodiment of the disclosure.

A polysaccharide can become slightly reduced in size during normal purification procedures. Additionally, as described herein, polysaccharide can be subjected to sizing techniques before conjugation. The molecular weight ranges mentioned above refer to purified polysaccharides before conjugation (e.g., before activation) after an eventual sizing step.

The presence of O-acetyl in a purified, isolated or activated serotype 23A capsular polysaccharide or in a serotype 23A polysaccharide-carrier protein conjugate is expressed as the number of mM of acetate per mM of said polysaccharide or as the number of O-acetyl group per polysaccharide repeating unit.

In an embodiment, the purified polysaccharides from S. pneumoniae serotype 23A has at least 0.2, 0.4, 0.6, 0.8, 1.0, 1.2, 1.4 or 1.6 μmol acetate per μmol of said serotype 23A capsular polysaccharide.

### 1.2.12 Pneumococcal Polysaccharide Serotype 23B

Serotype 23B polysaccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO2008/118752). In addition, they can be produced using synthetic protocols.

Serotype 23B S. pneumoniae strains may be obtained from established culture collections (such as for example the Streptococcal Reference Laboratory (Centers for Disease Control and Prevention, Atlanta, GA)) or clinical specimens.

Purified polysaccharides from serotype 23B may be activated (e.g., chemically activated) to make them capable of reacting and then incorporated into glycoconjugates of the invention, as further described herein.

The isolated serotype 23B capsular polysaccharide obtained by purification of serotype 23B polysaccharide from the S. pneumoniae lysate and optionally sizing of the purified polysaccharide can be characterized by different parameters including, for example, the molecular weight and the mM of acetate per mM of said serotype 23B capsular polysaccharide.

In some embodiments, the purified polysaccharides from S. pneumoniae serotype 23B before conjugation have a molecular weight of between 10 kDa and 2,000 kDa. In other such embodiments, the saccharide has a molecular weight of between 50 kDa and 2,000 kDa. In further such embodiments, the saccharide has a molecular weight of between 50 kDa and 1,750 kDa; between 50 kDa and 1,500 kDa; between 50 kDa and 1,250 kDa; between 50 kDa and 1,000 kDa; between 50 kDa and 750 kDa; between 50 kDa and 500 kDa; between 100 kDa and 2,000 kDa; between 100 kDa and 1,750 kDa; between 100 kDa and 1,500 kDa; between 100 kDa and 1,250 kDa; between 100 kDa and 1,000 kDa; between 100 kDa and 750 kDa; between 100 kDa and 500 kDa; between 200 kDa and 2,000 kDa; between 200 kDa and 1,750 kDa; between 200 kDa and 1,500 kDa; between 200 kDa and 1,250 kDa; between 200 kDa and 1,000 kDa; between 200 kDa and 750 kDa; or between 200 kDa and 500 kDa. Any whole number integer within any of the above ranges is contemplated as an embodiment of the disclosure.

A polysaccharide can become slightly reduced in size during normal purification procedures. Additionally, as described herein, polysaccharide can be subjected to sizing techniques before conjugation. The molecular weight ranges mentioned above refer to purified polysaccharides before conjugation (e.g., before activation) after an eventual sizing step.

The presence of O-acetyl in a purified, isolated or activated serotype 23B capsular polysaccharide or in a serotype 23B polysaccharide-carrier protein conjugate is expressed as the number of mM of acetate per mM of said polysaccharide or as the number of O-acetyl group per polysaccharide repeating unit.

In an embodiment, the purified polysaccharides from S. pneumoniae serotype 23B has at least 0.2, 0.4, 0.6, 0.8, 1.0, 1.2, 1.4 or 1.6 μmol acetate per μmol of said serotype 23B capsular polysaccharide.

### 1.2.13 Pneumococcal Polysaccharide Serotype 31

Serotype 31 polysaccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO2008/118752). In addition, they can be produced using synthetic protocols.

Serotype 31 S. pneumoniae strains may be obtained from established culture collections (such as for example the Streptococcal Reference Laboratory (Centers for Disease Control and Prevention, Atlanta, GA)) or clinical specimens.

Purified polysaccharides from serotype 31 may be activated (e.g., chemically activated) to make them capable of reacting and then incorporated into glycoconjugates of the invention, as further described herein.

The isolated serotype 31 capsular polysaccharide obtained by purification of serotype 31 polysaccharide from the S. pneumoniae lysate and optionally sizing of the purified polysaccharide can be characterized by different parameters including, for example, the molecular weight and the mM of acetate per mM of said serotype 31 capsular polysaccharide.

In some embodiments, the purified polysaccharides from S. pneumoniae serotype 31 before conjugation have a molecular weight of between 10 kDa and 2,000 kDa. In other such embodiments, the saccharide has a molecular weight of between 50 kDa and 2,000 kDa. In further such embodiments, the saccharide has a molecular weight of between 50 kDa and 1,750 kDa; between 50 kDa and 1,500 kDa; between 50 kDa and 1,250 kDa; between 50 kDa and 1,000 kDa; between 50 kDa and 750 kDa; between 50 kDa and 500 kDa; between 100 kDa and 2,000 kDa; between 100 kDa and 1,750 kDa; between 100 kDa and 1,500 kDa; between 100 kDa and 1,250 kDa; between 100 kDa and 1,000 kDa; between 100 kDa and 750 kDa; between 100 kDa and 500 kDa; between 200 kDa and 2,000 kDa; between 200 kDa and 1,750 kDa; between 200 kDa and 1,500 kDa; between 200 kDa and 1,250 kDa; between 200 kDa and 1,000 kDa; between 200 kDa and 750 kDa; or between 200 kDa and 500 kDa. Any whole number integer within any of the above ranges is contemplated as an embodiment of the disclosure.

A polysaccharide can become slightly reduced in size during normal purification procedures. Additionally, as described herein, polysaccharide can be subjected to sizing techniques before conjugation. The molecular weight ranges mentioned above refer to purified polysaccharides before conjugation (e.g., before activation) after an eventual sizing step.

The presence of O-acetyl in a purified, isolated or activated serotype 31 capsular polysaccharide or in a serotype 31 polysaccharide-carrier protein conjugate is expressed as the number of mM of acetate per mM of said polysaccharide or as the number of O-acetyl group per polysaccharide repeating unit.

In an embodiment, the purified polysaccharides from S. pneumoniae serotype 31 has at least 0.2, 0.4, 0.6, 0.8, 1.0, 1.2, 1.4 or 1.6 μmol acetate per μmol of said serotype 31 capsular polysaccharide.

### 1.2.14 Pneumococcal Polysaccharide Serotype 34

Serotype 34 polysaccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO2008/118752). In addition, they can be produced using synthetic protocols.

Serotype 34 S. pneumoniae strains may be obtained from established culture collections (such as for example the Streptococcal Reference Laboratory (Centers for Disease Control and Prevention, Atlanta, GA)) or clinical specimens.

Purified polysaccharides from serotype 34 may be activated (e.g., chemically activated) to make them capable of reacting and then incorporated into glycoconjugates of the invention, as further described herein.

The isolated serotype 34 capsular polysaccharide obtained by purification of serotype 34 polysaccharide from the S. pneumoniae lysate and optionally sizing of the purified polysaccharide can be characterized by different parameters including, for example, the molecular weight and the mM of acetate per mM of said serotype 34 capsular polysaccharide.

In some embodiments, the purified polysaccharides from S. pneumoniae serotype 34 before conjugation have a molecular weight of between 10 kDa and 2,000 kDa. In other such embodiments, the saccharide has a molecular weight of between 50 kDa and 2,000 kDa. In further such embodiments, the saccharide has a molecular weight of between 50 kDa and 1,750 kDa; between 50 kDa and 1,500 kDa; between 50 kDa and 1,250 kDa; between 50 kDa and 1,000 kDa; between 50 kDa and 750 kDa; between 50 kDa and 500 kDa; between 100 kDa and 2,000 kDa; between 100 kDa and 1,750 kDa; between 100 kDa and 1,500 kDa; between 100 kDa and 1,250 kDa; between 100 kDa and 1,000 kDa; between 100 kDa and 750 kDa; between 100 kDa and 500 kDa; between 200 kDa and 2,000 kDa; between 200 kDa and 1,750 kDa; between 200 kDa and 1,500 kDa; between 200 kDa and 1,250 kDa; between 200 kDa and 1,000 kDa; between 200 kDa and 750 kDa; or between 200 kDa and 500 kDa. Any whole number integer within any of the above ranges is contemplated as an embodiment of the disclosure.

A polysaccharide can become slightly reduced in size during normal purification procedures. Additionally, as described herein, polysaccharide can be subjected to sizing techniques before conjugation. The molecular weight ranges mentioned above refer to purified polysaccharides before conjugation (e.g., before activation) after an eventual sizing step.

The presence of O-acetyl in a purified, isolated or activated serotype 34 capsular polysaccharide or in a serotype 34 polysaccharide-carrier protein conjugate is expressed as the number of mM of acetate per mM of said polysaccharide or as the number of O-acetyl group per polysaccharide repeating unit.

In an embodiment, the purified polysaccharides from S. pneumoniae serotype 34 has at least 0.2, 0.4, 0.6, 0.8, 1.0, 1.2, 1.4 or 1.6 μmol acetate per μmol of said serotype 34 capsular polysaccharide.

### 1.2.15 Pneumococcal Polysaccharide Serotype 35B

Serotype 35B polysaccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO2008/118752). In addition, they can be produced using synthetic protocols.

Serotype 35B S. pneumoniae strains may be obtained from established culture collections (such as for example the Streptococcal Reference Laboratory (Centers for Disease Control and Prevention, Atlanta, GA)) or clinical specimens.

Purified polysaccharides from serotype 35B may be activated (e.g., chemically activated) to make them capable of reacting and then incorporated into glycoconjugates of the invention, as further described herein.

The isolated serotype 35B capsular polysaccharide obtained by purification of serotype 35B polysaccharide from the S. pneumoniae lysate and optionally sizing of the purified polysaccharide can be characterized by different parameters including, for example, the molecular weight and the mM of acetate per mM of said serotype 35B capsular polysaccharide.

In some embodiments, the purified polysaccharides from S. pneumoniae serotype 35B before conjugation have a molecular weight of between 10 kDa and 2,000 kDa. In other such embodiments, the saccharide has a molecular weight of between 50 kDa and 2,000 kDa. In further such embodiments, the saccharide has a molecular weight of between 50 kDa and 1,750 kDa; between 50 kDa and 1,500 kDa; between 50 kDa and 1,250 kDa; between 50 kDa and 1,000 kDa; between 50 kDa and 750 kDa; between 50 kDa and 500 kDa; between 100 kDa and 2,000 kDa; between 100 kDa and 1,750 kDa; between 100 kDa and 1,500 kDa; between 100 kDa and 1,250 kDa; between 100 kDa and 1,000 kDa; between 100 kDa and 750 kDa; between 100 kDa and 500 kDa; between 200 kDa and 2,000 kDa; between 200 kDa and 1,750 kDa; between 200 kDa and 1,500 kDa; between 200 kDa and 1,250 kDa; between 200 kDa and 1,000 kDa; between 200 kDa and 750 kDa; or between 200 kDa and 500 kDa. Any whole number integer within any of the above ranges is contemplated as an embodiment of the disclosure.

A polysaccharide can become slightly reduced in size during normal purification procedures. Additionally, as described herein, polysaccharide can be subjected to sizing techniques before conjugation. The molecular weight ranges mentioned above refer to purified polysaccharides before conjugation (e.g., before activation) after an eventual sizing step.

The presence of O-acetyl in a purified, isolated or activated serotype 35B capsular polysaccharide or in a serotype 35B polysaccharide-carrier protein conjugate is expressed as the number of mM of acetate per mM of said polysaccharide or as the number of O-acetyl group per polysaccharide repeating unit.

In an embodiment, the purified polysaccharides from S. pneumoniae serotype 35B has at least 0.2, 0.4, 0.6, 0.8, 1.0, 1.2, 1.4 or 1.6 μmol acetate per μmol of said serotype 35B capsular polysaccharide.

### 1.2.16 Pneumococcal Polysaccharide Serotype 35F

Serotype 35F polysaccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO2008/118752). In addition, they can be produced using synthetic protocols.

Serotype 35F S. pneumoniae strains may be obtained from established culture collections (such as for example the Streptococcal Reference Laboratory (Centers for Disease Control and Prevention, Atlanta, GA)) or clinical specimens.

Purified polysaccharides from serotype 35F may be activated (e.g., chemically activated) to make them capable of reacting and then incorporated into glycoconjugates of the invention, as further described herein.

The isolated serotype 35F capsular polysaccharide obtained by purification of serotype 35F polysaccharide from the S. pneumoniae lysate and optionally sizing of the purified polysaccharide can be characterized by different parameters including, for example, the molecular weight and the mM of acetate per mM of said serotype 35F capsular polysaccharide.

In some embodiments, the purified polysaccharides from S. pneumoniae serotype 35F before conjugation have a molecular weight of between 10 kDa and 2,000 kDa. In other such embodiments, the saccharide has a molecular weight of between 50 kDa and 2,000 kDa. In further such embodiments, the saccharide has a molecular weight of between 50 kDa and 1,750 kDa; between 50 kDa and 1,500 kDa; between 50 kDa and 1,250 kDa; between 50 kDa and 1,000 kDa; between 50 kDa and 750 kDa; between 50 kDa and 500 kDa; between 100 kDa and 2,000 kDa; between 100 kDa and 1,750 kDa; between 100 kDa and 1,500 kDa; between 100 kDa and 1,250 kDa; between 100 kDa and 1,000 kDa; between 100 kDa and 750 kDa; between 100 kDa and 500 kDa; between 200 kDa and 2,000 kDa; between 200 kDa and 1,750 kDa; between 200 kDa and 1,500 kDa; between 200 kDa and 1,250 kDa; between 200 kDa and 1,000 kDa; between 200 kDa and 750 kDa; or between 200 kDa and 500 kDa. Any whole number integer within any of the above ranges is contemplated as an embodiment of the disclosure.

A polysaccharide can become slightly reduced in size during normal purification procedures. Additionally, as described herein, polysaccharide can be subjected to sizing techniques before conjugation. The molecular weight ranges mentioned above refer to purified polysaccharides before conjugation (e.g., before activation) after an eventual sizing step.

The presence of O-acetyl in a purified, isolated or activated serotype 35F capsular polysaccharide or in a serotype 35F polysaccharide-carrier protein conjugate is expressed as the number of mM of acetate per mM of said polysaccharide or as the number of O-acetyl group per polysaccharide repeating unit.

In an embodiment, the purified polysaccharides from S. pneumoniae serotype 35F has at least 0.2, 0.4, 0.6, 0.8, 1.0, 1.2, 1.4 or 1.6 μmol acetate per μmol of said serotype 35F capsular polysaccharide.

### 1.2.17 Pneumococcal Polysaccharide Serotype 38

Serotype 38 polysaccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO2008/118752). In addition, they can be produced using synthetic protocols.

Serotype 38 S. pneumoniae strains may be obtained from established culture collections (such as for example the Streptococcal Reference Laboratory (Centers for Disease Control and Prevention, Atlanta, GA)) or clinical specimens.

Purified polysaccharides from serotype 38 may be activated (e.g., chemically activated) to make them capable of reacting and then incorporated into glycoconjugates of the invention, as further described herein.

The isolated serotype 38 capsular polysaccharide obtained by purification of serotype 38 polysaccharide from the S. pneumoniae lysate and optionally sizing of the purified polysaccharide can be characterized by different parameters including, for example, the molecular weight and the mM of acetate per mM of said serotype 38 capsular polysaccharide.

In some embodiments, the purified polysaccharides from S. pneumoniae serotype 38 before conjugation have a molecular weight of between 10 kDa and 2,000 kDa. In other such embodiments, the saccharide has a molecular weight of between 50 kDa and 2,000 kDa. In further such embodiments, the saccharide has a molecular weight of between 50 kDa and 1,750 kDa; between 50 kDa and 1,500 kDa; between 50 kDa and 1,250 kDa; between 50 kDa and 1,000 kDa; between 50 kDa and 750 kDa; between 50 kDa and 500 kDa; between 100 kDa and 2,000 kDa; between 100 kDa and 1,750 kDa; between 100 kDa and 1,500 kDa; between 100 kDa and 1,250 kDa; between 100 kDa and 1,000 kDa; between 100 kDa and 750 kDa; between 100 kDa and 500 kDa; between 200 kDa and 2,000 kDa; between 200 kDa and 1,750 kDa; between 200 kDa and 1,500 kDa; between 200 kDa and 1,250 kDa; between 200 kDa and 1,000 kDa; between 200 kDa and 750 kDa; or between 200 kDa and 500 kDa. Any whole number integer within any of the above ranges is contemplated as an embodiment of the disclosure.

A polysaccharide can become slightly reduced in size during normal purification procedures. Additionally, as described herein, polysaccharide can be subjected to sizing techniques before conjugation. The molecular weight ranges mentioned above refer to purified polysaccharides before conjugation (e.g., before activation) after an eventual sizing step.

The presence of O-acetyl in a purified, isolated or activated serotype 38 capsular polysaccharide or in a serotype 38 polysaccharide-carrier protein conjugate is expressed as the number of mM of acetate per mM of said polysaccharide or as the number of O-acetyl group per polysaccharide repeating unit.

In an embodiment, the purified polysaccharides from S. pneumoniae serotype 38 has at least 0.2, 0.4, 0.6, 0.8, 1.0, 1.2, 1.4 or 1.6 μmol acetate per μmol of said serotype 38 capsular polysaccharide.

### 1.3 Glycoconjugates of the Invention

The purified saccharides are chemically activated to make the saccharides (i.e., activated saccharides) capable of reacting with the carrier protein. Once activated, each capsular saccharide is separately conjugated to a carrier protein to form a glycoconjugate. In one embodiment, each capsular saccharide is conjugated to the same carrier protein. The chemical activation of the saccharides and subsequent conjugation to the carrier protein can be achieved by the activation and conjugation methods disclosed herein.

1.3.1 Glycoconjugates from S. pneumoniae Serotype 6C, 7C, 9N, 15A, 15B, 15C, 16F, 17F, 20, 23A, 23B, 31, 34, 35B, 35F and 38

Capsular polysaccharides from serotypes 6C, 7C, 9N, 15A, 15B, 15C, 16F, 17F, 20, 23A, 23B, 31, 34, 35B, 35F and/or 38 of S. pneumoniae may be prepared by standard techniques known to those of ordinary skill in the art (see for example WO2006/110381, WO2008/118752, WO2006/110352, and U.S. Patent App. Pub. Nos. 2006/0228380, 2006/0228381, 2008/0102498 and 2008/0286838).

In an embodiment, the polysaccharides are activated with 1-cyano-4-dimethylamino pyridinium tetrafluoroborate (CDAP) to form a cyanate ester. The activated polysaccharide is then coupled directly or via a spacer (linker) group to an amino group on the carrier protein (preferably CRM₁₉₇). For example, the spacer may be cystamine or cysteamine to give a thiolated polysaccharide which may be coupled to the carrier via a thioether linkage obtained after reaction with a maleimide-activated carrier protein (for example using N-[γ-maleimidobutyrloxy]succinimide ester (GMBS)) or a haloacetylated carrier protein (for example using iodoacetimide, N-succinimidyl bromoacetate (SBA; SIB), N-succinimidyl(4-iodoacetyl)aminobenzoate (SIAB), sulfosuccinimidyl(4-iodoacetyl)aminobenzoate (sulfo-SIAB), N-succinimidyl iodoacetate (SIA) or succinimidyl 3-[bromoacetamido]proprionate (SBAP)). Preferably, the cyanate ester (optionally made by CDAP chemistry) is coupled with hexane diamine or adipic acid dihydrazide (ADH) and the amino-derivatised saccharide is conjugated to the carrier protein (e.g., CRM₁₉₇) using carbodiimide (e.g., EDAC or EDC) chemistry via a carboxyl group on the protein carrier. Such conjugates are described, for example, in WO93/15760, WO95/08348 and WO96/129094.

Other suitable techniques for conjugation use carbodiimides, hydrazides, active esters, norborane, p-nitrobenzoic acid, N-hydroxysuccinimide, S—NHS, EDC, TSTU. Many are described in International Patent Application Publication No. WO98/42721. Conjugation may involve a carbonyl linker which may be formed by reaction of a free hydroxyl group of the saccharide with 1,1′-carbonyldiimidazole (CDI) (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In an embodiment, at least one of the capsular polysaccharides from serotypes 6C, 7C, 9N, 15A, 15B, 15C, 16F, 17F, 20, 23A, 23B, 31, 34, 35B, 35F and 38 of S. pneumoniae is conjugated to the carrier protein by reductive amination (such as described in U.S. Patent Appl. Pub. Nos. 2006/0228380, 2007/0231340, 2007/0184071 and 2007/0184072, WO2006/110381, WO2008/079653, and WO2008/143709). In an embodiment, the capsular polysaccharides from serotypes 6C, 7C, 9N, 15A, 15B, 15C, 16F, 17F, 20, 23A, 23B, 31, 34, 35B, 35F and 38 of S. pneumoniae are all conjugated to the carrier protein by reductive amination.

Reductive amination involves two steps: (1) oxidation of the polysaccharide and (2) reduction of the activated polysaccharide and a carrier protein to form a conjugate. Before oxidation, the polysaccharide is optionally hydrolyzed. Mechanical or chemical hydrolysis may be employed. Chemical hydrolysis may be conducted using acetic acid. The oxidation step may involve reaction with periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate).

In an embodiment, the capsular polysaccharide from serotype 6C, 7C, 9N, 15A, 15B, 15C, 16F, 17F, 20, 23A, 23B, 31, 34, 35B, 35F and/or 38 of S. pneumoniae is oxidized in the presence of metaperiodate, or in the presence of sodium periodate (NaIO₄). In another embodiment, the capsular polysaccharide from serotype 6C, 7C, 9N, 15A, 15B, 15C, 16F, 17F, 20, 23A, 23B, 31, 34, 35B, 35F and/or 38 of S. pneumoniae is oxidized in the presence of orthoperiodate, or in the presence of periodic acid.

Following the oxidation step of the polysaccharide, the polysaccharide is said to be activated and is referred to as “activated polysaccharide” herein. The activated polysaccharide and the carrier protein may be lyophilised (freeze-dried), either independently (discrete lyophilization) or together (co-lyophilized). In one embodiment, the activated polysaccharide and the carrier protein are co-lyophilized. In another embodiment, the activated polysaccharide and the carrier protein are lyophilized independently.

In one embodiment, the lyophilization takes place in the presence of a non-reducing sugar, possible non-reducing sugars include sucrose, trehalose, raffinose, stachyose, melezitose, dextran, mannitol, lactitol and palatinit.

The second step of the conjugation process is the reduction of the activated polysaccharide and a carrier protein to form a conjugate (referred to as reductive amination), using a reducing agent. Reducing agents which are suitable include the cyanoborohydrides, such as sodium cyanoborohydride, borane-pyridine, or borohydride exchange resin. In one embodiment, the reducing agent is sodium cyanoborohydride.

In an embodiment, the reduction reaction is carried out in aqueous solvent, in another embodiment, the reaction is carried out in aprotic solvent. In an embodiment, the reduction reaction is carried out in DMSO (dimethylsulfoxide) or in DMF (dimethylformamide) solvent. The DMSO or DMF solvent may be used to reconstitute the activated polysaccharide and carrier protein which has been lyophilized.

At the end of the reduction reaction, there may be unreacted aldehyde groups remaining in the conjugates, these may be capped using a suitable capping agent. In one embodiment, this capping agent is sodium borohydride (NaBH₄). Following the conjugation (the reduction reaction and optionally the capping), the glycoconjugates may be purified. The glycoconjugates may be purified by diafiltration and/or ion exchange chromatography and/or size exclusion chromatography. In an embodiment, the glycoconjugates are purified by diafiltration or ion exchange chromatography or size exclusion chromatography. In one embodiment, the glycoconjugates are sterile filtered.

In one or more embodiments, the glycoconjugate from S. pneumoniae serotypes 6C, 7C, 9N, 15A, 15B, 15C, 16F, 17F, 20, 23A, 23B, 31, 34, 35B, 35F and/or 38 comprise a saccharide which has a degree of O-acetylation of between 10% and 100%, between 20% and 100%, between 30% and 100%, between 40% and 100%, between 50% and 100%, between 60% and 100%, between 70% and 100%, between 75% and 100%, between 80% and 100%, between 90% and 100%, between 50% and 90%, between 60% and 90%, between 70% and 90% or between 80% and 90%. In other embodiments, the degree of O-acetylation is ≥10%, ≥20%, ≥30%, ≥40%, ≥50%, ≥60%, ≥70%, ≥80%, or ≥90%, or about 100%.

In some embodiments, the glycoconjugate from S. pneumoniae serotypes 6C, 7C, 9N, 15A, 15B, 15C, 16F, 17F, 20, 23A, 23B, 31, 34, 35B, 35F and/or 38 of the invention are O-acetylated. In some embodiments, the glycoconjugate from S. pneumoniae serotype 6C, 7C, 9N, 15A, 15B, 15C, 16F, 17F, 20, 23A, 23B, 31, 34, 35B, 35F and/or 38 is O-acetylated and the glycoconjugate from S. pneumoniae serotype 6C, 7C, 9N, 15A, 15B, 15C, 16F, 17F, 20, 23A, 23B, 31, 34, 35B, 35F and/or 38 is de-O-acetylated.

In one or more embodiments, the serotype 6C, 7C, 9N, 15A, 15B, 15C, 16F, 17F, 20, 23A, 23B, 31, 34, 35B, 35F and/or 38 glycoconjugates of the invention are prepared using eTEC conjugation (hereinafter “eTEC linked glycoconjugates”), such as described at Examples 1, 2 and 3 and in WO2014/027302. The 6C, 7C, 9N, 15A, 15B, 15C, 16F, 17F, 20, 23A, 23B, 31, 34, 35B, 35F and/or 38 glycoconjugates comprise a saccharide covalently conjugated to a carrier protein through one or more eTEC spacers, wherein the saccharide is covalently conjugated to the eTEC spacer through a carbamate linkage, and wherein the carrier protein is covalently conjugated to the eTEC spacer through an amide linkage. The eTEC linked glycoconjugates of the invention may be represented by the general formula (III):

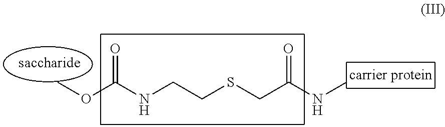

wherein the atoms that comprise the eTEC spacer are contained in the central box.

The eTEC spacer includes seven linear atoms (i.e., —C(O)NH(CH₂)₂SCH₂C(O)—) and provides stable thioether and amide bonds between the saccharide and carrier protein. Synthesis of the eTEC linked glycoconjugate involves reaction of an activated hydroxyl group of the saccharide with the amino group of a thioalkylamine reagent, e.g., cystamine or cysteinamine or a salt thereof, forming a carbamate linkage to the saccharide to provide a thiolated saccharide. Generation of one or more free sulfhydryl groups is accomplished by reaction with a reducing agent to provide an activated thiolated saccharide. Reaction of the free sulfhydryl groups of the activated thiolated saccharide with an activated carrier protein having one or more α-haloacetamide groups on amine containing residues generates a thioether bond to form the conjugate, wherein the carrier protein is attached to the eTEC spacer through an amide bond.

In serotype 6C, 7C, 9N, 15A, 15B, 15C, 16F, 17F, 20, 23A, 23B, 31, 34, 35B, 35F and/or 38 glycoconjugates of the invention, the saccharide may be a polysaccharide or an oligosaccharide. The carrier protein may be selected from any suitable carrier as described herein or known to one of skill in the art. In one or more embodiments, the saccharide is a polysaccharide. In some such embodiments, the carrier protein is CRM₁₉₇. In some such embodiments, the eTEC linked glycoconjugate comprises a S. pneumoniae serotype 6C, 7C, 9N, 15A, 15B, 15C, 16F, 17F, 20, 23A, 23B, 31, 34, 35B, 35F and/or 38 capsular polysaccharide.

In one or more embodiments, the eTEC linked glycoconjugate comprises a Pn-6C, 7C, 9N, 15A, 15B, 15C, 16F, 17F, 20, 23A, 23B, 31, 34, 35B, 35F and/or 38 capsular polysaccharide, which is covalently conjugated to CRM₁₉₇ through an eTEC spacer (serotype 6C, 7C, 9N, 15A, 15B, 15C, 16F, 17F, 20, 23A, 23B, 31, 34, 35B, 35F and/or 38 eTEC linked glycoconjugates).

In some embodiments, the serotype 6C, 7C, 9N, 15A, 15B, 15C, 16F, 17F, 20, 23A, 23B, 31, 34, 35B, 35F and/or 38 glycoconjugates of the present invention comprise a saccharide having a molecular weight of between 10 kDa and 2,000 kDa. In other such embodiments, the saccharide has a molecular weight of between 50 kDa and 2,000 kDa. In further embodiments, the saccharide has a molecular weight of between 50 kDa and 1,750 kDa; between 50 kDa and 1,500 kDa; between 50 kDa and 1,250 kDa; between 50 kDa and 1,000 kDa; between 50 kDa and 750 kDa; between 50 kDa and 500 kDa; between 100 kDa and 2,000 kDa; between 100 kDa and 1,750 kDa; between 100 kDa and 1,500 kDa; between 100 kDa and 1,250 kDa; between 100 kDa and 1,000 kDa; between 100 kDa and 750 kDa; between 100 kDa and 500 kDa; between 200 kDa and 2,000 kDa; between 200 kDa and 1,750 kDa; between 200 kDa and 1,500 kDa; between 200 kDa and 1,250 kDa; between 200 kDa and 1,000 kDa; between 200 kDa and 750 kDa; or between 200 kDa and 500 kDa. Any whole number integer within any of the above ranges is contemplated as an embodiment of the disclosure.

In some embodiments, the serotype 6C, 7C, 9N, 15A, 15B, 15C, 16F, 17F, 20, 23A, 23B, 31, 34, 35B, 35F and/or 38 glycoconjugate of the invention has a molecular weight of between 50 kDa and 20,000 kDa. In other embodiments, the serotype 6C, 7C, 9N, 15A, 15B, 15C, 16F, 17F, 20, 23A, 23B, 31, 34, 35B, 35F and/or 38 glycoconjugate has a molecular weight of between 500 kDa and 10,000 kDa. In other embodiments, the serotype 6C, 7C, 9N, 15A, 15B, 15C, 16F, 17F, 20, 23A, 23B, 31, 34, 35B, 35F and/or 38 glycoconjugate has a molecular weight of between 200 kDa and 10,000 kDa. In still other embodiments, the serotype 6C, 7C, 9N, 15A, 15B, 15C, 16F, 17F, 20, 23A, 23B, 31, 34, 35B, 35F and/or 38 glycoconjugate has a molecular weight of between 1,000 kDa and 3,000 kDa.

In further embodiments, the serotype 6C, 7C, 9N, 15A, 15B, 15C, 16F, 17F, 20, 23A, 23B, 31, 34, 35B, 35F and/or 38 glycoconjugate of the invention has a molecular weight of between 200 kDa and 20,000 kDa; between 200 kDa and 15,000 kDa; between 200 kDa and 10,000 kDa; between 200 kDa and 7,500 kDa; between 200 kDa and 5,000 kDa; between 200 kDa and 3,000 kDa; between 200 kDa and 1,000 kDa; between 500 kDa and 20,000 kDa; between 500 kDa and 15,000 kDa; between 500 kDa and 12,500 kDa; between 500 kDa and 10,000 kDa; between 500 kDa and 7,500 kDa; between 500 kDa and 6,000 kDa; between 500 kDa and 5,000 kDa; between 500 kDa and 4,000 kDa; between 500 kDa and 3,000 kDa; between 500 kDa and 2,000 kDa; between 500 kDa and 1,500 kDa; between 500 kDa and 1,000 kDa; between 750 kDa and 20,000 kDa; between 750 kDa and 15,000 kDa; between 750 kDa and 12,500 kDa; between 750 kDa and 10,000 kDa; between 750 kDa and 7,500 kDa; between 750 kDa and 6,000 kDa; between 750 kDa and 5,000 kDa; between 750 kDa and 4,000 kDa; between 750 kDa and 3,000 kDa; between 750 kDa and 2,000 kDa; between 750 kDa and 1,500 kDa; between 1,000 kDa and 15,000 kDa; between 1,000 kDa and 12,500 kDa; between 1,000 kDa and 10,000 kDa; between 1,000 kDa and 7,500 kDa; between 1,000 kDa and 6,000 kDa; between 1,000 kDa and 5,000 kDa; between 1,000 kDa and 4,000 kDa; between 1,000 kDa and 2,500 kDa; between 2,000 kDa and 15,000 kDa; between 2,000 kDa and 12,500 kDa; between 2,000 kDa and 10,000 kDa; between 2,000 kDa and 7,500 kDa; between 2,000 kDa and 6,000 kDa; between 2,000 kDa and 5,000 kDa; between 2,000 kDa and 4,000 kDa; between 2,000 kDa and 3,000 kDa; between 3,000 kDa and 20,000 kDa; between 3,000 kDa and 15,000 kDa; between 3,000 kDa and 12,500 kDa; between 3,000 kDa and 10,000 kDa; between 3,000 kDa and 9,000 kDa; between 3,000 kDa and 8,000 kDa; between 3,000 kDa and 7,000 kDa; between 3,000 kDa and 6,000 kDa; between 3,000 kDa and 5,000 kDa or between 3,000 kDa and 4,000 kDa. Any whole number integer within any of the above ranges is contemplated as an embodiment of the disclosure.

In an embodiment, the degree of conjugation of the serotype 6C, 7C, 9N, 15A, 15B, 15C, 16F, 17F, 20, 23A, 23B, 31, 34, 35B, 35F and/or 38 glycoconjugate of the invention is between 2 and 20, between 4 and 16, between 2 and 15, between 2 and 13, between 2 and 10, between 2 and 8, between 2 and 6, between 2 and 5, between 2 and 4, between 3 and 15, between 3 and 13, between 3 and 10, between 3 and 8, between 3 and 6, between 3 and 5, between 3 and 4, between 5 and 15, between 5 and 10, between 8 and 15, between 8 and 12, between 10 and 15 or between 10 and 12. In an embodiment, the degree of conjugation of the serotype 6C, 7C, 9N, 15A, 15B, 15C, 16F, 17F, 20, 23A, 23B, 31, 34, 35B, 35F and/or 38 glycoconjugate of the invention is about 2, about 3, about 4, about 5, about 6, about 7, about 8, about 9, about 10, about 11, about 12, about 13, about 14, about 15, about 16, about 17, about 18, about 19 or about 20. In another embodiment, the degree of conjugation of the serotype 6C, 7C, 9N, 15A, 15B, 15C, 16F, 17F, 20, 23A, 23B, 31, 34, 35B, 35F and/or 38 glycoconjugate of the invention is between 4 and 16. In some such embodiments, the carrier protein is CRM₁₉₇.

In an embodiment, the carrier protein comprises CRM₁₉₇, which contains 39 lysine residues. In some embodiments, the CRM₁₉₇ may comprise 4 to 16 lysine residues out of 39 covalently linked to the saccharide. Another way to express this parameter is that about 10% to about 41% of CRM₁₉₇ lysines are covalently linked to the saccharide. In another embodiment, the CRM₁₉₇ may comprise 2 to 20 lysine residues out of 39 covalently linked to the saccharide. Another way to express this parameter is that about 5% to about 50% of CRM₁₉₇ lysines are covalently linked to the saccharide. In some embodiments, the CRM₁₉₇ may comprise about 4, about 5, about 6, about 7, about 8, about 9, about 10, about 11, about 12, about 13, about 14, about 15, or about 16 lysine residues out of 39 covalently linked to the saccharide.

In one or more embodiments, the carrier protein is covalently conjugated to an eTEC spacer through an amide linkage to one or more-amino groups of lysine residues on the carrier protein. In some such embodiments, the carrier protein comprises 2 to 20 lysine residues covalently conjugated to the saccharide. In other embodiments, the carrier protein comprises 4 to 16 lysine residues covalently conjugated to the saccharide.

The serotype 6C, 7C, 9N, 15A, 15B, 15C, 16F, 17F, 20, 23A, 23B, 31, 34, 35B, 35F and/or 38 glycoconjugates of the invention may also be characterized by the ratio (weight/weight) of saccharide to carrier protein. In some embodiments, the saccharide to carrier protein ratio (w/w) is between 0.2 and 4.0 (e.g., about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9 or about 4.0). In other embodiments, the saccharide to carrier protein ratio (w/w) is between 1.0 and 2.5. In further embodiments, the saccharide to carrier protein ratio (w/w) is between 0.4 and 1.7. In some such embodiments, the carrier protein is CRM₁₉₇.

The frequency of attachment of the saccharide chain to a lysine on the carrier protein is another parameter for characterizing the serotype 6C, 7C, 9N, 15A, 15B, 15C, 16F, 17F, 20, 23A, 23B, 31, 34, 35B, 35F and/or 38 glycoconjugates of the invention. For example, in some embodiments, at least one covalent linkage between the carrier protein and the polysaccharide occurs for every 4 saccharide repeat units of the polysaccharide. In another embodiment, the covalent linkage between the carrier protein and the polysaccharide occurs at least once in every 10 saccharide repeat units of the polysaccharide. In another embodiment, the covalent linkage between the carrier protein and the polysaccharide occurs at least once in every 15 saccharide repeat units of the polysaccharide. In a further embodiment, the covalent linkage between the carrier protein and the polysaccharide occurs at least once in every 25 saccharide repeat units of the polysaccharide.

In one or more embodiments, the carrier protein is CRM₁₉₇ and the covalent linkage via an eTEC spacer between the CRM₁₉₇ and the polysaccharide occurs at least once in every 4, 10, 15 or 25 saccharide repeat units of the polysaccharide.

In other embodiments, the conjugate comprises at least one covalent linkage between the carrier protein and saccharide for every 5 to 10 saccharide repeat units; every 2 to 7 saccharide repeat units; every 3 to 8 saccharide repeat units; every 4 to 9 saccharide repeat units; every 6 to 11 saccharide repeat units; every 7 to 12 saccharide repeat units; every 8 to 13 saccharide repeat units; every 9 to 14 saccharide repeat units; every 10 to 15 saccharide repeat units; every 2 to 6 saccharide repeat units, every 3 to 7 saccharide repeat units; every 4 to 8 saccharide repeat units; every 6 to 10 saccharide repeat units; every 7 to 11 saccharide repeat units; every 8 to 12 saccharide repeat units; every 9 to 13 saccharide repeat units; every 10 to 14 saccharide repeat units; every 10 to 20 saccharide repeat units; every 4 to 25 saccharide repeat units or every 2 to 25 saccharide repeat units. In frequent embodiments, the carrier protein is CRM₁₉₇.

In another embodiment, at least one linkage between carrier protein and saccharide occurs for every 2, 3, 4, 5, 6, 7, 8, 9, 10, 11, 12, 13, 14, 15, 16, 17, 18, 19, 20, 21, 22, 23, 24 or 25 saccharide repeat units of the polysaccharide. In an embodiment, the carrier protein is CRM₁₉₇. Any whole number integer within any of the above ranges is contemplated as an embodiment of the disclosure.

An important consideration during conjugation is the development of conditions that permit the retention of potentially sensitive non-saccharide substituent functional groups of the individual components, such as O-Acyl, phosphate or glycerol phosphate side chains that may form part of the saccharide epitope.

In one embodiment, the serotype 6C, 7C, 9N, 15A, 15B, 15C, 16F, 17F, 20, 23A, 23B, 31, 34, 35B, 35F and/or 38 glycoconjugates of the invention comprise a saccharide which has a degree of O-acetylation between 10% and 100%. In some such embodiments, the saccharide has a degree of O-acetylation between 50% and 100%. In other such embodiments, the saccharide has a degree of O-acetylation between 75% and 100%. In further embodiments, the saccharide has a degree of O-acetylation greater than or equal to 70% (≥70%).

In an embodiment, the serotype 6C, 7C, 9N, 15A, 15B, 15C, 16F, 17F, 20, 23A, 23B, 31, 34, 35B, 35F and/or 38 glycoconjugate comprises at least 0.1, 0.2, 0.3, 0.4, 0.5, 0.6, 0.7 or 0.8 mM acetate per mM serotype 6C, 7C, 9N, 15A, 15B, 15C, 16F, 17F, 20, 23A, 23B, 31, 34, 35B, 35F and/or 38 capsular polysaccharide. In an embodiment, the glycoconjugate comprises at least 0.5, 0.6 or 0.7 mM acetate per mM serotype 6C, 7C, 9N, 15A, 15B, 15C, 16F, 17F, 20, 23A, 23B, 31, 34, 35B, 35F and/or 38 capsular polysaccharide. In another embodiment, the glycoconjugate comprises at least 0.6 mM acetate per mM serotype 6C, 7C, 9N, 15A, 15B, 15C, 16F, 17F, 20, 23A, 23B, 31, 34, 35B, 35F and/or 38 capsular polysaccharide. In an embodiment, the glycoconjugate comprises at least 0.7 mM acetate per mM serotype 6C, 7C, 9N, 15A, 15B, 15C, 16F, 17F, 20, 23A, 23B, 31, 34, 35B, 35F and/or 38 capsular polysaccharide. In an embodiment, the presence of O-acetyl groups is determined by ion-HPLC analysis.

In another embodiment, the ratio of mM acetate per mM serotype 6C, 7C, 9N, 15A, 15B, 15C, 16F, 17F, 20, 23A, 23B, 31, 34, 35B, 35F and/or 38 polysaccharide in the glycoconjugate to mM acetate per mM serotype 6C, 7C, 9N, 15A, 15B, 15C, 16F, 17F, 20, 23A, 23B, 31, 34, 35B, 35F and/or 38 polysaccharide in the isolated polysaccharide is at least 0.6, 0.65, 0.7, 0.75, 0.8, 0.85, 0.9, or 0.95. In an embodiment, the ratio of mM acetate per mM serotype 6C, 7C, 9N, 15A, 15B, 15C, 16F, 17F, 20, 23A, 23B, 31, 34, 35B, 35F and/or 38 polysaccharide in the glycoconjugate to mM acetate per mM serotype 6C, 7C, 9N, 15A, 15B, 15C, 16F, 17F, 20, 23A, 23B, 31, 34, 35B, 35F and/or 38 polysaccharide in the isolated polysaccharide is at least 0.7. In an embodiment, the ratio of mM acetate per mM serotype 6C, 7C, 9N, 15A, 15B, 15C, 16F, 17F, 20, 23A, 23B, 31, 34, 35B, 35F and/or 38 polysaccharide in the glycoconjugate to mM acetate per mM serotype 6C, 7C, 9N, 15A, 15B, 15C, 16F, 17F, 20, 23A, 23B, 31, 34, 35B, 35F and/or 38 polysaccharide in the isolated polysaccharide is at least 0.9.

In an embodiment, the ratio of mM acetate per mM serotype 6C, 7C, 9N, 15A, 15B, 15C, 16F, 17F, 20, 23A, 23B, 31, 34, 35B, 35F and/or 38 polysaccharide in the glycoconjugate to mM acetate per mM serotype 6C, 7C, 9N, 15A, 15B, 15C, 16F, 17F, 20, 23A, 23B, 31, 34, 35B, 35F and/or 38 polysaccharide in the activated polysaccharide is at least 0.6, 0.65, 0.7, 0.75, 0.8, 0.85, 0.9, or 0.95. In another embodiment, the ratio of mM acetate per mM serotype 6C, 7C, 9N, 15A, 15B, 15C, 16F, 17F, 20, 23A, 23B, 31, 34, 35B, 35F and/or 38 polysaccharide in the glycoconjugate to mM acetate per mM serotype 6C, 7C, 9N, 15A, 15B, 15C, 16F, 17F, 20, 23A, 23B, 31, 34, 35B, 35F and/or 38 polysaccharide in the activated polysaccharide is at least 0.7. In an embodiment, the ratio of mM acetate per mM serotype 6C, 7C, 9N, 15A, 15B, 15C, 16F, 17F, 20, 23A, 23B, 31, 34, 35B, 35F and/or 38 polysaccharide in the glycoconjugate to mM acetate per mM serotype 6C, 7C, 9N, 15A, 15B, 15C, 16F, 17F, 20, 23A, 23B, 31, 34, 35B, 35F and/or 38 polysaccharide in the activated polysaccharide is at least 0.9.

The serotype 6C, 7C, 9N, 15A, 15B, 15C, 16F, 17F, 20, 23A, 23B, 31, 34, 35B, 35F and/or 38 glycoconjugates and immunogenic compositions may contain free saccharide that is not covalently conjugated to the carrier protein, but is nevertheless present in the glycoconjugate composition. The free saccharide may be noncovalently associated with (i.e., noncovalently bound to, adsorbed to, or entrapped in or with) the glycoconjugate.

In some embodiments, the serotype 6C, 7C, 9N, 15A, 15B, 15C, 16F, 17F, 20, 23A, 23B, 31, 34, 35B, 35F and/or 38 glycoconjugates of the invention comprise less than about 50%, 45%, 40%, 35%, 30%, 25%, 20%, 15%, 10% or 5% of free serotype 33F polysaccharide compared to the total amount of serotype 6C, 7C, 9N, 15A, 15B, 15C, 16F, 17F, 20, 23A, 23B, 31, 34, 35B, 35F and/or 38 polysaccharide. The serotype 6C, 7C, 9N, 15A, 15B, 15C, 16F, 17F, 20, 23A, 23B, 31, 34, 35B, 35F and/or 38 glycoconjugate comprises less than 15% free saccharide, less than 10% free saccharide, or less than 5% of free saccharide. In an embodiment, the serotype 6C, 7C, 9N, 15A, 15B, 15C, 16F, 17F, 20, 23A, 23B, 31, 34, 35B, 35F and/or 38 glycoconjugate comprises less than about 25% of free serotype 6C, 7C, 9N, 15A, 15B, 15C, 16F, 17F, 20, 23A, 23B, 31, 34, 35B, 35F and/or 38 polysaccharide compared to the total amount of serotype 6C, 7C, 9N, 15A, 15B, 15C, 16F, 17F, 20, 23A, 23B, 31, 34, 35B, 35F and/or 38 polysaccharide. In another embodiment the serotype 6C, 7C, 9N, 15A, 15B, 15C, 16F, 17F, 20, 23A, 23B, 31, 34, 35B, 35F and/or 38 glycoconjugate comprises less than about 20% of free serotype 6C, 7C, 9N, 15A, 15B, 15C, 16F, 17F, 20, 23A, 23B, 31, 34, 35B, 35F and/or 38 polysaccharide compared to the total amount of serotype 33F polysaccharide. In an embodiment, the serotype 6C, 7C, 9N, 15A, 15B, 15C, 16F, 17F, 20, 23A, 23B, 31, 34, 35B, 35F and/or 38 glycoconjugate comprises less than about 15% of free serotype 6C, 7C, 9N, 15A, 15B, 15C, 16F, 17F, 20, 23A, 23B, 31, 34, 35B, 35F and/or 38 polysaccharide compared to the total amount of serotype 6C, 7C, 9N, 15A, 15B, 15C, 16F, 17F, 20, 23A, 23B, 31, 34, 35B, 35F and/or 38 polysaccharide.

In one or more embodiments, the invention provides a serotype 6C, 7C, 9N, 15A, 15B, 15C, 16F, 17F, 20, 23A, 23B, 31, 34, 35B, 35F and/or 38 glycoconjugate having one or more of the following features alone or in combination: the polysaccharide has a molecular weight of between 50 kDa and 2,000 kDa; the glycoconjugate has a molecular weight of between 500 kDa to 10,000 KDa; the carrier protein comprises 2 to 20 lysine residues covalently linked to the saccharide; the saccharide to carrier protein ratio (w/w) is between 0.2 and 4.0; the glycoconjugate comprises at least one covalent linkage between the carrier protein and the polysaccharide for every 4, 10, 15 or 25 saccharide repeat units of the polysaccharide; the saccharide has a degree of O-acetylation between 75% and 100%; the conjugate comprises less than about 15% free polysaccharide relative to total polysaccharide; the carrier protein is CRM₁₉₇.

The serotype 6C, 7C, 9N, 15A, 15B, 15C, 16F, 17F, 20, 23A, 23B, 31, 34, 35B, 35F and/or 38 glycoconjugates may also be characterized by their molecular size distribution (K_{d}). Size exclusion chromatography media (CL-4B) can be used to determine the relative molecular size distribution of the conjugate, as mentioned above.

In an embodiment, at least 15% of the serotype 6C, 7C, 9N, 15A, 15B, 15C, 16F, 17F, 20, 23A, 23B, 31, 34, 35B, 35F and/or 38 glycoconjugates of the invention have a K_{d} below or equal to 0.3 in a CL-4B column. In an embodiment, at least 15%, 20%, 25%, 30%, 35%, 40%, 45%, 50%, 60%, 70%, 80% or 90% of the serotype 6C, 7C, 9N, 15A, 15B, 15C, 16F, 17F, 20, 23A, 23B, 31, 34, 35B, 35F and/or 38 glycoconjugates of the invention have a K_{d} below or equal to 0.3 in a CL-4B column.

In another embodiment, at least 35% of the serotype 6C, 7C, 9N, 15A, 15B, 15C, 16F, 17F, 20, 23A, 23B, 31, 34, 35B, 35F and/or 38 glycoconjugates of the invention have a K_{d} below or equal to 0.3 in a CL-4B column. In one or more embodiments, at least 40%, 45%, 50%, 55%, 60%, 65%, 70%, 75%, 80%, or 85% of the serotype 6C, 7C, 9N, 15A, 15B, 15C, 16F, 17F, 20, 23A, 23B, 31, 34, 35B, 35F and/or 38 glycoconjugates of the invention have a K_{d} below or equal to 0.3 in a CL-4B column. In an embodiment, at least 60% of the serotype 6C, 7C, 9N, 15A, 15B, 15C, 16F, 17F, 20, 23A, 23B, 31, 34, 35B, 35F and/or 38 glycoconjugates of the invention have a K_{d} below or equal to 0.3 in a CL-4B column. In an embodiment, at least 70% of the serotype 6C, 7C, 9N, 15A, 15B, 15C, 16F, 17F, 20, 23A, 23B, 31, 34, 35B, 35F and/or 38 glycoconjugates of the invention have a K_{d} below or equal to 0.3 in a CL-4B column.

In an embodiment, between 40% and 90% of the serotype 6C, 7C, 9N, 15A, 15B, 15C, 16F, 17F, 20, 23A, 23B, 31, 34, 35B, 35F and/or 38 glycoconjugates have a K_{d} below or equal to 0.3 in a CL-4B column. In another embodiment, between 50% and 90% of the serotype 6C, 7C, 9N, 15A, 15B, 15C, 16F, 17F, 20, 23A, 23B, 31, 34, 35B, 35F and/or 38 glycoconjugates have a K_{d} below or equal to 0.3 in a CL-4B column. In still an embodiment, between 65% and 80% of the serotype 6C, 7C, 9N, 15A, 15B, 15C, 16F, 17F, 20, 23A, 23B, 31, 34, 35B, 35F and/or 38 glycoconjugates have a K_{d} below or equal to 0.3 in a CL-4B column.

1.3.2 Glycoconjugates from S. pneumoniae Serotype 6C

In an embodiment, the serotype 6C glycoconjugates are obtained by activating polysaccharide with 1-cyano-4-dimethylamino pyridinium tetrafluoroborate (CDAP) to form a cyanate ester. The activated polysaccharide may be coupled directly or via a spacer (linker) group to an amino group on the carrier protein. For example, the spacer may be cystamine or cysteamine to give a thiolated polysaccharide which may be coupled to the carrier via a thioether linkage obtained after reaction with a maleimide-activated carrier protein (for example using GMBS) or a haloacetylated carrier protein (for example using iodoacetimide, SIB, SIAB, sulfo-SIAB, SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO93/15760, WO95/08348 and WO96/129094.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO98/42721. Conjugation may involve a carbonyl linker which may be formed by reaction of a free hydroxyl group of the saccharide with CDI (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In one or more embodiments, the serotype 6C glycoconjugates of the invention are prepared using reductive amination. Reductive amination involves two steps: (1) oxidation of the polysaccharide to generate aldehyde functionalities from vicinal diols in individual hexasaccharide unit and (2) reduction of the activated polysaccharide and a carrier protein (e.g., CRM₁₉₇) to form a conjugate.

In an embodiment, before oxidation, sizing of the serotype 6C polysaccharide to a target molecular weight (MW) range is performed. Advantageously, the size of the purified serotype 6C polysaccharide is reduced while preserving critical features of the structure of the polysaccharide such as for example the presence of O-acetyl groups. In an embodiment, the size of the purified serotype 6C polysaccharide is reduced by mechanical homogenization as described herein.

In an embodiment, serotype polysaccharide is activated (oxidized) by a process comprising the step of:
(a) reacting isolated serotype 6C polysaccharide with an oxidizing agent; and
(b) quenching the oxidation reaction by addition of a quenching agent resulting in an activated serotype 6C polysaccharide.

In an embodiment, the oxidizing agent is periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate). In an embodiment, the oxidizing agent is sodium periodate. In an embodiment, the periodate used for the oxidation of serotype 6C polysaccharide is metaperiodate. In another embodiment, the periodate used for the oxidation of serotype 6C polysaccharide is sodium metaperiodate.

In one embodiment, the quenching agent is selected from vicinal diols, 1,2-aminoalcohols, amino acids, glutathione, sulfite, bisulfate, dithionite, metabisulfite, thiosulfate, phosphites, hypophosphites or phosphorous acid.

In one embodiment, the quenching agent is a 1,2-aminoalcohols of formula (I):

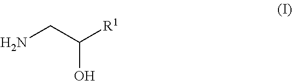

wherein R¹ is selected from H, methyl, ethyl, propyl or isopropyl.

In one embodiment, the quenching agent is selected from sodium and potassium salts of sulfite, bisulfate, dithionite, metabisulfite, thiosulfate, phosphites, hypophosphites or phosphorous acid.

In one embodiment, the quenching agent is an amino acid. In such embodiments, said amino acid may be selected from serine, threonine, cysteine, cystine, methionine, proline, hydroxyproline, tryptophan, tyrosine, and histidine.

In one embodiment, the quenching agent is a sulfite such as bisulfate, dithionite, metabisulfite, thiosulfate.

In one embodiment, the quenching agent is a compound comprising two vicinal hydroxyl groups (vicinal diols), i.e., two hydroxyl groups covalently linked to two adjacent carbon atoms.

In one or more embodiments, the quenching agent is a compound of formula (II)

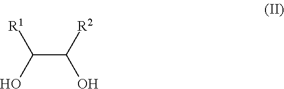

wherein R¹ and R² are each independently selected from H, methyl, ethyl, propyl or isopropyl.

In an embodiment, the quenching agent is glycerol, ethylene glycol, propan-1,2-diol, butan-1,2-diol or butan-2,3-diol, or ascorbic acid. In an embodiment, the quenching agent is butan-2,3-diol.

In another embodiment, the isolated serotype 6C polysaccharide is activated by a process comprising the steps of:
(a) reacting isolated serotype 6C polysaccharide with periodate; and
(b) quenching the oxidation reaction by addition of butan-2,3-diol resulting in an activated serotype 6C polysaccharide.

Following the oxidation step of the polysaccharide, the polysaccharide is said to be activated and is referred to as “activated polysaccharide” herein.

In an embodiment, the activated serotype 6C polysaccharide is purified. The activated serotype 6C polysaccharide is purified according to methods known to one skilled in the art such as gel permeation chromatography (GPC), dialysis or ultrafiltration/diafiltration. For example, the activated 6C polysaccharide is purified by concentration and diafiltration using an ultrafiltration device.

In another embodiment, the degree of oxidation of the activated serotype 6C polysaccharide is between 2 and 30, between 2 and 25, between 2 and 20, between 2 and 15, between 2 and 10, between 2 and 5, between 5 and 30, between 5 and 25, between 5 and 20, between 5 and 15, between 5 and 10, between 10 and 30, between 10 and 25, between 10 and 20, between 10 and 15, between 15 and 30, between 15 and 25, between 15 and 20, between 20 to 30, or between 20 to 25. In an embodiment the degree of oxidation of the activated serotype 6C polysaccharide is between 2 and 10, between 4 and 8, between 4 and 6, between 6 and 8, between 6 and 12, between 8 and 14, between 9 and 11, between 10 and 16, between 12 and 16, between 14 and 18, between 16 and 20, between 16 and 18, between 18 and 22, or between 18 and 20.

In an embodiment, the activated serotype 6C polysaccharide has a molecular weight between 25 kDa and 1,000 kDa, between 100 kDa and 1,000 kDa, between 300 kDa and 800 kDa, between 300 kDa and 700 kDa, between 300 kDa and 600 kDa, between 400 kDa and 1,000 kDa, between 400 kDa and 800 kDa, between 400 kDa and 700 kDa or between 400 kDa and 600 kDa. In an embodiment, the activated serotype 6C polysaccharide has a molecular weight between 300 kDa and 800 kDa. In an embodiment, the activated serotype 6C polysaccharide has a molecular weight between 400 kDa and 600 kDa. In another embodiment, the activated serotype 6C polysaccharide has a molecular weight between 400 kda and 600 kDa and a degree of oxidation between 10 and 25, between 10 and 20, between 12 and 20 or between 14 and 18. In another embodiment, the activated serotype 6C polysaccharide has a molecular weight between 400 kDa and 600 kDa and a degree of oxidation between 10 and 20.

In another embodiment, the activated serotype 6C polysaccharide comprises at least 0.1, 0.2, 0.3, 0.4, 0.5, 0.6 or 0.7 or about 0.8 mM acetate per mM serotype 6C polysaccharide. In an embodiment, the activated serotype 6C polysaccharide comprises at least 0.5, 0.6 or 0.7 mM acetate per mM serotype 6C polysaccharide. In another embodiment, the activated serotype 6C polysaccharide comprises at least 0.6 mM acetate per mM serotype 6C polysaccharide. In another embodiment, the activated serotype 6C polysaccharide comprises at least 0.7 mM acetate per mM serotype 6C polysaccharide.

In an embodiment, the activated serotype 6C polysaccharide has a molecular weight between 400 kDa and 800 kDa and comprises at least 0.6 mM acetate per mM serotype 6C polysaccharide.

In an embodiment, the activated serotype 6C polysaccharide has a molecular weight between 400 kDa and 800 kDa, a degree of oxidation between 12 and 20 and comprises at least 0.6 mM acetate per mM serotype 6C polysaccharide.

The activated polysaccharide and/or the carrier protein may be lyophilised (freeze-dried), either independently (discrete lyophilization) or together (co-lyophilized).

In another embodiment, the activated serotype 6C polysaccharide is lyophilized, optionally in the presence of saccharide. In an embodiment, the saccharide is selected from sucrose, trehalose, raffinose, stachyose, melezitose, dextran, mannitol, lactitol and palatinit. In an embodiment, the saccharide is sucrose. In one embodiment, the lyophilized activated polysaccharide is then compounded with a solution comprising the carrier protein.

In another embodiment, the activated polysaccharide and the carrier protein are co-lyophilised. In such embodiments, the activated serotype 6C polysaccharide is compounded with the carrier protein and lyophilized optionally in the presence of a saccharide. In an embodiment, the saccharide is selected from sucrose, trehalose, raffinose, stachyose, melezitose, dextran, mannitol, lactitol and palatinit. In an embodiment, the saccharide is sucrose. The co-lyophilized polysaccharide and carrier protein can then be resuspended in solution and reacted with a reducing agent.

The second step of the conjugation process is the reduction of the activated polysaccharide and a carrier protein to form a conjugate (reductive amination), using a reducing agent.

The activated serotype 6C polysaccharide can be conjugated to a carrier protein by a process comprising the steps of:
(c) compounding the activated serotype 6C polysaccharide with a carrier protein; and
(d) reacting the compounded activated serotype 6C polysaccharide and carrier protein with a reducing agent to form a serotype 6C polysaccharide-carrier protein conjugate.

In an embodiment, the reduction reaction is carried out in aqueous solvent. In another embodiment the reaction is carried out in aprotic solvent. In an embodiment, the reduction reaction is carried out in DMSO (dimethylsulfoxide) or in DMF (dimethylformamide)) solvent. The DMSO or DMF solvent may be used to reconstitute the activated polysaccharide and carrier protein which has been lyophilised.

The conjugation of activated serotype 6C polysaccharide with a protein carrier by reductive amination in dimethylsulfoxide (DMSO) is suitable to preserve the O-acetyl content of the polysaccharide as compared, for example, to reductive amination in aqueous phase where the level of O-acetylation of the polysaccharide may be significantly reduced. Therefore, in one or more embodiments, step (c) and step (d) are carried out in DMSO.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In an embodiment, the reducing agent is sodium cyanoborohydride.

At the end of the reduction reaction, there may be unreacted aldehyde groups remaining in the conjugates, these may be capped using a suitable capping agent. In one embodiment this capping agent is sodium borohydride (NaBH₄).

Following conjugation of serotype 6C polysaccharide to the carrier protein, the glycoconjugate can be purified (enriched with respect to the amount of polysaccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

In some embodiments, the serotype 6C glycoconjugates of the present invention comprise a saccharide having a molecular weight of between 10 kDa and 2,000 kDa. In other such embodiments, the saccharide has a molecular weight of between 50 kDa and 1,000 kDa. In other such embodiments, the saccharide has a molecular weight of between 70 kDa and 900 kDa. In other such embodiments, the saccharide has a molecular weight of between 100 kDa and 800 kDa. In other such embodiments, the saccharide has a molecular weight of between 200 kDa and 600 kDa. In further such embodiments, the saccharide has a molecular weight of 100 kDa to 1,000 kDa; 100 kDa to 900 kDa; 100 kDa to 800 kDa; 100 kDa to 700 kDa; 100 kDa to 600 kDa; 100 kDa to 500 kDa; 100 kDa to 400 kDa; 100 kDa to 300 kDa; 150 kDa to 1,000 kDa; 150 kDa to 900 kDa; 150 kDa to 800 kDa; 150 kDa to 700 kDa; 150 kDa to 600 kDa; 150 kDa to 500 kDa; 150 kDa to 400 kDa; 150 kDa to 300 kDa; 200 kDa to 1,000 kDa; 200 kDa to 900 kDa; 200 kDa to 800 kDa; 200 kDa to 700 kDa; 200 kDa to 600 kDa; 200 kDa to 500 kDa; 200 kDa to 400 kDa; 200 kDa to 300 kDa; 250 kDa to 1,000 kDa; 250 kDa to 900 kDa; 250 kDa to 800 kDa; 250 kDa to 700 kDa; 250 kDa to 600 kDa; 250 kDa to 500 kDa; 250 kDa to 400 kDa; 250 kDa to 350 kDa; 300 kDa to 1000 kDa; 300 kDa to 900 kDa; 300 kDa to 800 kDa; 300 kDa to 700 kDa; 300 kDa to 600 kDa; 300 kDa to 500 kDa; 300 kDa to 400 kDa; 400 kDa to 1,000 kDa; 400 kDa to 900 kDa; 400 kDa to 800 kDa; 400 kDa to 700 kDa; 400 kDa to 600 kDa; 500 kDa to 600 kDa. Any whole number integer within any of the above ranges is contemplated as an embodiment of the disclosure. In some such embodiments, the serotype 6C glycoconjugates are prepared using reductive amination.

In some embodiments, the serotype 6C glycoconjugate of the invention has a molecular weight of between 400 kDa and 15,000 kDa; between 500 kDa and 10,000 kDa; between 2,000 kDa and 10,000 kDa; between 3,000 kDa and 8,000 kDa; or between 3,000 kDa and 5,000 kDa. In other embodiments, the serotype 6C glycoconjugate has a molecular weight of between 500 kDa and 10,000 kDa. In other embodiments, the serotype 6C glycoconjugate has a molecular weight of between 1,000 kDa and 8,000 kDa. In still other embodiments, the serotype 6C glycoconjugate has a molecular weight of between 2,000 kDa and 8,000 kDa or between 3,000 kDa and 7,000 kDa. In further embodiments, the serotype 6C glycoconjugate of the invention has a molecular weight of between 200 kDa and 20,000 kDa; between 200 kDa and 15,000 kDa; between 200 kDa and 10,000 kDa; between 200 kDa and 7,500 kDa; between 200 kDa and 5,000 kDa; between 200 kDa and 3,000 kDa; between 200 kDa and 1,000 kDa; between 500 kDa and 20,000 kDa; between 500 kDa and 15,000 kDa; between 500 kDa and 12,500 kDa; between 500 kDa and 10,000 kDa; between 500 kDa and 7,500 kDa; between 500 kDa and 6,000 kDa; between 500 kDa and 5,000 kDa; between 500 kDa and 4,000 kDa; between 500 kDa and 3,000 kDa; between 500 kDa and 2,000 kDa; between 500 kDa and 1,500 kDa; between 500 kDa and 1,000 kDa; between 750 kDa and 20,000 kDa; between 750 kDa and 15,000 kDa; between 750 kDa and 12,500 kDa; between 750 kDa and 10,000 kDa; between 750 kDa and 7,500 kDa; between 750 kDa and 6,000 kDa; between 750 kDa and 5,000 kDa; between 750 kDa and 4,000 kDa; between 750 kDa and 3,000 kDa; between 750 kDa and 2,000 kDa; between 750 kDa and 1,500 kDa; between 1,000 kDa and 15,000 kDa; between 1,000 kDa and 12,500 kDa; between 1,000 kDa and 10,000 kDa; between 1,000 kDa and 7,500 kDa; between 1,000 kDa and 6,000 kDa; between 1,000 kDa and 5,000 kDa; between 1,000 kDa and 4,000 kDa; between 1,000 kDa and 2,500 kDa; between 2,000 kDa and 15,000 kDa; between 2,000 kDa and 12,500 kDa; between 2,000 kDa and 10,000 kDa; between 2,000 kDa and 7,500 kDa; between 2,000 kDa and 6,000 kDa; between 2,000 kDa and 5,000 kDa; between 2,000 kDa and 4,000 kDa; or between 2,000 kDa and 3,000 kDa.

In further embodiments, the serotype 6C glycoconjugate of the invention has a molecular weight of between 3,000 kDa and 20,000 kDa; between 3,000 kDa and 15,000 kDa; between 3,000 kDa and 10,000 kDa; between 3,000 kDa and 7,500 kDa; between 3,000 kDa and 5,000 kDa; between 4,000 kDa and 20,000 kDa; between 4,000 kDa and 15,000 kDa; between 4,000 kDa and 12,500 kDa; between 4,000 kDa and 10,000 kDa; between 4,000 kDa and 7,500 kDa; between 4,000 kDa and 6,000 kDa; or between 4,000 kDa and 5,000 kDa.

In further embodiments, the serotype 6C glycoconjugate of the invention has a molecular weight of between 5,000 kDa and 20,000 kDa; between 5,000 kDa and 15,000 kDa; between 5,000 kDa and 10,000 kDa; between 5,000 kDa and 7,500 kDa; between 6,000 kDa and 20,000 kDa; between 6,000 kDa and 15,000 kDa; between 6,000 kDa and 12,500 kDa; between 6,000 kDa and 10,000 kDa or between 6,000 kDa and 7,500 kDa.

The molecular weight of the glycoconjugate is measured by SEC-MALLS. Any whole number integer within any of the above ranges is contemplated as an embodiment of the disclosure.

In an embodiment, the serotype 6C 38 glycoconjugate of the invention comprises at least 0.1, 0.2, 0.3, 0.4, 0.5, 0.6 or 0.7 or about 0.8 mM acetate per mM serotype 6C polysaccharide. In an embodiment, the glycoconjugate comprises at least 0.5, 0.6 or 0.7 mM acetate per mM serotype 6C polysaccharide. In an embodiment, the glycoconjugate comprises at least 0.6 mM acetate per mM serotype 6C polysaccharide. In another embodiment, the glycoconjugate comprises at least 0.7 mM acetate per mM serotype 6C polysaccharide.

In another embodiment, the ratio of mM acetate per mM serotype 6C 7 polysaccharide in the glycoconjugate to mM acetate per mM serotype 6C polysaccharide in the isolated polysaccharide is at least 0.6, 0.65, 0.7, 0.75, 0.8, 0.85, 0.9, or 0.95. In an embodiment, the ratio of mM acetate per mM serotype 6C polysaccharide in the glycoconjugate to mM acetate per mM serotype 6C polysaccharide in the isolated polysaccharide is at least 0.7. In another embodiment, the ratio of mM acetate per mM serotype 6C polysaccharide in the glycoconjugate to mM acetate per mM serotype 6C polysaccharide in the isolated polysaccharide is at least 0.9.

In an embodiment, the ratio of mM acetate per mM serotype 6C polysaccharide in the glycoconjugate to mM acetate per mM serotype 6C polysaccharide in the activated polysaccharide is at least 0.6, 0.65, 0.7, 0.75, 0.8, 0.85, 0.9, or 0.95. In an embodiment, the ratio of mM acetate per mM serotype 6C polysaccharide in the glycoconjugate to mM acetate per mM serotype 6C polysaccharide in the activated polysaccharide is at least 0.7. In another embodiment, the ratio of mM acetate per mM serotype 6C polysaccharide in the glycoconjugate to mM acetate per mM serotype 6C polysaccharide in the activated polysaccharide is at least 0.9.

Another way to characterize the serotype 6C glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the CRM₁₉₇ protein starting material used to generate the conjugate materials. In an embodiment, the degree of conjugation of the serotype 6C glycoconjugate of the invention is between 2 and 15, between 2 and 13, between 2 and 10, between 2 and 8, between 2 and 6, between 2 and 5, between 2 and 4, between 3 and 15, between 3 and 13, between 3 and 10, between 3 and 8, between 3 and 6, between 3 and 5, between 3 and 4, between 5 and 15, between 5 and 10, between 8 and 15, between 8 and 12, between 10 and 15 or between 10 and 12. In an embodiment, the degree of conjugation of the serotype 6C glycoconjugate of the invention is about 2, about 3, about 4, about 5, about 6, about 7, about 8, about 9, about 10, about 11, about 12, about 13, about 14 or about 15. In an embodiment, the degree of conjugation of the serotype 6C glycoconjugate of the invention is between 4 and 7. In some such embodiments, the carrier protein is CRM₁₉₇.

The serotype 6C glycoconjugates of the invention may also be characterized by the ratio (weight/weight) of saccharide to carrier protein. In some embodiments, the ratio of serotype 6C polysaccharide to carrier protein in the glycoconjugate (w/w) is between 0.5 and 3.0 (e.g., about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0). In other embodiments, the saccharide to carrier protein ratio (w/w) is between 0.5 and 2.0, between 0.5 and 1.5, between 0.8 and 1.2, between 0.5 and 1.0, between 1.0 and 1.5 or between 1.0 and 2.0. In further embodiments, the saccharide to carrier protein ratio (w/w) is between 0.8 and 1.2. In an embodiment, the ratio of serotype 6C capsular polysaccharide to carrier protein in the conjugate is between 0.9 and 1.1. In some such embodiments, the carrier protein is CRM₁₉₇.

The serotype 6C glycoconjugates and immunogenic compositions of the invention may contain free saccharide that is not covalently conjugated to the carrier protein, but is nevertheless present in the glycoconjugate composition. The free saccharide may be noncovalently associated with (i.e., noncovalently bound to, adsorbed to, or entrapped in or with) the glycoconjugate.

In an embodiment, the serotype 6C glycoconjugate comprises less than about 50%, 45%, 40%, 35%, 30%, 25%, 20% or 15% of free serotype 6C polysaccharide compared to the total amount of serotype 6C polysaccharide. In another embodiment, the serotype 6C glycoconjugate comprises less than about 40% of free serotype 6C polysaccharide compared to the total amount of serotype 6C polysaccharide. In an embodiment, the serotype 6C glycoconjugate comprises less than about 25% of free serotype 6C polysaccharide compared to the total amount of serotype 6C polysaccharide. In an embodiment, the serotype 6C glycoconjugate comprises less than about 20% of free serotype 6C polysaccharide compared to the total amount of serotype 6C polysaccharide. In another embodiment the serotype 6C glycoconjugate comprises less than about 15% of free serotype 6C polysaccharide compared to the total amount of serotype 6C polysaccharide.

The serotype 6C glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In an embodiment, at least 30% of the serotype 6C glycoconjugate has a K_{d} below or equal to 0.3 in a CL-4B column. In another embodiment, at least 40% of the glycoconjugate has a K_{d} below or equal to 0.3 in a CL-4B column. In another embodiment, at least 45%, 50%, 55%, 60%, 65%, 70%, 75%, 80%, or 85% of the serotype 6C glycoconjugate has a K_{d} below or equal to 0.3 in a CL-4B column. In an embodiment, at least 60% of the serotype 6C glycoconjugate has a K_{d} below or equal to 0.3 in a CL-4B column. In another embodiment, between 50% and 80% of the serotype 6C glycoconjugate has a K_{d} below or equal to 0.3 in a CL-4B column. In an embodiment, between 65% and 80% of the serotype 6C glycoconjugate has a K_{d} below or equal to 0.3 in a CL-4B column.

1.3.3 Glycoconjugates from S. pneumoniae Serotype 7C

In an embodiment, the serotype 7C glycoconjugates are obtained by activating polysaccharide with 1-cyano-4-dimethylamino pyridinium tetrafluoroborate (CDAP) to form a cyanate ester. The activated polysaccharide may be coupled directly or via a spacer (linker) group to an amino group on the carrier protein. For example, the spacer may be cystamine or cysteamine to give a thiolated polysaccharide which may be coupled to the carrier via a thioether linkage obtained after reaction with a maleimide-activated carrier protein (for example using GMBS) or a haloacetylated carrier protein (for example using iodoacetimide, SIB, SIAB, sulfo-SIAB, SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO93/15760, WO95/08348 and WO96/129094.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO98/42721. Conjugation may involve a carbonyl linker which may be formed by reaction of a free hydroxyl group of the saccharide with CDI (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In one or more embodiments, the serotype 7C glycoconjugates of the invention are prepared using reductive amination. Reductive amination involves two steps: (1) oxidation of the polysaccharide to generate aldehyde functionalities from vicinal diols in individual hexasaccharide unit and (2) reduction of the activated polysaccharide and a carrier protein (e.g., CRM₁₉₇) to form a conjugate.

In an embodiment, before oxidation, sizing of the serotype 7C polysaccharide to a target molecular weight (MW) range is performed. Advantageously, the size of the purified serotype 7C polysaccharide is reduced while preserving critical features of the structure of the polysaccharide such as for example the presence of O-acetyl groups. In an embodiment, the size of the purified serotype 7C polysaccharide is reduced by mechanical homogenization as described herein.

In an embodiment, serotype polysaccharide is activated (oxidized) by a process comprising the step of:
(a) reacting isolated serotype 7C polysaccharide with an oxidizing agent; and
(b) quenching the oxidation reaction by addition of a quenching agent resulting in an activated serotype 7C polysaccharide.

In an embodiment, the oxidizing agent is periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate). In an embodiment, the oxidizing agent is sodium periodate. In an embodiment, the periodate used for the oxidation of serotype 7C polysaccharide is metaperiodate. In another embodiment, the periodate used for the oxidation of serotype 7C polysaccharide is sodium metaperiodate.

In one embodiment, the quenching agent is selected from vicinal diols, 1,2-aminoalcohols, amino acids, glutathione, sulfite, bisulfate, dithionite, metabisulfite, thiosulfate, phosphites, hypophosphites or phosphorous acid.

In one embodiment, the quenching agent is a 1,2-aminoalcohols of formula (I):

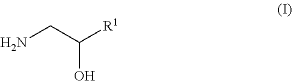

wherein R¹ is selected from H, methyl, ethyl, propyl or isopropyl.

In one embodiment, the quenching agent is selected from sodium and potassium salts of sulfite, bisulfate, dithionite, metabisulfite, thiosulfate, phosphites, hypophosphites or phosphorous acid.

In one embodiment, the quenching agent is an amino acid. In such embodiments, said amino acid may be selected from serine, threonine, cysteine, cystine, methionine, proline, hydroxyproline, tryptophan, tyrosine, and histidine.

In one embodiment, the quenching agent is a sulfite such as bisulfate, dithionite, metabisulfite, thiosulfate.

In one embodiment, the quenching agent is a compound comprising two vicinal hydroxyl groups (vicinal diols), i.e., two hydroxyl groups covalently linked to two adjacent carbon atoms.

In one or more embodiments, the quenching agent is a compound of formula (II):

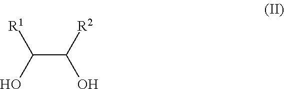

wherein R¹ and R² are each independently selected from H, methyl, ethyl, propyl or isopropyl.

In an embodiment, the quenching agent is glycerol, ethylene glycol, propan-1,2-diol, butan-1,2-diol or butan-2,3-diol, or ascorbic acid. In an embodiment, the quenching agent is butan-2,3-diol.

In another embodiment, the isolated serotype 7C polysaccharide is activated by a process comprising the steps of:
(a) reacting isolated serotype 7C polysaccharide with periodate; and
(b) quenching the oxidation reaction by addition of butan-2,3-diol resulting in an activated serotype 7C polysaccharide.

Following the oxidation step of the polysaccharide, the polysaccharide is said to be activated and is referred to as “activated polysaccharide” herein.

In an embodiment, the activated serotype 7C polysaccharide is purified. The activated serotype 7C polysaccharide is purified according to methods known to one skilled in the art such as gel permeation chromatography (GPC), dialysis or ultrafiltration/diafiltration. For example, the activated 7C polysaccharide is purified by concentration and diafiltration using an ultrafiltration device.

In another embodiment, the degree of oxidation of the activated serotype 7C polysaccharide is between 2 and 30, between 2 and 25, between 2 and 20, between 2 and 15, between 2 and 10, between 2 and 5, between 5 and 30, between 5 and 25, between 5 and 20, between 5 and 15, between 5 and 10, between 10 and 30, between 10 and 25, between 10 and 20, between 10 and 15, between 15 and 30, between 15 and 25, between 15 and 20, between 20 to 30, or between 20 to 25. In an embodiment the degree of oxidation of the activated serotype 7C polysaccharide is between 2 and 10, between 4 and 8, between 4 and 6, between 6 and 8, between 6 and 12, between 8 and 14, between 9 and 11, between 10 and 16, between 12 and 16, between 14 and 18, between 16 and 20, between 16 and 18, between 18 and 22, or between 18 and 20.

In an embodiment, the activated serotype 7C polysaccharide has a molecular weight between 25 kDa and 1,000 kDa, between 100 kDa and 1,000 kDa, between 300 kDa and 800 kDa, between 300 kDa and 700 kDa, between 300 kDa and 600 kDa, between 400 kDa and 1,000 kDa, between 400 kDa and 800 kDa, between 400 kDa and 700 kDa or between 400 kDa and 600 kDa. In an embodiment, the activated serotype 7C polysaccharide has a molecular weight between 300 kDa and 800 kDa. In an embodiment, the activated serotype 7C polysaccharide has a molecular weight between 400 kDa and 600 kDa. In another embodiment, the activated serotype 7C polysaccharide has a molecular weight between 400 kda and 600 kDa and a degree of oxidation between 10 and 25, between 10 and 20, between 12 and 20 or between 14 and 18. In another embodiment, the activated serotype 7C polysaccharide has a molecular weight between 400 kDa and 600 kDa and a degree of oxidation between 10 and 20.

In another embodiment, the activated serotype 7C polysaccharide comprises at least 0.1, 0.2, 0.3, 0.4, 0.5, 0.6 or 0.7 or about 0.8 mM acetate per mM serotype 7C polysaccharide. In an embodiment, the activated serotype 7C polysaccharide comprises at least 0.5, 0.6 or 0.7 mM acetate per mM serotype 7C polysaccharide. In another embodiment, the activated serotype 7C polysaccharide comprises at least 0.6 mM acetate per mM serotype 7C polysaccharide. In another embodiment, the activated serotype 7C polysaccharide comprises at least 0.7 mM acetate per mM serotype 7C polysaccharide.

In an embodiment, the activated serotype 7C polysaccharide has a molecular weight between 400 kDa and 800 kDa and comprises at least 0.6 mM acetate per mM serotype 7C polysaccharide.

In an embodiment, the activated serotype 7C polysaccharide has a molecular weight between 400 kDa and 800 kDa, a degree of oxidation between 12 and 20 and comprises at least 0.6 mM acetate per mM serotype 7C polysaccharide.

The activated polysaccharide and/or the carrier protein may be lyophilised (freeze-dried), either independently (discrete lyophilization) or together (co-lyophilized).

In another embodiment, the activated serotype 7C polysaccharide is lyophilized, optionally in the presence of saccharide. In an embodiment, the saccharide is selected from sucrose, trehalose, raffinose, stachyose, melezitose, dextran, mannitol, lactitol and palatinit. In an embodiment, the saccharide is sucrose. In one embodiment, the lyophilized activated polysaccharide is then compounded with a solution comprising the carrier protein.

In another embodiment, the activated polysaccharide and the carrier protein are co-lyophilised. In such embodiments, the activated serotype 7C polysaccharide is compounded with the carrier protein and lyophilized optionally in the presence of a saccharide. In an embodiment, the saccharide is selected from sucrose, trehalose, raffinose, stachyose, melezitose, dextran, mannitol, lactitol and palatinit. In an embodiment, the saccharide is sucrose. The co-lyophilized polysaccharide and carrier protein can then be resuspended in solution and reacted with a reducing agent.

The second step of the conjugation process is the reduction of the activated polysaccharide and a carrier protein to form a conjugate (reductive amination), using a reducing agent.

The activated serotype 7C polysaccharide can be conjugated to a carrier protein by a process comprising the steps of:
(c) compounding the activated serotype 7C polysaccharide with a carrier protein; and
(d) reacting the compounded activated serotype 7C polysaccharide and carrier protein with a reducing agent to form a serotype 7C polysaccharide-carrier protein conjugate.

In an embodiment, the reduction reaction is carried out in aqueous solvent. In another embodiment the reaction is carried out in aprotic solvent. In an embodiment, the reduction reaction is carried out in DMSO (dimethylsulfoxide) or in DMF (dimethylformamide)) solvent. The DMSO or DMF solvent may be used to reconstitute the activated polysaccharide and carrier protein which has been lyophilised.

The conjugation of activated serotype 7C polysaccharide with a protein carrier by reductive amination in dimethylsulfoxide (DMSO) is suitable to preserve the O-acetyl content of the polysaccharide as compared, for example, to reductive amination in aqueous phase where the level of O-acetylation of the polysaccharide may be significantly reduced. Therefore, in one or more embodiments, step (c) and step (d) are carried out in DMSO.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In an embodiment, the reducing agent is sodium cyanoborohydride.

At the end of the reduction reaction, there may be unreacted aldehyde groups remaining in the conjugates, these may be capped using a suitable capping agent. In one embodiment this capping agent is sodium borohydride (NaBH₄).

Following conjugation of serotype 7C polysaccharide to the carrier protein, the glycoconjugate can be purified (enriched with respect to the amount of polysaccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

In some embodiments, the serotype 7C glycoconjugates of the present invention comprise a saccharide having a molecular weight of between 10 kDa and 2,000 kDa. In other such embodiments, the saccharide has a molecular weight of between 50 kDa and 1,000 kDa. In other such embodiments, the saccharide has a molecular weight of between 70 kDa and 900 kDa. In other such embodiments, the saccharide has a molecular weight of between 100 kDa and 800 kDa. In other such embodiments, the saccharide has a molecular weight of between 200 kDa and 600 kDa. In further such embodiments, the saccharide has a molecular weight of 100 kDa to 1,000 kDa; 100 kDa to 900 kDa; 100 kDa to 800 kDa; 100 kDa to 700 kDa; 100 kDa to 600 kDa; 100 kDa to 500 kDa; 100 kDa to 400 kDa; 100 kDa to 300 kDa; 150 kDa to 1,000 kDa; 150 kDa to 900 kDa; 150 kDa to 800 kDa; 150 kDa to 700 kDa; 150 kDa to 600 kDa; 150 kDa to 500 kDa; 150 kDa to 400 kDa; 150 kDa to 300 kDa; 200 kDa to 1,000 kDa; 200 kDa to 900 kDa; 200 kDa to 800 kDa; 200 kDa to 700 kDa; 200 kDa to 600 kDa; 200 kDa to 500 kDa; 200 kDa to 400 kDa; 200 kDa to 300 kDa; 250 kDa to 1,000 kDa; 250 kDa to 900 kDa; 250 kDa to 800 kDa; 250 kDa to 700 kDa; 250 kDa to 600 kDa; 250 kDa to 500 kDa; 250 kDa to 400 kDa; 250 kDa to 350 kDa; 300 kDa to 1000 kDa; 300 kDa to 900 kDa; 300 kDa to 800 kDa; 300 kDa to 700 kDa; 300 kDa to 600 kDa; 300 kDa to 500 kDa; 300 kDa to 400 kDa; 400 kDa to 1,000 kDa; 400 kDa to 900 kDa; 400 kDa to 800 kDa; 400 kDa to 700 kDa; 400 kDa to 600 kDa; 500 kDa to 600 kDa. Any whole number integer within any of the above ranges is contemplated as an embodiment of the disclosure. In some such embodiments, the serotype 7C glycoconjugates are prepared using reductive amination.

In some embodiments, the serotype 7C glycoconjugate of the invention has a molecular weight of between 400 kDa and 15,000 kDa; between 500 kDa and 10,000 kDa; between 2,000 kDa and 10,000 kDa; between 3,000 kDa and 8,000 kDa; or between 3,000 kDa and 5,000 kDa. In other embodiments, the serotype 7C glycoconjugate has a molecular weight of between 500 kDa and 10,000 kDa. In other embodiments, the serotype 7C glycoconjugate has a molecular weight of between 1,000 kDa and 8,000 kDa. In still other embodiments, the serotype 7C glycoconjugate has a molecular weight of between 2,000 kDa and 8,000 kDa or between 3,000 kDa and 7,000 kDa. In further embodiments, the serotype 7C glycoconjugate of the invention has a molecular weight of between 200 kDa and 20,000 kDa; between 200 kDa and 15,000 kDa; between 200 kDa and 10,000 kDa; between 200 kDa and 7,500 kDa; between 200 kDa and 5,000 kDa; between 200 kDa and 3,000 kDa; between 200 kDa and 1,000 kDa; between 500 kDa and 20,000 kDa; between 500 kDa and 15,000 kDa; between 500 kDa and 12,500 kDa; between 500 kDa and 10,000 kDa; between 500 kDa and 7,500 kDa; between 500 kDa and 6,000 kDa; between 500 kDa and 5,000 kDa; between 500 kDa and 4,000 kDa; between 500 kDa and 3,000 kDa; between 500 kDa and 2,000 kDa; between 500 kDa and 1,500 kDa; between 500 kDa and 1,000 kDa; between 750 kDa and 20,000 kDa; between 750 kDa and 15,000 kDa; between 750 kDa and 12,500 kDa; between 750 kDa and 10,000 kDa; between 750 kDa and 7,500 kDa; between 750 kDa and 6,000 kDa; between 750 kDa and 5,000 kDa; between 750 kDa and 4,000 kDa; between 750 kDa and 3,000 kDa; between 750 kDa and 2,000 kDa; between 750 kDa and 1,500 kDa; between 1,000 kDa and 15,000 kDa; between 1,000 kDa and 12,500 kDa; between 1,000 kDa and 10,000 kDa; between 1,000 kDa and 7,500 kDa; between 1,000 kDa and 6,000 kDa; between 1,000 kDa and 5,000 kDa; between 1,000 kDa and 4,000 kDa; between 1,000 kDa and 2,500 kDa; between 2,000 kDa and 15,000 kDa; between 2,000 kDa and 12,500 kDa; between 2,000 kDa and 10,000 kDa; between 2,000 kDa and 7,500 kDa; between 2,000 kDa and 6,000 kDa; between 2,000 kDa and 5,000 kDa; between 2,000 kDa and 4,000 kDa; or between 2,000 kDa and 3,000 kDa.

In further embodiments, the serotype 7C glycoconjugate of the invention has a molecular weight of between 3,000 kDa and 20,000 kDa; between 3,000 kDa and 15,000 kDa; between 3,000 kDa and 10,000 kDa; between 3,000 kDa and 7,500 kDa; between 3,000 kDa and 5,000 kDa; between 4,000 kDa and 20,000 kDa; between 4,000 kDa and 15,000 kDa; between 4,000 kDa and 12,500 kDa; between 4,000 kDa and 10,000 kDa; between 4,000 kDa and 7,500 kDa; between 4,000 kDa and 6,000 kDa; or between 4,000 kDa and 5,000 kDa.

In further embodiments, the serotype 7C glycoconjugate of the invention has a molecular weight of between 5,000 kDa and 20,000 kDa; between 5,000 kDa and 15,000 kDa; between 5,000 kDa and 10,000 kDa; between 5,000 kDa and 7,500 kDa; between 6,000 kDa and 20,000 kDa; between 6,000 kDa and 15,000 kDa; between 6,000 kDa and 12,500 kDa; between 6,000 kDa and 10,000 kDa or between 6,000 kDa and 7,500 kDa.

The molecular weight of the glycoconjugate is measured by SEC-MALLS. Any whole number integer within any of the above ranges is contemplated as an embodiment of the disclosure.

In an embodiment, the serotype 7C glycoconjugate of the invention comprises at least 0.1, 0.2, 0.3, 0.4, 0.5, 0.6 or 0.7 or about 0.8 mM acetate per mM serotype 7C polysaccharide. In an embodiment, the glycoconjugate comprises at least 0.5, 0.6 or 0.7 mM acetate per mM serotype 7C polysaccharide. In an embodiment, the glycoconjugate comprises at least 0.6 mM acetate per mM serotype 7C polysaccharide. In another embodiment, the glycoconjugate comprises at least 0.7 mM acetate per mM serotype 7C polysaccharide.

In another embodiment, the ratio of mM acetate per mM serotype 7C polysaccharide in the glycoconjugate to mM acetate per mM serotype 7C polysaccharide in the isolated polysaccharide is at least 0.6, 0.65, 0.7, 0.75, 0.8, 0.85, 0.9, or 0.95. In an embodiment, the ratio of mM acetate per mM serotype 7C polysaccharide in the glycoconjugate to mM acetate per mM serotype 7C polysaccharide in the isolated polysaccharide is at least 0.7. In another embodiment, the ratio of mM acetate per mM serotype 7C polysaccharide in the glycoconjugate to mM acetate per mM serotype 7C polysaccharide in the isolated polysaccharide is at least 0.9.

In an embodiment, the ratio of mM acetate per mM serotype 7C polysaccharide in the glycoconjugate to mM acetate per mM serotype 7C polysaccharide in the activated polysaccharide is at least 0.6, 0.65, 0.7, 0.75, 0.8, 0.85, 0.9, or 0.95. In an embodiment, the ratio of mM acetate per mM serotype 7C polysaccharide in the glycoconjugate to mM acetate per mM serotype 7C polysaccharide in the activated polysaccharide is at least 0.7. In another embodiment, the ratio of mM acetate per mM serotype 7C polysaccharide in the glycoconjugate to mM acetate per mM serotype 7C polysaccharide in the activated polysaccharide is at least 0.9.

Another way to characterize the serotype 7C glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the CRM₁₉₇ protein starting material used to generate the conjugate materials. In an embodiment, the degree of conjugation of the serotype 7C glycoconjugate of the invention is between 2 and 15, between 2 and 13, between 2 and 10, between 2 and 8, between 2 and 6, between 2 and 5, between 2 and 4, between 3 and 15, between 3 and 13, between 3 and 10, between 3 and 8, between 3 and 6, between 3 and 5, between 3 and 4, between 5 and 15, between 5 and 10, between 8 and 15, between 8 and 12, between 10 and 15 or between 10 and 12. In an embodiment, the degree of conjugation of the serotype 7C glycoconjugate of the invention is about 2, about 3, about 4, about 5, about 6, about 7, about 8, about 9, about 10, about 11, about 12, about 13, about 14 or about 15. In an embodiment, the degree of conjugation of the serotype 7C glycoconjugate of the invention is between 4 and 7. In some such embodiments, the carrier protein is CRM₁₉₇.

The serotype 7C glycoconjugates of the invention may also be characterized by the ratio (weight/weight) of saccharide to carrier protein. In some embodiments, the ratio of serotype 7C polysaccharide to carrier protein in the glycoconjugate (w/w) is between 0.5 and 3.0 (e.g., about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0). In other embodiments, the saccharide to carrier protein ratio (w/w) is between 0.5 and 2.0, between 0.5 and 1.5, between 0.8 and 1.2, between 0.5 and 1.0, between 1.0 and 1.5 or between 1.0 and 2.0. In further embodiments, the saccharide to carrier protein ratio (w/w) is between 0.8 and 1.2. In an embodiment, the ratio of serotype 7C capsular polysaccharide to carrier protein in the conjugate is between 0.9 and 1.1. In some such embodiments, the carrier protein is CRM₁₉₇.

The serotype 7C glycoconjugates and immunogenic compositions of the invention may contain free saccharide that is not covalently conjugated to the carrier protein, but is nevertheless present in the glycoconjugate composition. The free saccharide may be noncovalently associated with (i.e., noncovalently bound to, adsorbed to, or entrapped in or with) the glycoconjugate.

In an embodiment, the serotype 7C glycoconjugate comprises less than about 50%, 45%, 40%, 35%, 30%, 25%, 20% or 15% of free serotype 7C polysaccharide compared to the total amount of serotype 7C polysaccharide. In another embodiment, the serotype 7C glycoconjugate comprises less than about 40% of free serotype 7C polysaccharide compared to the total amount of serotype 7C polysaccharide. In an embodiment, the serotype 7C glycoconjugate comprises less than about 25% of free serotype 7C polysaccharide compared to the total amount of serotype 7C polysaccharide. In an embodiment, the serotype 7C glycoconjugate comprises less than about 20% of free serotype 7C polysaccharide compared to the total amount of serotype 7C polysaccharide. In another embodiment the serotype 7C glycoconjugate comprises less than about 15% of free serotype 7C polysaccharide compared to the total amount of serotype 7C polysaccharide.

The serotype 7C glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In an embodiment, at least 30% of the serotype 7C glycoconjugate has a K_{d} below or equal to 0.3 in a CL-4B column. In another embodiment, at least 40% of the glycoconjugate has a K_{d} below or equal to 0.3 in a CL-4B column. In another embodiment, at least 45%, 50%, 55%, 60%, 65%, 70%, 75%, 80%, or 85% of the serotype 7C glycoconjugate has a K_{d} below or equal to 0.3 in a CL-4B column. In an embodiment, at least 60% of the serotype 7C glycoconjugate has a K_{d} below or equal to 0.3 in a CL-4B column. In another embodiment, between 50% and 80% of the serotype 7C glycoconjugate has a K_{d} below or equal to 0.3 in a CL-4B column. In an embodiment, between 65% and 80% of the serotype 7C glycoconjugate has a K_{d} below or equal to 0.3 in a CL-4B column.

1.3.4 Glycoconjugates from S. pneumoniae Serotype 9N

In an embodiment, the serotype 9N glycoconjugates are obtained by activating polysaccharide with 1-cyano-4-dimethylamino pyridinium tetrafluoroborate (CDAP) to form a cyanate ester. The activated polysaccharide may be coupled directly or via a spacer (linker) group to an amino group on the carrier protein. For example, the spacer may be cystamine or cysteamine to give a thiolated polysaccharide which may be coupled to the carrier via a thioether linkage obtained after reaction with a maleimide-activated carrier protein (for example using GMBS) or a haloacetylated carrier protein (for example using iodoacetimide, SIB, SIAB, sulfo-SIAB, SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO93/15760, WO95/08348 and WO96/129094.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO98/42721. Conjugation may involve a carbonyl linker which may be formed by reaction of a free hydroxyl group of the saccharide with CDI (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In one or more embodiments, the serotype 9N glycoconjugates of the invention are prepared using reductive amination. Reductive amination involves two steps: (1) oxidation of the polysaccharide to generate aldehyde functionalities from vicinal diols in individual hexasaccharide unit and (2) reduction of the activated polysaccharide and a carrier protein (e.g., CRM₁₉₇) to form a conjugate.

In an embodiment, before oxidation, sizing of the serotype 9N polysaccharide to a target molecular weight (MW) range is performed. Advantageously, the size of the purified serotype 9N polysaccharide is reduced while preserving critical features of the structure of the polysaccharide such as for example the presence of O-acetyl groups. In an embodiment, the size of the purified serotype 9N polysaccharide is reduced by mechanical homogenization as described herein.

In an embodiment, serotype polysaccharide is activated (oxidized) by a process comprising the step of:
(a) reacting isolated serotype 9N polysaccharide with an oxidizing agent; and
(b) quenching the oxidation reaction by addition of a quenching agent resulting in an activated serotype 9N polysaccharide.

In an embodiment, the oxidizing agent is periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate). In an embodiment, the oxidizing agent is sodium periodate. In an embodiment, the periodate used for the oxidation of serotype 9N polysaccharide is metaperiodate. In another embodiment, the periodate used for the oxidation of serotype 9N polysaccharide is sodium metaperiodate.

In one embodiment, the quenching agent is selected from vicinal diols, 1,2-aminoalcohols, amino acids, glutathione, sulfite, bisulfate, dithionite, metabisulfite, thiosulfate, phosphites, hypophosphites or phosphorous acid.

In one embodiment, the quenching agent is a 1,2-aminoalcohols of formula (I):

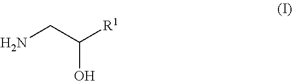

wherein R¹ is selected from H, methyl, ethyl, propyl or isopropyl.

In one embodiment, the quenching agent is selected from sodium and potassium salts of sulfite, bisulfate, dithionite, metabisulfite, thiosulfate, phosphites, hypophosphites or phosphorous acid.

In one embodiment, the quenching agent is an amino acid. In such embodiments, said amino acid may be selected from serine, threonine, cysteine, cystine, methionine, proline, hydroxyproline, tryptophan, tyrosine, and histidine.

In one embodiment, the quenching agent is a sulfite such as bisulfate, dithionite, metabisulfite, thiosulfate.

In one embodiment, the quenching agent is a compound comprising two vicinal hydroxyl groups (vicinal diols), i.e., two hydroxyl groups covalently linked to two adjacent carbon atoms.

In one or more embodiments, the quenching agent is a compound of formula (II):

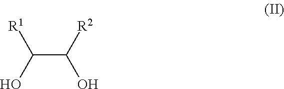

wherein R¹ and R² are each independently selected from H, methyl, ethyl, propyl or isopropyl.

In an embodiment, the quenching agent is glycerol, ethylene glycol, propan-1,2-diol, butan-1,2-diol or butan-2,3-diol, or ascorbic acid. In an embodiment, the quenching agent is butan-2,3-diol.

In another embodiment, the isolated serotype 9N polysaccharide is activated by a process comprising the steps of:
(a) reacting isolated serotype 9N polysaccharide with periodate; and
(b) quenching the oxidation reaction by addition of butan-2,3-diol resulting in an activated serotype 9N polysaccharide.

Following the oxidation step of the polysaccharide, the polysaccharide is said to be activated and is referred to as “activated polysaccharide” herein.

In an embodiment, the activated serotype 9N polysaccharide is purified. The activated serotype 9N polysaccharide is purified according to methods known to one skilled in the art such as gel permeation chromatography (GPC), dialysis or ultrafiltration/diafiltration. For example, the activated 9N polysaccharide is purified by concentration and diafiltration using an ultrafiltration device.

In another embodiment, the degree of oxidation of the activated serotype 9N polysaccharide is between 2 and 30, between 2 and 25, between 2 and 20, between 2 and 15, between 2 and 10, between 2 and 5, between 5 and 30, between 5 and 25, between 5 and 20, between 5 and 15, between 5 and 10, between 10 and 30, between 10 and 25, between 10 and 20, between 10 and 15, between 15 and 30, between 15 and 25, between 15 and 20, between 20 to 30, or between 20 to 25. In an embodiment the degree of oxidation of the activated serotype 9N polysaccharide is between 2 and 10, between 4 and 8, between 4 and 6, between 6 and 8, between 6 and 12, between 8 and 14, between 9 and 11, between 10 and 16, between 12 and 16, between 14 and 18, between 16 and 20, between 16 and 18, between 18 and 22, or between 18 and 20.

In an embodiment, the activated serotype 9N polysaccharide has a molecular weight between 25 kDa and 1,000 kDa, between 100 kDa and 1,000 kDa, between 300 kDa and 800 kDa, between 300 kDa and 700 kDa, between 300 kDa and 600 kDa, between 400 kDa and 1,000 kDa, between 400 kDa and 800 kDa, between 400 kDa and 700 kDa or between 400 kDa and 600 kDa. In an embodiment, the activated serotype 9N polysaccharide has a molecular weight between 300 kDa and 800 kDa. In an embodiment, the activated serotype 9N polysaccharide has a molecular weight between 400 kDa and 600 kDa. In another embodiment, the activated serotype 9N polysaccharide has a molecular weight between 400 kda and 600 kDa and a degree of oxidation between 10 and 25, between 10 and 20, between 12 and 20 or between 14 and 18. In another embodiment, the activated serotype 9N polysaccharide has a molecular weight between 400 kDa and 600 kDa and a degree of oxidation between 10 and 20.

In another embodiment, the activated serotype 9N polysaccharide comprises at least 0.1, 0.2, 0.3, 0.4, 0.5, 0.6 or 0.7 or about 0.8 mM acetate per mM serotype 9N polysaccharide. In an embodiment, the activated serotype 9N polysaccharide comprises at least 0.5, 0.6 or 0.7 mM acetate per mM serotype 9N polysaccharide. In another embodiment, the activated serotype 9N polysaccharide comprises at least 0.6 mM acetate per mM serotype 9N polysaccharide. In another embodiment, the activated serotype 9N polysaccharide comprises at least 0.7 mM acetate per mM serotype 9N polysaccharide.

In an embodiment, the activated serotype 9N polysaccharide has a molecular weight between 400 kDa and 800 kDa and comprises at least 0.6 mM acetate per mM serotype 9N polysaccharide.

In an embodiment, the activated serotype 9N polysaccharide has a molecular weight between 400 kDa and 800 kDa, a degree of oxidation between 12 and 20 and comprises at least 0.6 mM acetate per mM serotype 9N polysaccharide.

The activated polysaccharide and/or the carrier protein may be lyophilised (freeze-dried), either independently (discrete lyophilization) or together (co-lyophilized).

In another embodiment, the activated serotype 9N polysaccharide is lyophilized, optionally in the presence of saccharide. In an embodiment, the saccharide is selected from sucrose, trehalose, raffinose, stachyose, melezitose, dextran, mannitol, lactitol and palatinit. In an embodiment, the saccharide is sucrose. In one embodiment, the lyophilized activated polysaccharide is then compounded with a solution comprising the carrier protein.

In another embodiment, the activated polysaccharide and the carrier protein are co-lyophilised. In such embodiments, the activated serotype 9N polysaccharide is compounded with the carrier protein and lyophilized optionally in the presence of a saccharide. In an embodiment, the saccharide is selected from sucrose, trehalose, raffinose, stachyose, melezitose, dextran, mannitol, lactitol and palatinit. In an embodiment, the saccharide is sucrose. The co-lyophilized polysaccharide and carrier protein can then be resuspended in solution and reacted with a reducing agent.

The second step of the conjugation process is the reduction of the activated polysaccharide and a carrier protein to form a conjugate (reductive amination), using a reducing agent.

The activated serotype 9N polysaccharide can be conjugated to a carrier protein by a process comprising the steps of:
(c) compounding the activated serotype 9N polysaccharide with a carrier protein; and
(d) reacting the compounded activated serotype 9N polysaccharide and carrier protein with a reducing agent to form a serotype 9N polysaccharide-carrier protein conjugate.

In an embodiment, the reduction reaction is carried out in aqueous solvent. In another embodiment the reaction is carried out in aprotic solvent. In an embodiment, the reduction reaction is carried out in DMSO (dimethylsulfoxide) or in DMF (dimethylformamide)) solvent. The DMSO or DMF solvent may be used to reconstitute the activated polysaccharide and carrier protein which has been lyophilised.

The conjugation of activated serotype 9N polysaccharide with a protein carrier by reductive amination in dimethylsulfoxide (DMSO) is suitable to preserve the O-acetyl content of the polysaccharide as compared, for example, to reductive amination in aqueous phase where the level of O-acetylation of the polysaccharide may be significantly reduced. Therefore, in one or more embodiments, step (c) and step (d) are carried out in DMSO.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In an embodiment, the reducing agent is sodium cyanoborohydride.

At the end of the reduction reaction, there may be unreacted aldehyde groups remaining in the conjugates, these may be capped using a suitable capping agent. In one embodiment this capping agent is sodium borohydride (NaBH₄).

Following conjugation of serotype 9N polysaccharide to the carrier protein, the glycoconjugate can be purified (enriched with respect to the amount of polysaccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

In some embodiments, the serotype 9N glycoconjugates of the present invention comprise a saccharide having a molecular weight of between 10 kDa and 2,000 kDa. In other such embodiments, the saccharide has a molecular weight of between 50 kDa and 1,000 kDa. In other such embodiments, the saccharide has a molecular weight of between 70 kDa and 900 kDa. In other such embodiments, the saccharide has a molecular weight of between 100 kDa and 800 kDa. In other such embodiments, the saccharide has a molecular weight of between 200 kDa and 600 kDa. In further such embodiments, the saccharide has a molecular weight of 100 kDa to 1,000 kDa; 100 kDa to 900 kDa; 100 kDa to 800 kDa; 100 kDa to 700 kDa; 100 kDa to 600 kDa; 100 kDa to 500 kDa; 100 kDa to 400 kDa; 100 kDa to 300 kDa; 150 kDa to 1,000 kDa; 150 kDa to 900 kDa; 150 kDa to 800 kDa; 150 kDa to 700 kDa; 150 kDa to 600 kDa; 150 kDa to 500 kDa; 150 kDa to 400 kDa; 150 kDa to 300 kDa; 200 kDa to 1,000 kDa; 200 kDa to 900 kDa; 200 kDa to 800 kDa; 200 kDa to 700 kDa; 200 kDa to 600 kDa; 200 kDa to 500 kDa; 200 kDa to 400 kDa; 200 kDa to 300 kDa; 250 kDa to 1,000 kDa; 250 kDa to 900 kDa; 250 kDa to 800 kDa; 250 kDa to 700 kDa; 250 kDa to 600 kDa; 250 kDa to 500 kDa; 250 kDa to 400 kDa; 250 kDa to 350 kDa; 300 kDa to 1000 kDa; 300 kDa to 900 kDa; 300 kDa to 800 kDa; 300 kDa to 700 kDa; 300 kDa to 600 kDa; 300 kDa to 500 kDa; 300 kDa to 400 kDa; 400 kDa to 1,000 kDa; 400 kDa to 900 kDa; 400 kDa to 800 kDa; 400 kDa to 700 kDa; 400 kDa to 600 kDa; 500 kDa to 600 kDa. Any whole number integer within any of the above ranges is contemplated as an embodiment of the disclosure. In some such embodiments, the serotype 9N glycoconjugates are prepared using reductive amination.

In some embodiments, the serotype 9N glycoconjugate of the invention has a molecular weight of between 400 kDa and 15,000 kDa; between 500 kDa and 10,000 kDa; between 2,000 kDa and 10,000 kDa; between 3,000 kDa and 8,000 kDa; or between 3,000 kDa and 5,000 kDa. In other embodiments, the serotype 9N glycoconjugate has a molecular weight of between 500 kDa and 10,000 kDa. In other embodiments, the serotype 9N glycoconjugate has a molecular weight of between 1,000 kDa and 8,000 kDa. In still other embodiments, the serotype 9N glycoconjugate has a molecular weight of between 2,000 kDa and 8,000 kDa or between 3,000 kDa and 7,000 kDa. In further embodiments, the serotype 9N glycoconjugate of the invention has a molecular weight of between 200 kDa and 20,000 kDa; between 200 kDa and 15,000 kDa; between 200 kDa and 10,000 kDa; between 200 kDa and 7,500 kDa; between 200 kDa and 5,000 kDa; between 200 kDa and 3,000 kDa; between 200 kDa and 1,000 kDa; between 500 kDa and 20,000 kDa; between 500 kDa and 15,000 kDa; between 500 kDa and 12,500 kDa; between 500 kDa and 10,000 kDa; between 500 kDa and 7,500 kDa; between 500 kDa and 6,000 kDa; between 500 kDa and 5,000 kDa; between 500 kDa and 4,000 kDa; between 500 kDa and 3,000 kDa; between 500 kDa and 2,000 kDa; between 500 kDa and 1,500 kDa; between 500 kDa and 1,000 kDa; between 750 kDa and 20,000 kDa; between 750 kDa and 15,000 kDa; between 750 kDa and 12,500 kDa; between 750 kDa and 10,000 kDa; between 750 kDa and 7,500 kDa; between 750 kDa and 6,000 kDa; between 750 kDa and 5,000 kDa; between 750 kDa and 4,000 kDa; between 750 kDa and 3,000 kDa; between 750 kDa and 2,000 kDa; between 750 kDa and 1,500 kDa; between 1,000 kDa and 15,000 kDa; between 1,000 kDa and 12,500 kDa; between 1,000 kDa and 10,000 kDa; between 1,000 kDa and 7,500 kDa; between 1,000 kDa and 6,000 kDa; between 1,000 kDa and 5,000 kDa; between 1,000 kDa and 4,000 kDa; between 1,000 kDa and 2,500 kDa; between 2,000 kDa and 15,000 kDa; between 2,000 kDa and 12,500 kDa; between 2,000 kDa and 10,000 kDa; between 2,000 kDa and 7,500 kDa; between 2,000 kDa and 6,000 kDa; between 2,000 kDa and 5,000 kDa; between 2,000 kDa and 4,000 kDa; or between 2,000 kDa and 3,000 kDa.

In further embodiments, the serotype 9N glycoconjugate of the invention has a molecular weight of between 3,000 kDa and 20,000 kDa; between 3,000 kDa and 15,000 kDa; between 3,000 kDa and 10,000 kDa; between 3,000 kDa and 7,500 kDa; between 3,000 kDa and 5,000 kDa; between 4,000 kDa and 20,000 kDa; between 4,000 kDa and 15,000 kDa; between 4,000 kDa and 12,500 kDa; between 4,000 kDa and 10,000 kDa; between 4,000 kDa and 7,500 kDa; between 4,000 kDa and 6,000 kDa; or between 4,000 kDa and 5,000 kDa.

In further embodiments, the serotype 9N glycoconjugate of the invention has a molecular weight of between 5,000 kDa and 20,000 kDa; between 5,000 kDa and 15,000 kDa; between 5,000 kDa and 10,000 kDa; between 5,000 kDa and 7,500 kDa; between 6,000 kDa and 20,000 kDa; between 6,000 kDa and 15,000 kDa; between 6,000 kDa and 12,500 kDa; between 6,000 kDa and 10,000 kDa or between 6,000 kDa and 7,500 kDa.

The molecular weight of the glycoconjugate is measured by SEC-MALLS. Any whole number integer within any of the above ranges is contemplated as an embodiment of the disclosure.

In an embodiment, the serotype 9N glycoconjugate of the invention comprises at least 0.1, 0.2, 0.3, 0.4, 0.5, 0.6 or 0.7 or about 0.8 mM acetate per mM serotype 6C, 7C, 9N, 15A, 15B, 15C, 16F, 17F, 20, 23A, 23B, 31, 34, 35B, 35F and/or 38 polysaccharide. In an embodiment, the glycoconjugate comprises at least 0.5, 0.6 or 0.7 mM acetate per mM serotype 9N polysaccharide. In an embodiment, the glycoconjugate comprises at least 0.6 mM acetate per mM serotype 9N polysaccharide. In another embodiment, the glycoconjugate comprises at least 0.7 mM acetate per mM serotype 9N polysaccharide.

In another embodiment, the ratio of mM acetate per mM serotype 9N polysaccharide in the glycoconjugate to mM acetate per mM serotype 9N polysaccharide in the isolated polysaccharide is at least 0.6, 0.65, 0.7, 0.75, 0.8, 0.85, 0.9, or 0.95. In an embodiment, the ratio of mM acetate per mM serotype 9N polysaccharide in the glycoconjugate to mM acetate per mM serotype 9N polysaccharide in the isolated polysaccharide is at least 0.7. In another embodiment, the ratio of mM acetate per mM serotype 9N polysaccharide in the glycoconjugate to mM acetate per mM serotype 9N polysaccharide in the isolated polysaccharide is at least 0.9.

In an embodiment, the ratio of mM acetate per mM serotype 9N polysaccharide in the glycoconjugate to mM acetate per mM serotype 9N polysaccharide in the activated polysaccharide is at least 0.6, 0.65, 0.7, 0.75, 0.8, 0.85, 0.9, or 0.95. In an embodiment, the ratio of mM acetate per mM serotype 9N polysaccharide in the glycoconjugate to mM acetate per mM serotype 9N polysaccharide in the activated polysaccharide is at least 0.7. In another embodiment, the ratio of mM acetate per mM serotype 9N polysaccharide in the glycoconjugate to mM acetate per mM serotype 9N polysaccharide in the activated polysaccharide is at least 0.9.

Another way to characterize the serotype 9N glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the CRM₁₉₇ protein starting material used to generate the conjugate materials. In an embodiment, the degree of conjugation of the serotype 9N glycoconjugate of the invention is between 2 and 15, between 2 and 13, between 2 and 10, between 2 and 8, between 2 and 6, between 2 and 5, between 2 and 4, between 3 and 15, between 3 and 13, between 3 and 10, between 3 and 8, between 3 and 6, between 3 and 5, between 3 and 4, between 5 and 15, between 5 and 10, between 8 and 15, between 8 and 12, between 10 and 15 or between 10 and 12. In an embodiment, the degree of conjugation of the serotype 9N glycoconjugate of the invention is about 2, about 3, about 4, about 5, about 6, about 7, about 8, about 9, about 10, about 11, about 12, about 13, about 14 or about 15. In an embodiment, the degree of conjugation of the serotype 9N glycoconjugate of the invention is between 4 and 7. In some such embodiments, the carrier protein is CRM₁₉₇.

The serotype 9N glycoconjugates of the invention may also be characterized by the ratio (weight/weight) of saccharide to carrier protein. In some embodiments, the ratio of serotype 9N polysaccharide to carrier protein in the glycoconjugate (w/w) is between 0.5 and 3.0 (e.g., about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0). In other embodiments, the saccharide to carrier protein ratio (w/w) is between 0.5 and 2.0, between 0.5 and 1.5, between 0.8 and 1.2, between 0.5 and 1.0, between 1.0 and 1.5 or between 1.0 and 2.0. In further embodiments, the saccharide to carrier protein ratio (w/w) is between 0.8 and 1.2. In an embodiment, the ratio of serotype 9N capsular polysaccharide to carrier protein in the conjugate is between 0.9 and 1.1. In some such embodiments, the carrier protein is CRM₁₉₇.

The serotype 9N glycoconjugates and immunogenic compositions of the invention may contain free saccharide that is not covalently conjugated to the carrier protein, but is nevertheless present in the glycoconjugate composition. The free saccharide may be noncovalently associated with (i.e., noncovalently bound to, adsorbed to, or entrapped in or with) the glycoconjugate.

In an embodiment, the serotype 9N glycoconjugate comprises less than about 50%, 45%, 40%, 35%, 30%, 25%, 20% or 15% of free serotype 9N polysaccharide compared to the total amount of serotype 9N polysaccharide. In another embodiment, the serotype 9N glycoconjugate comprises less than about 40% of free serotype 9N polysaccharide compared to the total amount of serotype 9N polysaccharide. In an embodiment, the serotype 9N glycoconjugate comprises less than about 25% of free serotype 9N polysaccharide compared to the total amount of serotype 9N polysaccharide. In an embodiment, the serotype 9N glycoconjugate comprises less than about 20% of free serotype 9N polysaccharide compared to the total amount of serotype 9N polysaccharide. In another embodiment the serotype 9N glycoconjugate comprises less than about 15% of free serotype 9N polysaccharide compared to the total amount of serotype 9N polysaccharide.

The serotype 9N glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In an embodiment, at least 30% of the serotype 9N glycoconjugate has a K_{d} below or equal to 0.3 in a CL-4B column. In another embodiment, at least 40% of the glycoconjugate has a K_{d} below or equal to 0.3 in a CL-4B column. In another embodiment, at least 45%, 50%, 55%, 60%, 65%, 70%, 75%, 80%, or 85% of the serotype 9N glycoconjugate has a K_{d} below or equal to 0.3 in a CL-4B column. In an embodiment, at least 60% of the serotype 9N glycoconjugate has a K_{d} below or equal to 0.3 in a CL-4B column. In another embodiment, between 50% and 80% of the serotype 9N glycoconjugate has a K_{d} below or equal to 0.3 in a CL-4B column. In an embodiment, between 65% and 80% of the serotype 9N glycoconjugate has a K_{d} below or equal to 0.3 in a CL-4B column.

1.3.5 Glycoconjugates from S. pneumoniae Serotype 15A

In an embodiment, the serotype 15A glycoconjugates are obtained by activating polysaccharide with 1-cyano-4-dimethylamino pyridinium tetrafluoroborate (CDAP) to form a cyanate ester. The activated polysaccharide may be coupled directly or via a spacer (linker) group to an amino group on the carrier protein. For example, the spacer could be cystamine or cysteamine to give a thiolated polysaccharide which could be coupled to the carrier via a thioether linkage obtained after reaction with a maleimide-activated carrier protein (for example using GMBS) or a haloacetylated carrier protein (for example using iodoacetimide, SIB, SIAB, sulfo-SIAB, SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO93/15760, WO95/08348 and WO96/129094.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO98/42721. Conjugation may involve a carbonyl linker which may be formed by reaction of a free hydroxyl group of the saccharide with CDI (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In one or more embodiments, the serotype 15A glycoconjugates of the invention are prepared using reductive amination. Reductive amination involves two steps: (1) oxidation of the polysaccharide to generate aldehyde functionalities from vicinal diols in individual hexasaccharide unit and (2) reduction of the activated polysaccharide and a carrier protein to form a conjugate.

Preferably, before oxidation, sizing of the serotype 15A polysaccharide to a target molecular weight (MW) range is performed. Advantageously, the size of the purified serotype 15A polysaccharide is reduced while preserving critical features of the structure of the polysaccharide such as for example the presence of O-acetyl groups. Preferably, the size of the purified serotype 15A polysaccharide is reduced by mechanical homogenization (see section 1.2.6 above).

The oxidation step may involve reaction with periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate). In an embodiment the periodate used for the oxidation of serotype 15A capsular polysaccharide is metaperiodate. In an embodiment the periodate used for the oxidation of serotype 15A capsular polysaccharide is sodium metaperiodate.

In an embodiment, the polysaccharide is reacted with 0.01 to 10.0, 0.05 to 5.0, 0.1 to 1.0, 0.5 to 1.0, 0.7 to 0.8, 0.05 to 0.5, 0.1 to 0.3 molar equivalents of oxidizing agent. In an embodiment, the polysaccharide is reacted with about 0.1, 0.15, 0.2, 0.25, 0.3, 0.35, 0.4, 0.45, 0.5, 0.55, 0.6, 0.65, 0.7, 0.75, 0.8, 0.85, 0.9, 0.95 molar equivalents of oxidizing agent. In another embodiment, the polysaccharide is reacted with about 0.15 molar equivalents of oxidizing agent. In yet another embodiment, the polysaccharide is reacted with about 0.25 molar equivalents of oxidizing agent. In still another embodiment, the polysaccharide is reacted with about 0.5 molar equivalents of oxidizing agent. In an embodiment, the polysaccharide is reacted with about 0.6 molar equivalents of oxidizing agent. In another embodiment, the polysaccharide is reacted with about 0.7 molar equivalents of oxidizing agent.

In an embodiment, the duration of the reaction is between 1 hour and 50 hours, between 10 hours and 30 hours, between 15 hours and 20 hours, between 15 hours and 17 hours or about 16 hours.

In another embodiment, the temperature of the reaction is maintained between 15° C. and 45° C., between 15° C. and 30° C., between 20° C. and 25° C. In yet another embodiment, the temperature of the reaction is maintained at about 23° C.

In another embodiment, the oxidation reaction is carried out in a buffer selected from sodium phosphate, potassium phosphate, 2-(N-morpholino) ethanesulfonic acid (MES) or Bis-Tris. In an embodiment, the buffer is potassium phosphate.

In yet another embodiment, the buffer has a concentration of between 1 mM and 500 mM, between 1 mM and 300 mM, or between 50 mM and 200 mM. In still another embodiment the buffer has a concentration of about 100 mM.

In an embodiment, the oxidation reaction is carried out at a pH between 4.0 and 8.0, between 5.0 and 7.0, or between 5.5 and 6.5. In another embodiment, the pH is about 6.0.

In an embodiment, the activated serotype 15A capsular polysaccharide is obtained by reacting 0.5 mg/mL to 5 mg/mL of isolated serotype 15A capsular polysaccharide with 0.2 to 0.3 molar equivalents of periodate at a temperature between 20° C. and 25° C.

In another embodiment, the activated serotype 15A capsular polysaccharide is purified. The activated serotype 15A capsular polysaccharide is purified according to methods known to the man skilled in the art, such as gel permeation chromatography (GPC), dialysis or ultrafiltration/diafiltration. For example, the activated capsular polysaccharide is purified by concentration and diafiltration using an ultrafiltration device.

In yet another embodiment, the degree of oxidation of the activated serotype 15A capsular polysaccharide is between 2 and 20, between 2 and 15, between 2 and 10, between 2 and 5, between 5 and 20, between 5 and 15, between 5 and 10, between 10 and 20, between 10 and 15, or between 15 and 20. In another embodiment the degree of oxidation of the activated serotype 15A capsular polysaccharide is between 2 and 10, between 4 and 8, between 4 and 6, between 6 and 8, between 6 and 12, between 8 and 12, between 9 and 11, between 10 and 16, between 12 and 16, between 14 and 18, between 16 and 20, between 16 and 18, or between 18 and 20.

In still another embodiment, the activated serotype 15A capsular polysaccharide has a molecular weight between 5 kDa and 500 kDa, between 50 kDa and 500 kDa, between 50 kDa and 450 kDa, between 100 kDa and 400 kDa, between 100 kDa and 350 kDa. In an embodiment, the activated serotype 15A capsular polysaccharide has a molecular weight between 100 kDa and 350 kDa. In still another embodiment, the activated serotype 15A capsular polysaccharide has a molecular weight between 100 kDa and 300 kDa. In another embodiment, the activated serotype 15A capsular polysaccharide has a molecular weight between 100 kDa and 250 kDa.

In an embodiment, the activated serotype 15A capsular polysaccharide comprises at least 0.1, 0.2, 0.3, 0.4, 0.5, 0.6, 0.7 or 0.8 mM acetate per mM of said serotype 15A capsular polysaccharide. In another embodiment, the activated serotype 15A capsular polysaccharide comprises at least 0.5, 0.6 or 0.7 mM acetate per mM of said serotype 15A capsular polysaccharide. In yet another embodiment, the activated serotype 15A capsular polysaccharide comprises at least 0.6 mM acetate per mM of said serotype 15A capsular polysaccharide. In still another embodiment, the activated serotype 15A capsular polysaccharide comprises at least 0.7 mM acetate per mM of said serotype 15A capsular polysaccharide.

In an embodiment, the activated serotype 15A capsular polysaccharide comprises at least 0.1, 0.2, 0.3, 0.4, 0.5, 0.6, 0.7 or 0.8 mM glycerol per mM of said serotype 15A capsular polysaccharide. In another embodiment, the activated serotype 15A capsular polysaccharide comprises at least 0.5, 0.6 or 0.7 mM glycerol per mM of said serotype 15A capsular polysaccharide. In yet another embodiment, the activated serotype 15A capsular polysaccharide comprises at least 0.6 mM glycerol per mM of said serotype 15A capsular polysaccharide. In still another embodiment, the activated serotype 15A capsular polysaccharide comprises at least 0.7 mM glycerol per mM of said serotype 15A capsular polysaccharide.

In an embodiment, the activated serotype 15A capsular polysaccharide has a molecular weight between 100 kDa and 250 kDa and comprises at least 0.6 mM acetate per mM of said serotype 15A capsular polysaccharide.

In another embodiment, the activated serotype 15A capsular polysaccharide has a molecular weight between 100 kDa and 250 kDa and comprises at least 0.6 mM glycerol per mM of said serotype 15A capsular polysaccharide.

In still another embodiment, the activated serotype 15A capsular polysaccharide comprises at least 0.6 mM acetate per mM of said serotype 15A capsular polysaccharide and at least 0.6 mM glycerol per mM of said serotype 15A capsular polysaccharide.

In yet another embodiment, the activated serotype 15A capsular polysaccharide has a molecular weight between 100 kDa and 250 kDa and comprises at least 0.6 mM acetate per mM of said serotype 15A capsular polysaccharide and at least 0.6 mM glycerol per mM of said serotype 15A capsular polysaccharide.

In an embodiment, the activated serotype 15A capsular polysaccharide is lyophilized, optionally in the presence of saccharide. In an embodiment, the saccharide is selected from sucrose, trehalose, raffinose, stachyose, melezitose, dextran, mannitol, lactitol and palatinit. In an embodiment, the saccharide is sucrose. The lyophilized activated capsular polysaccharide can then be compounded with a solution comprising the carrier protein.

In another embodiment, the activated serotype 15A capsular polysaccharide is compounded with the carrier protein and lyophilized optionally in the presence of a saccharide. In an embodiment, the saccharide is selected from sucrose, trehalose, raffinose, stachyose, melezitose, dextran, mannitol, lactitol and palatinit. In an embodiment, the saccharide is sucrose. The co-lyophilized polysaccharide and carrier protein can then be resuspended in solution and reacted with a reducing agent.

The activated serotype 15A capsular polysaccharide can be conjugated to a carrier protein by a process comprising the step of:
(a) compounding the activated serotype 15A capsular polysaccharide with a carrier protein, and
(b) reacting the compounded activated serotype 15A capsular polysaccharide and carrier protein with a reducing agent to form a serotype 15A capsular polysaccharide-carrier protein conjugate.

The conjugation of activated serotype 15A capsular polysaccharide with a protein carrier by reductive amination in dimethylsulfoxide (DMSO) is suitable to preserve the O-acetyl content of the polysaccharide as compared for example to reductive amination in aqueous solution where the level of O-acetylation of the polysaccharide is significantly reduced. In another embodiment, step (a) and step (b) are carried out in DMSO.

In an embodiment, step (a) comprises dissolving lyophilized serotype 15A capsular polysaccharide in a solution comprising a carrier protein and DMSO. In an embodiment, step (a) comprises dissolving co-lyophilized serotype 15A capsular polysaccharide and carrier protein in DMSO.

When steps (a) and (b) are carried out in aqueous solution, steps (a) and (b) are carried out in a buffer, preferably selected from PBS, MES, HEPES, Bis-tris, ADA, PIPES, MOPSO, BES, MOPS, DIPSO, MOBS, HEPPSO, POPSO, TEA, EPPS, Bicine or HEPB, at a pH between 6.0 and 8.5, between 7.0 and 8.0 or between 7.0 and 7.5. In an embodiment the buffer is PBS. In an embodiment the pH is about 7.3.

In an embodiment, the concentration of activated serotype 15A capsular polysaccharide in step (b) is between 0.1 mg/mL and 10 mg/mL, between 0.5 mg/mL and 5 mg/mL, or between 0.5 mg/mL and 2 mg/mL. In another embodiment, the concentration of activated serotype 15A capsular polysaccharide in step (b) is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 mg/mL.

In yet another embodiment the initial input ratio (weight by weight) of activated serotype 15A capsular polysaccharide to carrier protein is between 5:1 and 0.1:1, between 2:1 and 0.1:1, between 2:1 and 1:1, between 1.5:1 and 1:1, between 0.1:1 and 1:1, between 0.3:1 and 1:1, or between 0.6:1 and 1:1.

In still another embodiment the initial input ratio of activated serotype 15A capsular polysaccharide to carrier protein is about 0.6:1 to 1:1. In another embodiment the initial input ratio of activated serotype 15A capsular polysaccharide to carrier protein is about 0.6:1 to 1.5:1. Such initial input ratio is particularly suitable to obtain low levels of free polysaccharide in the glycoconjugate.

In an embodiment the initial input ratio of activated serotype 15A capsular polysaccharide to carrier protein is about 0.4:1, 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1 or 2:1.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In an embodiment, the reducing agent is sodium cyanoborohydride. In an embodiment, the reducing agent is sodium 2-Picoline Borane.

In another embodiment, the quantity of reducing agent used in step (b) is between about 0.1 and 10.0 molar equivalents, between 0.5 and 5.0 molar equivalents, or between 1.0 and 2.0 molar equivalents. In an embodiment, the quantity of reducing agent used in step (b) is about 1.0, 1.1, 1.2, 1.3, 1.4, 1.5, 1.6, 1.7, 1.8, 1.9 or 2.0 molar equivalents.

In yet another embodiment, the duration of step (b) is between 1 hour and 60 hours, between 10 hours and 50 hours, between 40 hours and 50 hours, or between 42 hours and 46 hours. In an embodiment, the duration of step (b) is about 44 hours. In still another embodiment, the temperature of the reaction in step (b) is maintained between 10° C. and 40° C., between 15° C. and 30° C. or between 20° C. and

26° C. In another embodiment, the temperature of the reaction in step (b) is maintained at about 23° C.

In an embodiment, the process for the preparation of a glycoconjugate comprising S. pneumoniae serotype 15B capsular polysaccharide covalently linked to a carrier protein further comprises a step (step (c)) of capping unreacted aldehyde (quenching) by addition of NaBH₄.

In still another embodiment, the quantity of NaBH₄ used in step (c) is between 0.1 and 10 molar equivalents, between 0.5 and 5.0 molar equivalents or between 1.0 and 3.0 molar equivalents. In yet another embodiment, the quantity of NaBH₄ used in step (c) is about 2.0 molar equivalents.

In another embodiment, the duration of step (c) is between 0.1 hours and 10 hours, 0.5 hours and 5 hours, or between 2 hours and 4 hours. In an embodiment, the duration of step (c) is about 3 hours.

In another embodiment, the temperature of the reaction in step (c) is maintained between 15° C. and 45° C., between 15° C. and 30° C. or between 20° C. and 26° C. In still another embodiment, the temperature of the reaction in step (c) is maintained at about 23° C.

In another embodiment the yield of the conjugation step is greater than 50%, 55%, 60%, 65%, 70%, 75%, 80%, 85% or 90%. In still another embodiment the yield of the conjugation step (step b) is greater than 60%. In yet another embodiment the yield of the conjugation step (step b) is greater than 70%. The yield is the amount of serotype 15A polysaccharide in the conjugate×100)/amount of activated polysaccharide used in the conjugation step.

In an embodiment, the process for the preparation of a glycoconjugate comprising S. pneumoniae serotype 15A capsular polysaccharide covalently linked to a carrier protein comprises the steps of:
(a) sizing purified serotype 15A polysaccharide by high-pressure homogenization;
(b) reacting the sized serotype 15A polysaccharide with an oxidizing agent;
(c) compounding the activated serotype 15A polysaccharide with a carrier protein;
(d) reacting the compounded activated serotype 15A polysaccharide and carrier protein with a reducing agent to form a serotype 15A polysaccharide-carrier protein conjugate; and
(e) capping unreacted aldehyde (quenching) by addition of NaBH₄.

In still another embodiment, the yield of the conjugation step (step d) of the above process is greater than 50%, 55%, 60%, 65%, 70%, 75%, 80%, 85% or 90%. In another embodiment the yield of the conjugation step (step d) is greater than 60%. In still another embodiment the yield of the conjugation step (step d) is greater than 70%. The yield is the amount of serotype 15A polysaccharide in the conjugate×100)/amount of activated polysaccharide used in the conjugation step.

After conjugation of the serotype 15A capsular polysaccharide to the carrier protein, the polysaccharide-protein conjugate can be purified (enriched with respect to the amount of polysaccharide-protein conjugate) by a variety of techniques known to the skilled person. These techniques include dialysis, concentration/diafiltration operations, tangential flow filtration, precipitation/elution, column chromatography (DEAE or hydrophobic interaction chromatography), and depth filtration.

In an embodiment the carrier protein is as defined at section 1.1. In an embodiment the carrier protein is selected in the group consisting of: DT (Diphtheria toxin), TT (tetanus toxid), CRM₁₉₇, other DT mutants, PD (Haemophilus influenzae protein D), or immunologically functional equivalents thereof. In an embodiment the carrier protein is CRM₁₉₇.

In one or more embodiments, the serotype 15A glycoconjugates of the present invention are conjugated to the carrier protein (e.g., CRM₁₉₇) and comprise a saccharide having a molecular weight of between 5 kDa and 1,500 kDa. In other embodiments, the saccharide has a molecular weight of between 10 kDa and 1,500 kDa. In further such embodiments, the saccharide has a molecular weight of between 50 kDa and 1,500 kDa; between 50 kDa and 1,250 kDa; between 50 kDa and 1,000 kDa; between 50 kDa and 750 kDa; between 50 kDa and 500 kDa; between 50 kDa and 250 kDa; between 100 kDa and 1,500 kDa; between 100 kDa and 1,250 kDa; between 100 kDa and 1,000 kDa; between 100 kDa and 750 kDa; between 100 kDa and 500 kDa; between 100 kDa and 250 kDa; between 200 kDa and 1,500 kDa; between 200 kDa and 1,250 kDa; between 200 kDa and 1,000 kDa; between 200 kDa and 750 kDa; or between 200 kDa and 500 kDa; or between 200 kDa and 400 kDa. Any whole number integer within any of the above ranges is contemplated as an embodiment of the disclosure. In some embodiments, the serotype 15A glycoconjugate of the invention has a molecular weight of between 50 kDa and 20,000 kDa. In some embodiments, the serotype 15A glycoconjugate of the invention has a molecular weight of between 1,000 kDa and 20,000 kDa. In an embodiment, the serotype 15A glycoconjugate of the invention has a molecular weight between 3,000 kDa and 20,000 kDa, between 5,000 kDa and 10,000 kDa, between 5,000 kDa and 20,000 kDa, between 8,000 kDa and 20,000 kDa, between 8,000 kDa and 16,000 kDa or between 10,000 kDa and 16,000 kDa.

In one or more further embodiments, the serotype 15A glycoconjugate of the invention has a molecular weight of about 1,000 kDa, about 1,500 kDa, about 2,000 kDa, about 2,500 kDa, about 3,000 kDa, about 3,500 kDa, about 4,000 kDa, about 4,500 kDa, about 5,000 kDa, about 5,500 kDa, about 6,000 kDa, about 6,500 kDa, about 7,000 kDa, about 7,500 kDa, about 8,000 kDa, about 8,500 kDa, about 9,000 kDa, about 9,500 kDa about 10,000 kDa, about 10,500 kDa, about 11,000 kDa, about 11,500 kDa, about 12,000 kDa, about 12,500 kDa, about 13,000 kDa, about 13,500 kDa, about 14,000 kDa, about 14,500 kDa, about 15,000 kDa, about 15,500 kDa, about 16,000 kDa, about 16,500 kDa, about 17,000 kDa, about 17,500 kDa, about 18,000 kDa, about 18,500 kDa about 19,000 kDa, about 19,500 kDa or about 20,000 kDa.

In further embodiments, the serotype 15A glycoconjugate of the invention has a molecular weight of between 1,000 kDa and 20,000 kDa; between 1,000 kDa and 15,000 kDa; between 1,000 kDa and 10,000 kDa; between 1,000 kDa and 7,500 kDa; between 1,000 kDa and 5,000 kDa; between 1,000 kDa and 4,000 kDa; between 1,000 kDa and 3,000 kDa; between 2,000 kDa and 20,000 kDa; between 2,000 kDa and 15,000 kDa; between 2,000 kDa and 12,500 kDa; between 2,000 kDa and 10,000 kDa; between 2,000 kDa and 7,500 kDa; between 2,000 kDa and 6,000 kDa; between 2,000 kDa and 5,000 kDa; between 2,000 kDa and 4,000 kDa; or between 2,000 kDa and 3,000 kDa.

In further embodiments, the serotype 15A glycoconjugate of the invention has a molecular weight of between 3,000 kDa and 20,000 kDa; between 3,000 kDa and 15,000 kDa; between 3,000 kDa and 10,000 kDa; between 3,000 kDa and 7,500 kDa; between 3,000 kDa and 5,000 kDa; between 3,000 kDa and 4,000 kDa; between 4,000 kDa and 20,000 kDa; between 4,000 kDa and 15,000 kDa; between 4,000 kDa and 12,500 kDa; between 4,000 kDa and 10,000 kDa; between 4,000 kDa and 7,500 kDa; between 4,000 kDa and 6,000 kDa or between 4,000 kDa and 5,000 kDa. In further embodiments, the serotype 15A glycoconjugate of the invention has a molecular weight of between 5,000 kDa and 20,000 kDa; between 5,000 kDa and 15,000 kDa; between 5,000 kDa and 10,000 kDa; between 5,000 kDa and 7,500 kDa; between 6,000 kDa and 20,000 kDa; between 6,000 kDa and 15,000 kDa; between 6,000 kDa and 12,500 kDa; between 6,000 kDa and 10,000 kDa or between 6,000 kDa and 7,500 kDa.

The molecular weight of the glycoconjugate is measured by SEC-MALLS. Any whole number integer within any of the above ranges is contemplated as an embodiment of the disclosure. In an embodiment, said serotype 15A glycoconjugates are prepared using reductive amination.

The serotype 15A glycoconjugates of the invention may also be characterized by the ratio (weight/weight) of saccharide to carrier protein. In an embodiment, the ratio (weight by weight) of serotype 15A capsular polysaccharide to carrier protein in the conjugate is between 0.5 and 3.0 (e.g., about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9 or about 3.0). In an embodiment, the ratio of serotype 15A capsular polysaccharide to carrier protein in the conjugate is between 0.4 and 2. In an embodiment, the ratio of serotype 15A capsular polysaccharide to carrier protein in the conjugate is between 0.5 and 2.0, 0.5 and 1.5, 0.5 and 1.0, 1.0 and 1.5, 1.0 and 2.0. In an embodiment, the ratio of serotype 15B capsular polysaccharide to carrier protein in the conjugate is between 0.7 and 0.9.

The serotype 15A glycoconjugates and immunogenic compositions of the invention may contain free saccharide that is not covalently conjugated to the carrier protein, but is nevertheless present in the glycoconjugate composition. The free saccharide may be noncovalently associated with (i.e., noncovalently bound to, adsorbed to, or entrapped in or with) the glycoconjugate.

In an embodiment, the serotype 15A glycoconjugate of the invention comprises less than about 50%, 45%, 40%, 35%, 30%, 25%, 20% or 15% of free serotype 15A capsular polysaccharide compared to the total amount of serotype 15A capsular polysaccharide. In another embodiment the serotype 15A glycoconjugate of the invention comprises less than about 25% of free serotype 15A capsular polysaccharide compared to the total amount of serotype 15A capsular polysaccharide. In yet another embodiment the serotype 15A glycoconjugate of the invention comprises less than about 20% of free serotype 15A capsular polysaccharide compared to the total amount of serotype 15A capsular polysaccharide. In still another embodiment the serotype 15A glycoconjugates of the invention comprises less than about 15% of free serotype 15A capsular polysaccharide compared to the total amount of serotype 15A capsular polysaccharide.

The serotype 15A glycoconjugates may also be characterized by their molecular size distribution (K_{d}). Size exclusion chromatography media (CL-4B) can be used to determine the relative molecular size distribution of the conjugate, as mentioned above.

In an embodiment, at least 20% of the serotype 15A glycoconjugates of the invention have a K_{d} below or equal to 0.3 in a CL-4B column. In another embodiment, at least 30% of the immunogenic conjugate has a K_{d} below or equal to 0.3 in a CL-4B column. In still another embodiment, at least 40% of the serotype 15A glycoconjugates of the invention have a K_{d} below or equal to 0.3 in a CL-4B column. In an embodiment, at least 45%, 50%, 55%, 60%, 65%, 70%, 75%, 80%, or 85% of the serotype 15A glycoconjugates of the invention have a K_{d} below or equal to 0.3 in a CL-4B column. In another embodiment, at least 60% of the serotype 15A glycoconjugates of the invention have a K_{d} below or equal to 0.3 in a CL-4B column. In still another embodiment, at least 70% of the serotype 15A glycoconjugates of the invention have a K_{d} below or equal to 0.3 in a CL-4B column.

In an embodiment, between 40% and 90% of the serotype 15A glycoconjugates have a K_{d} below or equal to 0.3 in a CL-4B column. In an embodiment, between 50% and 90% of the serotype 15B glycoconjugates have a K_{d} below or equal to 0.3 in a CL-4B column. In an embodiment, between 65% and 80% of the serotype 15A glycoconjugates have a K_{d} below or equal to 0.3 in a CL-4B column.

In yet another embodiment, the serotype 15A glycoconjugate of the invention comprises at least 0.1, 0.2, 0.3, 0.4, 0.5, 0.6, 0.7 or 0.8 mM acetate per mM serotype 15A capsular polysaccharide. In an embodiment, the glycoconjugate comprises at least 0.5, 0.6 or 0.7 mM acetate per mM serotype 15A capsular polysaccharide. In another embodiment, the glycoconjugate comprises at least 0.6 mM acetate per mM serotype 15A capsular polysaccharide. In still another embodiment, the glycoconjugate comprises at least 0.7 mM acetate per mM serotype 15A capsular polysaccharide. In yet another embodiment, the presence of O-acetyl groups is determined by ion-HPLC analysis.

In another embodiment, the ratio of mM acetate per mM serotype 15A capsular polysaccharide in the serotype 15A glycoconjugate to mM acetate per mM serotype 15A capsular polysaccharide in the isolated polysaccharide is at least 0.6, 0.65, 0.7, 0.75, 0.8, 0.85, 0.9, or 0.95. In yet another embodiment, the ratio of mM acetate per mM serotype 15A capsular polysaccharide in the serotype 15A glycoconjugate to mM acetate per mM serotype 15A capsular polysaccharide in the isolated polysaccharide is at least 0.7. In another embodiment, the ratio of mM acetate per mM serotype 15A capsular polysaccharide in the serotype 15A glycoconjugate to mM acetate per mM serotype 15A capsular polysaccharide in the isolated polysaccharide is at least 0.9. In still another embodiment, the presence of O-acetyl groups is determined by ion-HPLC analysis.

In an embodiment, the ratio of mM acetate per mM serotype 15A capsular polysaccharide in the serotype 15A glycoconjugate to mM acetate per mM serotype 15A capsular polysaccharide in the activated polysaccharide is at least 0.6, 0.65, 0.7, 0.75, 0.8, 0.85, 0.9, or 0.95. In another embodiment, the ratio of mM acetate per mM serotype 15A capsular polysaccharide in the serotype 15A glycoconjugate to mM acetate per mM serotype 15A capsular polysaccharide in the activated polysaccharide is at least 0.7. In yet another embodiment, the ratio of mM acetate per mM serotype 15A capsular polysaccharide in the serotype 15A glycoconjugate to mM acetate per mM serotype 15A capsular polysaccharide in the activated polysaccharide is at least 0.9. In an embodiment, the presence of O-acetyl groups is determined by ion-HPLC analysis.

In an embodiment, the serotype 15A glycoconjugate of the invention comprises at least 0.1, 0.2, 0.3, 0.4, 0.5, 0.6, 0.7 or 0.8 mM glycerol per mM serotype 15A capsular polysaccharide. In another embodiment, the serotype 15A glycoconjugate of the invention comprises at least 0.5, 0.6 or 0.7 mM glycerol per mM serotype 15A capsular polysaccharide. In still another embodiment, the serotype 15A glycoconjugate of the invention comprises at least 0.6 mM glycerol per mM serotype 15A capsular polysaccharide. In yet another embodiment, the serotype 15A glycoconjugate of the invention comprises at least 0.7 mM glycerol per mM serotype 15A capsular polysaccharide.

Another way to characterize the serotype 15A glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the CRM₁₉₇ protein starting material used to generate the conjugate materials.

In an embodiment, the degree of conjugation of the serotype 15A glycoconjugate of the invention is between 2 and 15, between 2 and 13, between 2 and 10, between 2 and 8, between 2 and 6, between 2 and 5, between 2 and 4, between 3 and 15, between 3 and 13, between 3 and 10, between 3 and 8, between 3 and 6, between 3 and 5, between 3 and 4, between 5 and 15, between 5 and 10, between 8 and 15, between 8 and 12, between 10 and 15 or between 10 and 12. In an embodiment, the degree of conjugation of the serotype 15A glycoconjugate of the invention is about 2, about 3, about 4, about 5, about 6, about 7, about 8, about 9, about 10, about 11, about 12, about 13, about 14 or about 15. In another embodiment, the degree of conjugation of the serotype 15A glycoconjugate of the invention is between 2 and 5.

1.3.6 Glycoconjugates from S. pneumoniae Serotype 15B

In an embodiment, the serotype 15B glycoconjugates are obtained by activating polysaccharide with 1-cyano-4-dimethylamino pyridinium tetrafluoroborate (CDAP) to form a cyanate ester. The activated polysaccharide may be coupled directly or via a spacer (linker) group to an amino group on the carrier protein. For example, the spacer could be cystamine or cysteamine to give a thiolated polysaccharide which could be coupled to the carrier via a thioether linkage obtained after reaction with a maleimide-activated carrier protein (for example using GMBS) or a haloacetylated carrier protein (for example using iodoacetimide, SIB, SIAB, sulfo-SIAB, SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO93/15760, WO95/08348 and WO96/129094.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO98/42721. Conjugation may involve a carbonyl linker which may be formed by reaction of a free hydroxyl group of the saccharide with CDI (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In one or more embodiments, the serotype 15B glycoconjugates of the invention are prepared using reductive amination. Reductive amination involves two steps: (1) oxidation of the polysaccharide to generate aldehyde functionalities from vicinal diols in individual hexasaccharide unit and (2) reduction of the activated polysaccharide and a carrier protein to form a conjugate.

Preferably, before oxidation, sizing of the serotype 15B polysaccharide to a target molecular weight (MW) range is performed. Advantageously, the size of the purified serotype 15B polysaccharide is reduced while preserving critical features of the structure of the polysaccharide such as for example the presence of O-acetyl groups. Preferably, the size of the purified serotype 15B polysaccharide is reduced by mechanical homogenization (see section 1.2.6 above).

The oxidation step may involve reaction with periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate). In an embodiment the periodate used for the oxidation of serotype 15B capsular polysaccharide is metaperiodate. In an embodiment the periodate used for the oxidation of serotype 15B capsular polysaccharide is sodium metaperiodate.

In an embodiment, the polysaccharide is reacted with 0.01 to 10.0, 0.05 to 5.0, 0.1 to 1.0, 0.5 to 1.0, 0.7 to 0.8, 0.05 to 0.5, 0.1 to 0.3 molar equivalents of oxidizing agent. In an embodiment, the polysaccharide is reacted with about 0.1, 0.15, 0.2, 0.25, 0.3, 0.35, 0.4, 0.45, 0.5, 0.55, 0.6, 0.65, 0.7, 0.75, 0.8, 0.85, 0.9, 0.95 molar equivalents of oxidizing agent. In another embodiment, the polysaccharide is reacted with about 0.15 molar equivalents of oxidizing agent. In yet another embodiment, the polysaccharide is reacted with about 0.25 molar equivalents of oxidizing agent. In still another embodiment, the polysaccharide is reacted with about 0.5 molar equivalents of oxidizing agent. In an embodiment, the polysaccharide is reacted with about 0.6 molar equivalents of oxidizing agent. In another embodiment, the polysaccharide is reacted with about 0.7 molar equivalents of oxidizing agent.

In an embodiment, the duration of the reaction is between 1 hour and 50 hours, between 10 hours and 30 hours, between 15 hours and 20 hours, between 15 hours and 17 hours or about 16 hours.

In another embodiment, the temperature of the reaction is maintained between 15° C. and 45° C., between 15° C. and 30° C., between 20° C. and 25° C. In yet another embodiment, the temperature of the reaction is maintained at about 23° C.

In another embodiment, the oxidation reaction is carried out in a buffer selected from sodium phosphate, potassium phosphate, 2-(N-morpholino) ethanesulfonic acid (MES) or Bis-Tris. In an embodiment, the buffer is potassium phosphate.

In yet another embodiment, the buffer has a concentration of between 1 mM and 500 mM, between 1 mM and 300 mM, or between 50 mM and 200 mM. In still another embodiment the buffer has a concentration of about 100 mM.

In an embodiment, the oxidation reaction is carried out at a pH between 4.0 and 8.0, between 5.0 and 7.0, or between 5.5 and 6.5. In another embodiment, the pH is about 6.0.

In an embodiment, the activated serotype 15B capsular polysaccharide is obtained by reacting 0.5 mg/mL to 5 mg/ml of isolated serotype 15B capsular polysaccharide with 0.2 to 0.3 molar equivalents of periodate at a temperature between 20° C. and 25° C.

In another embodiment, the activated serotype 15B capsular polysaccharide is purified. The activated serotype 15B capsular polysaccharide is purified according to methods known to the man skilled in the art, such as gel permeation chromatography (GPC), dialysis or ultrafiltration/diafiltration. For example, the activated capsular polysaccharide is purified by concentration and diafiltration using an ultrafiltration device.

In yet another embodiment, the degree of oxidation of the activated serotype 15B capsular polysaccharide is between 2 and 20, between 2 and 15, between 2 and 10, between 2 and 5, between 5 and 20, between 5 and 15, between 5 and 10, between 10 and 20, between 10 and 15, or between 15 and 20. In another embodiment the degree of oxidation of the activated serotype 15B capsular polysaccharide is between 2 and 10, between 4 and 8, between 4 and 6, between 6 and 8, between 6 and 12, between 8 and 12, between 9 and 11, between 10 and 16, between 12 and 16, between 14 and 18, between 16 and 20, between 16 and 18, or between 18 and 20.

In still another embodiment, the activated serotype 15B capsular polysaccharide has a molecular weight between 5 kDa and 500 kDa, between 50 kDa and 500 kDa, between 50 kDa and 450 kDa, between 100 kDa and 400 kDa, between 100 kDa and 350 kDa. In an embodiment, the activated serotype 15B capsular polysaccharide has a molecular weight between 100 kDa and 350 kDa. In still another embodiment, the activated serotype 15B capsular polysaccharide has a molecular weight between 100 kDa and 300 kDa. In another embodiment, the activated serotype 15B capsular polysaccharide has a molecular weight between 100 kDa and 250 kDa.

In an embodiment, the activated serotype 15B capsular polysaccharide comprises at least 0.1, 0.2, 0.3, 0.4, 0.5, 0.6, 0.7 or 0.8 mM acetate per mM of said serotype 15B capsular polysaccharide. In another embodiment, the activated serotype 15B capsular polysaccharide comprises at least 0.5, 0.6 or 0.7 mM acetate per mM of said serotype 15B capsular polysaccharide. In yet another embodiment, the activated serotype 15B capsular polysaccharide comprises at least 0.6 mM acetate per mM of said serotype 15B capsular polysaccharide. In still another embodiment, the activated serotype 15B capsular polysaccharide comprises at least 0.7 mM acetate per mM of said serotype 15B capsular polysaccharide.

In an embodiment, the activated serotype 15B capsular polysaccharide comprises at least 0.1, 0.2, 0.3, 0.4, 0.5, 0.6, 0.7 or 0.8 mM glycerol per mM of said serotype 15B capsular polysaccharide. In another embodiment, the activated serotype 15B capsular polysaccharide comprises at least 0.5, 0.6 or 0.7 mM glycerol per mM of said serotype 15B capsular polysaccharide. In yet another embodiment, the activated serotype 15B capsular polysaccharide comprises at least 0.6 mM glycerol per mM of said serotype 15B capsular polysaccharide. In still another embodiment, the activated serotype 15B capsular polysaccharide comprises at least 0.7 mM glycerol per mM of said serotype 15B capsular polysaccharide.

In an embodiment, the activated serotype 15B capsular polysaccharide has a molecular weight between 100 kDa and 250 kDa and comprises at least 0.6 mM acetate per mM of said serotype 15B capsular polysaccharide.

In another embodiment, the activated serotype 15B capsular polysaccharide has a molecular weight between 100 kDa and 250 kDa and comprises at least 0.6 mM glycerol per mM of said serotype 15B capsular polysaccharide.

In still another embodiment, the activated serotype 15B capsular polysaccharide comprises at least 0.6 mM acetate per mM of said serotype 15B capsular polysaccharide and at least 0.6 mM glycerol per mM of said serotype 15B capsular polysaccharide.

In yet another embodiment, the activated serotype 15B capsular polysaccharide has a molecular weight between 100 kDa and 250 kDa and comprises at least 0.6 mM acetate per mM of said serotype 15B capsular polysaccharide and at least 0.6 mM glycerol per mM of said serotype 15B capsular polysaccharide.

In an embodiment, the activated serotype 15B capsular polysaccharide is lyophilized, optionally in the presence of saccharide. In an embodiment, the saccharide is selected from sucrose, trehalose, raffinose, stachyose, melezitose, dextran, mannitol, lactitol and palatinit. In an embodiment, the saccharide is sucrose. The lyophilized activated capsular polysaccharide can then be compounded with a solution comprising the carrier protein.

In another embodiment, the activated serotype 15B capsular polysaccharide is compounded with the carrier protein and lyophilized optionally in the presence of a saccharide. In an embodiment, the saccharide is selected from sucrose, trehalose, raffinose, stachyose, melezitose, dextran, mannitol, lactitol and palatinit. In an embodiment, the saccharide is sucrose. The co-lyophilized polysaccharide and carrier protein can then be resuspended in solution and reacted with a reducing agent.

The activated serotype 15B capsular polysaccharide can be conjugated to a carrier protein by a process comprising the step of:
(a) compounding the activated serotype 15B capsular polysaccharide with a carrier protein, and
(b) reacting the compounded activated serotype 15B capsular polysaccharide and carrier protein with a reducing agent to form a serotype 15B capsular polysaccharide-carrier protein conjugate.

The conjugation of activated serotype 15B capsular polysaccharide with a protein carrier by reductive amination in dimethylsulfoxide (DMSO) is suitable to preserve the O-acetyl content of the polysaccharide as compared for example to reductive amination in aqueous solution where the level of O-acetylation of the polysaccharide is significantly reduced. In another embodiment, step (a) and step (b) are carried out in DMSO.

In an embodiment, step (a) comprises dissolving lyophilized serotype 15B capsular polysaccharide in a solution comprising a carrier protein and DMSO. In an embodiment, step (a) comprises dissolving co-lyophilized serotype 15B capsular polysaccharide and carrier protein in DMSO.

When steps (a) and (b) are carried out in aqueous solution, steps (a) and (b) are carried out in a buffer, preferably selected from PBS, MES, HEPES, Bis-tris, ADA, PIPES, MOPSO, BES, MOPS, DIPSO, MOBS, HEPPSO, POPSO, TEA, EPPS, Bicine or HEPB, at a pH between 6.0 and 8.5, between 7.0 and 8.0 or between 7.0 and 7.5. In an embodiment the buffer is PBS. In an embodiment the pH is about 7.3.

In an embodiment, the concentration of activated serotype 15B capsular polysaccharide in step (b) is between 0.1 mg/mL and 10 mg/mL, between 0.5 mg/ml and 5 mg/mL, or between 0.5 mg/mL and 2 mg/mL. In another embodiment, the concentration of activated serotype 15B capsular polysaccharide in step (b) is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 mg/mL.

In yet another embodiment the initial input ratio (weight by weight) of activated serotype 15B capsular polysaccharide to carrier protein is between 5:1 and 0.1:1, between 2:1 and 0.1:1, between 2:1 and 1:1, between 1.5:1 and 1:1, between 0.1:1 and 1:1, between 0.3:1 and 1:1, or between 0.6:1 and 1:1.

In still another embodiment the initial input ratio of activated serotype 15B capsular polysaccharide to carrier protein is about 0.6:1 to 1:1. In another embodiment the initial input ratio of activated serotype 15B capsular polysaccharide to carrier protein is about 0.6:1 to 1.5:1. Such initial input ratio is particularly suitable to obtain low levels of free polysaccharide in the glycoconjugate.

In an embodiment the initial input ratio of activated serotype 15B capsular polysaccharide to carrier protein is about 0.4:1, 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1 or 2:1.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In an embodiment, the reducing agent is sodium cyanoborohydride. In an embodiment, the reducing agent is sodium 2-Picoline Borane.

In another embodiment, the quantity of reducing agent used in step (b) is between about 0.1 and 10.0 molar equivalents, between 0.5 and 5.0 molar equivalents, or between 1.0 and 2.0 molar equivalents. In an embodiment, the quantity of reducing agent used in step (b) is about 1.0, 1.1, 1.2, 1.3, 1.4, 1.5, 1.6, 1.7, 1.8, 1.9 or 2.0 molar equivalents.

In yet another embodiment, the duration of step (b) is between 1 hour and 60 hours, between 10 hours and 50 hours, between 40 hours and 50 hours, or between 42 hours and 46 hours. In an embodiment, the duration of step (b) is about 44 hours. In still another embodiment, the temperature of the reaction in step (b) is maintained between 10° C. and 40° C., between 15° C. and 30° C. or between 20° C. and

26° C. In another embodiment, the temperature of the reaction in step (b) is maintained at about 23° C.

In an embodiment, the process for the preparation of a glycoconjugate comprising S. pneumoniae serotype 15B capsular polysaccharide covalently linked to a carrier protein further comprises a step (step (c)) of capping unreacted aldehyde (quenching) by addition of NaBH₄.

In still another embodiment, the quantity of NaBH₄ used in step (c) is between 0.1 and 10 molar equivalents, between 0.5 and 5.0 molar equivalents or between 1.0 and 3.0 molar equivalents. In yet another embodiment, the quantity of NaBH₄ used in step (c) is about 2.0 molar equivalents.

In another embodiment, the duration of step (c) is between 0.1 hours and 10 hours, 0.5 hours and 5 hours, or between 2 hours and 4 hours. In an embodiment, the duration of step (c) is about 3 hours.

In another embodiment, the temperature of the reaction in step (c) is maintained between 15° C. and 45° C., between 15° C. and 30° C. or between 20° C. and 26° C. In still another embodiment, the temperature of the reaction in step (c) is maintained at about 23° C.

In another embodiment the yield of the conjugation step is greater than 50%, 55%, 60%, 65%, 70%, 75%, 80%, 85% or 90%. In still another embodiment the yield of the conjugation step (step b) is greater than 60%. In yet another embodiment the yield of the conjugation step (step b) is greater than 70%. The yield is the amount of serotype 15B polysaccharide in the conjugate×100)/amount of activated polysaccharide used in the conjugation step.

In an embodiment, the process for the preparation of a glycoconjugate comprising S. pneumoniae serotype 15B capsular polysaccharide covalently linked to a carrier protein comprises the steps of:
(a) sizing purified serotype 15B polysaccharide by high-pressure homogenization;
(b) reacting the sized serotype 15B polysaccharide with an oxidizing agent; (c) compounding the activated serotype 15B polysaccharide with a carrier protein;
(d) reacting the compounded activated serotype 15B polysaccharide and carrier protein with a reducing agent to form a serotype 15B polysaccharide-carrier protein conjugate; and
(e) capping unreacted aldehyde (quenching) by addition of NaBH₄.

In still another embodiment, the yield of the conjugation step (step d) of the above process is greater than 50%, 55%, 60%, 65%, 70%, 75%, 80%, 85% or 90%. In another embodiment the yield of the conjugation step (step d) is greater than 60%. In still another embodiment the yield of the conjugation step (step d) is greater than 70%. The yield is the amount of serotype 15B polysaccharide in the conjugate×100)/amount of activated polysaccharide used in the conjugation step.

After conjugation of the serotype 15B capsular polysaccharide to the carrier protein, the polysaccharide-protein conjugate can be purified (enriched with respect to the amount of polysaccharide-protein conjugate) by a variety of techniques known to the skilled person. These techniques include dialysis, concentration/diafiltration operations, tangential flow filtration, precipitation/elution, column chromatography (DEAE or hydrophobic interaction chromatography), and depth filtration.

In an embodiment the carrier protein is as defined at section 1.1. In an embodiment the carrier protein is selected in the group consisting of: DT (Diphtheria toxin), TT (tetanus toxid), CRM₁₉₇, other DT mutants, PD (Haemophilus influenzae protein D), or immunologically functional equivalents thereof. In an embodiment the carrier protein is CRM₁₉₇.

In one or more embodiments, the serotype 15B glycoconjugates of the present invention are conjugated to the carrier protein (e.g., CRM₁₉₇) and comprise a saccharide having a molecular weight of between 5 kDa and 1,500 kDa. In other embodiments, the saccharide has a molecular weight of between 10 kDa and 1,500 kDa. In further such embodiments, the saccharide has a molecular weight of between 50 kDa and 1,500 kDa; between 50 kDa and 1,250 kDa; between 50 kDa and 1,000 kDa; between 50 kDa and 750 kDa; between 50 kDa and 500 kDa; between 50 kDa and 250 kDa; between 100 kDa and 1,500 kDa; between 100 kDa and 1,250 kDa; between 100 kDa and 1,000 kDa; between 100 kDa and 750 kDa; between 100 kDa and 500 kDa; between 100 kDa and 250 kDa; between 200 kDa and 1,500 kDa; between 200 kDa and 1,250 kDa; between 200 kDa and 1,000 kDa; between 200 kDa and 750 kDa; or between 200 kDa and 500 kDa; or between 200 kDa and 400 kDa. Any whole number integer within any of the above ranges is contemplated as an embodiment of the disclosure. In some embodiments, the serotype 15B glycoconjugate of the invention has a molecular weight of between 50 kDa and 20,000 kDa. In some embodiments, the serotype 15B glycoconjugate of the invention has a molecular weight of between 1,000 kDa and 20,000 kDa. In an embodiment, the serotype 15B glycoconjugate of the invention has a molecular weight between 3,000 kDa and 20,000 kDa, between 5,000 kDa and 10,000 kDa, between 5,000 kDa and 20,000 kDa, between 8,000 kDa and 20,000 kDa, between 8,000 kDa and 16,000 kDa or between 10,000 kDa and 16,000 kDa.

In one or more further embodiments, the serotype 15B glycoconjugate of the invention has a molecular weight of about 1,000 kDa, about 1,500 kDa, about 2,000 kDa, about 2,500 kDa, about 3,000 kDa, about 3,500 kDa, about 4,000 kDa, about 4,500 kDa, about 5,000 kDa, about 5,500 kDa, about 6,000 kDa, about 6,500 kDa, about 7,000 kDa, about 7,500 kDa, about 8,000 kDa, about 8,500 kDa, about 9,000 kDa, about 9,500 kDa about 10,000 kDa, about 10,500 kDa, about 11,000 kDa, about 11,500 kDa, about 12,000 kDa, about 12,500 kDa, about 13,000 kDa, about 13,500 kDa, about 14,000 kDa, about 14,500 kDa, about 15,000 kDa, about 15,500 kDa, about 16,000 kDa, about 16,500 kDa, about 17,000 kDa, about 17,500 kDa, about 18,000 kDa, about 18,500 kDa about 19,000 kDa, about 19,500 kDa or about 20,000 kDa.

In further embodiments, the serotype 15B glycoconjugate of the invention has a molecular weight of between 1,000 kDa and 20,000 kDa; between 1,000 kDa and 15,000 kDa; between 1,000 kDa and 10,000 kDa; between 1,000 kDa and 7,500 kDa; between 1,000 kDa and 5,000 kDa; between 1,000 kDa and 4,000 kDa; between 1,000 kDa and 3,000 kDa; between 2,000 kDa and 20,000 kDa; between 2,000 kDa and 15,000 kDa; between 2,000 kDa and 12,500 kDa; between 2,000 kDa and 10,000 kDa; between 2,000 kDa and 7,500 kDa; between 2,000 kDa and 6,000 kDa; between 2,000 kDa and 5,000 kDa; between 2,000 kDa and 4,000 kDa; or between 2,000 kDa and 3,000 kDa.

In further embodiments, the serotype 15B glycoconjugate of the invention has a molecular weight of between 3,000 kDa and 20,000 kDa; between 3,000 kDa and 15,000 kDa; between 3,000 kDa and 10,000 kDa; between 3,000 kDa and 7,500 kDa; between 3,000 kDa and 5,000 kDa; between 3,000 kDa and 4,000 kDa; between 4,000 kDa and 20,000 kDa; between 4,000 kDa and 15,000 kDa; between 4,000 kDa and 12,500 kDa; between 4,000 kDa and 10,000 kDa; between 4,000 kDa and 7,500 kDa; between 4,000 kDa and 6,000 kDa or between 4,000 kDa and 5,000 kDa. In further embodiments, the serotype 15B glycoconjugate of the invention has a molecular weight of between 5,000 kDa and 20,000 kDa; between 5,000 kDa and 15,000 kDa; between 5,000 kDa and 10,000 kDa; between 5,000 kDa and 7,500 kDa; between 6,000 kDa and 20,000 kDa; between 6,000 kDa and 15,000 kDa; between 6,000 kDa and 12,500 kDa; between 6,000 kDa and 10,000 kDa or between 6,000 kDa and 7,500 kDa.

The molecular weight of the glycoconjugate is measured by SEC-MALLS. Any whole number integer within any of the above ranges is contemplated as an embodiment of the disclosure. In an embodiment, said serotype 15B glycoconjugates are prepared using reductive amination.

The serotype 15B glycoconjugates of the invention may also be characterized by the ratio (weight/weight) of saccharide to carrier protein. In an embodiment, the ratio (weight by weight) of serotype 15B capsular polysaccharide to carrier protein in the conjugate is between 0.5 and 3.0 (e.g., about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9 or about 3.0). In an embodiment, the ratio of serotype 15B capsular polysaccharide to carrier protein in the conjugate is between 0.4 and 2. In an embodiment, the ratio of serotype 15B capsular polysaccharide to carrier protein in the conjugate is between 0.5 and 2.0, 0.5 and 1.5, 0.5 and 1.0, 1.0 and 1.5, 1.0 and 2.0. In an embodiment, the ratio of serotype 15B capsular polysaccharide to carrier protein in the conjugate is between 0.7 and 0.9.

The serotype 15B glycoconjugates and immunogenic compositions of the invention may contain free saccharide that is not covalently conjugated to the carrier protein, but is nevertheless present in the glycoconjugate composition. The free saccharide may be noncovalently associated with (i.e., noncovalently bound to, adsorbed to, or entrapped in or with) the glycoconjugate.

In an embodiment, the serotype 15B glycoconjugate of the invention comprises less than about 50%, 45%, 40%, 35%, 30%, 25%, 20% or 15% of free serotype 15B capsular polysaccharide compared to the total amount of serotype 15B capsular polysaccharide. In another embodiment the serotype 15B glycoconjugate of the invention comprises less than about 25% of free serotype 15B capsular polysaccharide compared to the total amount of serotype 15B capsular polysaccharide. In yet another embodiment the serotype 15B glycoconjugate of the invention comprises less than about 20% of free serotype 15B capsular polysaccharide compared to the total amount of serotype 15B capsular polysaccharide. In still another embodiment the serotype 15B glycoconjugates of the invention comprises less than about 15% of free serotype 15B capsular polysaccharide compared to the total amount of serotype 15B capsular polysaccharide.

The serotype 15B glycoconjugates may also be characterized by their molecular size distribution (K_{d}). Size exclusion chromatography media (CL-4B) can be used to determine the relative molecular size distribution of the conjugate, as mentioned above.

In an embodiment, at least 20% of the serotype 15B glycoconjugates of the invention have a K_{d} below or equal to 0.3 in a CL-4B column. In another embodiment, at least 30% of the immunogenic conjugate has a K_{d} below or equal to 0.3 in a CL-4B column. In still another embodiment, at least 40% of the serotype 15B glycoconjugates of the invention have a K_{d} below or equal to 0.3 in a CL-4B column. In an embodiment, at least 45%, 50%, 55%, 60%, 65%, 70%, 75%, 80%, or 85% of the serotype 15 glycoconjugates of the invention have a K_{d} below or equal to 0.3 in a CL-4B column. In another embodiment, at least 60% of the serotype 15B glycoconjugates of the invention have a K_{d} below or equal to 0.3 in a CL-4B column. In still another embodiment, at least 70% of the serotype 15B glycoconjugates of the invention have a K_{d} below or equal to 0.3 in a CL-4B column.

In an embodiment, between 40% and 90% of the serotype 15B glycoconjugates have a K_{d} below or equal to 0.3 in a CL-4B column. In an embodiment, between 50% and 90% of the serotype 15B glycoconjugates have a K_{d} below or equal to 0.3 in a CL-4B column. In an embodiment, between 65% and 80% of the serotype 15B glycoconjugates have a K_{d} below or equal to 0.3 in a CL-4B column.

In yet another embodiment, the serotype 15B glycoconjugate of the invention comprises at least 0.1, 0.2, 0.3, 0.4, 0.5, 0.6, 0.7 or 0.8 mM acetate per mM serotype 15B capsular polysaccharide. In an embodiment, the glycoconjugate comprises at least 0.5, 0.6 or 0.7 mM acetate per mM serotype 15B capsular polysaccharide. In another embodiment, the glycoconjugate comprises at least 0.6 mM acetate per mM serotype 15B capsular polysaccharide. In still another embodiment, the glycoconjugate comprises at least 0.7 mM acetate per mM serotype 15B capsular polysaccharide. In yet another embodiment, the presence of O-acetyl groups is determined by ion-HPLC analysis.

In another embodiment, the ratio of mM acetate per mM serotype 15B capsular polysaccharide in the serotype 15B glycoconjugate to mM acetate per mM serotype 15B capsular polysaccharide in the isolated polysaccharide is at least 0.6, 0.65, 0.7, 0.75, 0.8, 0.85, 0.9, or 0.95. In yet another embodiment, the ratio of mM acetate per mM serotype 15B capsular polysaccharide in the serotype 15B glycoconjugate to mM acetate per mM serotype 15B capsular polysaccharide in the isolated polysaccharide is at least 0.7. In another embodiment, the ratio of mM acetate per mM serotype 15B capsular polysaccharide in the serotype 15B glycoconjugate to mM acetate per mM serotype 15B capsular polysaccharide in the isolated polysaccharide is at least 0.9. In still another embodiment, the presence of O-acetyl groups is determined by ion-HPLC analysis.

In an embodiment, the ratio of mM acetate per mM serotype 15B capsular polysaccharide in the serotype 15B glycoconjugate to mM acetate per mM serotype 15B capsular polysaccharide in the activated polysaccharide is at least 0.6, 0.65, 0.7, 0.75, 0.8, 0.85, 0.9, or 0.95. In another embodiment, the ratio of mM acetate per mM serotype 15B capsular polysaccharide in the serotype 15B glycoconjugate to mM acetate per mM serotype 15B capsular polysaccharide in the activated polysaccharide is at least 0.7. In yet another embodiment, the ratio of mM acetate per mM serotype 15B capsular polysaccharide in the serotype 15B glycoconjugate to mM acetate per mM serotype 15B capsular polysaccharide in the activated polysaccharide is at least 0.9. In an embodiment, the presence of O-acetyl groups is determined by ion-HPLC analysis.

In an embodiment, the serotype 15B glycoconjugate of the invention comprises at least 0.1, 0.2, 0.3, 0.4, 0.5, 0.6, 0.7 or 0.8 mM glycerol per mM serotype 15B capsular polysaccharide. In another embodiment, the serotype 15B glycoconjugate of the invention comprises at least 0.5, 0.6 or 0.7 mM glycerol per mM serotype 15B capsular polysaccharide. In still another embodiment, the serotype 15B glycoconjugate of the invention comprises at least 0.6 mM glycerol per mM serotype 15B capsular polysaccharide. In yet another embodiment, the serotype 15B glycoconjugate of the invention comprises at least 0.7 mM glycerol per mM serotype 15B capsular polysaccharide.

Another way to characterize the serotype 15B glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the CRM₁₉₇ protein starting material used to generate the conjugate materials.

In an embodiment, the degree of conjugation of the serotype 15B glycoconjugate of the invention is between 2 and 15, between 2 and 13, between 2 and 10, between 2 and 8, between 2 and 6, between 2 and 5, between 2 and 4, between 3 and 15, between 3 and 13, between 3 and 10, between 3 and 8, between 3 and 6, between 3 and 5, between 3 and 4, between 5 and 15, between 5 and 10, between 8 and 15, between 8 and 12, between 10 and 15 or between 10 and 12. In an embodiment, the degree of conjugation of the serotype 15B glycoconjugate of the invention is about 2, about 3, about 4, about 5, about 6, about 7, about 8, about 9, about 10, about 11, about 12, about 13, about 14 or about 15. In another embodiment, the degree of conjugation of the serotype 15B glycoconjugate of the invention is between 2 and 5.

1.3.7 Glycoconjugates from S. pneumoniae Serotype 15C

In an embodiment, the serotype 15C glycoconjugates are obtained by activating polysaccharide with 1-cyano-4-dimethylamino pyridinium tetrafluoroborate (CDAP) to form a cyanate ester. The activated polysaccharide may be coupled directly or via a spacer (linker) group to an amino group on the carrier protein. For example, the spacer could be cystamine or cysteamine to give a thiolated polysaccharide which could be coupled to the carrier via a thioether linkage obtained after reaction with a maleimide-activated carrier protein (for example using GMBS) or a haloacetylated carrier protein (for example using iodoacetimide, SIB, SIAB, sulfo-SIAB, SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO93/15760, WO95/08348 and WO96/129094.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO98/42721. Conjugation may involve a carbonyl linker which may be formed by reaction of a free hydroxyl group of the saccharide with CDI (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In one or more embodiments, the serotype 15C glycoconjugates of the invention are prepared using reductive amination. Reductive amination involves two steps: (1) oxidation of the polysaccharide to generate aldehyde functionalities from vicinal diols in individual hexasaccharide unit and (2) reduction of the activated polysaccharide and a carrier protein to form a conjugate.

Preferably, before oxidation, sizing of the serotype 15C polysaccharide to a target molecular weight (MW) range is performed. Advantageously, the size of the purified serotype 15C polysaccharide is reduced while preserving critical features of the structure of the polysaccharide such as for example the presence of O-acetyl groups. Preferably, the size of the purified serotype 15B polysaccharide is reduced by mechanical homogenization (see section 1.2.6 above).

The oxidation step may involve reaction with periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate). In an embodiment the periodate used for the oxidation of serotype 15C capsular polysaccharide is metaperiodate. In an embodiment the periodate used for the oxidation of serotype 15C capsular polysaccharide is sodium metaperiodate.

In an embodiment, the polysaccharide is reacted with 0.01 to 10.0, 0.05 to 5.0, 0.1 to 1.0, 0.5 to 1.0, 0.7 to 0.8, 0.05 to 0.5, 0.1 to 0.3 molar equivalents of oxidizing agent. In an embodiment, the polysaccharide is reacted with about 0.1, 0.15, 0.2, 0.25, 0.3, 0.35, 0.4, 0.45, 0.5, 0.55, 0.6, 0.65, 0.7, 0.75, 0.8, 0.85, 0.9, 0.95 molar equivalents of oxidizing agent. In another embodiment, the polysaccharide is reacted with about 0.15 molar equivalents of oxidizing agent. In yet another embodiment, the polysaccharide is reacted with about 0.25 molar equivalents of oxidizing agent. In still another embodiment, the polysaccharide is reacted with about 0.5 molar equivalents of oxidizing agent. In an embodiment, the polysaccharide is reacted with about 0.6 molar equivalents of oxidizing agent. In another embodiment, the polysaccharide is reacted with about 0.7 molar equivalents of oxidizing agent.

In an embodiment, the duration of the reaction is between 1 hour and 50 hours, between 10 hours and 30 hours, between 15 hours and 20 hours, between 15 hours and 17 hours or about 16 hours.

In another embodiment, the temperature of the reaction is maintained between 15° C. and 45° C., between 15° C. and 30° C., between 20° C. and 25° C. In yet another embodiment, the temperature of the reaction is maintained at about 23° C.

In another embodiment, the oxidation reaction is carried out in a buffer selected from sodium phosphate, potassium phosphate, 2-(N-morpholino) ethanesulfonic acid (MES) or Bis-Tris. In an embodiment, the buffer is potassium phosphate.

In yet another embodiment, the buffer has a concentration of between 1 mM and 500 mM, between 1 mM and 300 mM, or between 50 mM and 200 mM. In still another embodiment the buffer has a concentration of about 100 mM.

In an embodiment, the oxidation reaction is carried out at a pH between 4.0 and 8.0, between 5.0 and 7.0, or between 5.5 and 6.5. In another embodiment, the pH is about 6.0.

In an embodiment, the activated serotype 15C capsular polysaccharide is obtained by reacting 0.5 mg/mL to 5 mg/ml of isolated serotype 15C capsular polysaccharide with 0.2 to 0.3 molar equivalents of periodate at a temperature between 20° C. and 25° C.

In another embodiment, the activated serotype 15C capsular polysaccharide is purified. The activated serotype 15C capsular polysaccharide is purified according to methods known to the man skilled in the art, such as gel permeation chromatography (GPC), dialysis or ultrafiltration/diafiltration. For example, the activated capsular polysaccharide is purified by concentration and diafiltration using an ultrafiltration device.

In yet another embodiment, the degree of oxidation of the activated serotype 15C capsular polysaccharide is between 2 and 20, between 2 and 15, between 2 and 10, between 2 and 5, between 5 and 20, between 5 and 15, between 5 and 10, between 10 and 20, between 10 and 15, or between 15 and 20. In another embodiment the degree of oxidation of the activated serotype 15C capsular polysaccharide is between 2 and 10, between 4 and 8, between 4 and 6, between 6 and 8, between 6 and 12, between 8 and 12, between 9 and 11, between 10 and 16, between 12 and 16, between 14 and 18, between 16 and 20, between 16 and 18, or between 18 and 20.

In still another embodiment, the activated serotype 15C capsular polysaccharide has a molecular weight between 5 kDa and 500 kDa, between 50 kDa and 500 kDa, between 50 kDa and 450 kDa, between 100 kDa and 400 kDa, between 100 kDa and 350 kDa. In an embodiment, the activated serotype 15C capsular polysaccharide has a molecular weight between 100 kDa and 350 kDa. In still another embodiment, the activated serotype 15C capsular polysaccharide has a molecular weight between 100 kDa and 300 kDa. In another embodiment, the activated serotype 15C capsular polysaccharide has a molecular weight between 100 kDa and 250 kDa.

In an embodiment, the activated serotype 15C capsular polysaccharide comprises at least 0.1, 0.2, 0.3, 0.4, 0.5, 0.6, 0.7 or 0.8 mM acetate per mM of said serotype 15C capsular polysaccharide. In another embodiment, the activated serotype 15C capsular polysaccharide comprises at least 0.5, 0.6 or 0.7 mM acetate per mM of said serotype 15C capsular polysaccharide. In yet another embodiment, the activated serotype 15C capsular polysaccharide comprises at least 0.6 mM acetate per mM of said serotype 15C capsular polysaccharide. In still another embodiment, the activated serotype 15C capsular polysaccharide comprises at least 0.7 mM acetate per mM of said serotype 15C capsular polysaccharide.

In an embodiment, the activated serotype 15C capsular polysaccharide comprises at least 0.1, 0.2, 0.3, 0.4, 0.5, 0.6, 0.7 or 0.8 mM glycerol per mM of said serotype 15C capsular polysaccharide. In another embodiment, the activated serotype 15C capsular polysaccharide comprises at least 0.5, 0.6 or 0.7 mM glycerol per mM of said serotype 15C capsular polysaccharide. In yet another embodiment, the activated serotype 15C capsular polysaccharide comprises at least 0.6 mM glycerol per mM of said serotype 15C capsular polysaccharide. In still another embodiment, the activated serotype 15C capsular polysaccharide comprises at least 0.7 mM glycerol per mM of said serotype 15C capsular polysaccharide.

In an embodiment, the activated serotype 15C capsular polysaccharide has a molecular weight between 100 kDa and 250 kDa and comprises at least 0.6 mM acetate per mM of said serotype 15C capsular polysaccharide.

In another embodiment, the activated serotype 15C capsular polysaccharide has a molecular weight between 100 kDa and 250 kDa and comprises at least 0.6 mM glycerol per mM of said serotype 15C capsular polysaccharide.

In still another embodiment, the activated serotype 15C capsular polysaccharide comprises at least 0.6 mM acetate per mM of said serotype 15C capsular polysaccharide and at least 0.6 mM glycerol per mM of said serotype 15C capsular polysaccharide.

In yet another embodiment, the activated serotype 15C capsular polysaccharide has a molecular weight between 100 kDa and 250 kDa and comprises at least 0.6 mM acetate per mM of said serotype 15C capsular polysaccharide and at least 0.6 mM glycerol per mM of said serotype 15C capsular polysaccharide.

In an embodiment, the activated serotype 15C capsular polysaccharide is lyophilized, optionally in the presence of saccharide. In an embodiment, the saccharide is selected from sucrose, trehalose, raffinose, stachyose, melezitose, dextran, mannitol, lactitol and palatinit. In an embodiment, the saccharide is sucrose. The lyophilized activated capsular polysaccharide can then be compounded with a solution comprising the carrier protein.

In another embodiment, the activated serotype 15C capsular polysaccharide is compounded with the carrier protein and lyophilized optionally in the presence of a saccharide. In an embodiment, the saccharide is selected from sucrose, trehalose, raffinose, stachyose, melezitose, dextran, mannitol, lactitol and palatinit. In an embodiment, the saccharide is sucrose. The co-lyophilized polysaccharide and carrier protein can then be resuspended in solution and reacted with a reducing agent.

The activated serotype 15C capsular polysaccharide can be conjugated to a carrier protein by a process comprising the step of:
(a) compounding the activated serotype 15C capsular polysaccharide with a carrier protein, and
(b) reacting the compounded activated serotype 15C capsular polysaccharide and carrier protein with a reducing agent to form a serotype 15C capsular polysaccharide-carrier protein conjugate.

The conjugation of activated serotype 15C capsular polysaccharide with a protein carrier by reductive amination in dimethylsulfoxide (DMSO) is suitable to preserve the O-acetyl content of the polysaccharide as compared for example to reductive amination in aqueous solution where the level of O-acetylation of the polysaccharide is significantly reduced. In another embodiment, step (a) and step (b) are carried out in DMSO.

In an embodiment, step (a) comprises dissolving lyophilized serotype 15C capsular polysaccharide in a solution comprising a carrier protein and DMSO. In an embodiment, step (a) comprises dissolving co-lyophilized serotype 15C capsular polysaccharide and carrier protein in DMSO.

When steps (a) and (b) are carried out in aqueous solution, steps (a) and (b) are carried out in a buffer, preferably selected from PBS, MES, HEPES, Bis-tris, ADA, PIPES, MOPSO, BES, MOPS, DIPSO, MOBS, HEPPSO, POPSO, TEA, EPPS, Bicine or HEPB, at a pH between 6.0 and 8.5, between 7.0 and 8.0 or between 7.0 and 7.5. In an embodiment the buffer is PBS. In an embodiment the pH is about 7.3.

In an embodiment, the concentration of activated serotype 15C capsular polysaccharide in step (b) is between 0.1 mg/mL and 10 mg/mL, between 0.5 mg/mL and 5 mg/mL, or between 0.5 mg/mL and 2 mg/mL. In another embodiment, the concentration of activated serotype 15C capsular polysaccharide in step (b) is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 mg/mL.

In yet another embodiment the initial input ratio (weight by weight) of activated serotype 15C capsular polysaccharide to carrier protein is between 5:1 and 0.1:1, between 2:1 and 0.1:1, between 2:1 and 1:1, between 1.5:1 and 1:1, between 0.1:1 and 1:1, between 0.3:1 and 1:1, or between 0.6:1 and 1:1.

In still another embodiment the initial input ratio of activated serotype 15C capsular polysaccharide to carrier protein is about 0.6:1 to 1:1. In another embodiment the initial input ratio of activated serotype 15C capsular polysaccharide to carrier protein is about 0.6:1 to 1.5:1. Such initial input ratio is particularly suitable to obtain low levels of free polysaccharide in the glycoconjugate.

In an embodiment the initial input ratio of activated serotype 15C capsular polysaccharide to carrier protein is about 0.4:1, 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1 or 2:1.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In an embodiment, the reducing agent is sodium cyanoborohydride. In an embodiment, the reducing agent is sodium 2-Picoline Borane.

In another embodiment, the quantity of reducing agent used in step (b) is between about 0.1 and 10.0 molar equivalents, between 0.5 and 5.0 molar equivalents, or between 1.0 and 2.0 molar equivalents. In an embodiment, the quantity of reducing agent used in step (b) is about 1.0, 1.1, 1.2, 1.3, 1.4, 1.5, 1.6, 1.7, 1.8, 1.9 or 2.0 molar equivalents.

In yet another embodiment, the duration of step (b) is between 1 hour and 60 hours, between 10 hours and 50 hours, between 40 hours and 50 hours, or between 42 hours and 46 hours. In an embodiment, the duration of step (b) is about 44 hours.

In still another embodiment, the temperature of the reaction in step (b) is maintained between 10° C. and 40° C., between 15° C. and 30° C. or between 20° C. and 26° C. In another embodiment, the temperature of the reaction in step (b) is maintained at about 23° C.

In an embodiment, the process for the preparation of a glycoconjugate comprising S. pneumoniae serotype 15C capsular polysaccharide covalently linked to a carrier protein further comprises a step (step (c)) of capping unreacted aldehyde (quenching) by addition of NaBH₄.

In still another embodiment, the quantity of NaBH₄ used in step (c) is between 0.1 and 10 molar equivalents, between 0.5 and 5.0 molar equivalents or between 1.0 and 3.0 molar equivalents. In yet another embodiment, the quantity of NaBH₄ used in step (c) is about 2.0 molar equivalents.

In another embodiment, the duration of step (c) is between 0.1 hours and 10 hours, 0.5 hours and 5 hours, or between 2 hours and 4 hours. In an embodiment, the duration of step (c) is about 3 hours.

In another embodiment, the temperature of the reaction in step (c) is maintained between 15° C. and 45° C., between 15° C. and 30° C. or between 20° C. and 26° C. In still another embodiment, the temperature of the reaction in step (c) is maintained at about 23° C.

In another embodiment the yield of the conjugation step is greater than 50%, 55%, 60%, 65%, 70%, 75%, 80%, 85% or 90%. In still another embodiment the yield of the conjugation step (step b) is greater than 60%. In yet another embodiment the yield of the conjugation step (step b) is greater than 70%. The yield is the amount of serotype 15B polysaccharide in the conjugate×100)/amount of activated polysaccharide used in the conjugation step.

In an embodiment, the process for the preparation of a glycoconjugate comprising S. pneumoniae serotype 15C capsular polysaccharide covalently linked to a carrier protein comprises the steps of:
(a) sizing purified serotype 15C polysaccharide by high-pressure homogenization;
(b) reacting the sized serotype 15C polysaccharide with an oxidizing agent;
(c) compounding the activated serotype 15C polysaccharide with a carrier protein;
(d) reacting the compounded activated serotype 15C polysaccharide and carrier protein with a reducing agent to form a serotype 15C polysaccharide-carrier protein conjugate; and
(e) capping unreacted aldehyde (quenching) by addition of NaBH₄.

In still another embodiment, the yield of the conjugation step (step d) of the above process is greater than 50%, 55%, 60%, 65%, 70%, 75%, 80%, 85% or 90%. In another embodiment the yield of the conjugation step (step d) is greater than 60%. In still another embodiment the yield of the conjugation step (step d) is greater than 70%. The yield is the amount of serotype 15C polysaccharide in the conjugate×100)/amount of activated polysaccharide used in the conjugation step.

After conjugation of the serotype 15C capsular polysaccharide to the carrier protein, the polysaccharide-protein conjugate can be purified (enriched with respect to the amount of polysaccharide-protein conjugate) by a variety of techniques known to the skilled person. These techniques include dialysis, concentration/diafiltration operations, tangential flow filtration, precipitation/elution, column chromatography (DEAE or hydrophobic interaction chromatography), and depth filtration.

In an embodiment the carrier protein is as defined at section 1.1. In an embodiment the carrier protein is selected in the group consisting of: DT (Diphtheria toxin), TT (tetanus toxid), CRM₁₉₇, other DT mutants, PD (Haemophilus influenzae protein D), or immunologically functional equivalents thereof. In an embodiment the carrier protein is CRM₁₉₇.

In one or more embodiments, the serotype 15C glycoconjugates of the present invention are conjugated to the carrier protein (e.g., CRM₁₉₇) and comprise a saccharide having a molecular weight of between 5 kDa and 1,500 kDa. In other embodiments, the saccharide has a molecular weight of between 10 kDa and 1,500 kDa. In further such embodiments, the saccharide has a molecular weight of between 50 kDa and 1,500 kDa; between 50 kDa and 1,250 kDa; between 50 kDa and 1,000 kDa; between 50 kDa and 750 kDa; between 50 kDa and 500 kDa; between 50 kDa and 250 kDa; between 100 kDa and 1,500 kDa; between 100 kDa and 1,250 kDa; between 100 kDa and 1,000 kDa; between 100 kDa and 750 kDa; between 100 kDa and 500 kDa; between 100 kDa and 250 kDa; between 200 kDa and 1,500 kDa; between 200 kDa and 1,250 kDa; between 200 kDa and 1,000 kDa; between 200 kDa and 750 kDa; or between 200 kDa and 500 kDa; or between 200 kDa and 400 kDa. Any whole number integer within any of the above ranges is contemplated as an embodiment of the disclosure. In some embodiments, the serotype 15C glycoconjugate of the invention has a molecular weight of between 50 kDa and 20,000 kDa. In some embodiments, the serotype 15C glycoconjugate of the invention has a molecular weight of between 1,000 kDa and 20,000 kDa. In an embodiment, the serotype 15C glycoconjugate of the invention has a molecular weight between 3,000 kDa and 20,000 kDa, between 5,000 kDa and 10,000 kDa, between 5,000 kDa and 20,000 kDa, between 8,000 kDa and 20,000 kDa, between 8,000 kDa and 16,000 kDa or between 10,000 kDa and 16,000 kDa.

In one or more further embodiments, the serotype 15C glycoconjugate of the invention has a molecular weight of about 1,000 kDa, about 1,500 kDa, about 2,000 kDa, about 2,500 kDa, about 3,000 kDa, about 3,500 kDa, about 4,000 kDa, about 4,500 kDa, about 5,000 kDa, about 5,500 kDa, about 6,000 kDa, about 6,500 kDa, about 7,000 kDa, about 7,500 kDa, about 8,000 kDa, about 8,500 kDa, about 9,000 kDa, about 9,500 kDa about 10,000 kDa, about 10,500 kDa, about 11,000 kDa, about 11,500 kDa, about 12,000 kDa, about 12,500 kDa, about 13,000 kDa, about 13,500 kDa, about 14,000 kDa, about 14,500 kDa, about 15,000 kDa, about 15,500 kDa, about 16,000 kDa, about 16,500 kDa, about 17,000 kDa, about 17,500 kDa, about 18,000 kDa, about 18,500 kDa about 19,000 kDa, about 19,500 kDa or about 20,000 kDa.

In further embodiments, the serotype 15C glycoconjugate of the invention has a molecular weight of between 1,000 kDa and 20,000 kDa; between 1,000 kDa and 15,000 kDa; between 1,000 kDa and 10,000 kDa; between 1,000 kDa and 7,500 kDa; between 1,000 kDa and 5,000 kDa; between 1,000 kDa and 4,000 kDa; between 1,000 kDa and 3,000 kDa; between 2,000 kDa and 20,000 kDa; between 2,000 kDa and 15,000 kDa; between 2,000 kDa and 12,500 kDa; between 2,000 kDa and 10,000 kDa; between 2,000 kDa and 7,500 kDa; between 2,000 kDa and 6,000 kDa; between 2,000 kDa and 5,000 kDa; between 2,000 kDa and 4,000 kDa; or between 2,000 kDa and 3,000 kDa.

In further embodiments, the serotype 15C glycoconjugate of the invention has a molecular weight of between 3,000 kDa and 20,000 kDa; between 3,000 kDa and 15,000 kDa; between 3,000 kDa and 10,000 kDa; between 3,000 kDa and 7,500 kDa; between 3,000 kDa and 5,000 kDa; between 3,000 kDa and 4,000 kDa; between 4,000 kDa and 20,000 kDa; between 4,000 kDa and 15,000 kDa; between 4,000 kDa and 12,500 kDa; between 4,000 kDa and 10,000 kDa; between 4,000 kDa and 7,500 kDa; between 4,000 kDa and 6,000 kDa or between 4,000 kDa and 5,000 kDa. In further embodiments, the serotype 15C glycoconjugate of the invention has a molecular weight of between 5,000 kDa and 20,000 kDa; between 5,000 kDa and 15,000 kDa; between 5,000 kDa and 10,000 kDa; between 5,000 kDa and 7,500 kDa; between 6,000 kDa and 20,000 kDa; between 6,000 kDa and 15,000 kDa; between 6,000 kDa and 12,500 kDa; between 6,000 kDa and 10,000 kDa or between 6,000 kDa and 7,500 kDa.

The molecular weight of the glycoconjugate is measured by SEC-MALLS. Any whole number integer within any of the above ranges is contemplated as an embodiment of the disclosure. In an embodiment, said serotype 15C glycoconjugates are prepared using reductive amination.

The serotype 15C glycoconjugates of the invention may also be characterized by the ratio (weight/weight) of saccharide to carrier protein. In an embodiment, the ratio (weight by weight) of serotype 15C capsular polysaccharide to carrier protein in the conjugate is between 0.5 and 3.0 (e.g., about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9 or about 3.0). In an embodiment, the ratio of serotype 15C capsular polysaccharide to carrier protein in the conjugate is between 0.4 and 2. In an embodiment, the ratio of serotype 15C capsular polysaccharide to carrier protein in the conjugate is between 0.5 and 2.0, 0.5 and 1.5, 0.5 and 1.0, 1.0 and 1.5, 1.0 and 2.0. In an embodiment, the ratio of serotype 15C capsular polysaccharide to carrier protein in the conjugate is between 0.7 and 0.9.

The serotype 15C glycoconjugates and immunogenic compositions of the invention may contain free saccharide that is not covalently conjugated to the carrier protein, but is nevertheless present in the glycoconjugate composition. The free saccharide may be noncovalently associated with (i.e., noncovalently bound to, adsorbed to, or entrapped in or with) the glycoconjugate.

In an embodiment, the serotype 15C glycoconjugate of the invention comprises less than about 50%, 45%, 40%, 35%, 30%, 25%, 20% or 15% of free serotype 15C capsular polysaccharide compared to the total amount of serotype 15C capsular polysaccharide. In another embodiment the serotype 15C glycoconjugate of the invention comprises less than about 25% of free serotype 15C capsular polysaccharide compared to the total amount of serotype 15C capsular polysaccharide. In yet another embodiment the serotype 15C glycoconjugate of the invention comprises less than about 20% of free serotype 15C capsular polysaccharide compared to the total amount of serotype 15C capsular polysaccharide. In still another embodiment the serotype 15C glycoconjugates of the invention comprises less than about 15% of free serotype 15C capsular polysaccharide compared to the total amount of serotype 15C capsular polysaccharide.

The serotype 15C glycoconjugates may also be characterized by their molecular size distribution (K_{d}). Size exclusion chromatography media (CL-4B) can be used to determine the relative molecular size distribution of the conjugate, as mentioned above.

In an embodiment, at least 20% of the serotype 15C glycoconjugates of the invention have a K_{d} below or equal to 0.3 in a CL-4B column. In another embodiment, at least 30% of the immunogenic conjugate has a K_{d} below or equal to 0.3 in a CL-4B column. In still another embodiment, at least 40% of the serotype 15C glycoconjugates of the invention have a K_{d} below or equal to 0.3 in a CL-4B column. In an embodiment, at least 45%, 50%, 55%, 60%, 65%, 70%, 75%, 80%, or 85% of the serotype 15C glycoconjugates of the invention have a K_{d} below or equal to 0.3 in a CL-4B column. In another embodiment, at least 60% of the serotype 15C glycoconjugates of the invention have a K_{d} below or equal to 0.3 in a CL-4B column. In still another embodiment, at least 70% of the serotype 15B glycoconjugates of the invention have a K_{d} below or equal to 0.3 in a CL-4B column.

In an embodiment, between 40% and 90% of the serotype 15C glycoconjugates have a K_{d} below or equal to 0.3 in a CL-4B column. In an embodiment, between 50% and 90% of the serotype 15C glycoconjugates have a K_{d} below or equal to 0.3 in a CL-4B column. In an embodiment, between 65% and 80% of the serotype 15C glycoconjugates have a K_{d} below or equal to 0.3 in a CL-4B column.

In yet another embodiment, the serotype 15C glycoconjugate of the invention comprises at least 0.1, 0.2, 0.3, 0.4, 0.5, 0.6, 0.7 or 0.8 mM acetate per mM serotype 15C capsular polysaccharide. In an embodiment, the glycoconjugate comprises at least 0.5, 0.6 or 0.7 mM acetate per mM serotype 15C capsular polysaccharide. In another embodiment, the glycoconjugate comprises at least 0.6 mM acetate per mM serotype 15C capsular polysaccharide. In still another embodiment, the glycoconjugate comprises at least 0.7 mM acetate per mM serotype 15C capsular polysaccharide. In yet another embodiment, the presence of O-acetyl groups is determined by ion-HPLC analysis.

In another embodiment, the ratio of mM acetate per mM serotype 15C capsular polysaccharide in the serotype 15C glycoconjugate to mM acetate per mM serotype 15C capsular polysaccharide in the isolated polysaccharide is at least 0.6, 0.65, 0.7, 0.75, 0.8, 0.85, 0.9, or 0.95. In yet another embodiment, the ratio of mM acetate per mM serotype 15C capsular polysaccharide in the serotype 15C glycoconjugate to mM acetate per mM serotype 15C capsular polysaccharide in the isolated polysaccharide is at least 0.7. In another embodiment, the ratio of mM acetate per mM serotype 15C capsular polysaccharide in the serotype 15C glycoconjugate to mM acetate per mM serotype 15C capsular polysaccharide in the isolated polysaccharide is at least 0.9. In still another embodiment, the presence of O-acetyl groups is determined by ion-HPLC analysis.

In an embodiment, the ratio of mM acetate per mM serotype 15C capsular polysaccharide in the serotype 15C glycoconjugate to mM acetate per mM serotype 15C capsular polysaccharide in the activated polysaccharide is at least 0.6, 0.65, 0.7, 0.75, 0.8, 0.85, 0.9, or 0.95. In another embodiment, the ratio of mM acetate per mM serotype 15C capsular polysaccharide in the serotype 15C glycoconjugate to mM acetate per mM serotype 15C capsular polysaccharide in the activated polysaccharide is at least 0.7. In yet another embodiment, the ratio of mM acetate per mM serotype 15C capsular polysaccharide in the serotype 15C glycoconjugate to mM acetate per mM serotype 15C capsular polysaccharide in the activated polysaccharide is at least 0.9. In an embodiment, the presence of O-acetyl groups is determined by ion-HPLC analysis.

In an embodiment, the serotype 15C glycoconjugate of the invention comprises at least 0.1, 0.2, 0.3, 0.4, 0.5, 0.6, 0.7 or 0.8 mM glycerol per mM serotype 15C capsular polysaccharide. In another embodiment, the serotype 15C glycoconjugate of the invention comprises at least 0.5, 0.6 or 0.7 mM glycerol per mM serotype 15C capsular polysaccharide. In still another embodiment, the serotype 15C glycoconjugate of the invention comprises at least 0.6 mM glycerol per mM serotype 15C capsular polysaccharide. In yet another embodiment, the serotype 15C glycoconjugate of the invention comprises at least 0.7 mM glycerol per mM serotype 15C capsular polysaccharide.

Another way to characterize the serotype 15C glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the CRM₁₉₇ protein starting material used to generate the conjugate materials.

In an embodiment, the degree of conjugation of the serotype 15C glycoconjugate of the invention is between 2 and 15, between 2 and 13, between 2 and 10, between 2 and 8, between 2 and 6, between 2 and 5, between 2 and 4, between 3 and 15, between 3 and 13, between 3 and 10, between 3 and 8, between 3 and 6, between 3 and 5, between 3 and 4, between 5 and 15, between 5 and 10, between 8 and 15, between 8 and 12, between 10 and 15 or between 10 and 12. In an embodiment, the degree of conjugation of the serotype 15C glycoconjugate of the invention is about 2, about 3, about 4, about 5, about 6, about 7, about 8, about 9, about 10, about 11, about 12, about 13, about 14 or about 15. In another embodiment, the degree of conjugation of the serotype 15C glycoconjugate of the invention is between 2 and 5.

1.3.8 Glycoconjugates from S. pneumoniae Serotype 16F

In an embodiment, the serotype 16F glycoconjugates are obtained by activating polysaccharide with 1-cyano-4-dimethylamino pyridinium tetrafluoroborate (CDAP) to form a cyanate ester. The activated polysaccharide may be coupled directly or via a spacer (linker) group to an amino group on the carrier protein. For example, the spacer may be cystamine or cysteamine to give a thiolated polysaccharide which may be coupled to the carrier via a thioether linkage obtained after reaction with a maleimide-activated carrier protein (for example using GMBS) or a haloacetylated carrier protein (for example using iodoacetimide, SIB, SIAB, sulfo-SIAB, SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO93/15760, WO95/08348 and WO96/129094.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO98/42721. Conjugation may involve a carbonyl linker which may be formed by reaction of a free hydroxyl group of the saccharide with CDI (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In one or more embodiments, the serotype 16F glycoconjugates of the invention are prepared using reductive amination. Reductive amination involves two steps: (1) oxidation of the polysaccharide to generate aldehyde functionalities from vicinal diols in individual hexasaccharide unit and (2) reduction of the activated polysaccharide and a carrier protein (e.g., CRM₁₉₇) to form a conjugate.

In an embodiment, before oxidation, sizing of the serotype 16F polysaccharide to a target molecular weight (MW) range is performed. Advantageously, the size of the purified serotype 16F polysaccharide is reduced while preserving critical features of the structure of the polysaccharide such as for example the presence of O-acetyl groups. In an embodiment, the size of the purified serotype 16F polysaccharide is reduced by mechanical homogenization as described herein.

In an embodiment, serotype polysaccharide is activated (oxidized) by a process comprising the step of:
(a) reacting isolated serotype 16F polysaccharide with an oxidizing agent; and
(b) quenching the oxidation reaction by addition of a quenching agent resulting in an activated serotype 16F polysaccharide.

In an embodiment, the oxidizing agent is periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate). In an embodiment, the oxidizing agent is sodium periodate. In an embodiment, the periodate used for the oxidation of serotype 16F polysaccharide is metaperiodate. In another embodiment, the periodate used for the oxidation of serotype 16F polysaccharide is sodium metaperiodate.

In one embodiment, the quenching agent is selected from vicinal diols, 1,2-aminoalcohols, amino acids, glutathione, sulfite, bisulfate, dithionite, metabisulfite, thiosulfate, phosphites, hypophosphites or phosphorous acid.

In one embodiment, the quenching agent is a 1,2-aminoalcohols of formula (I):

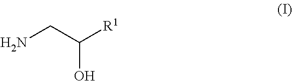

wherein R¹ is selected from H, methyl, ethyl, propyl or isopropyl.

In one embodiment, the quenching agent is selected from sodium and potassium salts of sulfite, bisulfate, dithionite, metabisulfite, thiosulfate, phosphites, hypophosphites or phosphorous acid.

In one embodiment, the quenching agent is an amino acid. In such embodiments, said amino acid may be selected from serine, threonine, cysteine, cystine, methionine, proline, hydroxyproline, tryptophan, tyrosine, and histidine.

In one embodiment, the quenching agent is a sulfite such as bisulfate, dithionite, metabisulfite, thiosulfate.

In one embodiment, the quenching agent is a compound comprising two vicinal hydroxyl groups (vicinal diols), i.e., two hydroxyl groups covalently linked to two adjacent carbon atoms.

In one or more embodiments, the quenching agent is a compound of formula (II):

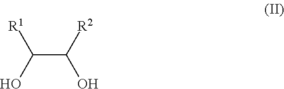

wherein R¹ and R² are each independently selected from H, methyl, ethyl, propyl or isopropyl.

In an embodiment, the quenching agent is glycerol, ethylene glycol, propan-1,2-diol, butan-1,2-diol or butan-2,3-diol, or ascorbic acid. In an embodiment, the quenching agent is butan-2,3-diol.

In another embodiment, the isolated serotype 16F polysaccharide is activated by a process comprising the steps of:
(a) reacting isolated serotype 16F polysaccharide with periodate; and
(b) quenching the oxidation reaction by addition of butan-2,3-diol resulting in an activated serotype 16F polysaccharide.

Following the oxidation step of the polysaccharide, the polysaccharide is said to be activated and is referred to as “activated polysaccharide” herein.

In an embodiment, the activated serotype 16F polysaccharide is purified. The activated serotype 16F polysaccharide is purified according to methods known to one skilled in the art such as gel permeation chromatography (GPC), dialysis or ultrafiltration/diafiltration. For example, the activated 16F polysaccharide is purified by concentration and diafiltration using an ultrafiltration device.

In another embodiment, the degree of oxidation of the activated serotype 16F polysaccharide is between 2 and 30, between 2 and 25, between 2 and 20, between 2 and 15, between 2 and 10, between 2 and 5, between 5 and 30, between 5 and 25, between 5 and 20, between 5 and 15, between 5 and 10, between 10 and 30, between 10 and 25, between 10 and 20, between 10 and 15, between 15 and 30, between 15 and 25, between 15 and 20, between 20 to 30, or between 20 to 25. In an embodiment the degree of oxidation of the activated serotype 16F polysaccharide is between 2 and 10, between 4 and 8, between 4 and 6, between 6 and 8, between 6 and 12, between 8 and 14, between 9 and 11, between 10 and 16, between 12 and 16, between 14 and 18, between 16 and 20, between 16 and 18, between 18 and 22, or between 18 and 20.

In an embodiment, the activated serotype 16F polysaccharide has a molecular weight between 25 kDa and 1,000 kDa, between 100 kDa and 1,000 kDa, between 300 kDa and 800 kDa, between 300 kDa and 700 kDa, between 300 kDa and 600 kDa, between 400 kDa and 1,000 kDa, between 400 kDa and 800 kDa, between 400 kDa and 700 kDa or between 400 kDa and 600 kDa. In an embodiment, the activated serotype 16F polysaccharide has a molecular weight between 300 kDa and 800 kDa. In an embodiment, the activated serotype 16F polysaccharide has a molecular weight between 400 kDa and 600 kDa. In another embodiment, the activated serotype 16F polysaccharide has a molecular weight between 400 kda and 600 kDa and a degree of oxidation between 10 and 25, between 10 and 20, between 12 and 20 or between 14 and 18. In another embodiment, the activated serotype 16F polysaccharide has a molecular weight between 400 kDa and 600 kDa and a degree of oxidation between 10 and 20.

In another embodiment, the activated serotype 16F polysaccharide comprises at least 0.1, 0.2, 0.3, 0.4, 0.5, 0.6 or 0.7 or about 0.8 mM acetate per mM serotype 16F polysaccharide. In an embodiment, the activated serotype 16F polysaccharide comprises at least 0.5, 0.6 or 0.7 mM acetate per mM serotype 16F polysaccharide. In another embodiment, the activated serotype 16F polysaccharide comprises at least 0.6 mM acetate per mM serotype 16F polysaccharide. In another embodiment, the activated serotype 16F polysaccharide comprises at least 0.7 mM acetate per mM serotype 16F polysaccharide.

In an embodiment, the activated serotype 16F polysaccharide has a molecular weight between 400 kDa and 800 kDa and comprises at least 0.6 mM acetate per mM serotype 16F polysaccharide.

In an embodiment, the activated serotype 16F polysaccharide has a molecular weight between 400 kDa and 800 kDa, a degree of oxidation between 12 and 20 and comprises at least 0.6 mM acetate per mM serotype 16F polysaccharide.

The activated polysaccharide and/or the carrier protein may be lyophilised (freeze-dried), either independently (discrete lyophilization) or together (co-lyophilized).

In another embodiment, the activated serotype 16F polysaccharide is lyophilized, optionally in the presence of saccharide. In an embodiment, the saccharide is selected from sucrose, trehalose, raffinose, stachyose, melezitose, dextran, mannitol, lactitol and palatinit. In an embodiment, the saccharide is sucrose. In one embodiment, the lyophilized activated polysaccharide is then compounded with a solution comprising the carrier protein.

In another embodiment, the activated polysaccharide and the carrier protein are co-lyophilised. In such embodiments, the activated serotype 16F polysaccharide is compounded with the carrier protein and lyophilized optionally in the presence of a saccharide. In an embodiment, the saccharide is selected from sucrose, trehalose, raffinose, stachyose, melezitose, dextran, mannitol, lactitol and palatinit. In an embodiment, the saccharide is sucrose. The co-lyophilized polysaccharide and carrier protein can then be resuspended in solution and reacted with a reducing agent.

The second step of the conjugation process is the reduction of the activated polysaccharide and a carrier protein to form a conjugate (reductive amination), using a reducing agent.

The activated serotype 16F polysaccharide can be conjugated to a carrier protein by a process comprising the steps of:
(c) compounding the activated serotype 16F polysaccharide with a carrier protein; and
(d) reacting the compounded activated serotype 16F polysaccharide and carrier protein with a reducing agent to form a serotype 16F polysaccharide-carrier protein conjugate.

In an embodiment, the reduction reaction is carried out in aqueous solvent. In another embodiment the reaction is carried out in aprotic solvent. In an embodiment, the reduction reaction is carried out in DMSO (dimethylsulfoxide) or in DMF (dimethylformamide)) solvent. The DMSO or DMF solvent may be used to reconstitute the activated polysaccharide and carrier protein which has been lyophilised.

The conjugation of activated serotype 16F polysaccharide with a protein carrier by reductive amination in dimethylsulfoxide (DMSO) is suitable to preserve the O-acetyl content of the polysaccharide as compared, for example, to reductive amination in aqueous phase where the level of O-acetylation of the polysaccharide may be significantly reduced. Therefore, in one or more embodiments, step (c) and step (d) are carried out in DMSO.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In an embodiment, the reducing agent is sodium cyanoborohydride.

At the end of the reduction reaction, there may be unreacted aldehyde groups remaining in the conjugates, these may be capped using a suitable capping agent. In one embodiment this capping agent is sodium borohydride (NaBH₄).

Following conjugation of serotype 16F polysaccharide to the carrier protein, the glycoconjugate can be purified (enriched with respect to the amount of polysaccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

In some embodiments, the serotype 16F glycoconjugates of the present invention comprise a saccharide having a molecular weight of between 10 kDa and 2,000 kDa. In other such embodiments, the saccharide has a molecular weight of between 50 kDa and 1,000 kDa. In other such embodiments, the saccharide has a molecular weight of between 70 kDa and 900 kDa. In other such embodiments, the saccharide has a molecular weight of between 100 kDa and 800 kDa. In other such embodiments, the saccharide has a molecular weight of between 200 kDa and 600 kDa. In further such embodiments, the saccharide has a molecular weight of 100 kDa to 1,000 kDa; 100 kDa to 900 kDa; 100 kDa to 800 kDa; 100 kDa to 700 kDa; 100 kDa to 600 kDa; 100 kDa to 500 kDa; 100 kDa to 400 kDa; 100 kDa to 300 kDa; 150 kDa to 1,000 kDa; 150 kDa to 900 kDa; 150 kDa to 800 kDa; 150 kDa to 700 kDa; 150 kDa to 600 kDa; 150 kDa to 500 kDa; 150 kDa to 400 kDa; 150 kDa to 300 kDa; 200 kDa to 1,000 kDa; 200 kDa to 900 kDa; 200 kDa to 800 kDa; 200 kDa to 700 kDa; 200 kDa to 600 kDa; 200 kDa to 500 kDa; 200 kDa to 400 kDa; 200 kDa to 300 kDa; 250 kDa to 1,000 kDa; 250 kDa to 900 kDa; 250 kDa to 800 kDa; 250 kDa to 700 kDa; 250 kDa to 600 kDa; 250 kDa to 500 kDa; 250 kDa to 400 kDa; 250 kDa to 350 kDa; 300 kDa to 1000 kDa; 300 kDa to 900 kDa; 300 kDa to 800 kDa; 300 kDa to 700 kDa; 300 kDa to 600 kDa; 300 kDa to 500 kDa; 300 kDa to 400 kDa; 400 kDa to 1,000 kDa; 400 kDa to 900 kDa; 400 kDa to 800 kDa; 400 kDa to 700 kDa; 400 kDa to 600 kDa; 500 kDa to 600 kDa. Any whole number integer within any of the above ranges is contemplated as an embodiment of the disclosure. In some such embodiments, the serotype 16F glycoconjugates are prepared using reductive amination.

In some embodiments, the serotype 16F glycoconjugate of the invention has a molecular weight of between 400 kDa and 15,000 kDa; between 500 kDa and 10,000 kDa; between 2,000 kDa and 10,000 kDa; between 3,000 kDa and 8,000 kDa; or between 3,000 kDa and 5,000 kDa. In other embodiments, the serotype 16F glycoconjugate has a molecular weight of between 500 kDa and 10,000 kDa. In other embodiments, the serotype 16F glycoconjugate has a molecular weight of between 1,000 kDa and 8,000 kDa. In still other embodiments, the serotype 16F glycoconjugate has a molecular weight of between 2,000 kDa and 8,000 kDa or between 3,000 kDa and 7,000 kDa. In further embodiments, the serotype 16F glycoconjugate of the invention has a molecular weight of between 200 kDa and 20,000 kDa; between 200 kDa and 15,000 kDa; between 200 kDa and 10,000 kDa; between 200 kDa and 7,500 kDa; between 200 kDa and 5,000 kDa; between 200 kDa and 3,000 kDa; between 200 kDa and 1,000 kDa; between 500 kDa and 20,000 kDa; between 500 kDa and 15,000 kDa; between 500 kDa and 12,500 kDa; between 500 kDa and 10,000 kDa; between 500 kDa and 7,500 kDa; between 500 kDa and 6,000 kDa; between 500 kDa and 5,000 kDa; between 500 kDa and 4,000 kDa; between 500 kDa and 3,000 kDa; between 500 kDa and 2,000 kDa; between 500 kDa and 1,500 kDa; between 500 kDa and 1,000 kDa; between 750 kDa and 20,000 kDa; between 750 kDa and 15,000 kDa; between 750 kDa and 12,500 kDa; between 750 kDa and 10,000 kDa; between 750 kDa and 7,500 kDa; between 750 kDa and 6,000 kDa; between 750 kDa and 5,000 kDa; between 750 kDa and 4,000 kDa; between 750 kDa and 3,000 kDa; between 750 kDa and 2,000 kDa; between 750 kDa and 1,500 kDa; between 1,000 kDa and 15,000 kDa; between 1,000 kDa and 12,500 kDa; between 1,000 kDa and 10,000 kDa; between 1,000 kDa and 7,500 kDa; between 1,000 kDa and 6,000 kDa; between 1,000 kDa and 5,000 kDa; between 1,000 kDa and 4,000 kDa; between 1,000 kDa and 2,500 kDa; between 2,000 kDa and 15,000 kDa; between 2,000 kDa and 12,500 kDa; between 2,000 kDa and 10,000 kDa; between 2,000 kDa and 7,500 kDa; between 2,000 kDa and 6,000 kDa; between 2,000 kDa and 5,000 kDa; between 2,000 kDa and 4,000 kDa; or between 2,000 kDa and 3,000 kDa.

In further embodiments, the serotype 16F glycoconjugate of the invention has a molecular weight of between 3,000 kDa and 20,000 kDa; between 3,000 kDa and 15,000 kDa; between 3,000 kDa and 10,000 kDa; between 3,000 kDa and 7,500 kDa; between 3,000 kDa and 5,000 kDa; between 4,000 kDa and 20,000 kDa; between 4,000 kDa and 15,000 kDa; between 4,000 kDa and 12,500 kDa; between 4,000 kDa and 10,000 kDa; between 4,000 kDa and 7,500 kDa; between 4,000 kDa and 6,000 kDa; or between 4,000 kDa and 5,000 kDa.

In further embodiments, the serotype 16F glycoconjugate of the invention has a molecular weight of between 5,000 kDa and 20,000 kDa; between 5,000 kDa and 15,000 kDa; between 5,000 kDa and 10,000 kDa; between 5,000 kDa and 7,500 kDa; between 6,000 kDa and 20,000 kDa; between 6,000 kDa and 15,000 kDa; between 6,000 kDa and 12,500 kDa; between 6,000 kDa and 10,000 kDa or between 6,000 kDa and 7,500 kDa.

The molecular weight of the glycoconjugate is measured by SEC-MALLS. Any whole number integer within any of the above ranges is contemplated as an embodiment of the disclosure.

In an embodiment, the serotype 16F glycoconjugate of the invention comprises at least 0.1, 0.2, 0.3, 0.4, 0.5, 0.6 or 0.7 or about 0.8 mM acetate per mM serotype 16F polysaccharide. In an embodiment, the glycoconjugate comprises at least 0.5, 0.6 or 0.7 mM acetate per mM serotype 16F polysaccharide. In an embodiment, the glycoconjugate comprises at least 0.6 mM acetate per mM serotype 16F polysaccharide. In another embodiment, the glycoconjugate comprises at least 0.7 mM acetate per mM serotype 16F polysaccharide.

In another embodiment, the ratio of mM acetate per mM serotype 16F polysaccharide in the glycoconjugate to mM acetate per mM serotype 16F polysaccharide in the isolated polysaccharide is at least 0.6, 0.65, 0.7, 0.75, 0.8, 0.85, 0.9, or 0.95. In an embodiment, the ratio of mM acetate per mM serotype 16F polysaccharide in the glycoconjugate to mM acetate per mM serotype 16F polysaccharide in the isolated polysaccharide is at least 0.7. In another embodiment, the ratio of mM acetate per mM serotype 16F polysaccharide in the glycoconjugate to mM acetate per mM serotype 16F polysaccharide in the isolated polysaccharide is at least 0.9.

In an embodiment, the ratio of mM acetate per mM serotype 16F polysaccharide in the glycoconjugate to mM acetate per mM serotype 16F polysaccharide in the activated polysaccharide is at least 0.6, 0.65, 0.7, 0.75, 0.8, 0.85, 0.9, or 0.95. In an embodiment, the ratio of mM acetate per mM serotype 16F polysaccharide in the glycoconjugate to mM acetate per mM serotype 16F polysaccharide in the activated polysaccharide is at least 0.7. In another embodiment, the ratio of mM acetate per mM serotype 16F polysaccharide in the glycoconjugate to mM acetate per mM serotype 16F polysaccharide in the activated polysaccharide is at least 0.9.

Another way to characterize the serotype 16F glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the CRM₁₉₇ protein starting material used to generate the conjugate materials. In an embodiment, the degree of conjugation of the serotype 16F glycoconjugate of the invention is between 2 and 15, between 2 and 13, between 2 and 10, between 2 and 8, between 2 and 6, between 2 and 5, between 2 and 4, between 3 and 15, between 3 and 13, between 3 and 10, between 3 and 8, between 3 and 6, between 3 and 5, between 3 and 4, between 5 and 15, between 5 and 10, between 8 and 15, between 8 and 12, between 10 and 15 or between 10 and 12. In an embodiment, the degree of conjugation of the serotype 16F glycoconjugate of the invention is about 2, about 3, about 4, about 5, about 6, about 7, about 8, about 9, about 10, about 11, about 12, about 13, about 14 or about 15. In an embodiment, the degree of conjugation of the serotype 16F glycoconjugate of the invention is between 4 and 7. In some such embodiments, the carrier protein is CRM₁₉₇.

The serotype 16F glycoconjugates of the invention may also be characterized by the ratio (weight/weight) of saccharide to carrier protein. In some embodiments, the ratio of serotype 16F polysaccharide to carrier protein in the glycoconjugate (w/w) is between 0.5 and 3.0 (e.g., about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0). In other embodiments, the saccharide to carrier protein ratio (w/w) is between 0.5 and 2.0, between 0.5 and 1.5, between 0.8 and 1.2, between 0.5 and 1.0, between 1.0 and 1.5 or between 1.0 and 2.0. In further embodiments, the saccharide to carrier protein ratio (w/w) is between 0.8 and 1.2. In an embodiment, the ratio of serotype 16F capsular polysaccharide to carrier protein in the conjugate is between 0.9 and 1.1. In some such embodiments, the carrier protein is CRM₁₉₇.

The serotype 16F glycoconjugates and immunogenic compositions of the invention may contain free saccharide that is not covalently conjugated to the carrier protein, but is nevertheless present in the glycoconjugate composition. The free saccharide may be noncovalently associated with (i.e., noncovalently bound to, adsorbed to, or entrapped in or with) the glycoconjugate.

In an embodiment, the serotype 16F glycoconjugate comprises less than about 50%, 45%, 40%, 35%, 30%, 25%, 20% or 15% of free serotype 16F polysaccharide compared to the total amount of serotype 16F polysaccharide. In another embodiment, the serotype 16F glycoconjugate comprises less than about 40% of free serotype 16F polysaccharide compared to the total amount of serotype 16F polysaccharide. In an embodiment, the serotype 16F glycoconjugate comprises less than about 25% of free serotype 16F polysaccharide compared to the total amount of serotype 16F polysaccharide. In an embodiment, the serotype 16F glycoconjugate comprises less than about 20% of free serotype 16F polysaccharide compared to the total amount of serotype 16F polysaccharide. In another embodiment the serotype 16F glycoconjugate comprises less than about 15% of free serotype 16F polysaccharide compared to the total amount of serotype 16F polysaccharide.

The serotype 16F glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In an embodiment, at least 30% of the serotype 16F glycoconjugate has a K_{d} below or equal to 0.3 in a CL-4B column. In another embodiment, at least 40% of the glycoconjugate has a K_{d} below or equal to 0.3 in a CL-4B column. In another embodiment, at least 45%, 50%, 55%, 60%, 65%, 70%, 75%, 80%, or 85% of the serotype 16F glycoconjugate has a K_{d} below or equal to 0.3 in a CL-4B column. In an embodiment, at least 60% of the serotype 16F glycoconjugate has a K_{d} below or equal to 0.3 in a CL-4B column. In another embodiment, between 50% and 80% of the serotype 16F glycoconjugate has a K_{d} below or equal to 0.3 in a CL-4B column. In an embodiment, between 65% and 80% of the serotype 16F glycoconjugate has a K_{d} below or equal to 0.3 in a CL-4B column.

1.3.9 Glycoconjugates from S. pneumoniae Serotype 17F

In an embodiment, the serotype 17F glycoconjugates are obtained by activating polysaccharide with 1-cyano-4-dimethylamino pyridinium tetrafluoroborate (CDAP) to form a cyanate ester. The activated polysaccharide may be coupled directly or via a spacer (linker) group to an amino group on the carrier protein. For example, the spacer may be cystamine or cysteamine to give a thiolated polysaccharide which may be coupled to the carrier via a thioether linkage obtained after reaction with a maleimide-activated carrier protein (for example using GMBS) or a haloacetylated carrier protein (for example using iodoacetimide, SIB, SIAB, sulfo-SIAB, SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO93/15760, WO95/08348 and WO96/129094.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO98/42721. Conjugation may involve a carbonyl linker which may be formed by reaction of a free hydroxyl group of the saccharide with CDI (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In one or more embodiments, the serotype 17F glycoconjugates of the invention are prepared using reductive amination. Reductive amination involves two steps: (1) oxidation of the polysaccharide to generate aldehyde functionalities from vicinal diols in individual hexasaccharide unit and (2) reduction of the activated polysaccharide and a carrier protein (e.g., CRM₁₉₇) to form a conjugate.

In an embodiment, before oxidation, sizing of the serotype 17F polysaccharide to a target molecular weight (MW) range is performed. Advantageously, the size of the purified serotype 17F polysaccharide is reduced while preserving critical features of the structure of the polysaccharide such as for example the presence of O-acetyl groups. In an embodiment, the size of the purified serotype 17F polysaccharide is reduced by mechanical homogenization as described herein.

In an embodiment, serotype polysaccharide is activated (oxidized) by a process comprising the step of:
(a) reacting isolated serotype 17F polysaccharide with an oxidizing agent; and
(b) quenching the oxidation reaction by addition of a quenching agent resulting in an activated serotype 17F polysaccharide.

In an embodiment, the oxidizing agent is periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate). In an embodiment, the oxidizing agent is sodium periodate. In an embodiment, the periodate used for the oxidation of serotype 17F polysaccharide is metaperiodate. In another embodiment, the periodate used for the oxidation of serotype 17F polysaccharide is sodium metaperiodate.

In one embodiment, the quenching agent is selected from vicinal diols, 1,2-aminoalcohols, amino acids, glutathione, sulfite, bisulfate, dithionite, metabisulfite, thiosulfate, phosphites, hypophosphites or phosphorous acid.

In one embodiment, the quenching agent is a 1,2-aminoalcohols of formula (I):

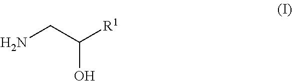

wherein R¹ is selected from H, methyl, ethyl, propyl or isopropyl.

In one embodiment, the quenching agent is selected from sodium and potassium salts of sulfite, bisulfate, dithionite, metabisulfite, thiosulfate, phosphites, hypophosphites or phosphorous acid.

In one embodiment, the quenching agent is an amino acid. In such embodiments, said amino acid may be selected from serine, threonine, cysteine, cystine, methionine, proline, hydroxyproline, tryptophan, tyrosine, and histidine.

In one embodiment, the quenching agent is a sulfite such as bisulfate, dithionite, metabisulfite, thiosulfate.

In one embodiment, the quenching agent is a compound comprising two vicinal hydroxyl groups (vicinal diols), i.e., two hydroxyl groups covalently linked to two adjacent carbon atoms.

In one or more embodiments, the quenching agent is a compound of formula (II):

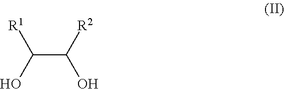

wherein R¹ and R² are each independently selected from H, methyl, ethyl, propyl or isopropyl.

In an embodiment, the quenching agent is glycerol, ethylene glycol, propan-1,2-diol, butan-1,2-diol or butan-2,3-diol, or ascorbic acid. In an embodiment, the quenching agent is butan-2,3-diol.

In another embodiment, the isolated serotype 17F polysaccharide is activated by a process comprising the steps of:
(a) reacting isolated serotype 17F polysaccharide with periodate; and
(b) quenching the oxidation reaction by addition of butan-2,3-diol resulting in an activated serotype 17F polysaccharide.

Following the oxidation step of the polysaccharide, the polysaccharide is said to be activated and is referred to as “activated polysaccharide” herein.

In an embodiment, the activated serotype 17F polysaccharide is purified. The activated serotype 17F polysaccharide is purified according to methods known to one skilled in the art such as gel permeation chromatography (GPC), dialysis or ultrafiltration/diafiltration. For example, the activated 17F polysaccharide is purified by concentration and diafiltration using an ultrafiltration device.

In another embodiment, the degree of oxidation of the activated serotype 17F polysaccharide is between 2 and 30, between 2 and 25, between 2 and 20, between 2 and 15, between 2 and 10, between 2 and 5, between 5 and 30, between 5 and 25, between 5 and 20, between 5 and 15, between 5 and 10, between 10 and 30, between 10 and 25, between 10 and 20, between 10 and 15, between 15 and 30, between 15 and 25, between 15 and 20, between 20 to 30, or between 20 to 25. In an embodiment the degree of oxidation of the activated serotype 17F polysaccharide is between 2 and 10, between 4 and 8, between 4 and 6, between 6 and 8, between 6 and 12, between 8 and 14, between 9 and 11, between 10 and 16, between 12 and 16, between 14 and 18, between 16 and 20, between 16 and 18, between 18 and 22, or between 18 and 20.

In an embodiment, the activated serotype 17F polysaccharide has a molecular weight between 25 kDa and 1,000 kDa, between 100 kDa and 1,000 kDa, between 300 kDa and 800 kDa, between 300 kDa and 700 kDa, between 300 kDa and 600 kDa, between 400 kDa and 1,000 kDa, between 400 kDa and 800 kDa, between 400 kDa and 700 kDa or between 400 kDa and 600 kDa. In an embodiment, the activated serotype 17F polysaccharide has a molecular weight between 300 kDa and 800 kDa. In an embodiment, the activated serotype 17F polysaccharide has a molecular weight between 400 kDa and 600 kDa. In another embodiment, the activated serotype 17F polysaccharide has a molecular weight between 400 kda and 600 kDa and a degree of oxidation between 10 and 25, between 10 and 20, between 12 and 20 or between 14 and 18. In another embodiment, the activated serotype 17F polysaccharide has a molecular weight between 400 kDa and 600 kDa and a degree of oxidation between 10 and 20.

In another embodiment, the activated serotype 17F polysaccharide comprises at least 0.1, 0.2, 0.3, 0.4, 0.5, 0.6 or 0.7 or about 0.8 mM acetate per mM serotype 17F polysaccharide. In an embodiment, the activated serotype 17F polysaccharide comprises at least 0.5, 0.6 or 0.7 mM acetate per mM serotype 17F polysaccharide. In another embodiment, the activated serotype 17F polysaccharide comprises at least 0.6 mM acetate per mM serotype 17F polysaccharide. In another embodiment, the activated serotype 17F polysaccharide comprises at least 0.7 mM acetate per mM serotype 17F polysaccharide.

In an embodiment, the activated serotype 17F polysaccharide has a molecular weight between 400 kDa and 800 kDa and comprises at least 0.6 mM acetate per mM serotype 17F polysaccharide.

In an embodiment, the activated serotype 17F polysaccharide has a molecular weight between 400 kDa and 800 kDa, a degree of oxidation between 12 and 20 and comprises at least 0.6 mM acetate per mM serotype 17F polysaccharide.

The activated polysaccharide and/or the carrier protein may be lyophilised (freeze-dried), either independently (discrete lyophilization) or together (co-lyophilized).

In another embodiment, the activated serotype 17F polysaccharide is lyophilized, optionally in the presence of saccharide. In an embodiment, the saccharide is selected from sucrose, trehalose, raffinose, stachyose, melezitose, dextran, mannitol, lactitol and palatinit. In an embodiment, the saccharide is sucrose. In one embodiment, the lyophilized activated polysaccharide is then compounded with a solution comprising the carrier protein.

In another embodiment, the activated polysaccharide and the carrier protein are co-lyophilised. In such embodiments, the activated serotype 17F polysaccharide is compounded with the carrier protein and lyophilized optionally in the presence of a saccharide. In an embodiment, the saccharide is selected from sucrose, trehalose, raffinose, stachyose, melezitose, dextran, mannitol, lactitol and palatinit. In an embodiment, the saccharide is sucrose. The co-lyophilized polysaccharide and carrier protein can then be resuspended in solution and reacted with a reducing agent.

The second step of the conjugation process is the reduction of the activated polysaccharide and a carrier protein to form a conjugate (reductive amination), using a reducing agent.

The activated serotype 17F polysaccharide can be conjugated to a carrier protein by a process comprising the steps of:
(c) compounding the activated serotype 17F polysaccharide with a carrier protein; and
(d) reacting the compounded activated serotype 17F polysaccharide and carrier protein with a reducing agent to form a serotype 17F polysaccharide-carrier protein conjugate.

In an embodiment, the reduction reaction is carried out in aqueous solvent. In another embodiment the reaction is carried out in aprotic solvent. In an embodiment, the reduction reaction is carried out in DMSO (dimethylsulfoxide) or in DMF (dimethylformamide)) solvent. The DMSO or DMF solvent may be used to reconstitute the activated polysaccharide and carrier protein which has been lyophilised.

The conjugation of activated serotype 17F polysaccharide with a protein carrier by reductive amination in dimethylsulfoxide (DMSO) is suitable to preserve the O-acetyl content of the polysaccharide as compared, for example, to reductive amination in aqueous phase where the level of O-acetylation of the polysaccharide may be significantly reduced. Therefore, in one or more embodiments, step (c) and step (d) are carried out in DMSO.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In an embodiment, the reducing agent is sodium cyanoborohydride.

At the end of the reduction reaction, there may be unreacted aldehyde groups remaining in the conjugates, these may be capped using a suitable capping agent. In one embodiment this capping agent is sodium borohydride (NaBH₄).

Following conjugation of serotype 17F polysaccharide to the carrier protein, the glycoconjugate can be purified (enriched with respect to the amount of polysaccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

In some embodiments, the serotype 17F glycoconjugates of the present invention comprise a saccharide having a molecular weight of between 10 kDa and 2,000 kDa. In other such embodiments, the saccharide has a molecular weight of between 50 kDa and 1,000 kDa. In other such embodiments, the saccharide has a molecular weight of between 70 kDa and 900 kDa. In other such embodiments, the saccharide has a molecular weight of between 100 kDa and 800 kDa. In other such embodiments, the saccharide has a molecular weight of between 200 kDa and 600 kDa. In further such embodiments, the saccharide has a molecular weight of 100 kDa to 1,000 kDa; 100 kDa to 900 kDa; 100 kDa to 800 kDa; 100 kDa to 700 kDa; 100 kDa to 600 kDa; 100 kDa to 500 kDa; 100 kDa to 400 kDa; 100 kDa to 300 kDa; 150 kDa to 1,000 kDa; 150 kDa to 900 kDa; 150 kDa to 800 kDa; 150 kDa to 700 kDa; 150 kDa to 600 kDa; 150 kDa to 500 kDa; 150 kDa to 400 kDa; 150 kDa to 300 kDa; 200 kDa to 1,000 kDa; 200 kDa to 900 kDa; 200 kDa to 800 kDa; 200 kDa to 700 kDa; 200 kDa to 600 kDa; 200 kDa to 500 kDa; 200 kDa to 400 kDa; 200 kDa to 300 kDa; 250 kDa to 1,000 kDa; 250 kDa to 900 kDa; 250 kDa to 800 kDa; 250 kDa to 700 kDa; 250 kDa to 600 kDa; 250 kDa to 500 kDa; 250 kDa to 400 kDa; 250 kDa to 350 kDa; 300 kDa to 1000 kDa; 300 kDa to 900 kDa; 300 kDa to 800 kDa; 300 kDa to 700 kDa; 300 kDa to 600 kDa; 300 kDa to 500 kDa; 300 kDa to 400 kDa; 400 kDa to 1,000 kDa; 400 kDa to 900 kDa; 400 kDa to 800 kDa; 400 kDa to 700 kDa; 400 kDa to 600 kDa; 500 kDa to 600 kDa. Any whole number integer within any of the above ranges is contemplated as an embodiment of the disclosure. In some such embodiments, the serotype 6C, 7C, 9N, 15A, 15B, 15C, 16F, 17F, 20, 23A, 23B, 31, 34, 35B, 35F and/or 38 glycoconjugates are prepared using reductive amination.

In some embodiments, the serotype 17F glycoconjugate of the invention has a molecular weight of between 400 kDa and 15,000 kDa; between 500 kDa and 10,000 kDa; between 2,000 kDa and 10,000 kDa; between 3,000 kDa and 8,000 kDa; or between 3,000 kDa and 5,000 kDa. In other embodiments, the serotype 17F glycoconjugate has a molecular weight of between 500 kDa and 10,000 kDa. In other embodiments, the serotype 17F glycoconjugate has a molecular weight of between 1,000 kDa and 8,000 kDa. In still other embodiments, the serotype 17F glycoconjugate has a molecular weight of between 2,000 kDa and 8,000 kDa or between 3,000 kDa and 7,000 kDa. In further embodiments, the serotype 17F glycoconjugate of the invention has a molecular weight of between 200 kDa and 20,000 kDa; between 200 kDa and 15,000 kDa; between 200 kDa and 10,000 kDa; between 200 kDa and 7,500 kDa; between 200 kDa and 5,000 kDa; between 200 kDa and 3,000 kDa; between 200 kDa and 1,000 kDa; between 500 kDa and 20,000 kDa; between 500 kDa and 15,000 kDa; between 500 kDa and 12,500 kDa; between 500 kDa and 10,000 kDa; between 500 kDa and 7,500 kDa; between 500 kDa and 6,000 kDa; between 500 kDa and 5,000 kDa; between 500 kDa and 4,000 kDa; between 500 kDa and 3,000 kDa; between 500 kDa and 2,000 kDa; between 500 kDa and 1,500 kDa; between 500 kDa and 1,000 kDa; between 750 kDa and 20,000 kDa; between 750 kDa and 15,000 kDa; between 750 kDa and 12,500 kDa; between 750 kDa and 10,000 kDa; between 750 kDa and 7,500 kDa; between 750 kDa and 6,000 kDa; between 750 kDa and 5,000 kDa; between 750 kDa and 4,000 kDa; between 750 kDa and 3,000 kDa; between 750 kDa and 2,000 kDa; between 750 kDa and 1,500 kDa; between 1,000 kDa and 15,000 kDa; between 1,000 kDa and 12,500 kDa; between 1,000 kDa and 10,000 kDa; between 1,000 kDa and 7,500 kDa; between 1,000 kDa and 6,000 kDa; between 1,000 kDa and 5,000 kDa; between 1,000 kDa and 4,000 kDa; between 1,000 kDa and 2,500 kDa; between 2,000 kDa and 15,000 kDa; between 2,000 kDa and 12,500 kDa; between 2,000 kDa and 10,000 kDa; between 2,000 kDa and 7,500 kDa; between 2,000 kDa and 6,000 kDa; between 2,000 kDa and 5,000 kDa; between 2,000 kDa and 4,000 kDa; or between 2,000 kDa and 3,000 kDa.

In further embodiments, the serotype 17F glycoconjugate of the invention has a molecular weight of between 3,000 kDa and 20,000 kDa; between 3,000 kDa and 15,000 kDa; between 3,000 kDa and 10,000 kDa; between 3,000 kDa and 7,500 kDa; between 3,000 kDa and 5,000 kDa; between 4,000 kDa and 20,000 kDa; between 4,000 kDa and 15,000 kDa; between 4,000 kDa and 12,500 kDa; between 4,000 kDa and 10,000 kDa; between 4,000 kDa and 7,500 kDa; between 4,000 kDa and 6,000 kDa; or between 4,000 kDa and 5,000 kDa.

In further embodiments, the serotype 17F glycoconjugate of the invention has a molecular weight of between 5,000 kDa and 20,000 kDa; between 5,000 kDa and 15,000 kDa; between 5,000 kDa and 10,000 kDa; between 5,000 kDa and 7,500 kDa; between 6,000 kDa and 20,000 kDa; between 6,000 kDa and 15,000 kDa; between 6,000 kDa and 12,500 kDa; between 6,000 kDa and 10,000 kDa or between 6,000 kDa and 7,500 kDa.

The molecular weight of the glycoconjugate is measured by SEC-MALLS. Any whole number integer within any of the above ranges is contemplated as an embodiment of the disclosure.

In an embodiment, the serotype 17F glycoconjugate of the invention comprises at least 0.1, 0.2, 0.3, 0.4, 0.5, 0.6 or 0.7 or about 0.8 mM acetate per mM serotype 17F polysaccharide. In an embodiment, the glycoconjugate comprises at least 0.5, 0.6 or 0.7 mM acetate per mM serotype 17F polysaccharide. In an embodiment, the glycoconjugate comprises at least 0.6 mM acetate per mM serotype 17F polysaccharide. In another embodiment, the glycoconjugate comprises at least 0.7 mM acetate per mM serotype 17F polysaccharide.

In another embodiment, the ratio of mM acetate per mM serotype 17F polysaccharide in the glycoconjugate to mM acetate per mM serotype 17F polysaccharide in the isolated polysaccharide is at least 0.6, 0.65, 0.7, 0.75, 0.8, 0.85, 0.9, or 0.95. In an embodiment, the ratio of mM acetate per mM serotype 17F polysaccharide in the glycoconjugate to mM acetate per mM serotype 17F polysaccharide in the isolated polysaccharide is at least 0.7. In another embodiment, the ratio of mM acetate per mM serotype 17F polysaccharide in the glycoconjugate to mM acetate per mM serotype 17F polysaccharide in the isolated polysaccharide is at least 0.9.

In an embodiment, the ratio of mM acetate per mM serotype 17F polysaccharide in the glycoconjugate to mM acetate per mM serotype 17F polysaccharide in the activated polysaccharide is at least 0.6, 0.65, 0.7, 0.75, 0.8, 0.85, 0.9, or 0.95. In an embodiment, the ratio of mM acetate per mM serotype 17F polysaccharide in the glycoconjugate to mM acetate per mM serotype 17F polysaccharide in the activated polysaccharide is at least 0.7. In another embodiment, the ratio of mM acetate per mM serotype 17F polysaccharide in the glycoconjugate to mM acetate per mM serotype 17F polysaccharide in the activated polysaccharide is at least 0.9.

Another way to characterize the serotype 17F glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the CRM₁₉₇ protein starting material used to generate the conjugate materials. In an embodiment, the degree of conjugation of the serotype 17F glycoconjugate of the invention is between 2 and 15, between 2 and 13, between 2 and 10, between 2 and 8, between 2 and 6, between 2 and 5, between 2 and 4, between 3 and 15, between 3 and 13, between 3 and 10, between 3 and 8, between 3 and 6, between 3 and 5, between 3 and 4, between 5 and 15, between 5 and 10, between 8 and 15, between 8 and 12, between 10 and 15 or between 10 and 12. In an embodiment, the degree of conjugation of the serotype 17F glycoconjugate of the invention is about 2, about 3, about 4, about 5, about 6, about 7, about 8, about 9, about 10, about 11, about 12, about 13, about 14 or about 15. In an embodiment, the degree of conjugation of the serotype 17F glycoconjugate of the invention is between 4 and 7. In some such embodiments, the carrier protein is CRM₁₉₇.

The serotype 17F glycoconjugates of the invention may also be characterized by the ratio (weight/weight) of saccharide to carrier protein. In some embodiments, the ratio of serotype 17F polysaccharide to carrier protein in the glycoconjugate (w/w) is between 0.5 and 3.0 (e.g., about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0). In other embodiments, the saccharide to carrier protein ratio (w/w) is between 0.5 and 2.0, between 0.5 and 1.5, between 0.8 and 1.2, between 0.5 and 1.0, between 1.0 and 1.5 or between 1.0 and 2.0. In further embodiments, the saccharide to carrier protein ratio (w/w) is between 0.8 and 1.2. In an embodiment, the ratio of serotype 17F capsular polysaccharide to carrier protein in the conjugate is between 0.9 and 1.1. In some such embodiments, the carrier protein is CRM₁₉₇.

The serotype 17F glycoconjugates and immunogenic compositions of the invention may contain free saccharide that is not covalently conjugated to the carrier protein, but is nevertheless present in the glycoconjugate composition. The free saccharide may be noncovalently associated with (i.e., noncovalently bound to, adsorbed to, or entrapped in or with) the glycoconjugate.

In an embodiment, the serotype 17F glycoconjugate comprises less than about 50%, 45%, 40%, 35%, 30%, 25%, 20% or 15% of free serotype 17F polysaccharide compared to the total amount of serotype 17F polysaccharide. In another embodiment, the serotype 17F glycoconjugate comprises less than about 40% of free serotype 17F polysaccharide compared to the total amount of serotype 17F polysaccharide. In an embodiment, the serotype 17F glycoconjugate comprises less than about 25% of free serotype 17F polysaccharide compared to the total amount of serotype 17F polysaccharide. In an embodiment, the serotype 17F glycoconjugate comprises less than about 20% of free serotype 17F polysaccharide compared to the total amount of serotype 17F polysaccharide. In another embodiment the serotype 17F glycoconjugate comprises less than about 15% of free serotype 17F polysaccharide compared to the total amount of serotype 17F polysaccharide.

The serotype 17F glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In an embodiment, at least 30% of the serotype 17F glycoconjugate has a K_{d} below or equal to 0.3 in a CL-4B column. In another embodiment, at least 40% of the glycoconjugate has a K_{d} below or equal to 0.3 in a CL-4B column. In another embodiment, at least 45%, 50%, 55%, 60%, 65%, 70%, 75%, 80%, or 85% of the serotype 17F glycoconjugate has a K_{d} below or equal to 0.3 in a CL-4B column. In an embodiment, at least 60% of the serotype 17F glycoconjugate has a K_{d} below or equal to 0.3 in a CL-4B column. In another embodiment, between 50% and 80% of the serotype 17F glycoconjugate has a K_{d} below or equal to 0.3 in a CL-4B column. In an embodiment, between 65% and 80% of the serotype 17F glycoconjugate has a K_{d} below or equal to 0.3 in a CL-4B column.

1.3.10 Glycoconjugates from S. pneumoniae Serotype 20

In an embodiment, the serotype 20 glycoconjugates are obtained by activating polysaccharide with 1-cyano-4-dimethylamino pyridinium tetrafluoroborate (CDAP) to form a cyanate ester. The activated polysaccharide may be coupled directly or via a spacer (linker) group to an amino group on the carrier protein. For example, the spacer may be cystamine or cysteamine to give a thiolated polysaccharide which may be coupled to the carrier via a thioether linkage obtained after reaction with a maleimide-activated carrier protein (for example using GMBS) or a haloacetylated carrier protein (for example using iodoacetimide, SIB, SIAB, sulfo-SIAB, SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO93/15760, WO95/08348 and WO96/129094.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO98/42721. Conjugation may involve a carbonyl linker which may be formed by reaction of a free hydroxyl group of the saccharide with CDI (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In one or more embodiments, the serotype 20 glycoconjugates of the invention are prepared using reductive amination. Reductive amination involves two steps: (1) oxidation of the polysaccharide to generate aldehyde functionalities from vicinal diols in individual hexasaccharide unit and (2) reduction of the activated polysaccharide and a carrier protein (e.g., CRM₁₉₇) to form a conjugate.

In an embodiment, before oxidation, sizing of the serotype 20 polysaccharide to a target molecular weight (MW) range is performed. Advantageously, the size of the purified serotype 20 polysaccharide is reduced while preserving critical features of the structure of the polysaccharide such as for example the presence of O-acetyl groups. In an embodiment, the size of the purified serotype 20 polysaccharide is reduced by mechanical homogenization as described herein.

In an embodiment, serotype polysaccharide is activated (oxidized) by a process comprising the step of:
(a) reacting isolated serotype 20 polysaccharide with an oxidizing agent; and
(b) quenching the oxidation reaction by addition of a quenching agent resulting in an activated serotype 20 polysaccharide.

In an embodiment, the oxidizing agent is periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate). In an embodiment, the oxidizing agent is sodium periodate. In an embodiment, the periodate used for the oxidation of serotype 20 polysaccharide is metaperiodate. In another embodiment, the periodate used for the oxidation of serotype 20 polysaccharide is sodium metaperiodate.

In one embodiment, the quenching agent is selected from vicinal diols, 1,2-aminoalcohols, amino acids, glutathione, sulfite, bisulfate, dithionite, metabisulfite, thiosulfate, phosphites, hypophosphites or phosphorous acid.

In one embodiment, the quenching agent is a 1,2-aminoalcohols of formula (I):

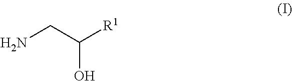

wherein R¹ is selected from H, methyl, ethyl, propyl or isopropyl.

In one embodiment, the quenching agent is selected from sodium and potassium salts of sulfite, bisulfate, dithionite, metabisulfite, thiosulfate, phosphites, hypophosphites or phosphorous acid.

In one embodiment, the quenching agent is an amino acid. In such embodiments, said amino acid may be selected from serine, threonine, cysteine, cystine, methionine, proline, hydroxyproline, tryptophan, tyrosine, and histidine.

In one embodiment, the quenching agent is a sulfite such as bisulfate, dithionite, metabisulfite, thiosulfate.

In one embodiment, the quenching agent is a compound comprising two vicinal hydroxyl groups (vicinal diols), i.e., two hydroxyl groups covalently linked to two adjacent carbon atoms.

In one or more embodiments, the quenching agent is a compound of formula (II):

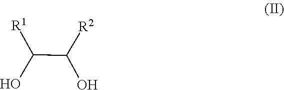

wherein R¹ and R² are each independently selected from H, methyl, ethyl, propyl or isopropyl.

In an embodiment, the quenching agent is glycerol, ethylene glycol, propan-1,2-diol, butan-1,2-diol or butan-2,3-diol, or ascorbic acid. In an embodiment, the quenching agent is butan-2,3-diol.

In another embodiment, the isolated serotype 20 polysaccharide is activated by a process comprising the steps of:
(a) reacting isolated serotype 20 polysaccharide with periodate; and
(b) quenching the oxidation reaction by addition of butan-2,3-diol resulting in an activated serotype 20 polysaccharide.

Following the oxidation step of the polysaccharide, the polysaccharide is said to be activated and is referred to as “activated polysaccharide” herein.

In an embodiment, the activated serotype 20 polysaccharide is purified. The activated serotype 20 polysaccharide is purified according to methods known to one skilled in the art such as gel permeation chromatography (GPC), dialysis or ultrafiltration/diafiltration. For example, the activated 20 polysaccharide is purified by concentration and diafiltration using an ultrafiltration device.

In another embodiment, the degree of oxidation of the activated serotype 20 polysaccharide is between 2 and 30, between 2 and 25, between 2 and 20, between 2 and 15, between 2 and 10, between 2 and 5, between 5 and 30, between 5 and 25, between 5 and 20, between 5 and 15, between 5 and 10, between 10 and 30, between 10 and 25, between 10 and 20, between 10 and 15, between 15 and 30, between 15 and 25, between 15 and 20, between 20 to 30, or between 20 to 25. In an embodiment the degree of oxidation of the activated serotype 20 polysaccharide is between 2 and 10, between 4 and 8, between 4 and 6, between 6 and 8, between 6 and 12, between 8 and 14, between 9 and 11, between 10 and 16, between 12 and 16, between 14 and 18, between 16 and 20, between 16 and 18, between 18 and 22, or between 18 and 20.

In an embodiment, the activated serotype 20 polysaccharide has a molecular weight between 25 kDa and 1,000 kDa, between 100 kDa and 1,000 kDa, between 300 kDa and 800 kDa, between 300 kDa and 700 kDa, between 300 kDa and 600 kDa, between 400 kDa and 1,000 kDa, between 400 kDa and 800 kDa, between 400 kDa and 700 kDa or between 400 kDa and 600 kDa. In an embodiment, the activated serotype 20 polysaccharide has a molecular weight between 300 kDa and 800 kDa. In an embodiment, the activated serotype 20 polysaccharide has a molecular weight between 400 kDa and 600 kDa. In another embodiment, the activated serotype 20 polysaccharide has a molecular weight between 400 kda and 600 kDa and a degree of oxidation between 10 and 25, between 10 and 20, between 12 and 20 or between 14 and 18. In another embodiment, the activated serotype 20 polysaccharide has a molecular weight between 400 kDa and 600 kDa and a degree of oxidation between 10 and 20.

In another embodiment, the activated serotype 20 polysaccharide comprises at least 0.1, 0.2, 0.3, 0.4, 0.5, 0.6 or 0.7 or about 0.8 mM acetate per mM serotype 20 polysaccharide. In an embodiment, the activated serotype 20 polysaccharide comprises at least 0.5, 0.6 or 0.7 mM acetate per mM serotype 20 polysaccharide. In another embodiment, the activated serotype 20 polysaccharide comprises at least 0.6 mM acetate per mM serotype 20 polysaccharide. In another embodiment, the activated serotype 20 polysaccharide comprises at least 0.7 mM acetate per mM serotype 20 polysaccharide.

In an embodiment, the activated serotype 20 polysaccharide has a molecular weight between 400 kDa and 800 kDa and comprises at least 0.6 mM acetate per mM serotype 20 polysaccharide.

In an embodiment, the activated serotype 20 polysaccharide has a molecular weight between 400 kDa and 800 kDa, a degree of oxidation between 12 and 20 and comprises at least 0.6 mM acetate per mM serotype 20 polysaccharide.

The activated polysaccharide and/or the carrier protein may be lyophilised (freeze-dried), either independently (discrete lyophilization) or together (co-lyophilized).

In another embodiment, the activated serotype 20 polysaccharide is lyophilized, optionally in the presence of saccharide. In an embodiment, the saccharide is selected from sucrose, trehalose, raffinose, stachyose, melezitose, dextran, mannitol, lactitol and palatinit. In an embodiment, the saccharide is sucrose. In one embodiment, the lyophilized activated polysaccharide is then compounded with a solution comprising the carrier protein.

In another embodiment, the activated polysaccharide and the carrier protein are co-lyophilised. In such embodiments, the activated serotype 20 polysaccharide is compounded with the carrier protein and lyophilized optionally in the presence of a saccharide. In an embodiment, the saccharide is selected from sucrose, trehalose, raffinose, stachyose, melezitose, dextran, mannitol, lactitol and palatinit. In an embodiment, the saccharide is sucrose. The co-lyophilized polysaccharide and carrier protein can then be resuspended in solution and reacted with a reducing agent.

The second step of the conjugation process is the reduction of the activated polysaccharide and a carrier protein to form a conjugate (reductive amination), using a reducing agent.

The activated serotype 20 polysaccharide can be conjugated to a carrier protein by a process comprising the steps of:
(c) compounding the activated serotype 20 polysaccharide with a carrier protein; and
(d) reacting the compounded activated serotype 20 polysaccharide and carrier protein with a reducing agent to form a serotype 20 polysaccharide-carrier protein conjugate.

In an embodiment, the reduction reaction is carried out in aqueous solvent. In another embodiment the reaction is carried out in aprotic solvent. In an embodiment, the reduction reaction is carried out in DMSO (dimethylsulfoxide) or in DMF (dimethylformamide)) solvent. The DMSO or DMF solvent may be used to reconstitute the activated polysaccharide and carrier protein which has been lyophilised.

The conjugation of activated serotype 20 polysaccharide with a protein carrier by reductive amination in dimethylsulfoxide (DMSO) is suitable to preserve the O-acetyl content of the polysaccharide as compared, for example, to reductive amination in aqueous phase where the level of O-acetylation of the polysaccharide may be significantly reduced. Therefore, in one or more embodiments, step (c) and step (d) are carried out in DMSO.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In an embodiment, the reducing agent is sodium cyanoborohydride.

At the end of the reduction reaction, there may be unreacted aldehyde groups remaining in the conjugates, these may be capped using a suitable capping agent. In one embodiment this capping agent is sodium borohydride (NaBH₄).

Following conjugation of serotype 20 polysaccharide to the carrier protein, the glycoconjugate can be purified (enriched with respect to the amount of polysaccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

In some embodiments, the serotype 20 glycoconjugates of the present invention comprise a saccharide having a molecular weight of between 10 kDa and 2,000 kDa. In other such embodiments, the saccharide has a molecular weight of between 50 kDa and 1,000 kDa. In other such embodiments, the saccharide has a molecular weight of between 70 kDa and 900 kDa. In other such embodiments, the saccharide has a molecular weight of between 100 kDa and 800 kDa. In other such embodiments, the saccharide has a molecular weight of between 200 kDa and 600 kDa. In further such embodiments, the saccharide has a molecular weight of 100 kDa to 1,000 kDa; 100 kDa to 900 kDa; 100 kDa to 800 kDa; 100 kDa to 700 kDa; 100 kDa to 600 kDa; 100 kDa to 500 kDa; 100 kDa to 400 kDa; 100 kDa to 300 kDa; 150 kDa to 1,000 kDa; 150 kDa to 900 kDa; 150 kDa to 800 kDa; 150 kDa to 700 kDa; 150 kDa to 600 kDa; 150 kDa to 500 kDa; 150 kDa to 400 kDa; 150 kDa to 300 kDa; 200 kDa to 1,000 kDa; 200 kDa to 900 kDa; 200 kDa to 800 kDa; 200 kDa to 700 kDa; 200 kDa to 600 kDa; 200 kDa to 500 kDa; 200 kDa to 400 kDa; 200 kDa to 300 kDa; 250 kDa to 1,000 kDa; 250 kDa to 900 kDa; 250 kDa to 800 kDa; 250 kDa to 700 kDa; 250 kDa to 600 kDa; 250 kDa to 500 kDa; 250 kDa to 400 kDa; 250 kDa to 350 kDa; 300 kDa to 1000 kDa; 300 kDa to 900 kDa; 300 kDa to 800 kDa; 300 kDa to 700 kDa; 300 kDa to 600 kDa; 300 kDa to 500 kDa; 300 kDa to 400 kDa; 400 kDa to 1,000 kDa; 400 kDa to 900 kDa; 400 kDa to 800 kDa; 400 kDa to 700 kDa; 400 kDa to 600 kDa; 500 kDa to 600 kDa. Any whole number integer within any of the above ranges is contemplated as an embodiment of the disclosure. In some such embodiments, the serotype 20 glycoconjugates are prepared using reductive amination.

In some embodiments, the serotype 20 glycoconjugate of the invention has a molecular weight of between 400 kDa and 15,000 kDa; between 500 kDa and 10,000 kDa; between 2,000 kDa and 10,000 kDa; between 3,000 kDa and 8,000 kDa; or between 3,000 kDa and 5,000 kDa. In other embodiments, the serotype 20 glycoconjugate has a molecular weight of between 500 kDa and 10,000 kDa. In other embodiments, the serotype 20 glycoconjugate has a molecular weight of between 1,000 kDa and 8,000 kDa. In still other embodiments, the serotype 20 glycoconjugate has a molecular weight of between 2,000 kDa and 8,000 kDa or between 3,000 kDa and 7,000 kDa. In further embodiments, the serotype 20 glycoconjugate of the invention has a molecular weight of between 200 kDa and 20,000 kDa; between 200 kDa and 15,000 kDa; between 200 kDa and 10,000 kDa; between 200 kDa and 7,500 kDa; between 200 kDa and 5,000 kDa; between 200 kDa and 3,000 kDa; between 200 kDa and 1,000 kDa; between 500 kDa and 20,000 kDa; between 500 kDa and 15,000 kDa; between 500 kDa and 12,500 kDa; between 500 kDa and 10,000 kDa; between 500 kDa and 7,500 kDa; between 500 kDa and 6,000 kDa; between 500 kDa and 5,000 kDa; between 500 kDa and 4,000 kDa; between 500 kDa and 3,000 kDa; between 500 kDa and 2,000 kDa; between 500 kDa and 1,500 kDa; between 500 kDa and 1,000 kDa; between 750 kDa and 20,000 kDa; between 750 kDa and 15,000 kDa; between 750 kDa and 12,500 kDa; between 750 kDa and 10,000 kDa; between 750 kDa and 7,500 kDa; between 750 kDa and 6,000 kDa; between 750 kDa and 5,000 kDa; between 750 kDa and 4,000 kDa; between 750 kDa and 3,000 kDa; between 750 kDa and 2,000 kDa; between 750 kDa and 1,500 kDa; between 1,000 kDa and 15,000 kDa; between 1,000 kDa and 12,500 kDa; between 1,000 kDa and 10,000 kDa; between 1,000 kDa and 7,500 kDa; between 1,000 kDa and 6,000 kDa; between 1,000 kDa and 5,000 kDa; between 1,000 kDa and 4,000 kDa; between 1,000 kDa and 2,500 kDa; between 2,000 kDa and 15,000 kDa; between 2,000 kDa and 12,500 kDa; between 2,000 kDa and 10,000 kDa; between 2,000 kDa and 7,500 kDa; between 2,000 kDa and 6,000 kDa; between 2,000 kDa and 5,000 kDa; between 2,000 kDa and 4,000 kDa; or between 2,000 kDa and 3,000 kDa.

In further embodiments, the serotype 20 glycoconjugate of the invention has a molecular weight of between 3,000 kDa and 20,000 kDa; between 3,000 kDa and 15,000 kDa; between 3,000 kDa and 10,000 kDa; between 3,000 kDa and 7,500 kDa; between 3,000 kDa and 5,000 kDa; between 4,000 kDa and 20,000 kDa; between 4,000 kDa and 15,000 kDa; between 4,000 kDa and 12,500 kDa; between 4,000 kDa and 10,000 kDa; between 4,000 kDa and 7,500 kDa; between 4,000 kDa and 6,000 kDa; or between 4,000 kDa and 5,000 kDa.

In further embodiments, the serotype 20 glycoconjugate of the invention has a molecular weight of between 5,000 kDa and 20,000 kDa; between 5,000 kDa and 15,000 kDa; between 5,000 kDa and 10,000 kDa; between 5,000 kDa and 7,500 kDa; between 6,000 kDa and 20,000 kDa; between 6,000 kDa and 15,000 kDa; between 6,000 kDa and 12,500 kDa; between 6,000 kDa and 10,000 kDa or between 6,000 kDa and 7,500 kDa.

The molecular weight of the glycoconjugate is measured by SEC-MALLS. Any whole number integer within any of the above ranges is contemplated as an embodiment of the disclosure.

In an embodiment, the serotype 20 glycoconjugate of the invention comprises at least 0.1, 0.2, 0.3, 0.4, 0.5, 0.6 or 0.7 or about 0.8 mM acetate per mM serotype 20 polysaccharide. In an embodiment, the glycoconjugate comprises at least 0.5, 0.6 or 0.7 mM acetate per mM serotype 20 polysaccharide. In an embodiment, the glycoconjugate comprises at least 0.6 mM acetate per mM serotype 20 polysaccharide. In another embodiment, the glycoconjugate comprises at least 0.7 mM acetate per mM serotype 20 polysaccharide.

In another embodiment, the ratio of mM acetate per mM serotype 20 polysaccharide in the glycoconjugate to mM acetate per mM serotype 20 polysaccharide in the isolated polysaccharide is at least 0.6, 0.65, 0.7, 0.75, 0.8, 0.85, 0.9, or 0.95. In an embodiment, the ratio of mM acetate per mM serotype 20 polysaccharide in the glycoconjugate to mM acetate per mM serotype 20 polysaccharide in the isolated polysaccharide is at least 0.7. In another embodiment, the ratio of mM acetate per mM serotype 20 polysaccharide in the glycoconjugate to mM acetate per mM serotype 20 polysaccharide in the isolated polysaccharide is at least 0.9.

In an embodiment, the ratio of mM acetate per mM serotype 20 polysaccharide in the glycoconjugate to mM acetate per mM serotype 20 polysaccharide in the activated polysaccharide is at least 0.6, 0.65, 0.7, 0.75, 0.8, 0.85, 0.9, or 0.95. In an embodiment, the ratio of mM acetate per mM serotype 20 polysaccharide in the glycoconjugate to mM acetate per mM serotype 20 polysaccharide in the activated polysaccharide is at least 0.7. In another embodiment, the ratio of mM acetate per mM serotype 20 polysaccharide in the glycoconjugate to mM acetate per mM serotype 20 polysaccharide in the activated polysaccharide is at least 0.9.

Another way to characterize the serotype 20 glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the CRM₁₉₇ protein starting material used to generate the conjugate materials. In an embodiment, the degree of conjugation of the serotype 20 glycoconjugate of the invention is between 2 and 15, between 2 and 13, between 2 and 10, between 2 and 8, between 2 and 6, between 2 and 5, between 2 and 4, between 3 and 15, between 3 and 13, between 3 and 10, between 3 and 8, between 3 and 6, between 3 and 5, between 3 and 4, between 5 and 15, between 5 and 10, between 8 and 15, between 8 and 12, between 10 and 15 or between 10 and 12. In an embodiment, the degree of conjugation of the serotype 20 glycoconjugate of the invention is about 2, about 3, about 4, about 5, about 6, about 7, about 8, about 9, about 10, about 11, about 12, about 13, about 14 or about 15. In an embodiment, the degree of conjugation of the serotype 20 glycoconjugate of the invention is between 4 and 7. In some such embodiments, the carrier protein is CRM₁₉₇.

The serotype 20 glycoconjugates of the invention may also be characterized by the ratio (weight/weight) of saccharide to carrier protein. In some embodiments, the ratio of serotype 20 polysaccharide to carrier protein in the glycoconjugate (w/w) is between 0.5 and 3.0 (e.g., about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0). In other embodiments, the saccharide to carrier protein ratio (w/w) is between 0.5 and 2.0, between 0.5 and 1.5, between 0.8 and 1.2, between 0.5 and 1.0, between 1.0 and 1.5 or between 1.0 and 2.0. In further embodiments, the saccharide to carrier protein ratio (w/w) is between 0.8 and 1.2. In an embodiment, the ratio of serotype 20 capsular polysaccharide to carrier protein in the conjugate is between 0.9 and 1.1. In some such embodiments, the carrier protein is CRM₁₉₇.

The serotype 20 glycoconjugates and immunogenic compositions of the invention may contain free saccharide that is not covalently conjugated to the carrier protein, but is nevertheless present in the glycoconjugate composition. The free saccharide may be noncovalently associated with (i.e., noncovalently bound to, adsorbed to, or entrapped in or with) the glycoconjugate.

In an embodiment, the serotype 20 glycoconjugate comprises less than about 50%, 45%, 40%, 35%, 30%, 25%, 20% or 15% of free serotype 20 polysaccharide compared to the total amount of serotype 20 polysaccharide. In another embodiment, the serotype 20 glycoconjugate comprises less than about 40% of free serotype 20 polysaccharide compared to the total amount of serotype 20 polysaccharide. In an embodiment, the serotype 20 glycoconjugate comprises less than about 25% of free serotype 20 polysaccharide compared to the total amount of serotype 20 polysaccharide. In an embodiment, the serotype 20 glycoconjugate comprises less than about 20% of free serotype 20 polysaccharide compared to the total amount of serotype 20 polysaccharide. In another embodiment the serotype 20 glycoconjugate comprises less than about 15% of free serotype 20 polysaccharide compared to the total amount of serotype 20 polysaccharide.

The serotype 20 glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In an embodiment, at least 30% of the serotype 20 glycoconjugate has a K_{d} below or equal to 0.3 in a CL-4B column. In another embodiment, at least 40% of the glycoconjugate has a K_{d} below or equal to 0.3 in a CL-4B column. In another embodiment, at least 45%, 50%, 55%, 60%, 65%, 70%, 75%, 80%, or 85% of the serotype 20 glycoconjugate has a K_{d} below or equal to 0.3 in a CL-4B column. In an embodiment, at least 60% of the serotype 20 glycoconjugate has a K_{d} below or equal to 0.3 in a CL-4B column. In another embodiment, between 50% and 80% of the serotype 20 glycoconjugate has a K_{d} below or equal to 0.3 in a CL-4B column. In an embodiment, between 65% and 80% of the serotype 20 glycoconjugate has a K_{d} below or equal to 0.3 in a CL-4B column.

1.3.11 Glycoconjugates from S. pneumoniae Serotype 23A

In an embodiment, the serotype 23A glycoconjugates are obtained by activating polysaccharide with 1-cyano-4-dimethylamino pyridinium tetrafluoroborate (CDAP) to form a cyanate ester. The activated polysaccharide may be coupled directly or via a spacer (linker) group to an amino group on the carrier protein. For example, the spacer may be cystamine or cysteamine to give a thiolated polysaccharide which may be coupled to the carrier via a thioether linkage obtained after reaction with a maleimide-activated carrier protein (for example using GMBS) or a haloacetylated carrier protein (for example using iodoacetimide, SIB, SIAB, sulfo-SIAB, SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO93/15760, WO95/08348 and WO96/129094.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO98/42721. Conjugation may involve a carbonyl linker which may be formed by reaction of a free hydroxyl group of the saccharide with CDI (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In one or more embodiments, the serotype 23A glycoconjugates of the invention are prepared using reductive amination. Reductive amination involves two steps: (1) oxidation of the polysaccharide to generate aldehyde functionalities from vicinal diols in individual hexasaccharide unit and (2) reduction of the activated polysaccharide and a carrier protein (e.g., CRM₁₉₇) to form a conjugate.

In an embodiment, before oxidation, sizing of the serotype 23A polysaccharide to a target molecular weight (MW) range is performed. Advantageously, the size of the purified serotype 23A polysaccharide is reduced while preserving critical features of the structure of the polysaccharide such as for example the presence of O-acetyl groups. In an embodiment, the size of the purified serotype 23A polysaccharide is reduced by mechanical homogenization as described herein.

In an embodiment, serotype polysaccharide is activated (oxidized) by a process comprising the step of:
(a) reacting isolated serotype 23A polysaccharide with an oxidizing agent; and
(b) quenching the oxidation reaction by addition of a quenching agent resulting in an activated serotype 23A polysaccharide.

In an embodiment, the oxidizing agent is periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate). In an embodiment, the oxidizing agent is sodium periodate. In an embodiment, the periodate used for the oxidation of serotype 23A polysaccharide is metaperiodate. In another embodiment, the periodate used for the oxidation of serotype 23A polysaccharide is sodium metaperiodate.

In one embodiment, the quenching agent is selected from vicinal diols, 1,2-aminoalcohols, amino acids, glutathione, sulfite, bisulfate, dithionite, metabisulfite, thiosulfate, phosphites, hypophosphites or phosphorous acid.

In one embodiment, the quenching agent is a 1,2-aminoalcohols of formula (I):

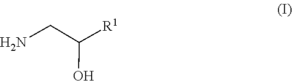

wherein R¹ is selected from H, methyl, ethyl, propyl or isopropyl.

In one embodiment, the quenching agent is selected from sodium and potassium salts of sulfite, bisulfate, dithionite, metabisulfite, thiosulfate, phosphites, hypophosphites or phosphorous acid.

In one embodiment, the quenching agent is an amino acid. In such embodiments, said amino acid may be selected from serine, threonine, cysteine, cystine, methionine, proline, hydroxyproline, tryptophan, tyrosine, and histidine.

In one embodiment, the quenching agent is a sulfite such as bisulfate, dithionite, metabisulfite, thiosulfate.

In one embodiment, the quenching agent is a compound comprising two vicinal hydroxyl groups (vicinal diols), i.e., two hydroxyl groups covalently linked to two adjacent carbon atoms.

In one or more embodiments, the quenching agent is a compound of formula (II):

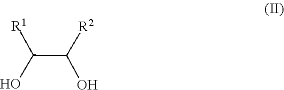

wherein R¹ and R² are each independently selected from H, methyl, ethyl, propyl or isopropyl.

In an embodiment, the quenching agent is glycerol, ethylene glycol, propan-1,2-diol, butan-1,2-diol or butan-2,3-diol, or ascorbic acid. In an embodiment, the quenching agent is butan-2,3-diol.

In another embodiment, the isolated serotype 23A polysaccharide is activated by a process comprising the steps of:
(a) reacting isolated serotype 23A polysaccharide with periodate; and
(b) quenching the oxidation reaction by addition of butan-2,3-diol resulting in an activated serotype 23A polysaccharide.

Following the oxidation step of the polysaccharide, the polysaccharide is said to be activated and is referred to as “activated polysaccharide” herein.

In an embodiment, the activated serotype 23A polysaccharide is purified. The activated serotype 23A polysaccharide is purified according to methods known to one skilled in the art such as gel permeation chromatography (GPC), dialysis or ultrafiltration/diafiltration. For example, the activated 23A polysaccharide is purified by concentration and diafiltration using an ultrafiltration device.

In another embodiment, the degree of oxidation of the activated serotype 23A polysaccharide is between 2 and 30, between 2 and 25, between 2 and 20, between 2 and 15, between 2 and 10, between 2 and 5, between 5 and 30, between 5 and 25, between 5 and 20, between 5 and 15, between 5 and 10, between 10 and 30, between 10 and 25, between 10 and 20, between 10 and 15, between 15 and 30, between 15 and 25, between 15 and 20, between 20 to 30, or between 20 to 25. In an embodiment the degree of oxidation of the activated serotype 23A polysaccharide is between 2 and 10, between 4 and 8, between 4 and 6, between 6 and 8, between 6 and 12, between 8 and 14, between 9 and 11, between 10 and 16, between 12 and 16, between 14 and 18, between 16 and 20, between 16 and 18, between 18 and 22, or between 18 and 20.

In an embodiment, the activated serotype 23A polysaccharide has a molecular weight between 25 kDa and 1,000 kDa, between 100 kDa and 1,000 kDa, between 300 kDa and 800 kDa, between 300 kDa and 700 kDa, between 300 kDa and 600 kDa, between 400 kDa and 1,000 kDa, between 400 kDa and 800 kDa, between 400 kDa and 700 kDa or between 400 kDa and 600 kDa. In an embodiment, the activated serotype 23A polysaccharide has a molecular weight between 300 kDa and 800 kDa. In an embodiment, the activated serotype 23A polysaccharide has a molecular weight between 400 kDa and 600 kDa. In another embodiment, the activated serotype 23A polysaccharide has a molecular weight between 400 kda and 600 kDa and a degree of oxidation between 10 and 25, between 10 and 20, between 12 and 20 or between 14 and 18. In another embodiment, the activated serotype 23A polysaccharide has a molecular weight between 400 kDa and 600 kDa and a degree of oxidation between 10 and 20.

In another embodiment, the activated serotype 23A polysaccharide comprises at least 0.1, 0.2, 0.3, 0.4, 0.5, 0.6 or 0.7 or about 0.8 mM acetate per mM serotype 23 AN polysaccharide. In an embodiment, the activated serotype 23A polysaccharide comprises at least 0.5, 0.6 or 0.7 mM acetate per mM serotype 23A polysaccharide. In another embodiment, the activated serotype 23A polysaccharide comprises at least 0.6 mM acetate per mM serotype 23A polysaccharide. In another embodiment, the activated serotype 23A polysaccharide comprises at least 0.7 mM acetate per mM serotype 23A polysaccharide.

In an embodiment, the activated serotype 23A polysaccharide has a molecular weight between 400 kDa and 800 kDa and comprises at least 0.6 mM acetate per mM serotype 23A polysaccharide.

In an embodiment, the activated serotype 23A polysaccharide has a molecular weight between 400 kDa and 800 kDa, a degree of oxidation between 12 and 20 and comprises at least 0.6 mM acetate per mM serotype 23A polysaccharide.

The activated polysaccharide and/or the carrier protein may be lyophilised (freeze-dried), either independently (discrete lyophilization) or together (co-lyophilized).

In another embodiment, the activated serotype 23A polysaccharide is lyophilized, optionally in the presence of saccharide. In an embodiment, the saccharide is selected from sucrose, trehalose, raffinose, stachyose, melezitose, dextran, mannitol, lactitol and palatinit. In an embodiment, the saccharide is sucrose. In one embodiment, the lyophilized activated polysaccharide is then compounded with a solution comprising the carrier protein.

In another embodiment, the activated polysaccharide and the carrier protein are co-lyophilised. In such embodiments, the activated serotype 23A polysaccharide is compounded with the carrier protein and lyophilized optionally in the presence of a saccharide. In an embodiment, the saccharide is selected from sucrose, trehalose, raffinose, stachyose, melezitose, dextran, mannitol, lactitol and palatinit. In an embodiment, the saccharide is sucrose. The co-lyophilized polysaccharide and carrier protein can then be resuspended in solution and reacted with a reducing agent.

The second step of the conjugation process is the reduction of the activated polysaccharide and a carrier protein to form a conjugate (reductive amination), using a reducing agent.

The activated serotype 23A polysaccharide can be conjugated to a carrier protein by a process comprising the steps of:
(c) compounding the activated serotype 23A polysaccharide with a carrier protein; and
(d) reacting the compounded activated serotype 23A polysaccharide and carrier protein with a reducing agent to form a serotype 23A polysaccharide-carrier protein conjugate.

In an embodiment, the reduction reaction is carried out in aqueous solvent. In another embodiment the reaction is carried out in aprotic solvent. In an embodiment, the reduction reaction is carried out in DMSO (dimethylsulfoxide) or in DMF (dimethylformamide)) solvent. The DMSO or DMF solvent may be used to reconstitute the activated polysaccharide and carrier protein which has been lyophilised.

The conjugation of activated serotype 23A polysaccharide with a protein carrier by reductive amination in dimethylsulfoxide (DMSO) is suitable to preserve the O-acetyl content of the polysaccharide as compared, for example, to reductive amination in aqueous phase where the level of O-acetylation of the polysaccharide may be significantly reduced. Therefore, in one or more embodiments, step (c) and step (d) are carried out in DMSO.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In an embodiment, the reducing agent is sodium cyanoborohydride.

At the end of the reduction reaction, there may be unreacted aldehyde groups remaining in the conjugates, these may be capped using a suitable capping agent. In one embodiment this capping agent is sodium borohydride (NaBH₄).

Following conjugation of serotype 23A polysaccharide to the carrier protein, the glycoconjugate can be purified (enriched with respect to the amount of polysaccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

In some embodiments, the serotype 23A glycoconjugates of the present invention comprise a saccharide having a molecular weight of between 10 kDa and 2,000 kDa. In other such embodiments, the saccharide has a molecular weight of between 50 kDa and 1,000 kDa. In other such embodiments, the saccharide has a molecular weight of between 70 kDa and 900 kDa. In other such embodiments, the saccharide has a molecular weight of between 100 kDa and 800 kDa. In other such embodiments, the saccharide has a molecular weight of between 200 kDa and 600 kDa. In further such embodiments, the saccharide has a molecular weight of 100 kDa to 1,000 kDa; 100 kDa to 900 kDa; 100 kDa to 800 kDa; 100 kDa to 700 kDa; 100 kDa to 600 kDa; 100 kDa to 500 kDa; 100 kDa to 400 kDa; 100 kDa to 300 kDa; 150 kDa to 1,000 kDa; 150 kDa to 900 kDa; 150 kDa to 800 kDa; 150 kDa to 700 kDa; 150 kDa to 600 kDa; 150 kDa to 500 kDa; 150 kDa to 400 kDa; 150 kDa to 300 kDa; 200 kDa to 1,000 kDa; 200 kDa to 900 kDa; 200 kDa to 800 kDa; 200 kDa to 700 kDa; 200 kDa to 600 kDa; 200 kDa to 500 kDa; 200 kDa to 400 kDa; 200 kDa to 300 kDa; 250 kDa to 1,000 kDa; 250 kDa to 900 kDa; 250 kDa to 800 kDa; 250 kDa to 700 kDa; 250 kDa to 600 kDa; 250 kDa to 500 kDa; 250 kDa to 400 kDa; 250 kDa to 350 kDa; 300 kDa to 1000 kDa; 300 kDa to 900 kDa; 300 kDa to 800 kDa; 300 kDa to 700 kDa; 300 kDa to 600 kDa; 300 kDa to 500 kDa; 300 kDa to 400 kDa; 400 kDa to 1,000 kDa; 400 kDa to 900 kDa; 400 kDa to 800 kDa; 400 kDa to 700 kDa; 400 kDa to 600 kDa; 500 kDa to 600 kDa. Any whole number integer within any of the above ranges is contemplated as an embodiment of the disclosure. In some such embodiments, the serotype 23A glycoconjugates are prepared using reductive amination.

In some embodiments, the serotype 23A glycoconjugate of the invention has a molecular weight of between 400 kDa and 15,000 kDa; between 500 kDa and 10,000 kDa; between 2,000 kDa and 10,000 kDa; between 3,000 kDa and 8,000 kDa; or between 3,000 kDa and 5,000 kDa. In other embodiments, the serotype 23A glycoconjugate has a molecular weight of between 500 kDa and 10,000 kDa. In other embodiments, the serotype 23A glycoconjugate has a molecular weight of between 1,000 kDa and 8,000 kDa. In still other embodiments, the serotype 23A glycoconjugate has a molecular weight of between 2,000 kDa and 8,000 kDa or between 3,000 kDa and 7,000 kDa. In further embodiments, the serotype 23A glycoconjugate of the invention has a molecular weight of between 200 kDa and 20,000 kDa; between 200 kDa and 15,000 kDa; between 200 kDa and 10,000 kDa; between 200 kDa and 7,500 kDa; between 200 kDa and 5,000 kDa; between 200 kDa and 3,000 kDa; between 200 kDa and 1,000 kDa; between 500 kDa and 20,000 kDa; between 500 kDa and 15,000 kDa; between 500 kDa and 12,500 kDa; between 500 kDa and 10,000 kDa; between 500 kDa and 7,500 kDa; between 500 kDa and 6,000 kDa; between 500 kDa and 5,000 kDa; between 500 kDa and 4,000 kDa; between 500 kDa and 3,000 kDa; between 500 kDa and 2,000 kDa; between 500 kDa and 1,500 kDa; between 500 kDa and 1,000 kDa; between 750 kDa and 20,000 kDa; between 750 kDa and 15,000 kDa; between 750 kDa and 12,500 kDa; between 750 kDa and 10,000 kDa; between 750 kDa and 7,500 kDa; between 750 kDa and 6,000 kDa; between 750 kDa and 5,000 kDa; between 750 kDa and 4,000 kDa; between 750 kDa and 3,000 kDa; between 750 kDa and 2,000 kDa; between 750 kDa and 1,500 kDa; between 1,000 kDa and 15,000 kDa; between 1,000 kDa and 12,500 kDa; between 1,000 kDa and 10,000 kDa; between 5,000 kDa; between 1,000 kDa and 4,000 kDa; between 1,000 kDa and 2,500 kDa; between 2,000 kDa and 15,000 kDa; between 2,000 kDa and 12,500 kDa; between 2,000 kDa and 10,000 kDa; between 2,000 kDa and 7,500 kDa; between 2,000 kDa and 6,000 kDa; between 2,000 kDa and 5,000 kDa; between 2,000 kDa and 4,000 kDa; or between 2,000 kDa and 3,000 kDa.

In further embodiments, the serotype 23A glycoconjugate of the invention has a molecular weight of between 3,000 kDa and 20,000 kDa; between 3,000 kDa and 15,000 kDa; between 3,000 kDa and 10,000 kDa; between 3,000 kDa and 7,500 kDa; between 3,000 kDa and 5,000 kDa; between 4,000 kDa and 20,000 kDa; between 4,000 kDa and 15,000 kDa; between 4,000 kDa and 12,500 kDa; between 4,000 kDa and 10,000 kDa; between 4,000 kDa and 7,500 kDa; between 4,000 kDa and 6,000 kDa; or between 4,000 kDa and 5,000 kDa.

In further embodiments, the serotype 23A glycoconjugate of the invention has a molecular weight of between 5,000 kDa and 20,000 kDa; between 5,000 kDa and 15,000 kDa; between 5,000 kDa and 10,000 kDa; between 5,000 kDa and 7,500 kDa; between 6,000 kDa and 20,000 kDa; between 6,000 kDa and 15,000 kDa; between 6,000 kDa and 12,500 kDa; between 6,000 kDa and 10,000 kDa or between 6,000 kDa and 7,500 kDa.

The molecular weight of the glycoconjugate is measured by SEC-MALLS. Any whole number integer within any of the above ranges is contemplated as an embodiment of the disclosure.

In an embodiment, the serotype 23A glycoconjugate of the invention comprises at least 0.1, 0.2, 0.3, 0.4, 0.5, 0.6 or 0.7 or about 0.8 mM acetate per mM serotype 23A polysaccharide. In an embodiment, the glycoconjugate comprises at least 0.5, 0.6 or 0.7 mM acetate per mM serotype 23A polysaccharide. In an embodiment, the glycoconjugate comprises at least 0.6 mM acetate per mM serotype 23A polysaccharide. In another embodiment, the glycoconjugate comprises at least 0.7 mM acetate per mM serotype 23A polysaccharide.

In another embodiment, the ratio of mM acetate per mM serotype 23A polysaccharide in the glycoconjugate to mM acetate per mM serotype 23A polysaccharide in the isolated polysaccharide is at least 0.6, 0.65, 0.7, 0.75, 0.8, 0.85, 0.9, or 0.95. In an embodiment, the ratio of mM acetate per mM serotype 23A polysaccharide in the glycoconjugate to mM acetate per mM serotype 23A polysaccharide in the isolated polysaccharide is at least 0.7. In another embodiment, the ratio of mM acetate per mM serotype 23A polysaccharide in the glycoconjugate to mM acetate per mM serotype 23A polysaccharide in the isolated polysaccharide is at least 0.9.

In an embodiment, the ratio of mM acetate per mM serotype 23A polysaccharide in the glycoconjugate to mM acetate per mM serotype 23A polysaccharide in the activated polysaccharide is at least 0.6, 0.65, 0.7, 0.75, 0.8, 0.85, 0.9, or 0.95. In an embodiment, the ratio of mM acetate per mM serotype 23A polysaccharide in the glycoconjugate to mM acetate per mM serotype 23A polysaccharide in the activated polysaccharide is at least 0.7. In another embodiment, the ratio of mM acetate per mM serotype 23A polysaccharide in the glycoconjugate to mM acetate per mM serotype 23A polysaccharide in the activated polysaccharide is at least 0.9.

Another way to characterize the serotype 23A glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the CRM₁₉₇ protein starting material used to generate the conjugate materials. In an embodiment, the degree of conjugation of the serotype 23A glycoconjugate of the invention is between 2 and 15, between 2 and 13, between 2 and 10, between 2 and 8, between 2 and 6, between 2 and 5, between 2 and 4, between 3 and 15, between 3 and 13, between 3 and 10, between 3 and 8, between 3 and 6, between 3 and 5, between 3 and 4, between 5 and 15, between 5 and 10, between 8 and 15, between 8 and 12, between 10 and 15 or between 10 and 12. In an embodiment, the degree of conjugation of the serotype 23A glycoconjugate of the invention is about 2, about 3, about 4, about 5, about 6, about 7, about 8, about 9, about 10, about 11, about 12, about 13, about 14 or about 15. In an embodiment, the degree of conjugation of the serotype 23A glycoconjugate of the invention is between 4 and 7. In some such embodiments, the carrier protein is CRM₁₉₇.

The serotype 23A glycoconjugates of the invention may also be characterized by the ratio (weight/weight) of saccharide to carrier protein. In some embodiments, the ratio of serotype 23A polysaccharide to carrier protein in the glycoconjugate (w/w) is between 0.5 and 3.0 (e.g., about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0). In another embodiments, the saccharide to carrier protein ratio (w/w) is between 0.5 and 2.0, between 0.5 and 1.5, between 0.8 and 1.2, between 0.5 and 1.0, between 1.0 and 1.5 or between 1.0 and 2.0. In further embodiments, the saccharide to carrier protein ratio (w/w) is between 0.8 and 1.2. In an embodiment, the ratio of serotype 23A capsular polysaccharide to carrier protein in the conjugate is between 0.9 and 1.1. In some such embodiments, the carrier protein is CRM₁₉₇.

The serotype 23A glycoconjugates and immunogenic compositions of the invention may contain free saccharide that is not covalently conjugated to the carrier protein, but is nevertheless present in the glycoconjugate composition. The free saccharide may be noncovalently associated with (i.e., noncovalently bound to, adsorbed to, or entrapped in or with) the glycoconjugate.

In an embodiment, the serotype 23A glycoconjugate comprises less than about 50%, 45%, 40%, 35%, 30%, 25%, 20% or 15% of free serotype 23A polysaccharide compared to the total amount of serotype 23A polysaccharide. In another embodiment, the serotype 23A glycoconjugate comprises less than about 40% of free serotype 23A polysaccharide compared to the total amount of serotype 23A polysaccharide. In an embodiment, the serotype 23A glycoconjugate comprises less than about 25% of free serotype 23A polysaccharide compared to the total amount of serotype 23A polysaccharide. In an embodiment, the serotype 23A glycoconjugate comprises less than about 20% of free serotype 23A polysaccharide compared to the total amount of serotype 23A polysaccharide. In another embodiment the serotype 23A glycoconjugate comprises less than about 15% of free serotype 23A polysaccharide compared to the total amount of serotype 23A polysaccharide.

The serotype 23A glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In an embodiment, at least 30% of the serotype 23A glycoconjugate has a K_{d} below or equal to 0.3 in a CL-4B column. In another embodiment, at least 40% of the glycoconjugate has a K_{d} below or equal to 0.3 in a CL-4B column. In another embodiment, at least 45%, 50%, 55%, 60%, 65%, 70%, 75%, 80%, or 85% of the serotype 23A glycoconjugate has a K_{d} below or equal to 0.3 in a CL-4B column. In an embodiment, at least 60% of the serotype 23A glycoconjugate has a K_{d} below or equal to 0.3 in a CL-4B column. In another embodiment, between 50% and 80% of the serotype 23A glycoconjugate has a K_{d} below or equal to 0.3 in a CL-4B column. In an embodiment, between 65% and 80% of the serotype 23A glycoconjugate has a K_{d} below or equal to 0.3 in a CL-4B column.

1.3.12 Glycoconjugates from S. pneumoniae Serotype 23B

In an embodiment, the serotype 23B glycoconjugates are obtained by activating polysaccharide with 1-cyano-4-dimethylamino pyridinium tetrafluoroborate (CDAP) to form a cyanate ester. The activated polysaccharide may be coupled directly or via a spacer (linker) group to an amino group on the carrier protein. For example, the spacer may be cystamine or cysteamine to give a thiolated polysaccharide which may be coupled to the carrier via a thioether linkage obtained after reaction with a maleimide-activated carrier protein (for example using GMBS) or a haloacetylated carrier protein (for example using iodoacetimide, SIB, SIAB, sulfo-SIAB, SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO93/15760, WO95/08348 and WO96/129094.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO98/42721. Conjugation may involve a carbonyl linker which may be formed by reaction of a free hydroxyl group of the saccharide with CDI (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In one or more embodiments, the serotype 23B glycoconjugates of the invention are prepared using reductive amination. Reductive amination involves two steps: (1) oxidation of the polysaccharide to generate aldehyde functionalities from vicinal diols in individual hexasaccharide unit and (2) reduction of the activated polysaccharide and a carrier protein (e.g., CRM₁₉₇) to form a conjugate.

In an embodiment, before oxidation, sizing of the serotype 23B polysaccharide to a target molecular weight (MW) range is performed. Advantageously, the size of the purified serotype 23B polysaccharide is reduced while preserving critical features of the structure of the polysaccharide such as for example the presence of O-acetyl groups. In an embodiment, the size of the purified serotype 23B polysaccharide is reduced by mechanical homogenization as described herein.

In an embodiment, serotype polysaccharide is activated (oxidized) by a process comprising the step of:
(a) reacting isolated serotype 23B polysaccharide with an oxidizing agent; and
(b) quenching the oxidation reaction by addition of a quenching agent resulting in an activated serotype 23B polysaccharide.

In an embodiment, the oxidizing agent is periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate). In an embodiment, the oxidizing agent is sodium periodate. In an embodiment, the periodate used for the oxidation of serotype 23B polysaccharide is metaperiodate. In another embodiment, the periodate used for the oxidation of serotype 23B polysaccharide is sodium metaperiodate.

In one embodiment, the quenching agent is selected from vicinal diols, 1,2-aminoalcohols, amino acids, glutathione, sulfite, bisulfate, dithionite, metabisulfite, thiosulfate, phosphites, hypophosphites or phosphorous acid.

In one embodiment, the quenching agent is a 1,2-aminoalcohols of formula (I):

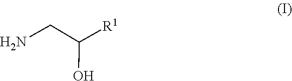

wherein R¹ is selected from H, methyl, ethyl, propyl or isopropyl.

In one embodiment, the quenching agent is selected from sodium and potassium salts of sulfite, bisulfate, dithionite, metabisulfite, thiosulfate, phosphites, hypophosphites or phosphorous acid.

In one embodiment, the quenching agent is an amino acid. In such embodiments, said amino acid may be selected from serine, threonine, cysteine, cystine, methionine, proline, hydroxyproline, tryptophan, tyrosine, and histidine.

In one embodiment, the quenching agent is a sulfite such as bisulfate, dithionite, metabisulfite, thiosulfate.

In one embodiment, the quenching agent is a compound comprising two vicinal hydroxyl groups (vicinal diols), i.e., two hydroxyl groups covalently linked to two adjacent carbon atoms.

In one or more embodiments, the quenching agent is a compound of formula (II):

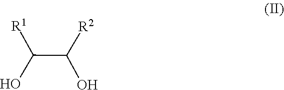

wherein R¹ and R² are each independently selected from H, methyl, ethyl, propyl or isopropyl.

In an embodiment, the quenching agent is glycerol, ethylene glycol, propan-1,2-diol, butan-1,2-diol or butan-2,3-diol, or ascorbic acid. In an embodiment, the quenching agent is butan-2,3-diol.

In another embodiment, the isolated serotype 23B polysaccharide is activated by a process comprising the steps of:
(a) reacting isolated serotype 23B polysaccharide with periodate; and
(b) quenching the oxidation reaction by addition of butan-2,3-diol resulting in an activated serotype 23B polysaccharide.

Following the oxidation step of the polysaccharide, the polysaccharide is said to be activated and is referred to as “activated polysaccharide” herein.

In an embodiment, the activated serotype 23B polysaccharide is purified. The activated serotype 23B polysaccharide is purified according to methods known to one skilled in the art such as gel permeation chromatography (GPC), dialysis or ultrafiltration/diafiltration. For example, the activated 23B polysaccharide is purified by concentration and diafiltration using an ultrafiltration device.

In another embodiment, the degree of oxidation of the activated serotype 23B polysaccharide is between 2 and 30, between 2 and 25, between 2 and 20, between 2 and 15, between 2 and 10, between 2 and 5, between 5 and 30, between 5 and 25, between 5 and 20, between 5 and 15, between 5 and 10, between 10 and 30, between 10 and 25, between 10 and 20, between 10 and 15, between 15 and 30, between 15 and 25, between 15 and 20, between 20 to 30, or between 20 to 25. In an embodiment the degree of oxidation of the activated serotype 23B polysaccharide is between 2 and 10, between 4 and 8, between 4 and 6, between 6 and 8, between 6 and 12, between 8 and 14, between 9 and 11, between 10 and 16, between 12 and 16, between 14 and 18, between 16 and 20, between 16 and 18, between 18 and 22, or between 18 and 20.

In an embodiment, the activated serotype 23B polysaccharide has a molecular weight between 25 kDa and 1,000 kDa, between 100 kDa and 1,000 kDa, between 300 kDa and 800 kDa, between 300 kDa and 700 kDa, between 300 kDa and 600 kDa, between 400 kDa and 1,000 kDa, between 400 kDa and 800 kDa, between 400 kDa and 700 kDa or between 400 kDa and 600 kDa. In an embodiment, the activated serotype 23B polysaccharide has a molecular weight between 300 kDa and 800 kDa. In an embodiment, the activated serotype 23B polysaccharide has a molecular weight between 400 kDa and 600 kDa. In another embodiment, the activated serotype 23B polysaccharide has a molecular weight between 400 kda and 600 kDa and a degree of oxidation between 10 and 25, between 10 and 20, between 12 and 20 or between 14 and 18. In another embodiment, the activated serotype 23B polysaccharide has a molecular weight between 400 kDa and 600 kDa and a degree of oxidation between 10 and 20.

In another embodiment, the activated serotype 23B polysaccharide comprises at least 0.1, 0.2, 0.3, 0.4, 0.5, 0.6 or 0.7 or about 0.8 mM acetate per mM serotype 23B polysaccharide. In an embodiment, the activated serotype 23B polysaccharide comprises at least 0.5, 0.6 or 0.7 mM acetate per mM serotype 23B polysaccharide. In another embodiment, the activated serotype 23B polysaccharide comprises at least 0.6 mM acetate per mM serotype 23B polysaccharide. In another embodiment, the activated serotype 23B polysaccharide comprises at least 0.7 mM acetate per mM serotype 23B polysaccharide.

In an embodiment, the activated serotype 23B polysaccharide has a molecular weight between 400 kDa and 800 kDa and comprises at least 0.6 mM acetate per mM serotype 23B polysaccharide.

In an embodiment, the activated serotype 23B polysaccharide has a molecular weight between 400 kDa and 800 kDa, a degree of oxidation between 12 and 20 and comprises at least 0.6 mM acetate per mM serotype 23B polysaccharide.

The activated polysaccharide and/or the carrier protein may be lyophilised (freeze-dried), either independently (discrete lyophilization) or together (co-lyophilized).

In another embodiment, the activated serotype 23B polysaccharide is lyophilized, optionally in the presence of saccharide. In an embodiment, the saccharide is selected from sucrose, trehalose, raffinose, stachyose, melezitose, dextran, mannitol, lactitol and palatinit. In an embodiment, the saccharide is sucrose. In one embodiment, the lyophilized activated polysaccharide is then compounded with a solution comprising the carrier protein.

In another embodiment, the activated polysaccharide and the carrier protein are co-lyophilised. In such embodiments, the activated serotype 23B polysaccharide is compounded with the carrier protein and lyophilized optionally in the presence of a saccharide. In an embodiment, the saccharide is selected from sucrose, trehalose, raffinose, stachyose, melezitose, dextran, mannitol, lactitol and palatinit. In an embodiment, the saccharide is sucrose. The co-lyophilized polysaccharide and carrier protein can then be resuspended in solution and reacted with a reducing agent.

The second step of the conjugation process is the reduction of the activated polysaccharide and a carrier protein to form a conjugate (reductive amination), using a reducing agent.

The activated serotype 23B polysaccharide can be conjugated to a carrier protein by a process comprising the steps of:
(c) compounding the activated serotype 23B polysaccharide with a carrier protein; and
(d) reacting the compounded activated serotype 23B polysaccharide and carrier protein with a reducing agent to form a serotype 23B polysaccharide-carrier protein conjugate.

In an embodiment, the reduction reaction is carried out in aqueous solvent. In another embodiment the reaction is carried out in aprotic solvent. In an embodiment, the reduction reaction is carried out in DMSO (dimethylsulfoxide) or in DMF (dimethylformamide)) solvent. The DMSO or DMF solvent may be used to reconstitute the activated polysaccharide and carrier protein which has been lyophilised.

The conjugation of activated serotype 23B polysaccharide with a protein carrier by reductive amination in dimethylsulfoxide (DMSO) is suitable to preserve the O-acetyl content of the polysaccharide as compared, for example, to reductive amination in aqueous phase where the level of O-acetylation of the polysaccharide may be significantly reduced. Therefore, in one or more embodiments, step (c) and step (d) are carried out in DMSO.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In an embodiment, the reducing agent is sodium cyanoborohydride.

At the end of the reduction reaction, there may be unreacted aldehyde groups remaining in the conjugates, these may be capped using a suitable capping agent. In one embodiment this capping agent is sodium borohydride (NaBH₄).

Following conjugation of serotype 23B polysaccharide to the carrier protein, the glycoconjugate can be purified (enriched with respect to the amount of polysaccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

In some embodiments, the serotype 23B glycoconjugates of the present invention comprise a saccharide having a molecular weight of between 10 kDa and 2,000 kDa. In other such embodiments, the saccharide has a molecular weight of between 50 kDa and 1,000 kDa. In other such embodiments, the saccharide has a molecular weight of between 70 kDa and 900 kDa. In other such embodiments, the saccharide has a molecular weight of between 100 kDa and 800 kDa. In other such embodiments, the saccharide has a molecular weight of between 200 kDa and 600 kDa. In further such embodiments, the saccharide has a molecular weight of 100 kDa to 1,000 kDa; 100 kDa to 900 kDa; 100 kDa to 800 kDa; 100 kDa to 700 kDa; 100 kDa to 600 kDa; 100 kDa to 500 kDa; 100 kDa to 400 kDa; 100 kDa to 300 kDa; 150 kDa to 1,000 kDa; 150 kDa to 900 kDa; 150 kDa to 800 kDa; 150 kDa to 700 kDa; 150 kDa to 600 kDa; 150 kDa to 500 kDa; 150 kDa to 400 kDa; 150 kDa to 300 kDa; 200 kDa to 1,000 kDa; 200 kDa to 900 kDa; 200 kDa to 800 kDa; 200 kDa to 700 kDa; 200 kDa to 600 kDa; 200 kDa to 500 kDa; 200 kDa to 400 kDa; 200 kDa to 300 kDa; 250 kDa to 1,000 kDa; 250 kDa to 900 kDa; 250 kDa to 800 kDa; 250 kDa to 700 kDa; 250 kDa to 600 kDa; 250 kDa to 500 kDa; 250 kDa to 400 kDa; 250 kDa to 350 kDa; 300 kDa to 1000 kDa; 300 kDa to 900 kDa; 300 kDa to 800 kDa; 300 kDa to 700 kDa; 300 kDa to 600 kDa; 300 kDa to 500 kDa; 300 kDa to 400 kDa; 400 kDa to 1,000 kDa; 400 kDa to 900 kDa; 400 kDa to 800 kDa; 400 kDa to 700 kDa; 400 kDa to 600 kDa; 500 kDa to 600 kDa. Any whole number integer within any of the above ranges is contemplated as an embodiment of the disclosure. In some such embodiments, the serotype 23A glycoconjugates are prepared using reductive amination.

In some embodiments, the serotype 23B glycoconjugate of the invention has a molecular weight of between 400 kDa and 15,000 kDa; between 500 kDa and 10,000 kDa; between 2,000 kDa and 10,000 kDa; between 3,000 kDa and 8,000 kDa; or between 3,000 kDa and 5,000 kDa. In other embodiments, the serotype 23B glycoconjugate has a molecular weight of between 500 kDa and 10,000 kDa. In other embodiments, the serotype 23A glycoconjugate has a molecular weight of between 1,000 kDa and 8,000 kDa. In still other embodiments, the serotype 23B glycoconjugate has a molecular weight of between 2,000 kDa and 8,000 kDa or between 3,000 kDa and 7,000 kDa. In further embodiments, the serotype 23B glycoconjugate of the invention has a molecular weight of between 200 kDa and 20,000 kDa; between 200 kDa and 15,000 kDa; between 200 kDa and 10,000 kDa; between 200 kDa and 7,500 kDa; between 200 kDa and 5,000 kDa; between 200 kDa and 3,000 kDa; between 200 kDa and 1,000 kDa; between 500 kDa and 20,000 kDa; between 500 kDa and 15,000 kDa; between 500 kDa and 12,500 kDa; between 500 kDa and 10,000 kDa; between 500 kDa and 7,500 kDa; between 500 kDa and 6,000 kDa; between 500 kDa and 5,000 kDa; between 500 kDa and 4,000 kDa; between 500 kDa and 3,000 kDa; between 500 kDa and 2,000 kDa; between 500 kDa and 1,500 kDa; between 500 kDa and 1,000 kDa; between 750 kDa and 20,000 kDa; between 750 kDa and 15,000 kDa; between 750 kDa and 12,500 kDa; between 750 kDa and 10,000 kDa; between 750 kDa and 7,500 kDa; between 750 kDa and 6,000 kDa; between 750 kDa and 5,000 kDa; between 750 kDa and 4,000 kDa; between 750 kDa and 3,000 kDa; between 750 kDa and 2,000 kDa; between 750 kDa and 1,500 kDa; between 1,000 kDa and 15,000 kDa; between 1,000 kDa and 12,500 kDa; between 1,000 kDa and 10,000 kDa; between 1,000 kDa and 7,500 kDa; between 1,000 kDa and 6,000 kDa; between 1,000 kDa and 5,000 kDa; between 1,000 kDa and 4,000 kDa; between 1,000 kDa and 2,500 kDa; between 2,000 kDa and 15,000 kDa; between 2,000 kDa and 12,500 kDa; between 2,000 kDa and 10,000 kDa; between 2,000 kDa and 7,500 kDa; between 2,000 kDa and 6,000 kDa; between 2,000 kDa and 5,000 kDa; between 2,000 kDa and 4,000 kDa; or between 2,000 kDa and 3,000 kDa.

In further embodiments, the serotype 23B glycoconjugate of the invention has a molecular weight of between 3,000 kDa and 20,000 kDa; between 3,000 kDa and 15,000 kDa; between 3,000 kDa and 10,000 kDa; between 3,000 kDa and 7,500 kDa; between 3,000 kDa and 5,000 kDa; between 4,000 kDa and 20,000 kDa; between 4,000 kDa and 15,000 kDa; between 4,000 kDa and 12,500 kDa; between 4,000 kDa and 10,000 kDa; between 4,000 kDa and 7,500 kDa; between 4,000 kDa and 6,000 kDa; or between 4,000 kDa and 5,000 kDa.

In further embodiments, the serotype 23B glycoconjugate of the invention has a molecular weight of between 5,000 kDa and 20,000 kDa; between 5,000 kDa and 15,000 kDa; between 5,000 kDa and 10,000 kDa; between 5,000 kDa and 7,500 kDa; between 6,000 kDa and 20,000 kDa; between 6,000 kDa and 15,000 kDa; between 6,000 kDa and 12,500 kDa; between 6,000 kDa and 10,000 kDa or between 6,000 kDa and 7,500 kDa.

The molecular weight of the glycoconjugate is measured by SEC-MALLS. Any whole number integer within any of the above ranges is contemplated as an embodiment of the disclosure.

In an embodiment, the serotype 23B glycoconjugate of the invention comprises at least 0.1, 0.2, 0.3, 0.4, 0.5, 0.6 or 0.7 or about 0.8 mM acetate per mM serotype 23B polysaccharide. In an embodiment, the glycoconjugate comprises at least 0.5, 0.6 or 0.7 mM acetate per mM serotype 23B polysaccharide. In an embodiment, the glycoconjugate comprises at least 0.6 mM acetate per mM serotype 23B polysaccharide. In another embodiment, the glycoconjugate comprises at least 0.7 mM acetate per mM serotype 23B polysaccharide.

In another embodiment, the ratio of mM acetate per mM serotype 23B polysaccharide in the glycoconjugate to mM acetate per mM serotype 23B polysaccharide in the isolated polysaccharide is at least 0.6, 0.65, 0.7, 0.75, 0.8, 0.85, 0.9, or 0.95. In an embodiment, the ratio of mM acetate per mM serotype 23B polysaccharide in the glycoconjugate to mM acetate per mM serotype 23B polysaccharide in the isolated polysaccharide is at least 0.7. In another embodiment, the ratio of mM acetate per mM serotype 23B polysaccharide in the glycoconjugate to mM acetate per mM serotype 23B polysaccharide in the isolated polysaccharide is at least 0.9.

In an embodiment, the ratio of mM acetate per mM serotype 23B polysaccharide in the glycoconjugate to mM acetate per mM serotype 23B polysaccharide in the activated polysaccharide is at least 0.6, 0.65, 0.7, 0.75, 0.8, 0.85, 0.9, or 0.95. In an embodiment, the ratio of mM acetate per mM serotype 23B polysaccharide in the glycoconjugate to mM acetate per mM serotype 23B polysaccharide in the activated polysaccharide is at least 0.7. In another embodiment, the ratio of mM acetate per mM serotype 23B polysaccharide in the glycoconjugate to mM acetate per mM serotype 23B polysaccharide in the activated polysaccharide is at least 0.9.

Another way to characterize the serotype 23B glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the CRM₁₉₇ protein starting material used to generate the conjugate materials. In an embodiment, the degree of conjugation of the serotype 23B glycoconjugate of the invention is between 2 and 15, between 2 and 13, between 2 and 10, between 2 and 8, between 2 and 6, between 2 and 5, between 2 and 4, between 3 and 15, between 3 and 13, between 3 and 10, between 3 and 8, between 3 and 6, between 3 and 5, between 3 and 4, between 5 and 15, between 5 and 10, between 8 and 15, between 8 and 12, between 10 and 15 or between 10 and 12. In an embodiment, the degree of conjugation of the serotype 23B glycoconjugate of the invention is about 2, about 3, about 4, about 5, about 6, about 7, about 8, about 9, about 10, about 11, about 12, about 13, about 14 or about 15. In an embodiment, the degree of conjugation of the serotype 23B glycoconjugate of the invention is between 4 and 7. In some such embodiments, the carrier protein is CRM₁₉₇.

The serotype 23B glycoconjugates of the invention may also be characterized by the ratio (weight/weight) of saccharide to carrier protein. In some embodiments, the ratio of serotype 23B polysaccharide to carrier protein in the glycoconjugate (w/w) is between 0.5 and 3.0 (e.g., about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0). In other embodiments, the saccharide to carrier protein ratio (w/w) is between 0.5 and 2.0, between 0.5 and 1.5, between 0.8 and 1.2, between 0.5 and 1.0, between 1.0 and 1.5 or between 1.0 and 2.0. In further embodiments, the saccharide to carrier protein ratio (w/w) is between 0.8 and 1.2. In an embodiment, the ratio of serotype 23A capsular polysaccharide to carrier protein in the conjugate is between 0.9 and 1.1. In some such embodiments, the carrier protein is CRM₁₉₇.

The serotype 23B glycoconjugates and immunogenic compositions of the invention may contain free saccharide that is not covalently conjugated to the carrier protein, but is nevertheless present in the glycoconjugate composition. The free saccharide may be noncovalently associated with (i.e., noncovalently bound to, adsorbed to, or entrapped in or with) the glycoconjugate.

In an embodiment, the serotype 23B glycoconjugate comprises less than about 50%, 45%, 40%, 35%, 30%, 25%, 20% or 15% of free serotype 23B polysaccharide compared to the total amount of serotype 23B polysaccharide. In another embodiment, the serotype 23B glycoconjugate comprises less than about 40% of free serotype 23B polysaccharide compared to the total amount of serotype 23B polysaccharide. In an embodiment, the serotype 23B glycoconjugate comprises less than about 25% of free serotype 23B polysaccharide compared to the total amount of serotype 23B polysaccharide. In an embodiment, the serotype 23B glycoconjugate comprises less than about 20% of free serotype 23B polysaccharide compared to the total amount of serotype 23B polysaccharide. In another embodiment the serotype 23B glycoconjugate comprises less than about 15% of free serotype 23B polysaccharide compared to the total amount of serotype 23B polysaccharide.

The serotype 23B glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In an embodiment, at least 30% of the serotype 23B glycoconjugate has a K_{d} below or equal to 0.3 in a CL-4B column. In another embodiment, at least 40% of the glycoconjugate has a K_{d} below or equal to 0.3 in a CL-4B column. In another embodiment, at least 45%, 50%, 55%, 60%, 65%, 70%, 75%, 80%, or 85% of the serotype 23B glycoconjugate has a K_{d} below or equal to 0.3 in a CL-4B column. In an embodiment, at least 60% of the serotype 23B glycoconjugate has a K_{d} below or equal to 0.3 in a CL-4B column. In another embodiment, between 50% and 80% of the serotype 23B glycoconjugate has a K_{d} below or equal to 0.3 in a CL-4B column. In an embodiment, between 65% and 80% of the serotype 23B glycoconjugate has a K_{d} below or equal to 0.3 in a CL-4B column.

1.3.13 Glycoconjugates from S. pneumoniae Serotype 31

In an embodiment, the serotype 31 glycoconjugates are obtained by activating polysaccharide with 1-cyano-4-dimethylamino pyridinium tetrafluoroborate (CDAP) to form a cyanate ester. The activated polysaccharide may be coupled directly or via a spacer (linker) group to an amino group on the carrier protein. For example, the spacer may be cystamine or cysteamine to give a thiolated polysaccharide which may be coupled to the carrier via a thioether linkage obtained after reaction with a maleimide-activated carrier protein (for example using GMBS) or a haloacetylated carrier protein (for example using iodoacetimide, SIB, SIAB, sulfo-SIAB, SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO93/15760, WO95/08348 and WO96/129094.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO98/42721. Conjugation may involve a carbonyl linker which may be formed by reaction of a free hydroxyl group of the saccharide with CDI (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In one or more embodiments, the serotype 31 glycoconjugates of the invention are prepared using reductive amination. Reductive amination involves two steps: (1) oxidation of the polysaccharide to generate aldehyde functionalities from vicinal diols in individual hexasaccharide unit and (2) reduction of the activated polysaccharide and a carrier protein (e.g., CRM₁₉₇) to form a conjugate.

In an embodiment, before oxidation, sizing of the serotype 31 polysaccharide to a target molecular weight (MW) range is performed. Advantageously, the size of the purified serotype 31 polysaccharide is reduced while preserving critical features of the structure of the polysaccharide such as for example the presence of O-acetyl groups. In an embodiment, the size of the purified serotype 31 polysaccharide is reduced by mechanical homogenization as described herein.

In an embodiment, serotype polysaccharide is activated (oxidized) by a process comprising the step of:
(a) reacting isolated serotype 31 polysaccharide with an oxidizing agent; and
(b) quenching the oxidation reaction by addition of a quenching agent resulting in an activated serotype 31 polysaccharide.

In an embodiment, the oxidizing agent is periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate). In an embodiment, the oxidizing agent is sodium periodate. In an embodiment, the periodate used for the oxidation of serotype 31 polysaccharide is metaperiodate. In another embodiment, the periodate used for the oxidation of serotype 31 polysaccharide is sodium metaperiodate.

In one embodiment, the quenching agent is selected from vicinal diols, 1,2-aminoalcohols, amino acids, glutathione, sulfite, bisulfate, dithionite, metabisulfite, thiosulfate, phosphites, hypophosphites or phosphorous acid.

In one embodiment, the quenching agent is a 1,2-aminoalcohols of formula (I):

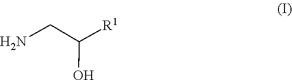

wherein R¹ is selected from H, methyl, ethyl, propyl or isopropyl.

In one embodiment, the quenching agent is selected from sodium and potassium salts of sulfite, bisulfate, dithionite, metabisulfite, thiosulfate, phosphites, hypophosphites or phosphorous acid.

In one embodiment, the quenching agent is an amino acid. In such embodiments, said amino acid may be selected from serine, threonine, cysteine, cystine, methionine, proline, hydroxyproline, tryptophan, tyrosine, and histidine.

In one embodiment, the quenching agent is a sulfite such as bisulfate, dithionite, metabisulfite, thiosulfate.

In one embodiment, the quenching agent is a compound comprising two vicinal hydroxyl groups (vicinal diols), i.e., two hydroxyl groups covalently linked to two adjacent carbon atoms.

In one or more embodiments, the quenching agent is a compound of formula (II):

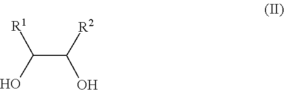

wherein R¹ and R² are each independently selected from H, methyl, ethyl, propyl or isopropyl.

In an embodiment, the quenching agent is glycerol, ethylene glycol, propan-1,2-diol, butan-1,2-diol or butan-2,3-diol, or ascorbic acid. In an embodiment, the quenching agent is butan-2,3-diol.

In another embodiment, the isolated serotype 31 polysaccharide is activated by a process comprising the steps of:
(a) reacting isolated serotype 31 polysaccharide with periodate; and
(b) quenching the oxidation reaction by addition of butan-2,3-diol resulting in an activated serotype 31 polysaccharide.

Following the oxidation step of the polysaccharide, the polysaccharide is said to be activated and is referred to as “activated polysaccharide” herein.

In an embodiment, the activated serotype 31 polysaccharide is purified. The activated serotype 31 polysaccharide is purified according to methods known to one skilled in the art such as gel permeation chromatography (GPC), dialysis or ultrafiltration/diafiltration. For example, the activated 31 polysaccharide is purified by concentration and diafiltration using an ultrafiltration device.

In another embodiment, the degree of oxidation of the activated serotype 31 polysaccharide is between 2 and 30, between 2 and 25, between 2 and 20, between 2 and 15, between 2 and 10, between 2 and 5, between 5 and 30, between 5 and 25, between 5 and 20, between 5 and 15, between 5 and 10, between 10 and 30, between 10 and 25, between 10 and 20, between 10 and 15, between 15 and 30, between 15 and 25, between 15 and 20, between 20 to 30, or between 20 to 25. In an embodiment the degree of oxidation of the activated serotype 31 polysaccharide is between 2 and 10, between 4 and 8, between 4 and 6, between 6 and 8, between 6 and 12, between 8 and 14, between 9 and 11, between 10 and 16, between 12 and 16, between 14 and 18, between 16 and 20, between 16 and 18, between 18 and 22, or between 18 and 20.

In an embodiment, the activated serotype 31 polysaccharide has a molecular weight between 25 kDa and 1,000 kDa, between 100 kDa and 1,000 kDa, between 300 kDa and 800 kDa, between 300 kDa and 700 kDa, between 300 kDa and 600 kDa, between 400 kDa and 1,000 kDa, between 400 kDa and 800 kDa, between 400 kDa and 700 kDa or between 400 kDa and 600 kDa. In an embodiment, the activated serotype 31 polysaccharide has a molecular weight between 300 kDa and 800 kDa. In an embodiment, the activated serotype 31 polysaccharide has a molecular weight between 400 kDa and 600 kDa. In another embodiment, the activated serotype 31 polysaccharide has a molecular weight between 400 kda and 600 kDa and a degree of oxidation between 10 and 25, between 10 and 20, between 12 and 20 or between 14 and 18. In another embodiment, the activated serotype 31 polysaccharide has a molecular weight between 400 kDa and 600 kDa and a degree of oxidation between 10 and 20.

In another embodiment, the activated serotype 31 polysaccharide comprises at least 0.1, 0.2, 0.3, 0.4, 0.5, 0.6 or 0.7 or about 0.8 mM acetate per mM serotype 31 polysaccharide. In an embodiment, the activated serotype 31 polysaccharide comprises at least 0.5, 0.6 or 0.7 mM acetate per mM serotype 31 polysaccharide. In another embodiment, the activated serotype 31 polysaccharide comprises at least 0.6 mM acetate per mM serotype 31 polysaccharide. In another embodiment, the activated serotype 31 polysaccharide comprises at least 0.7 mM acetate per mM serotype 31 polysaccharide.

In an embodiment, the activated serotype 31 polysaccharide has a molecular weight between 400 kDa and 800 kDa and comprises at least 0.6 mM acetate per mM serotype 31 polysaccharide.

In an embodiment, the activated serotype 31 polysaccharide has a molecular weight between 400 kDa and 800 kDa, a degree of oxidation between 12 and 20 and comprises at least 0.6 mM acetate per mM serotype 31 polysaccharide.

The activated polysaccharide and/or the carrier protein may be lyophilised (freeze-dried), either independently (discrete lyophilization) or together (co-lyophilized).

In another embodiment, the activated serotype 31 polysaccharide is lyophilized, optionally in the presence of saccharide. In an embodiment, the saccharide is selected from sucrose, trehalose, raffinose, stachyose, melezitose, dextran, mannitol, lactitol and palatinit. In an embodiment, the saccharide is sucrose. In one embodiment, the lyophilized activated polysaccharide is then compounded with a solution comprising the carrier protein.

In another embodiment, the activated polysaccharide and the carrier protein are co-lyophilised. In such embodiments, the activated serotype 31 polysaccharide is compounded with the carrier protein and lyophilized optionally in the presence of a saccharide. In an embodiment, the saccharide is selected from sucrose, trehalose, raffinose, stachyose, melezitose, dextran, mannitol, lactitol and palatinit. In an embodiment, the saccharide is sucrose. The co-lyophilized polysaccharide and carrier protein can then be resuspended in solution and reacted with a reducing agent.

The second step of the conjugation process is the reduction of the activated polysaccharide and a carrier protein to form a conjugate (reductive amination), using a reducing agent.

The activated serotype 31 polysaccharide can be conjugated to a carrier protein by a process comprising the steps of:
(c) compounding the activated serotype 31 polysaccharide with a carrier protein; and
(d) reacting the compounded activated serotype 31 polysaccharide and carrier protein with a reducing agent to form a serotype 31 polysaccharide-carrier protein conjugate.

In an embodiment, the reduction reaction is carried out in aqueous solvent. In another embodiment the reaction is carried out in aprotic solvent. In an embodiment, the reduction reaction is carried out in DMSO (dimethylsulfoxide) or in DMF (dimethylformamide)) solvent. The DMSO or DMF solvent may be used to reconstitute the activated polysaccharide and carrier protein which has been lyophilised.

The conjugation of activated serotype 31 polysaccharide with a protein carrier by reductive amination in dimethylsulfoxide (DMSO) is suitable to preserve the O-acetyl content of the polysaccharide as compared, for example, to reductive amination in aqueous phase where the level of O-acetylation of the polysaccharide may be significantly reduced. Therefore, in one or more embodiments, step (c) and step (d) are carried out in DMSO.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In an embodiment, the reducing agent is sodium cyanoborohydride.

At the end of the reduction reaction, there may be unreacted aldehyde groups remaining in the conjugates, these may be capped using a suitable capping agent. In one embodiment this capping agent is sodium borohydride (NaBH₄).

Following conjugation of serotype 31 polysaccharide to the carrier protein, the glycoconjugate can be purified (enriched with respect to the amount of polysaccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

In some embodiments, the serotype 31 glycoconjugates of the present invention comprise a saccharide having a molecular weight of between 10 kDa and 2,000 kDa. In other such embodiments, the saccharide has a molecular weight of between 50 kDa and 1,000 kDa. In other such embodiments, the saccharide has a molecular weight of between 70 kDa and 900 kDa. In other such embodiments, the saccharide has a molecular weight of between 100 kDa and 800 kDa. In other such embodiments, the saccharide has a molecular weight of between 200 kDa and 600 kDa. In further such embodiments, the saccharide has a molecular weight of 100 kDa to 1,000 kDa; 100 kDa to 900 kDa; 100 kDa to 800 kDa; 100 kDa to 700 kDa; 100 kDa to 600 kDa; 100 kDa to 500 kDa; 100 kDa to 400 kDa; 100 kDa to 300 kDa; 150 kDa to 1,000 kDa; 150 kDa to 900 kDa; 150 kDa to 800 kDa; 150 kDa to 700 kDa; 150 kDa to 600 kDa; 150 kDa to 500 kDa; 150 kDa to 400 kDa; 150 kDa to 300 kDa; 200 kDa to 1,000 kDa; 200 kDa to 900 kDa; 200 kDa to 800 kDa; 200 kDa to 700 kDa; 200 kDa to 600 kDa; 200 kDa to 500 kDa; 200 kDa to 400 kDa; 200 kDa to 300 kDa; 250 kDa to 1,000 kDa; 250 kDa to 900 kDa; 250 kDa to 800 kDa; 250 kDa to 700 kDa; 250 kDa to 600 kDa; 250 kDa to 500 kDa; 250 kDa to 400 kDa; 250 kDa to 350 kDa; 300 kDa to 1000 kDa; 300 kDa to 900 kDa; 300 kDa to 800 kDa; 300 kDa to 700 kDa; 300 kDa to 600 kDa; 300 kDa to 500 kDa; 300 kDa to 400 kDa; 400 kDa to 1,000 kDa; 400 kDa to 900 kDa; 400 kDa to 800 kDa; 400 kDa to 700 kDa; 400 kDa to 600 kDa; 500 kDa to 600 kDa. Any whole number integer within any of the above ranges is contemplated as an embodiment of the disclosure. In some such embodiments, the serotype 31 glycoconjugates are prepared using reductive amination.

In some embodiments, the serotype 31 glycoconjugate of the invention has a molecular weight of between 400 kDa and 15,000 kDa; between 500 kDa and 10,000 kDa; between 2,000 kDa and 10,000 kDa; between 3,000 kDa and 8,000 kDa; or between 3,000 kDa and 5,000 kDa. In other embodiments, the serotype 31 glycoconjugate has a molecular weight of between 500 kDa and 10,000 kDa. In other embodiments, the serotype 31 glycoconjugate has a molecular weight of between 1,000 kDa and 8,000 kDa. In still other embodiments, the serotype 31 glycoconjugate has a molecular weight of between 2,000 kDa and 8,000 kDa or between 3,000 kDa and 7,000 kDa. In further embodiments, the serotype 31 glycoconjugate of the invention has a molecular weight of between 200 kDa and 20,000 kDa; between 200 kDa and 15,000 kDa; between 200 kDa and 10,000 kDa; between 200 kDa and 7,500 kDa; between 200 kDa and 5,000 kDa; between 200 kDa and 3,000 kDa; between 200 kDa and 1,000 kDa; between 500 kDa and 20,000 kDa; between 500 kDa and 15,000 kDa; between 500 kDa and 12,500 kDa; between 500 kDa and 10,000 kDa; between 500 kDa and 7,500 kDa; between 500 kDa and 6,000 kDa; between 500 kDa and 5,000 kDa; between 500 kDa and 4,000 kDa; between 500 kDa and 3,000 kDa; between 500 kDa and 2,000 kDa; between 500 kDa and 1,500 kDa; between 500 kDa and 1,000 kDa; between 750 kDa and 20,000 kDa; between 750 kDa and 15,000 kDa; between 750 kDa and 12,500 kDa; between 750 kDa and 10,000 kDa; between 750 kDa and 7,500 kDa; between 750 kDa and 6,000 kDa; between 750 kDa and 5,000 kDa; between 750 kDa and 4,000 kDa; between 750 kDa and 3,000 kDa; between 750 kDa and 2,000 kDa; between 750 kDa and 1,500 kDa; between 1,000 kDa and 15,000 kDa; between 1,000 kDa and 12,500 kDa; between 1,000 kDa and 10,000 kDa; between 1,000 kDa and 7,500 kDa; between 1,000 kDa and 6,000 kDa; between 1,000 kDa and 5,000 kDa; between 1,000 kDa and 4,000 kDa; between 1,000 kDa and 2,500 kDa; between 2,000 kDa and 15,000 kDa; between 2,000 kDa and 12,500 kDa; between 2,000 kDa and 10,000 kDa; between 2,000 kDa and 7,500 kDa; between 2,000 kDa and 6,000 kDa; between 2,000 kDa and 5,000 kDa; between 2,000 kDa and 4,000 kDa; or between 2,000 kDa and 3,000 kDa.

In further embodiments, the serotype 31 glycoconjugate of the invention has a molecular weight of between 3,000 kDa and 20,000 kDa; between 3,000 kDa and 15,000 kDa; between 3,000 kDa and 10,000 kDa; between 3,000 kDa and 7,500 kDa; between 3,000 kDa and 5,000 kDa; between 4,000 kDa and 20,000 kDa; between 4,000 kDa and 15,000 kDa; between 4,000 kDa and 12,500 kDa; between 4,000 kDa and 10,000 kDa; between 4,000 kDa and 7,500 kDa; between 4,000 kDa and 6,000 kDa; or between 4,000 kDa and 5,000 kDa.

In further embodiments, the serotype 31 glycoconjugate of the invention has a molecular weight of between 5,000 kDa and 20,000 kDa; between 5,000 kDa and 15,000 kDa; between 5,000 kDa and 10,000 kDa; between 5,000 kDa and 7,500 kDa; between 6,000 kDa and 20,000 kDa; between 6,000 kDa and 15,000 kDa; between 6,000 kDa and 12,500 kDa; between 6,000 kDa and 10,000 kDa or between 6,000 kDa and 7,500 kDa.

The molecular weight of the glycoconjugate is measured by SEC-MALLS. Any whole number integer within any of the above ranges is contemplated as an embodiment of the disclosure.

In an embodiment, the serotype 31 glycoconjugate of the invention comprises at least 0.1, 0.2, 0.3, 0.4, 0.5, 0.6 or 0.7 or about 0.8 mM acetate per mM serotype 31 polysaccharide. In an embodiment, the glycoconjugate comprises at least 0.5, 0.6 or 0.7 mM acetate per mM serotype 31 polysaccharide. In an embodiment, the glycoconjugate comprises at least 0.6 mM acetate per mM serotype 31 polysaccharide. In another embodiment, the glycoconjugate comprises at least 0.7 mM acetate per mM serotype 31 polysaccharide.

In another embodiment, the ratio of mM acetate per mM serotype 31 polysaccharide in the glycoconjugate to mM acetate per mM serotype 31 polysaccharide in the isolated polysaccharide is at least 0.6, 0.65, 0.7, 0.75, 0.8, 0.85, 0.9, or 0.95. In an embodiment, the ratio of mM acetate per mM serotype 31 polysaccharide in the glycoconjugate to mM acetate per mM serotype 31 polysaccharide in the isolated polysaccharide is at least 0.7. In another embodiment, the ratio of mM acetate per mM serotype 31 polysaccharide in the glycoconjugate to mM acetate per mM serotype 31 polysaccharide in the isolated polysaccharide is at least 0.9.

In an embodiment, the ratio of mM acetate per mM serotype 31 polysaccharide in the glycoconjugate to mM acetate per mM serotype 31 polysaccharide in the activated polysaccharide is at least 0.6, 0.65, 0.7, 0.75, 0.8, 0.85, 0.9, or 0.95. In an embodiment, the ratio of mM acetate per mM serotype 31 polysaccharide in the glycoconjugate to mM acetate per mM serotype 31 polysaccharide in the activated polysaccharide is at least 0.7. In another embodiment, the ratio of mM acetate per mM serotype 31 polysaccharide in the glycoconjugate to mM acetate per mM serotype 31 polysaccharide in the activated polysaccharide is at least 0.9.

Another way to characterize the serotype 31 glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the CRM₁₉₇ protein starting material used to generate the conjugate materials. In an embodiment, the degree of conjugation of the serotype 31 glycoconjugate of the invention is between 2 and 15, between 2 and 13, between 2 and 10, between 2 and 8, between 2 and 6, between 2 and 5, between 2 and 4, between 3 and 15, between 3 and 13, between 3 and 10, between 3 and 8, between 3 and 6, between 3 and 5, between 3 and 4, between 5 and 15, between 5 and 10, between 8 and 15, between 8 and 12, between 10 and 15 or between 10 and 12. In an embodiment, the degree of conjugation of the serotype 31 glycoconjugate of the invention is about 2, about 3, about 4, about 5, about 6, about 7, about 8, about 9, about 10, about 11, about 12, about 13, about 14 or about 15. In an embodiment, the degree of conjugation of the serotype 31 glycoconjugate of the invention is between 4 and 7. In some such embodiments, the carrier protein is CRM₁₉₇.

The serotype 31 glycoconjugates of the invention may also be characterized by the ratio (weight/weight) of saccharide to carrier protein. In some embodiments, the ratio of serotype 31 polysaccharide to carrier protein in the glycoconjugate (w/w) is between 0.5 and 3.0 (e.g., about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0). In other embodiments, the saccharide to carrier protein ratio (w/w) is between 0.5 and 2.0, between 0.5 and 1.5, between 0.8 and 1.2, between 0.5 and 1.0, between 1.0 and 1.5 or between 1.0 and 2.0. In further embodiments, the saccharide to carrier protein ratio (w/w) is between 0.8 and 1.2. In an embodiment, the ratio of serotype 31 capsular polysaccharide to carrier protein in the conjugate is between 0.9 and 1.1. In some such embodiments, the carrier protein is CRM₁₉₇.

The serotype 31 glycoconjugates and immunogenic compositions of the invention may contain free saccharide that is not covalently conjugated to the carrier protein, but is nevertheless present in the glycoconjugate composition. The free saccharide may be noncovalently associated with (i.e., noncovalently bound to, adsorbed to, or entrapped in or with) the glycoconjugate.

In an embodiment, the serotype 31 glycoconjugate comprises less than about 50%, 45%, 40%, 35%, 30%, 25%, 20% or 15% of free serotype 31 polysaccharide compared to the total amount of serotype 31 polysaccharide. In another embodiment, the serotype 31 glycoconjugate comprises less than about 40% of free serotype 31 polysaccharide compared to the total amount of serotype 31 polysaccharide. In an embodiment, the serotype 31 glycoconjugate comprises less than about 25% of free serotype 31 polysaccharide compared to the total amount of serotype 31 polysaccharide. In an embodiment, the serotype 31 glycoconjugate comprises less than about 20% of free serotype 31 polysaccharide compared to the total amount of serotype 31 polysaccharide. In another embodiment the serotype 31 glycoconjugate comprises less than about 15% of free serotype 31 polysaccharide compared to the total amount of serotype 31 polysaccharide.

The serotype 31 glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In an embodiment, at least 30% of the serotype 31 glycoconjugate has a K_{d} below or equal to 0.3 in a CL-4B column. In another embodiment, at least 40% of the glycoconjugate has a K_{d} below or equal to 0.3 in a CL-4B column. In another embodiment, at least 45%, 50%, 55%, 60%, 65%, 70%, 75%, 80%, or 85% of the serotype 31 glycoconjugate has a K_{d} below or equal to 0.3 in a CL-4B column. In an embodiment, at least 60% of the serotype 31 glycoconjugate has a K_{d} below or equal to 0.3 in a CL-4B column. In another embodiment, between 50% and 80% of the serotype 31 glycoconjugate has a K_{d} below or equal to 0.3 in a CL-4B column. In an embodiment, between 65% and 80% of the serotype 31 glycoconjugate has a K_{d} below or equal to 0.3 in a CL-4B column.

1.3.14 Glycoconjugates from S. pneumoniae Serotype 34

In an embodiment, the serotype 34 glycoconjugates are obtained by activating polysaccharide with 1-cyano-4-dimethylamino pyridinium tetrafluoroborate (CDAP) to form a cyanate ester. The activated polysaccharide may be coupled directly or via a spacer (linker) group to an amino group on the carrier protein. For example, the spacer may be cystamine or cysteamine to give a thiolated polysaccharide which may be coupled to the carrier via a thioether linkage obtained after reaction with a maleimide-activated carrier protein (for example using GMBS) or a haloacetylated carrier protein (for example using iodoacetimide, SIB, SIAB, sulfo-SIAB, SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO93/15760, WO95/08348 and WO96/129094.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO98/42721. Conjugation may involve a carbonyl linker which may be formed by reaction of a free hydroxyl group of the saccharide with CDI (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In one or more embodiments, the serotype 34 glycoconjugates of the invention are prepared using reductive amination. Reductive amination involves two steps: (1) oxidation of the polysaccharide to generate aldehyde functionalities from vicinal diols in individual hexasaccharide unit and (2) reduction of the activated polysaccharide and a carrier protein (e.g., CRM₁₉₇) to form a conjugate.

In an embodiment, before oxidation, sizing of the serotype 34 polysaccharide to a target molecular weight (MW) range is performed. Advantageously, the size of the purified serotype 34 polysaccharide is reduced while preserving critical features of the structure of the polysaccharide such as for example the presence of O-acetyl groups. In an embodiment, the size of the purified serotype 34 polysaccharide is reduced by mechanical homogenization as described herein.

In an embodiment, serotype polysaccharide is activated (oxidized) by a process comprising the step of:
(a) reacting isolated serotype 34 polysaccharide with an oxidizing agent; and
(b) quenching the oxidation reaction by addition of a quenching agent resulting in an activated serotype 34 polysaccharide.

In an embodiment, the oxidizing agent is periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate). In an embodiment, the oxidizing agent is sodium periodate. In an embodiment, the periodate used for the oxidation of serotype 34 polysaccharide is metaperiodate. In another embodiment, the periodate used for the oxidation of serotype 34 polysaccharide is sodium metaperiodate.

In one embodiment, the quenching agent is selected from vicinal diols, 1,2-aminoalcohols, amino acids, glutathione, sulfite, bisulfate, dithionite, metabisulfite, thiosulfate, phosphites, hypophosphites or phosphorous acid.

In one embodiment, the quenching agent is a 1,2-aminoalcohols of formula (I):

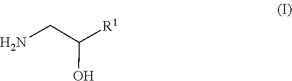

wherein R¹ is selected from H, methyl, ethyl, propyl or isopropyl.

In one embodiment, the quenching agent is selected from sodium and potassium salts of sulfite, bisulfate, dithionite, metabisulfite, thiosulfate, phosphites, hypophosphites or phosphorous acid.

In one embodiment, the quenching agent is an amino acid. In such embodiments, said amino acid may be selected from serine, threonine, cysteine, cystine, methionine, proline, hydroxyproline, tryptophan, tyrosine, and histidine.

In one embodiment, the quenching agent is a sulfite such as bisulfate, dithionite, metabisulfite, thiosulfate.

In one embodiment, the quenching agent is a compound comprising two vicinal hydroxyl groups (vicinal diols), i.e., two hydroxyl groups covalently linked to two adjacent carbon atoms.

In one or more embodiments, the quenching agent is a compound of formula (II):

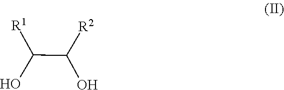

wherein R¹ and R² are each independently selected from H, methyl, ethyl, propyl or isopropyl.

In an embodiment, the quenching agent is glycerol, ethylene glycol, propan-1,2-diol, butan-1,2-diol or butan-2,3-diol, or ascorbic acid. In an embodiment, the quenching agent is butan-2,3-diol.

In another embodiment, the isolated serotype 34 polysaccharide is activated by a process comprising the steps of:
(a) reacting isolated serotype 34 polysaccharide with periodate; and
(b) quenching the oxidation reaction by addition of butan-2,3-diol resulting in an activated serotype 34 polysaccharide.

Following the oxidation step of the polysaccharide, the polysaccharide is said to be activated and is referred to as “activated polysaccharide” herein.

In an embodiment, the activated serotype 34 polysaccharide is purified. The activated serotype 34 polysaccharide is purified according to methods known to one skilled in the art such as gel permeation chromatography (GPC), dialysis or ultrafiltration/diafiltration. For example, the activated serotype 34 polysaccharide is purified by concentration and diafiltration using an ultrafiltration device.

In another embodiment, the degree of oxidation of the activated serotype 34 polysaccharide is between 2 and 30, between 2 and 25, between 2 and 20, between 2 and 15, between 2 and 10, between 2 and 5, between 5 and 30, between 5 and 25, between 5 and 20, between 5 and 15, between 5 and 10, between 10 and 30, between 10 and 25, between 10 and 20, between 10 and 15, between 15 and 30, between 15 and 25, between 15 and 20, between 20 to 30, or between 20 to 25. In an embodiment the degree of oxidation of the activated serotype 34 polysaccharide is between 2 and 10, between 4 and 8, between 4 and 6, between 6 and 8, between 6 and 12, between 8 and 14, between 9 and 11, between 10 and 16, between 12 and 16, between 14 and 18, between 16 and 20, between 16 and 18, between 18 and 22, or between 18 and 20.

In an embodiment, the activated serotype 34 polysaccharide has a molecular weight between 25 kDa and 1,000 kDa, between 100 kDa and 1,000 kDa, between 300 kDa and 800 kDa, between 300 kDa and 700 kDa, between 300 kDa and 600 kDa, between 400 kDa and 1,000 kDa, between 400 kDa and 800 kDa, between 400 kDa and 700 kDa or between 400 kDa and 600 kDa. In an embodiment, the activated serotype 34 polysaccharide has a molecular weight between 300 kDa and 800 kDa. In an embodiment, the activated serotype 34 polysaccharide has a molecular weight between 400 kDa and 600 kDa. In another embodiment, the activated serotype 34 polysaccharide has a molecular weight between 400 kda and 600 kDa and a degree of oxidation between 10 and 25, between 10 and 20, between 12 and 20 or between 14 and 18. In another embodiment, the activated serotype 34 polysaccharide has a molecular weight between 400 kDa and 600 kDa and a degree of oxidation between 10 and 20.

In another embodiment, the activated serotype 34 polysaccharide comprises at least 0.1, 0.2, 0.3, 0.4, 0.5, 0.6 or 0.7 or about 0.8 mM acetate per mM serotype 34 polysaccharide. In an embodiment, the activated serotype 34 polysaccharide comprises at least 0.5, 0.6 or 0.7 mM acetate per mM serotype 34 polysaccharide. In another embodiment, the activated serotype 34 polysaccharide comprises at least 0.6 mM acetate per mM serotype 34 polysaccharide. In another embodiment, the activated serotype 34 polysaccharide comprises at least 0.7 mM acetate per mM serotype 34 polysaccharide.

In an embodiment, the activated serotype 34 polysaccharide has a molecular weight between 400 kDa and 800 kDa and comprises at least 0.6 mM acetate per mM serotype 34 polysaccharide.

In an embodiment, the activated serotype 34 polysaccharide has a molecular weight between 400 kDa and 800 kDa, a degree of oxidation between 12 and 20 and comprises at least 0.6 mM acetate per mM serotype 34 polysaccharide.

The activated polysaccharide and/or the carrier protein may be lyophilised (freeze-dried), either independently (discrete lyophilization) or together (co-lyophilized).

In another embodiment, the activated serotype 34 polysaccharide is lyophilized, optionally in the presence of saccharide. In an embodiment, the saccharide is selected from sucrose, trehalose, raffinose, stachyose, melezitose, dextran, mannitol, lactitol and palatinit. In an embodiment, the saccharide is sucrose. In one embodiment, the lyophilized activated polysaccharide is then compounded with a solution comprising the carrier protein.

In another embodiment, the activated polysaccharide and the carrier protein are co-lyophilised. In such embodiments, the activated serotype 34 polysaccharide is compounded with the carrier protein and lyophilized optionally in the presence of a saccharide. In an embodiment, the saccharide is selected from sucrose, trehalose, raffinose, stachyose, melezitose, dextran, mannitol, lactitol and palatinit. In an embodiment, the saccharide is sucrose. The co-lyophilized polysaccharide and carrier protein can then be resuspended in solution and reacted with a reducing agent.

The second step of the conjugation process is the reduction of the activated polysaccharide and a carrier protein to form a conjugate (reductive amination), using a reducing agent.

The activated serotype 34 polysaccharide can be conjugated to a carrier protein by a process comprising the steps of:
(c) compounding the activated serotype 34 polysaccharide with a carrier protein; and
(d) reacting the compounded activated serotype 34 polysaccharide and carrier protein with a reducing agent to form a serotype 34 polysaccharide-carrier protein conjugate.

In an embodiment, the reduction reaction is carried out in aqueous solvent. In another embodiment the reaction is carried out in aprotic solvent. In an embodiment, the reduction reaction is carried out in DMSO (dimethylsulfoxide) or in DMF (dimethylformamide)) solvent. The DMSO or DMF solvent may be used to reconstitute the activated polysaccharide and carrier protein which has been lyophilised.

The conjugation of activated serotype 34 polysaccharide with a protein carrier by reductive amination in dimethylsulfoxide (DMSO) is suitable to preserve the O-acetyl content of the polysaccharide as compared, for example, to reductive amination in aqueous phase where the level of O-acetylation of the polysaccharide may be significantly reduced. Therefore, in one or more embodiments, step (c) and step (d) are carried out in DMSO.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In an embodiment, the reducing agent is sodium cyanoborohydride.

At the end of the reduction reaction, there may be unreacted aldehyde groups remaining in the conjugates, these may be capped using a suitable capping agent. In one embodiment this capping agent is sodium borohydride (NaBH₄).

Following conjugation of serotype 34 polysaccharide to the carrier protein, the glycoconjugate can be purified (enriched with respect to the amount of polysaccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

In some embodiments, the serotype 34 glycoconjugates of the present invention comprise a saccharide having a molecular weight of between 10 kDa and 2,000 kDa. In other such embodiments, the saccharide has a molecular weight of between 50 kDa and 1,000 kDa. In other such embodiments, the saccharide has a molecular weight of between 70 kDa and 900 kDa. In other such embodiments, the saccharide has a molecular weight of between 100 kDa and 800 kDa. In other such embodiments, the saccharide has a molecular weight of between 200 kDa and 600 kDa. In further such embodiments, the saccharide has a molecular weight of 100 kDa to 1,000 kDa; 100 kDa to 900 kDa; 100 kDa to 800 kDa; 100 kDa to 700 kDa; 100 kDa to 600 kDa; 100 kDa to 500 kDa; 100 kDa to 400 kDa; 100 kDa to 300 kDa; 150 kDa to 1,000 kDa; 150 kDa to 900 kDa; 150 kDa to 800 kDa; 150 kDa to 700 kDa; 150 kDa to 600 kDa; 150 kDa to 500 kDa; 150 kDa to 400 kDa; 150 kDa to 300 kDa; 200 kDa to 1,000 kDa; 200 kDa to 900 kDa; 200 kDa to 800 kDa; 200 kDa to 700 kDa; 200 kDa to 600 kDa; 200 kDa to 500 kDa; 200 kDa to 400 kDa; 200 kDa to 300 kDa; 250 kDa to 1,000 kDa; 250 kDa to 900 kDa; 250 kDa to 800 kDa; 250 kDa to 700 kDa; 250 kDa to 600 kDa; 250 kDa to 500 kDa; 250 kDa to 400 kDa; 250 kDa to 350 kDa; 300 kDa to 1000 kDa; 300 kDa to 900 kDa; 300 kDa to 800 kDa; 300 kDa to 700 kDa; 300 kDa to 600 kDa; 300 kDa to 500 kDa; 300 kDa to 400 kDa; 400 kDa to 1,000 kDa; 400 kDa to 900 kDa; 400 kDa to 800 kDa; 400 kDa to 700 kDa; 400 kDa to 600 kDa; 500 kDa to 600 kDa. Any whole number integer within any of the above ranges is contemplated as an embodiment of the disclosure. In some such embodiments, the serotype 34 glycoconjugates are prepared using reductive amination.

In some embodiments, the serotype 34 glycoconjugate of the invention has a molecular weight of between 400 kDa and 15,000 kDa; between 500 kDa and 10,000 kDa; between 2,000 kDa and 10,000 kDa; between 3,000 kDa and 8,000 kDa; or between 3,000 kDa and 5,000 kDa. In other embodiments, the serotype 34 glycoconjugate has a molecular weight of between 500 kDa and 10,000 kDa. In other embodiments, the serotype 34 glycoconjugate has a molecular weight of between 1,000 kDa and 8,000 kDa. In still other embodiments, the serotype 34 glycoconjugate has a molecular weight of between 2,000 kDa and 8,000 kDa or between 3,000 kDa and 7,000 kDa. In further embodiments, the serotype 34 glycoconjugate of the invention has a molecular weight of between 200 kDa and 20,000 kDa; between 200 kDa and 15,000 kDa; between 200 kDa and 10,000 kDa; between 200 kDa and 7,500 kDa; between 200 kDa and 5,000 kDa; between 200 kDa and 3,000 kDa; between 200 kDa and 1,000 kDa; between 500 kDa and 20,000 kDa; between 500 kDa and 15,000 kDa; between 500 kDa and 12,500 kDa; between 500 kDa and 10,000 kDa; between 500 kDa and 7,500 kDa; between 500 kDa and 6,000 kDa; between 500 kDa and 5,000 kDa; between 500 kDa and 4,000 kDa; between 500 kDa and 3,000 kDa; between 500 kDa and 2,000 kDa; between 500 kDa and 1,500 kDa; between 500 kDa and 1,000 kDa; between 750 kDa and 20,000 kDa; between 750 kDa and 15,000 kDa; between 750 kDa and 12,500 kDa; between 750 kDa and 10,000 kDa; between 750 kDa and 7,500 kDa; between 750 kDa and 6,000 kDa; between 750 kDa and 5,000 kDa; between 750 kDa and 4,000 kDa; between 750 kDa and 3,000 kDa; between 750 kDa and 2,000 kDa; between 750 kDa and 1,500 kDa; between 1,000 kDa and 15,000 kDa; between 1,000 kDa and 12,500 kDa; between 1,000 kDa and 10,000 kDa; between 1,000 kDa and 7,500 kDa; between 1,000 kDa and 6,000 kDa; between 1,000 kDa and 5,000 kDa; between 1,000 kDa and 4,000 kDa; between 1,000 kDa and 2,500 kDa; between 2,000 kDa and 15,000 kDa; between 2,000 kDa and 12,500 kDa; between 2,000 kDa and 10,000 kDa; between 2,000 kDa and 7,500 kDa; between 2,000 kDa and 6,000 kDa; between 2,000 kDa and 5,000 kDa; between 2,000 kDa and 4,000 kDa; or between 2,000 kDa and 3,000 kDa.

In further embodiments, the serotype 34 glycoconjugate of the invention has a molecular weight of between 3,000 kDa and 20,000 kDa; between 3,000 kDa and 15,000 kDa; between 3,000 kDa and 10,000 kDa; between 3,000 kDa and 7,500 kDa; between 3,000 kDa and 5,000 kDa; between 4,000 kDa and 20,000 kDa; between 4,000 kDa and 15,000 kDa; between 4,000 kDa and 12,500 kDa; between 4,000 kDa and 10,000 kDa; between 4,000 kDa and 7,500 kDa; between 4,000 kDa and 6,000 kDa; or between 4,000 kDa and 5,000 kDa.

In further embodiments, the serotype 34 glycoconjugate of the invention has a molecular weight of between 5,000 kDa and 20,000 kDa; between 5,000 kDa and 15,000 kDa; between 5,000 kDa and 10,000 kDa; between 5,000 kDa and 7,500 kDa; between 6,000 kDa and 20,000 kDa; between 6,000 kDa and 15,000 kDa; between 6,000 kDa and 12,500 kDa; between 6,000 kDa and 10,000 kDa or between 6,000 kDa and 7,500 kDa.

The molecular weight of the glycoconjugate is measured by SEC-MALLS. Any whole number integer within any of the above ranges is contemplated as an embodiment of the disclosure.

In an embodiment, the serotype 34 glycoconjugate of the invention comprises at least 0.1, 0.2, 0.3, 0.4, 0.5, 0.6 or 0.7 or about 0.8 mM acetate per mM serotype 34 polysaccharide. In an embodiment, the glycoconjugate comprises at least 0.5, 0.6 or 0.7 mM acetate per mM serotype 34 polysaccharide. In an embodiment, the glycoconjugate comprises at least 0.6 mM acetate per mM serotype 34 polysaccharide. In another embodiment, the glycoconjugate comprises at least 0.7 mM acetate per mM serotype 34 polysaccharide.

In another embodiment, the ratio of mM acetate per mM serotype 34 polysaccharide in the glycoconjugate to mM acetate per mM serotype 34 polysaccharide in the isolated polysaccharide is at least 0.6, 0.65, 0.7, 0.75, 0.8, 0.85, 0.9, or 0.95. In an embodiment, the ratio of mM acetate per mM serotype 34 polysaccharide in the glycoconjugate to mM acetate per mM serotype 34 polysaccharide in the isolated polysaccharide is at least 0.7. In another embodiment, the ratio of mM acetate per mM serotype 34 polysaccharide in the glycoconjugate to mM acetate per mM serotype 34 polysaccharide in the isolated polysaccharide is at least 0.9.

In an embodiment, the ratio of mM acetate per mM serotype 34 polysaccharide in the glycoconjugate to mM acetate per mM serotype 34 polysaccharide in the activated polysaccharide is at least 0.6, 0.65, 0.7, 0.75, 0.8, 0.85, 0.9, or 0.95. In an embodiment, the ratio of mM acetate per mM serotype 34 polysaccharide in the glycoconjugate to mM acetate per mM serotype 34 polysaccharide in the activated polysaccharide is at least 0.7. In another embodiment, the ratio of mM acetate per mM serotype 34 polysaccharide in the glycoconjugate to mM acetate per mM serotype 34 polysaccharide in the activated polysaccharide is at least 0.9.

Another way to characterize the serotype 34 glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the CRM₁₉₇ protein starting material used to generate the conjugate materials. In an embodiment, the degree of conjugation of the serotype 34 glycoconjugate of the invention is between 2 and 15, between 2 and 13, between 2 and 10, between 2 and 8, between 2 and 6, between 2 and 5, between 2 and 4, between 3 and 15, between 3 and 13, between 3 and 10, between 3 and 8, between 3 and 6, between 3 and 5, between 3 and 4, between 5 and 15, between 5 and 10, between 8 and 15, between 8 and 12, between 10 and 15 or between 10 and 12. In an embodiment, the degree of conjugation of the serotype 34 glycoconjugate of the invention is about 2, about 3, about 4, about 5, about 6, about 7, about 8, about 9, about 10, about 11, about 12, about 13, about 14 or about 15. In an embodiment, the degree of conjugation of the serotype 34 glycoconjugate of the invention is between 4 and 7. In some such embodiments, the carrier protein is CRM₁₉₇.

The serotype 34 glycoconjugates of the invention may also be characterized by the ratio (weight/weight) of saccharide to carrier protein. In some embodiments, the ratio of serotype 34 polysaccharide to carrier protein in the glycoconjugate (w/w) is between 0.5 and 3.0 (e.g., about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0). In other embodiments, the saccharide to carrier protein ratio (w/w) is between 0.5 and 2.0, between 0.5 and 1.5, between 0.8 and 1.2, between 0.5 and 1.0, between 1.0 and 1.5 or between 1.0 and 2.0. In further embodiments, the saccharide to carrier protein ratio (w/w) is between 0.8 and 1.2. In an embodiment, the ratio of serotype 34 capsular polysaccharide to carrier protein in the conjugate is between 0.9 and 1.1. In some such embodiments, the carrier protein is CRM₁₉₇.

The serotype 34 glycoconjugates and immunogenic compositions of the invention may contain free saccharide that is not covalently conjugated to the carrier protein, but is nevertheless present in the glycoconjugate composition. The free saccharide may be noncovalently associated with (i.e., noncovalently bound to, adsorbed to, or entrapped in or with) the glycoconjugate.

In an embodiment, the serotype 34 glycoconjugate comprises less than about 50%, 45%, 40%, 35%, 30%, 25%, 20% or 15% of free serotype 34 polysaccharide compared to the total amount of serotype 34 polysaccharide. In another embodiment, the serotype 34 glycoconjugate comprises less than about 40% of free serotype 34 polysaccharide compared to the total amount of serotype 34 polysaccharide. In an embodiment, the serotype 34 glycoconjugate comprises less than about 25% of free serotype 34 polysaccharide compared to the total amount of serotype 34 polysaccharide. In an embodiment, the serotype 34 glycoconjugate comprises less than about 20% of free serotype 34 polysaccharide compared to the total amount of serotype 34 polysaccharide. In another embodiment the serotype 34 glycoconjugate comprises less than about 15% of free serotype 34 polysaccharide compared to the total amount of serotype 34 polysaccharide.

The serotype 34 glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In an embodiment, at least 30% of the serotype 34 glycoconjugate has a K_{d} below or equal to 0.3 in a CL-4B column. In another embodiment, at least 40% of the glycoconjugate has a K_{d} below or equal to 0.3 in a CL-4B column. In another embodiment, at least 45%, 50%, 55%, 60%, 65%, 70%, 75%, 80%, or 85% of the serotype 34 glycoconjugate has a K_{d} below or equal to 0.3 in a CL-4B column. In an embodiment, at least 60% of the serotype 34 glycoconjugate has a K_{d} below or equal to 0.3 in a CL-4B column. In another embodiment, between 50% and 80% of the serotype 34 glycoconjugate has a K_{d} below or equal to 0.3 in a CL-4B column. In an embodiment, between 65% and 80% of the serotype 34 glycoconjugate has a K_{d} below or equal to 0.3 in a CL-4B column.

1.3.15 Glycoconjugates from S. pneumoniae Serotype 35B

In an embodiment, the serotype 35B glycoconjugates are obtained by activating polysaccharide with 1-cyano-4-dimethylamino pyridinium tetrafluoroborate (CDAP) to form a cyanate ester. The activated polysaccharide may be coupled directly or via a spacer (linker) group to an amino group on the carrier protein. For example, the spacer may be cystamine or cysteamine to give a thiolated polysaccharide which may be coupled to the carrier via a thioether linkage obtained after reaction with a maleimide-activated carrier protein (for example using GMBS) or a haloacetylated carrier protein (for example using iodoacetimide, SIB, SIAB, sulfo-SIAB, SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO93/15760, WO95/08348 and WO96/129094.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO98/42721. Conjugation may involve a carbonyl linker which may be formed by reaction of a free hydroxyl group of the saccharide with CDI (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In one or more embodiments, the serotype 35B glycoconjugates of the invention are prepared using reductive amination. Reductive amination involves two steps: (1) oxidation of the polysaccharide to generate aldehyde functionalities from vicinal diols in individual hexasaccharide unit and (2) reduction of the activated polysaccharide and a carrier protein (e.g., CRM₁₉₇) to form a conjugate.

In an embodiment, before oxidation, sizing of the serotype 35B polysaccharide to a target molecular weight (MW) range is performed. Advantageously, the size of the purified serotype 35B polysaccharide is reduced while preserving critical features of the structure of the polysaccharide such as for example the presence of O-acetyl groups. In an embodiment, the size of the purified serotype 35B polysaccharide is reduced by mechanical homogenization as described herein.

In an embodiment, serotype polysaccharide is activated (oxidized) by a process comprising the step of:
(a) reacting isolated serotype 35B polysaccharide with an oxidizing agent; and
(b) quenching the oxidation reaction by addition of a quenching agent resulting in an activated serotype 35B polysaccharide.

In an embodiment, the oxidizing agent is periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate). In an embodiment, the oxidizing agent is sodium periodate. In an embodiment, the periodate used for the oxidation of serotype 35B polysaccharide is metaperiodate. In another embodiment, the periodate used for the oxidation of serotype 35B polysaccharide is sodium metaperiodate.

In one embodiment, the quenching agent is selected from vicinal diols, 1,2-aminoalcohols, amino acids, glutathione, sulfite, bisulfate, dithionite, metabisulfite, thiosulfate, phosphites, hypophosphites or phosphorous acid.

In one embodiment, the quenching agent is a 1,2-aminoalcohols of formula (I):

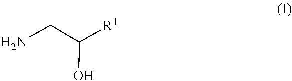

wherein R¹ is selected from H, methyl, ethyl, propyl or isopropyl.

In one embodiment, the quenching agent is selected from sodium and potassium salts of sulfite, bisulfate, dithionite, metabisulfite, thiosulfate, phosphites, hypophosphites or phosphorous acid.

In one embodiment, the quenching agent is an amino acid. In such embodiments, said amino acid may be selected from serine, threonine, cysteine, cystine, methionine, proline, hydroxyproline, tryptophan, tyrosine, and histidine.

In one embodiment, the quenching agent is a sulfite such as bisulfate, dithionite, metabisulfite, thiosulfate.

In one embodiment, the quenching agent is a compound comprising two vicinal hydroxyl groups (vicinal diols), i.e., two hydroxyl groups covalently linked to two adjacent carbon atoms.

In one or more embodiments, the quenching agent is a compound of formula (II):

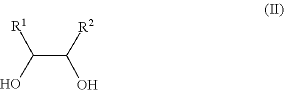

wherein R¹ and R² are each independently selected from H, methyl, ethyl, propyl or isopropyl.

In an embodiment, the quenching agent is glycerol, ethylene glycol, propan-1,2-diol, butan-1,2-diol or butan-2,3-diol, or ascorbic acid. In an embodiment, the quenching agent is butan-2,3-diol.

In another embodiment, the isolated serotype 35B polysaccharide is activated by a process comprising the steps of:
(a) reacting isolated serotype 35B polysaccharide with periodate; and
(b) quenching the oxidation reaction by addition of butan-2,3-diol resulting in an activated serotype 35B polysaccharide.

Following the oxidation step of the polysaccharide, the polysaccharide is said to be activated and is referred to as “activated polysaccharide” herein.

In an embodiment, the activated serotype 35B polysaccharide is purified. The activated serotype 35B polysaccharide is purified according to methods known to one skilled in the art such as gel permeation chromatography (GPC), dialysis or ultrafiltration/diafiltration. For example, the activated 35B polysaccharide is purified by concentration and diafiltration using an ultrafiltration device.

In another embodiment, the degree of oxidation of the activated serotype 35B polysaccharide is between 2 and 30, between 2 and 25, between 2 and 20, between 2 and 15, between 2 and 10, between 2 and 5, between 5 and 30, between 5 and 25, between 5 and 20, between 5 and 15, between 5 and 10, between 10 and 30, between 10 and 25, between 10 and 20, between 10 and 15, between 15 and 30, between 15 and 25, between 15 and 20, between 20 to 30, or between 20 to 25. In an embodiment the degree of oxidation of the activated serotype 35B polysaccharide is between 2 and 10, between 4 and 8, between 4 and 6, between 6 and 8, between 6 and 12, between 8 and 14, between 9 and 11, between 10 and 16, between 12 and 16, between 14 and 18, between 16 and 20, between 16 and 18, between 18 and 22, or between 18 and 20.

In an embodiment, the activated serotype 35B polysaccharide has a molecular weight between 25 kDa and 1,000 kDa, between 100 kDa and 1,000 kDa, between 300 kDa and 800 kDa, between 300 kDa and 700 kDa, between 300 kDa and 600 kDa, between 400 kDa and 1,000 kDa, between 400 kDa and 800 kDa, between 400 kDa and 700 kDa or between 400 kDa and 600 kDa. In an embodiment, the activated serotype 35B polysaccharide has a molecular weight between 300 kDa and 800 kDa. In an embodiment, the activated serotype 35B polysaccharide has a molecular weight between 400 kDa and 600 kDa. In another embodiment, the activated serotype 35B polysaccharide has a molecular weight between 400 kda and 600 kDa and a degree of oxidation between 10 and 25, between 10 and 20, between 12 and 20 or between 14 and 18. In another embodiment, the activated serotype 35B polysaccharide has a molecular weight between 400 kDa and 600 kDa and a degree of oxidation between 10 and 20.

In another embodiment, the activated serotype 35B polysaccharide comprises at least 0.1, 0.2, 0.3, 0.4, 0.5, 0.6 or 0.7 or about 0.8 mM acetate per mM serotype 35B polysaccharide. In an embodiment, the activated serotype 35B polysaccharide comprises at least 0.5, 0.6 or 0.7 mM acetate per mM serotype 35B polysaccharide. In another embodiment, the activated serotype 35B polysaccharide comprises at least 0.6 mM acetate per mM serotype 35B polysaccharide. In another embodiment, the activated serotype 35B polysaccharide comprises at least 0.7 mM acetate per mM serotype 35B polysaccharide.

In an embodiment, the activated serotype 35B polysaccharide has a molecular weight between 400 kDa and 800 kDa and comprises at least 0.6 mM acetate per mM serotype 35B polysaccharide.

In an embodiment, the activated serotype 35B polysaccharide has a molecular weight between 400 kDa and 800 kDa, a degree of oxidation between 12 and 20 and comprises at least 0.6 mM acetate per mM serotype 35B polysaccharide.

The activated polysaccharide and/or the carrier protein may be lyophilised (freeze-dried), either independently (discrete lyophilization) or together (co-lyophilized).

In another embodiment, the activated serotype 35B polysaccharide is lyophilized, optionally in the presence of saccharide. In an embodiment, the saccharide is selected from sucrose, trehalose, raffinose, stachyose, melezitose, dextran, mannitol, lactitol and palatinit. In an embodiment, the saccharide is sucrose. In one embodiment, the lyophilized activated polysaccharide is then compounded with a solution comprising the carrier protein.

In another embodiment, the activated polysaccharide and the carrier protein are co-lyophilised. In such embodiments, the activated serotype 35B polysaccharide is compounded with the carrier protein and lyophilized optionally in the presence of a saccharide. In an embodiment, the saccharide is selected from sucrose, trehalose, raffinose, stachyose, melezitose, dextran, mannitol, lactitol and palatinit. In an embodiment, the saccharide is sucrose. The co-lyophilized polysaccharide and carrier protein can then be resuspended in solution and reacted with a reducing agent.

The second step of the conjugation process is the reduction of the activated polysaccharide and a carrier protein to form a conjugate (reductive amination), using a reducing agent.

The activated serotype 35B polysaccharide can be conjugated to a carrier protein by a process comprising the steps of:
(c) compounding the activated serotype 35B polysaccharide with a carrier protein; and
(d) reacting the compounded activated serotype 35B polysaccharide and carrier protein with a reducing agent to form a serotype 35B polysaccharide-carrier protein conjugate.

In an embodiment, the reduction reaction is carried out in aqueous solvent. In another embodiment the reaction is carried out in aprotic solvent. In an embodiment, the reduction reaction is carried out in DMSO (dimethylsulfoxide) or in DMF (dimethylformamide)) solvent. The DMSO or DMF solvent may be used to reconstitute the activated polysaccharide and carrier protein which has been lyophilised.

The conjugation of activated serotype 35B polysaccharide with a protein carrier by reductive amination in dimethylsulfoxide (DMSO) is suitable to preserve the O-acetyl content of the polysaccharide as compared, for example, to reductive amination in aqueous phase where the level of O-acetylation of the polysaccharide may be significantly reduced. Therefore, in one or more embodiments, step (c) and step (d) are carried out in DMSO.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In an embodiment, the reducing agent is sodium cyanoborohydride.

At the end of the reduction reaction, there may be unreacted aldehyde groups remaining in the conjugates, these may be capped using a suitable capping agent. In one embodiment this capping agent is sodium borohydride (NaBH₄).

Following conjugation of serotype 35B polysaccharide to the carrier protein, the glycoconjugate can be purified (enriched with respect to the amount of polysaccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

In some embodiments, the serotype 35B glycoconjugates of the present invention comprise a saccharide having a molecular weight of between 10 kDa and 2,000 kDa. In other such embodiments, the saccharide has a molecular weight of between 50 kDa and 1,000 kDa. In other such embodiments, the saccharide has a molecular weight of between 70 kDa and 900 kDa. In other such embodiments, the saccharide has a molecular weight of between 100 kDa and 800 kDa. In other such embodiments, the saccharide has a molecular weight of between 200 kDa and 600 kDa. In further such embodiments, the saccharide has a molecular weight of 100 kDa to 1,000 kDa; 100 kDa to 900 kDa; 100 kDa to 800 kDa; 100 kDa to 700 kDa; 100 kDa to 600 kDa; 100 kDa to 500 kDa; 100 kDa to 400 kDa; 100 kDa to 300 kDa; 150 kDa to 1,000 kDa; 150 kDa to 900 kDa; 150 kDa to 800 kDa; 150 kDa to 700 kDa; 150 kDa to 600 kDa; 150 kDa to 500 kDa; 150 kDa to 400 kDa; 150 kDa to 300 kDa; 200 kDa to 1,000 kDa; 200 kDa to 900 kDa; 200 kDa to 800 kDa; 200 kDa to 700 kDa; 200 kDa to 600 kDa; 200 kDa to 500 kDa; 200 kDa to 400 kDa; 200 kDa to 300 kDa; 250 kDa to 1,000 kDa; 250 kDa to 900 kDa; 250 kDa to 800 kDa; 250 kDa to 700 kDa; 250 kDa to 600 kDa; 250 kDa to 500 kDa; 250 kDa to 400 kDa; 250 kDa to 350 kDa; 300 kDa to 1000 kDa; 300 kDa to 900 kDa; 300 kDa to 800 kDa; 300 kDa to 700 kDa; 300 kDa to 600 kDa; 300 kDa to 500 kDa; 300 kDa to 400 kDa; 400 kDa to 1,000 kDa; 400 kDa to 900 kDa; 400 kDa to 800 kDa; 400 kDa to 700 kDa; 400 kDa to 600 kDa; 500 kDa to 600 kDa. Any whole number integer within any of the above ranges is contemplated as an embodiment of the disclosure. In some such embodiments, the serotype 35B glycoconjugates are prepared using reductive amination.

In some embodiments, the serotype 35B glycoconjugate of the invention has a molecular weight of between 400 kDa and 15,000 kDa; between 500 kDa and 10,000 kDa; between 2,000 kDa and 10,000 kDa; between 3,000 kDa and 8,000 kDa; or between 3,000 kDa and 5,000 kDa. In other embodiments, the serotype 35B glycoconjugate has a molecular weight of between 500 kDa and 10,000 kDa. In other embodiments, the serotype 35B glycoconjugate has a molecular weight of between 1,000 kDa and 8,000 kDa. In still other embodiments, the serotype 35B glycoconjugate has a molecular weight of between 2,000 kDa and 8,000 kDa or between 3,000 kDa and 7,000 kDa. In further embodiments, the serotype 35B glycoconjugate of the invention has a molecular weight of between 200 kDa and 20,000 kDa; between 200 kDa and 15,000 kDa; between 200 kDa and 10,000 kDa; between 200 kDa and 7,500 kDa; between 200 kDa and 5,000 kDa; between 200 kDa and 3,000 kDa; between 200 kDa and 1,000 kDa; between 500 kDa and 20,000 kDa; between 500 kDa and 15,000 kDa; between 500 kDa and 12,500 kDa; between 500 kDa and 10,000 kDa; between 500 kDa and 7,500 kDa; between 500 kDa and 6,000 kDa; between 500 kDa and 5,000 kDa; between 500 kDa and 4,000 kDa; between 500 kDa and 3,000 kDa; between 500 kDa and 2,000 kDa; between 500 kDa and 1,500 kDa; between 500 kDa and 1,000 kDa; between 750 kDa and 20,000 kDa; between 750 kDa and 15,000 kDa; between 750 kDa and 12,500 kDa; between 750 kDa and 10,000 kDa; between 750 kDa and 7,500 kDa; between 750 kDa and 6,000 kDa; between 750 kDa and 5,000 kDa; between 750 kDa and 4,000 kDa; between 750 kDa and 3,000 kDa; between 750 kDa and 2,000 kDa; between 750 kDa and 1,500 kDa; between 1,000 kDa and 15,000 kDa; between 1,000 kDa and 12,500 kDa; between 1,000 kDa and 10,000 kDa; between 1,000 kDa and 7,500 kDa; between 1,000 kDa and 6,000 kDa; between 1,000 kDa and 5,000 kDa; between 1,000 kDa and 4,000 kDa; between 1,000 kDa and 2,500 kDa; between 2,000 kDa and 15,000 kDa; between 2,000 kDa and 12,500 kDa; between 2,000 kDa and 10,000 kDa; between 2,000 kDa and 7,500 kDa; between 2,000 kDa and 6,000 kDa; between 2,000 kDa and 5,000 kDa; between 2,000 kDa and 4,000 kDa; or between 2,000 kDa and 3,000 kDa.

In further embodiments, the serotype 35B glycoconjugate of the invention has a molecular weight of between 3,000 kDa and 20,000 kDa; between 3,000 kDa and 15,000 kDa; between 3,000 kDa and 10,000 kDa; between 3,000 kDa and 7,500 kDa; between 3,000 kDa and 5,000 kDa; between 4,000 kDa and 20,000 kDa; between 4,000 kDa and 15,000 kDa; between 4,000 kDa and 12,500 kDa; between 4,000 kDa and 10,000 kDa; between 4,000 kDa and 7,500 kDa; between 4,000 kDa and 6,000 kDa; or between 4,000 kDa and 5,000 kDa.

In further embodiments, the serotype 35B glycoconjugate of the invention has a molecular weight of between 5,000 kDa and 20,000 kDa; between 5,000 kDa and 15,000 kDa; between 5,000 kDa and 10,000 kDa; between 5,000 kDa and 7,500 kDa; between 6,000 kDa and 20,000 kDa; between 6,000 kDa and 15,000 kDa; between 6,000 kDa and 12,500 kDa; between 6,000 kDa and 10,000 kDa or between 6,000 kDa and 7,500 kDa.

The molecular weight of the glycoconjugate is measured by SEC-MALLS. Any whole number integer within any of the above ranges is contemplated as an embodiment of the disclosure.

In an embodiment, the serotype 35B glycoconjugate of the invention comprises at least 0.1, 0.2, 0.3, 0.4, 0.5, 0.6 or 0.7 or about 0.8 mM acetate per mM serotype 35B polysaccharide. In an embodiment, the glycoconjugate comprises at least 0.5, 0.6 or 0.7 mM acetate per mM serotype 35B polysaccharide. In an embodiment, the glycoconjugate comprises at least 0.6 mM acetate per mM serotype 35B polysaccharide. In another embodiment, the glycoconjugate comprises at least 0.7 mM acetate per mM serotype 35B polysaccharide.

In another embodiment, the ratio of mM acetate per mM serotype 35B polysaccharide in the glycoconjugate to mM acetate per mM serotype 35B polysaccharide in the isolated polysaccharide is at least 0.6, 0.65, 0.7, 0.75, 0.8, 0.85, 0.9, or 0.95. In an embodiment, the ratio of mM acetate per mM serotype 35B polysaccharide in the glycoconjugate to mM acetate per mM serotype 35B polysaccharide in the isolated polysaccharide is at least 0.7. In another embodiment, the ratio of mM acetate per mM serotype 35B polysaccharide in the glycoconjugate to mM acetate per mM serotype 35B polysaccharide in the isolated polysaccharide is at least 0.9.

In an embodiment, the ratio of mM acetate per mM serotype 35B polysaccharide in the glycoconjugate to mM acetate per mM serotype 35B polysaccharide in the activated polysaccharide is at least 0.6, 0.65, 0.7, 0.75, 0.8, 0.85, 0.9, or 0.95. In an embodiment, the ratio of mM acetate per mM serotype 34 polysaccharide in the glycoconjugate to mM acetate per mM serotype 35B polysaccharide in the activated polysaccharide is at least 0.7. In another embodiment, the ratio of mM acetate per mM serotype 35B polysaccharide in the glycoconjugate to mM acetate per mM serotype 35B polysaccharide in the activated polysaccharide is at least 0.9.

Another way to characterize the serotype 35B glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the CRM₁₉₇ protein starting material used to generate the conjugate materials. In an embodiment, the degree of conjugation of the serotype 35B glycoconjugate of the invention is between 2 and 15, between 2 and 13, between 2 and 10, between 2 and 8, between 2 and 6, between 2 and 5, between 2 and 4, between 3 and 15, between 3 and 13, between 3 and 10, between 3 and 8, between 3 and 6, between 3 and 5, between 3 and 4, between 5 and 15, between 5 and 10, between 8 and 15, between 8 and 12, between 10 and 15 or between 10 and 12. In an embodiment, the degree of conjugation of the serotype 35B glycoconjugate of the invention is about 2, about 3, about 4, about 5, about 6, about 7, about 8, about 9, about 10, about 11, about 12, about 13, about 14 or about 15. In an embodiment, the degree of conjugation of the serotype 35B glycoconjugate of the invention is between 4 and 7. In some such embodiments, the carrier protein is CRM₁₉₇.

The serotype 35B glycoconjugates of the invention may also be characterized by the ratio (weight/weight) of saccharide to carrier protein. In some embodiments, the ratio of serotype 35B polysaccharide to carrier protein in the glycoconjugate (w/w) is between 0.5 and 3.0 (e.g., about 0.5, about 0.6, about 0.7, about 0.8, about 0.9, about 1.0, and 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0). In other embodiments, the saccharide to carrier protein ratio (w/w) is between 0.5 and 2.0, between, 0.5 and 1.5, between 0.8 and 1.2, between 0.5 and 1.0, between 1.0 and 1.5 or between 1.0 and 2.0. In further embodiments, the saccharide to carrier protein ratio (w/w) is between 0.8 and 1.2. In an embodiment, the ratio of serotype 35B capsular polysaccharide to carrier protein in the conjugate is between 0.9 and 1.1. In some such embodiments, the carrier protein is CRM₁₉₇.

The serotype 35B glycoconjugates and immunogenic compositions of the invention may contain free saccharide that is not covalently conjugated to the carrier protein, but is nevertheless present in the glycoconjugate composition. The free saccharide may be noncovalently associated with (i.e., noncovalently bound to, adsorbed to, or entrapped in or with) the glycoconjugate.

In an embodiment, the serotype 35B glycoconjugate comprises less than about 50%, 45%, 40%, 35%, 30%, 25%, 20% or 15% of free serotype 35B polysaccharide compared to the total amount of serotype 35B polysaccharide. In another embodiment, the serotype 35B glycoconjugate comprises less than about 40% of free serotype 35B polysaccharide compared to the total amount of serotype 35B polysaccharide. In an embodiment, the serotype 35B glycoconjugate comprises less than about 25% of free serotype 35B polysaccharide compared to the total amount of serotype 35B polysaccharide. In an embodiment, the serotype 35B glycoconjugate comprises less than about 20% of free serotype 35B polysaccharide compared to the total amount of serotype 35B polysaccharide. In another embodiment the serotype 35B glycoconjugate comprises less than about 15% of free serotype 35B polysaccharide compared to the total amount of serotype 35B polysaccharide.

The serotype 35B glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In an embodiment, at least 30% of the serotype 35B glycoconjugate has a K_{d} below or equal to 0.3 in a CL-4B column. In another embodiment, at least 40% of the glycoconjugate has a K_{d} below or equal to 0.3 in a CL-4B column. In another embodiment, at least 45%, 50%, 55%, 60%, 65%, 70%, 75%, 80%, or 85% of the serotype 35B glycoconjugate has a K_{d} below or equal to 0.3 in a CL-4B column. In an embodiment, at least 60% of the serotype 35B glycoconjugate has a K_{d} below or equal to 0.3 in a CL-4B column. In another embodiment, between 50% and 80% of the serotype 35B glycoconjugate has a K_{d} below or equal to 0.3 in a CL-4B column. In an embodiment, between 65% and 80% of the serotype 35B glycoconjugate has a K_{d} below or equal to 0.3 in a CL-4B column.

1.3.16 Glycoconjugates from S. pneumoniae Serotype 35F

In an embodiment, the serotype 35F glycoconjugates are obtained by activating polysaccharide with 1-cyano-4-dimethylamino pyridinium tetrafluoroborate (CDAP) to form a cyanate ester. The activated polysaccharide may be coupled directly or via a spacer (linker) group to an amino group on the carrier protein. For example, the spacer may be cystamine or cysteamine to give a thiolated polysaccharide which may be coupled to the carrier via a thioether linkage obtained after reaction with a maleimide-activated carrier protein (for example using GMBS) or a haloacetylated carrier protein (for example using iodoacetimide, SIB, SIAB, sulfo-SIAB, SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO93/15760, WO95/08348 and WO96/129094.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO98/42721. Conjugation may involve a carbonyl linker which may be formed by reaction of a free hydroxyl group of the saccharide with CDI (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In one or more embodiments, the serotype 35F glycoconjugates of the invention are prepared using reductive amination. Reductive amination involves two steps: (1) oxidation of the polysaccharide to generate aldehyde functionalities from vicinal diols in individual hexasaccharide unit and (2) reduction of the activated polysaccharide and a carrier protein (e.g., CRM₁₉₇) to form a conjugate.

In an embodiment, before oxidation, sizing of the serotype 35F polysaccharide to a target molecular weight (MW) range is performed. Advantageously, the size of the purified serotype 35F polysaccharide is reduced while preserving critical features of the structure of the polysaccharide such as for example the presence of O-acetyl groups. In an embodiment, the size of the purified serotype 35F polysaccharide is reduced by mechanical homogenization as described herein.

In an embodiment, serotype polysaccharide is activated (oxidized) by a process comprising the step of:
(a) reacting isolated serotype 35F polysaccharide with an oxidizing agent; and
(b) quenching the oxidation reaction by addition of a quenching agent resulting in an activated serotype 35F polysaccharide.

In an embodiment, the oxidizing agent is periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate). In an embodiment, the oxidizing agent is sodium periodate. In an embodiment, the periodate used for the oxidation of serotype 35F polysaccharide is metaperiodate. In another embodiment, the periodate used for the oxidation of serotype 35F polysaccharide is sodium metaperiodate.

In one embodiment, the quenching agent is selected from vicinal diols, 1,2-aminoalcohols, amino acids, glutathione, sulfite, bisulfate, dithionite, metabisulfite, thiosulfate, phosphites, hypophosphites or phosphorous acid.

In one embodiment, the quenching agent is a 1,2-aminoalcohols of formula (I):

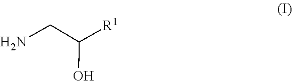

wherein R¹ is selected from H, methyl, ethyl, propyl or isopropyl.

In one embodiment, the quenching agent is selected from sodium and potassium salts of sulfite, bisulfate, dithionite, metabisulfite, thiosulfate, phosphites, hypophosphites or phosphorous acid.

In one embodiment, the quenching agent is an amino acid. In such embodiments, said amino acid may be selected from serine, threonine, cysteine, cystine, methionine, proline, hydroxyproline, tryptophan, tyrosine, and histidine.

In one embodiment, the quenching agent is a sulfite such as bisulfate, dithionite, metabisulfite, thiosulfate.

In one embodiment, the quenching agent is a compound comprising two vicinal hydroxyl groups (vicinal diols), i.e., two hydroxyl groups covalently linked to two adjacent carbon atoms.

In one or more embodiments, the quenching agent is a compound of formula (II):

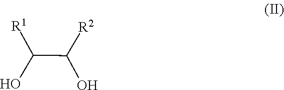

wherein R¹ and R² are each independently selected from H, methyl, ethyl, propyl or isopropyl.

In an embodiment, the quenching agent is glycerol, ethylene glycol, propan-1,2-diol, butan-1,2-diol or butan-2,3-diol, or ascorbic acid. In an embodiment, the quenching agent is butan-2,3-diol.

In another embodiment, the isolated serotype 35F polysaccharide is activated by a process comprising the steps of:
(a) reacting isolated serotype 35F polysaccharide with periodate; and
(b) quenching the oxidation reaction by addition of butan-2,3-diol resulting in an activated serotype 35F polysaccharide.

Following the oxidation step of the polysaccharide, the polysaccharide is said to be activated and is referred to as “activated polysaccharide” herein.

In an embodiment, the activated serotype 35F polysaccharide is purified. The activated serotype 35F polysaccharide is purified according to methods known to one skilled in the art such as gel permeation chromatography (GPC), dialysis or ultrafiltration/diafiltration. For example, the activated 35F polysaccharide is purified by concentration and diafiltration using an ultrafiltration device.

In another embodiment, the degree of oxidation of the activated serotype 35F polysaccharide is between 2 and 30, between 2 and 25, between 2 and 20, between 2 and 15, between 2 and 10, between 2 and 5, between 5 and 30, between 5 and 25, between 5 and 20, between 5 and 15, between 5 and 10, between 10 and 30, between 10 and 25, between 10 and 20, between 10 and 15, between 15 and 30, between 15 and 25, between 15 and 20, between 20 to 30, or between 20 to 25. In an embodiment the degree of oxidation of the activated serotype 35F polysaccharide is between 2 and 10, between 4 and 8, between 4 and 6, between 6 and 8, between 6 and 12, between 8 and 14, between 9 and 11, between 10 and 16, between 12 and 16, between 14 and 18, between 16 and 20, between 16 and 18, between 18 and 22, or between 18 and 20.

In an embodiment, the activated serotype 35F polysaccharide has a molecular weight between 25 kDa and 1,000 kDa, between 100 kDa and 1,000 kDa, between 300 kDa and 800 kDa, between 300 kDa and 700 kDa, between 300 kDa and 600 kDa, between 400 kDa and 1,000 kDa, between 400 kDa and 800 kDa, between 400 kDa and 700 kDa or between 400 kDa and 600 kDa. In an embodiment, the activated serotype 35F polysaccharide has a molecular weight between 300 kDa and 800 kDa. In an embodiment, the activated serotype 35F polysaccharide has a molecular weight between 400 kDa and 600 kDa. In another embodiment, the activated serotype 35F polysaccharide has a molecular weight between 400 kda and 600 kDa and a degree of oxidation between 10 and 25, between 10 and 20, between 12 and 20 or between 14 and 18. In another embodiment, the activated serotype 35F polysaccharide has a molecular weight between 400 kDa and 600 kDa and a degree of oxidation between 10 and 20.

In another embodiment, the activated serotype 35F polysaccharide comprises at least 0.1, 0.2, 0.3, 0.4, 0.5, 0.6 or 0.7 or about 0.8 mM acetate per mM serotype 35F polysaccharide. In an embodiment, the activated serotype 35F polysaccharide comprises at least 0.5, 0.6 or 0.7 mM acetate per mM serotype 35F polysaccharide. In another embodiment, the activated serotype 35F polysaccharide comprises at least 0.6 mM acetate per mM serotype 35F polysaccharide. In another embodiment, the activated serotype 35F polysaccharide comprises at least 0.7 mM acetate per mM serotype 35F polysaccharide.

In an embodiment, the activated serotype 35F polysaccharide has a molecular weight between 400 kDa and 800 kDa and comprises at least 0.6 mM acetate per mM serotype 35F polysaccharide.

In an embodiment, the activated serotype 35F polysaccharide has a molecular weight between 400 kDa and 800 kDa, a degree of oxidation between 12 and 20 and comprises at least 0.6 mM acetate per mM serotype 35F polysaccharide.

The activated polysaccharide and/or the carrier protein may be lyophilised (freeze-dried), either independently (discrete lyophilization) or together (co-lyophilized).

In another embodiment, the activated serotype 35F polysaccharide is lyophilized, optionally in the presence of saccharide. In an embodiment, the saccharide is selected from sucrose, trehalose, raffinose, stachyose, melezitose, dextran, mannitol, lactitol and palatinit. In an embodiment, the saccharide is sucrose. In one embodiment, the lyophilized activated polysaccharide is then compounded with a solution comprising the carrier protein.

In another embodiment, the activated polysaccharide and the carrier protein are co-lyophilised. In such embodiments, the activated serotype 35F polysaccharide is compounded with the carrier protein and lyophilized optionally in the presence of a saccharide. In an embodiment, the saccharide is selected from sucrose, trehalose, raffinose, stachyose, melezitose, dextran, mannitol, lactitol and palatinit. In an embodiment, the saccharide is sucrose. The co-lyophilized polysaccharide and carrier protein can then be resuspended in solution and reacted with a reducing agent.

The second step of the conjugation process is the reduction of the activated polysaccharide and a carrier protein to form a conjugate (reductive amination), using a reducing agent.

The activated serotype 35F polysaccharide can be conjugated to a carrier protein by a process comprising the steps of:
(c) compounding the activated serotype 35F polysaccharide with a carrier protein; and
(d) reacting the compounded activated serotype 35F polysaccharide and carrier protein with a reducing agent to form a serotype 35F polysaccharide-carrier protein conjugate.

In an embodiment, the reduction reaction is carried out in aqueous solvent. In another embodiment the reaction is carried out in aprotic solvent. In an embodiment, the reduction reaction is carried out in DMSO (dimethylsulfoxide) or in DMF (dimethylformamide)) solvent. The DMSO or DMF solvent may be used to reconstitute the activated polysaccharide and carrier protein which has been lyophilised.

The conjugation of activated serotype 35F polysaccharide with a protein carrier by reductive amination in dimethylsulfoxide (DMSO) is suitable to preserve the O-acetyl content of the polysaccharide as compared, for example, to reductive amination in aqueous phase where the level of O-acetylation of the polysaccharide may be significantly reduced. Therefore, in one or more embodiments, step (c) and step (d) are carried out in DMSO.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In an embodiment, the reducing agent is sodium cyanoborohydride.

At the end of the reduction reaction, there may be unreacted aldehyde groups remaining in the conjugates, these may be capped using a suitable capping agent. In one embodiment this capping agent is sodium borohydride (NaBH₄).

Following conjugation of serotype 35F polysaccharide to the carrier protein, the glycoconjugate can be purified (enriched with respect to the amount of polysaccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

In some embodiments, the serotype 35F glycoconjugates of the present invention comprise a saccharide having a molecular weight of between 10 kDa and 2,000 kDa. In other such embodiments, the saccharide has a molecular weight of between 50 kDa and 1,000 kDa. In other such embodiments, the saccharide has a molecular weight of between 70 kDa and 900 kDa. In other such embodiments, the saccharide has a molecular weight of between 100 kDa and 800 kDa. In other such embodiments, the saccharide has a molecular weight of between 200 kDa and 600 kDa. In further such embodiments, the saccharide has a molecular weight of 100 kDa to 1,000 kDa; 100 kDa to 900 kDa; 100 kDa to 800 kDa; 100 kDa to 700 kDa; 100 kDa to 600 kDa; 100 kDa to 500 kDa; 100 kDa to 400 kDa; 100 kDa to 300 kDa; 150 kDa to 1,000 kDa; 150 kDa to 900 kDa; 150 kDa to 800 kDa; 150 kDa to 700 kDa; 150 kDa to 600 kDa; 150 kDa to 500 kDa; 150 kDa to 400 kDa; 150 kDa to 300 kDa; 200 kDa to 1,000 kDa; 200 kDa to 900 kDa; 200 kDa to 800 kDa; 200 kDa to 700 kDa; 200 kDa to 600 kDa; 200 kDa to 500 kDa; 200 kDa to 400 kDa; 200 kDa to 300 kDa; 250 kDa to 1,000 kDa; 250 kDa to 900 kDa; 250 kDa to 800 kDa; 250 kDa to 700 kDa; 250 kDa to 600 kDa; 250 kDa to 500 kDa; 250 kDa to 400 kDa; 250 kDa to 350 kDa; 300 kDa to 1000 kDa; 300 kDa to 900 kDa; 300 kDa to 800 kDa; 300 kDa to 700 kDa; 300 kDa to 600 kDa; 300 kDa to 500 kDa; 300 kDa to 400 kDa; 400 kDa to 1,000 kDa; 400 kDa to 900 kDa; 400 kDa to 800 kDa; 400 kDa to 700 kDa; 400 kDa to 600 kDa; 500 kDa to 600 kDa. Any whole number integer within any of the above ranges is contemplated as an embodiment of the disclosure. In some such embodiments, the serotype 35F glycoconjugates are prepared using reductive amination.

In some embodiments, the serotype 35F glycoconjugate of the invention has a molecular weight of between 400 kDa and 15,000 kDa; between 500 kDa and 10,000 kDa; between 2,000 kDa and 10,000 kDa; between 3,000 kDa and 8,000 kDa; or between 3,000 kDa and 5,000 kDa. In other embodiments, the serotype 35F glycoconjugate has a molecular weight of between 500 kDa and 10,000 kDa. In other embodiments, the serotype 35F glycoconjugate has a molecular weight of between 1,000 kDa and 8,000 kDa. In still other embodiments, the serotype 35F glycoconjugate has a molecular weight of between 2,000 kDa and 8,000 kDa or between 3,000 kDa and 7,000 kDa. In further embodiments, the serotype 35F glycoconjugate of the invention has a molecular weight of between 200 kDa and 20,000 kDa; between 200 kDa and 15,000 kDa; between 200 kDa and 10,000 kDa; between 200 kDa and 7,500 kDa; between 200 kDa and 5,000 kDa; between 200 kDa and 3,000 kDa; between 200 kDa and 1,000 kDa; between 500 kDa and 20,000 kDa; between 500 kDa and 15,000 kDa; between 500 kDa and 12,500 kDa; between 500 kDa and 10,000 kDa; between 500 kDa and 7,500 kDa; between 500 kDa and 6,000 kDa; between 500 kDa and 5,000 kDa; between 500 kDa and 4,000 kDa; between 500 kDa and 3,000 kDa; between 500 kDa and 2,000 kDa; between 500 kDa and 1,500 kDa; between 500 kDa and 1,000 kDa; between 750 kDa and 20,000 kDa; between 750 kDa and 15,000 kDa; between 750 kDa and 12,500 kDa; between 750 kDa and 10,000 kDa; between 750 kDa and 7,500 kDa; between 750 kDa and 6,000 kDa; between 750 kDa and 5,000 kDa; between 750 kDa and 4,000 kDa; between 750 kDa and 3,000 kDa; between 750 kDa and 2,000 kDa; between 750 kDa and 1,500 kDa; between 1,000 kDa and 15,000 kDa; between 1,000 kDa and 12,500 kDa; between 1,000 kDa and 10,000 kDa; between 1,000 kDa and 7,500 kDa; between 1,000 kDa and 6,000 kDa; between 1,000 kDa and 5,000 kDa; between 1,000 kDa and 4,000 kDa; between 1,000 kDa and 2,500 kDa; between 2,000 kDa and 15,000 kDa; between 2,000 kDa and 12,500 kDa; between 2,000 kDa and 10,000 kDa; between 2,000 kDa and 7,500 kDa; between 2,000 kDa and 6,000 kDa; between 2,000 kDa and 5,000 kDa; between 2,000 kDa and 4,000 kDa; or between 2,000 kDa and 3,000 kDa.

In further embodiments, the serotype 35F glycoconjugate of the invention has a molecular weight of between 3,000 kDa and 20,000 kDa; between 3,000 kDa and 15,000 kDa; between 3,000 kDa and 10,000 kDa; between 3,000 kDa and 7,500 kDa; between 3,000 kDa and 5,000 kDa; between 4,000 kDa and 20,000 kDa; between 4,000 kDa and 15,000 kDa; between 4,000 kDa and 12,500 kDa; between 4,000 kDa and 10,000 kDa; between 4,000 kDa and 7,500 kDa; between 4,000 kDa and 6,000 kDa; or between 4,000 kDa and 5,000 kDa.

In further embodiments, the serotype 35F glycoconjugate of the invention has a molecular weight of between 5,000 kDa and 20,000 kDa; between 5,000 kDa and 15,000 kDa; between 5,000 kDa and 10,000 kDa; between 5,000 kDa and 7,500 kDa; between 6,000 kDa and 20,000 kDa; between 6,000 kDa and 15,000 kDa; between 6,000 kDa and 12,500 kDa; between 6,000 kDa and 10,000 kDa or between 6,000 kDa and 7,500 kDa.

The molecular weight of the glycoconjugate is measured by SEC-MALLS. Any whole number integer within any of the above ranges is contemplated as an embodiment of the disclosure.

In an embodiment, the serotype 35F glycoconjugate of the invention comprises at least 0.1, 0.2, 0.3, 0.4, 0.5, 0.6 or 0.7 or about 0.8 mM acetate per mM serotype 34 polysaccharide. In an embodiment, the glycoconjugate comprises at least 0.5, 0.6 or 0.7 mM acetate per mM serotype 34 polysaccharide. In an embodiment, the glycoconjugate comprises at least 0.6 mM acetate per mM serotype 35F polysaccharide. In another embodiment, the glycoconjugate comprises at least 0.7 mM acetate per mM serotype 35F polysaccharide.

In another embodiment, the ratio of mM acetate per mM serotype 35F polysaccharide in the glycoconjugate to mM acetate per mM serotype 35F polysaccharide in the isolated polysaccharide is at least 0.6, 0.65, 0.7, 0.75, 0.8, 0.85, 0.9, or 0.95. In an embodiment, the ratio of mM acetate per mM serotype 35F polysaccharide in the glycoconjugate to mM acetate per mM serotype 35F polysaccharide in the isolated polysaccharide is at least 0.7. In another embodiment, the ratio of mM acetate per mM serotype 35F polysaccharide in the glycoconjugate to mM acetate per mM serotype 35F polysaccharide in the isolated polysaccharide is at least 0.9.

In an embodiment, the ratio of mM acetate per mM serotype 35F polysaccharide in the glycoconjugate to mM acetate per mM serotype 35F polysaccharide in the activated polysaccharide is at least 0.6, 0.65, 0.7, 0.75, 0.8, 0.85, 0.9, or 0.95. In an embodiment, the ratio of mM acetate per mM serotype 35F polysaccharide in the glycoconjugate to mM acetate per mM serotype 35F polysaccharide in the activated polysaccharide is at least 0.7. In another embodiment, the ratio of mM acetate per mM serotype 35F polysaccharide in the glycoconjugate to mM acetate per mM serotype 35F polysaccharide in the activated polysaccharide is at least 0.9.

Another way to characterize the serotype 35F glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the CRM₁₉₇ protein starting material used to generate the conjugate materials. In an embodiment, the degree of conjugation of the serotype 35F glycoconjugate of the invention is between 2 and 15, between 2 and 13, between 2 and 10, between 2 and 8, between 2 and 6, between 2 and 5, between 2 and 4, between 3 and 15, between 3 and 13, between 3 and 10, between 3 and 8, between 3 and 6, between 3 and 5, between 3 and 4, between 5 and 15, between 5 and 10, between 8 and 15, between 8 and 12, between 10 and 15 or between 10 and 12. In an embodiment, the degree of conjugation of the serotype 35F glycoconjugate of the invention is about 2, about 3, about 4, about 5, about 6, about 7, about 8, about 9, about 10, about 11, about 12, about 13, about 14 or about 15. In an embodiment, the degree of conjugation of the serotype 35F glycoconjugate of the invention is between 4 and 7. In some such embodiments, the carrier protein is CRM₁₉₇.

The serotype 35F glycoconjugates of the invention may also be characterized by the ratio (weight/weight) of saccharide to carrier protein. In some embodiments, the ratio of serotype 35F polysaccharide to carrier protein in the glycoconjugate (w/w) is between 0.5 and 3.0 (e.g., about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0). In other embodiments, the saccharide to carrier protein ratio (w/w) is between 0.5 and 2.0, between 0.5 and 1.5, between 0.8 and 1.2, between 0.5 and 1.0, between 1.0 and 1.5 or between 1.0 and 2.0. In further embodiments, the saccharide to carrier protein ratio (w/w) is between 0.8 and 1.2. In an embodiment, the ratio of serotype 35F capsular polysaccharide to carrier protein in the conjugate is between 0.9 and 1.1. In some such embodiments, the carrier protein is CRM₁₉₇.

The serotype 35F glycoconjugates and immunogenic compositions of the invention may contain free saccharide that is not covalently conjugated to the carrier protein, but is nevertheless present in the glycoconjugate composition. The free saccharide may be noncovalently associated with (i.e., noncovalently bound to, adsorbed to, or entrapped in or with) the glycoconjugate.

In an embodiment, the serotype 35F glycoconjugate comprises less than about 50%, 45%, 40%, 35%, 30%, 25%, 20% or 15% of free serotype 35F polysaccharide compared to the total amount of serotype 35F polysaccharide. In another embodiment, the serotype 35F glycoconjugate comprises less than about 40% of free serotype 35F polysaccharide compared to the total amount of serotype 35F polysaccharide. In an embodiment, the serotype 35F glycoconjugate comprises less than about 25% of free serotype 35F polysaccharide compared to the total amount of serotype 35F polysaccharide. In an embodiment, the serotype 35F glycoconjugate comprises less than about 20% of free serotype 35F polysaccharide compared to the total amount of serotype 35F polysaccharide. In another embodiment the serotype 35F glycoconjugate comprises less than about 15% of free serotype 35F polysaccharide compared to the total amount of serotype 35F polysaccharide.

The serotype 35F glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In an embodiment, at least 30% of the serotype 35F glycoconjugate has a K_{d} below or equal to 0.3 in a CL-4B column. In another embodiment, at least 40% of the glycoconjugate has a K_{d} below or equal to 0.3 in a CL-4B column. In another embodiment, at least 45%, 50%, 55%, 60%, 65%, 70%, 75%, 80%, or 85% of the serotype 35F glycoconjugate has a K_{d} below or equal to 0.3 in a CL-4B column. In an embodiment, at least 60% of the serotype 35F glycoconjugate has a K_{d} below or equal to 0.3 in a CL-4B column. In another embodiment, between 50% and 80% of the serotype 35F glycoconjugate has a K_{d} below or equal to 0.3 in a CL-4B column. In an embodiment, between 65% and 80% of the serotype 35F glycoconjugate has a K_{d} below or equal to 0.3 in a CL-4B column.

1.3.17 Glycoconjugates from S. pneumoniae Serotype 38

In an embodiment, the serotype 38 glycoconjugates are obtained by activating polysaccharide with 1-cyano-4-dimethylamino pyridinium tetrafluoroborate (CDAP) to form a cyanate ester. The activated polysaccharide may be coupled directly or via a spacer (linker) group to an amino group on the carrier protein. For example, the spacer may be cystamine or cysteamine to give a thiolated polysaccharide which may be coupled to the carrier via a thioether linkage obtained after reaction with a maleimide-activated carrier protein (for example using GMBS) or a haloacetylated carrier protein (for example using iodoacetimide, SIB, SIAB, sulfo-SIAB, SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO93/15760, WO95/08348 and WO96/129094.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO98/42721. Conjugation may involve a carbonyl linker which may be formed by reaction of a free hydroxyl group of the saccharide with CDI (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In one or more embodiments, the serotype 38 glycoconjugates of the invention are prepared using reductive amination. Reductive amination involves two steps: (1) oxidation of the polysaccharide to generate aldehyde functionalities from vicinal diols in individual hexasaccharide unit and (2) reduction of the activated polysaccharide and a carrier protein (e.g., CRM₁₉₇) to form a conjugate.

In an embodiment, before oxidation, sizing of the serotype 38 polysaccharide to a target molecular weight (MW) range is performed. Advantageously, the size of the purified serotype 38 polysaccharide is reduced while preserving critical features of the structure of the polysaccharide such as for example the presence of O-acetyl groups. In an embodiment, the size of the purified serotype 38 polysaccharide is reduced by mechanical homogenization as described herein.

In an embodiment, serotype polysaccharide is activated (oxidized) by a process comprising the step of:
(a) reacting isolated serotype 38 polysaccharide with an oxidizing agent; and
(b) quenching the oxidation reaction by addition of a quenching agent resulting in an activated serotype 38 polysaccharide.

In an embodiment, the oxidizing agent is periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate). In an embodiment, the oxidizing agent is sodium periodate. In an embodiment, the periodate used for the oxidation of serotype 38 polysaccharide is metaperiodate. In another embodiment, the periodate used for the oxidation of serotype 38 polysaccharide is sodium metaperiodate.

In one embodiment, the quenching agent is selected from vicinal diols, 1,2-aminoalcohols, amino acids, glutathione, sulfite, bisulfate, dithionite, metabisulfite, thiosulfate, phosphites, hypophosphites or phosphorous acid.

In one embodiment, the quenching agent is a 1,2-aminoalcohols of formula (I):

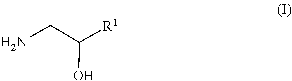

wherein R¹ is selected from H, methyl, ethyl, propyl or isopropyl.

In one embodiment, the quenching agent is selected from sodium and potassium salts of sulfite, bisulfate, dithionite, metabisulfite, thiosulfate, phosphites, hypophosphites or phosphorous acid.

In one embodiment, the quenching agent is an amino acid. In such embodiments, said amino acid may be selected from serine, threonine, cysteine, cystine, methionine, proline, hydroxyproline, tryptophan, tyrosine, and histidine.

In one embodiment, the quenching agent is a sulfite such as bisulfate, dithionite, metabisulfite, thiosulfate.

In one embodiment, the quenching agent is a compound comprising two vicinal hydroxyl groups (vicinal diols), i.e., two hydroxyl groups covalently linked to two adjacent carbon atoms.

In one or more embodiments, the quenching agent is a compound of formula (II):

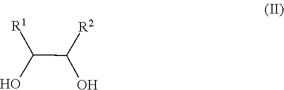

wherein R¹ and R² are each independently selected from H, methyl, ethyl, propyl or isopropyl.

In an embodiment, the quenching agent is glycerol, ethylene glycol, propan-1,2-diol, butan-1,2-diol or butan-2,3-diol, or ascorbic acid. In an embodiment, the quenching agent is butan-2,3-diol.

In another embodiment, the isolated serotype 38 polysaccharide is activated by a process comprising the steps of:
(a) reacting isolated serotype 38 polysaccharide with periodate; and
(b) quenching the oxidation reaction by addition of butan-2,3-diol resulting in an activated serotype 38 polysaccharide.

Following the oxidation step of the polysaccharide, the polysaccharide is said to be activated and is referred to as “activated polysaccharide” herein.

In an embodiment, the activated serotype 38 polysaccharide is purified. The activated serotype 38 polysaccharide is purified according to methods known to one skilled in the art such as gel permeation chromatography (GPC), dialysis or ultrafiltration/diafiltration. For example, the activated 38 polysaccharide is purified by concentration and diafiltration using an ultrafiltration device.

In another embodiment, the degree of oxidation of the activated serotype 38 polysaccharide is between 2 and 30, between 2 and 25, between 2 and 20, between 2 and 15, between 2 and 10, between 2 and 5, between 5 and 30, between 5 and 25, between 5 and 20, between 5 and 15, between 5 and 10, between 10 and 30, between 10 and 25, between 10 and 20, between 10 and 15, between 15 and 30, between 15 and 25, between 15 and 20, between 20 to 30, or between 20 to 25. In an embodiment the degree of oxidation of the activated serotype 38 polysaccharide is between 2 and 10, between 4 and 8, between 4 and 6, between 6 and 8, between 6 and 12, between 8 and 14, between 9 and 11, between 10 and 16, between 12 and 16, between 14 and 18, between 16 and 20, between 16 and 18, between 18 and 22, or between 18 and 20.

In an embodiment, the activated serotype 38 polysaccharide has a molecular weight between 25 kDa and 1,000 kDa, between 100 kDa and 1,000 kDa, between 300 kDa and 800 kDa, between 300 kDa and 700 kDa, between 300 kDa and 600 kDa, between 400 kDa and 1,000 kDa, between 400 kDa and 800 kDa, between 400 kDa and 700 kDa or between 400 kDa and 600 kDa. In an embodiment, the activated serotype 38 polysaccharide has a molecular weight between 300 kDa and 800 kDa. In an embodiment, the activated serotype 38 polysaccharide has a molecular weight between 400 kDa and 600 kDa. In another embodiment, the activated serotype 38 polysaccharide has a molecular weight between 400 kda and 600 kDa and a degree of oxidation between 10 and 25, between 10 and 20, between 12 and 20 or between 14 and 18. In another embodiment, the activated serotype 38 polysaccharide has a molecular weight between 400 kDa and 600 kDa and a degree of oxidation between 10 and 20.

In another embodiment, the activated serotype 38 polysaccharide comprises at least 0.1, 0.2, 0.3, 0.4, 0.5, 0.6 or 0.7 or about 0.8 mM acetate per mM serotype 38 polysaccharide. In an embodiment, the activated serotype 38 polysaccharide comprises at least 0.5, 0.6 or 0.7 mM acetate per mM serotype 38 polysaccharide. In another embodiment, the activated serotype 38 polysaccharide comprises at least 0.6 mM acetate per mM serotype 38 polysaccharide. In another embodiment, the activated serotype 38 polysaccharide comprises at least 0.7 mM acetate per mM serotype 38 polysaccharide.

In an embodiment, the activated serotype 38 polysaccharide has a molecular weight between 400 kDa and 800 kDa and comprises at least 0.6 mM acetate per mM serotype 38 polysaccharide.

In an embodiment, the activated serotype 38 polysaccharide has a molecular weight between 400 kDa and 800 kDa, a degree of oxidation between 12 and 20 and comprises at least 0.6 mM acetate per mM serotype 38 polysaccharide.

The activated polysaccharide and/or the carrier protein may be lyophilised (freeze-dried), either independently (discrete lyophilization) or together (co-lyophilized).

In another embodiment, the activated serotype 38 polysaccharide is lyophilized, optionally in the presence of saccharide. In an embodiment, the saccharide is selected from sucrose, trehalose, raffinose, stachyose, melezitose, dextran, mannitol, lactitol and palatinit. In an embodiment, the saccharide is sucrose. In one embodiment, the lyophilized activated polysaccharide is then compounded with a solution comprising the carrier protein.

In another embodiment, the activated polysaccharide and the carrier protein are co-lyophilised. In such embodiments, the activated serotype 34 polysaccharide is compounded with the carrier protein and lyophilized optionally in the presence of a saccharide. In an embodiment, the saccharide is selected from sucrose, trehalose, raffinose, stachyose, melezitose, dextran, mannitol, lactitol and palatinit. In an embodiment, the saccharide is sucrose. The co-lyophilized polysaccharide and carrier protein can then be resuspended in solution and reacted with a reducing agent.

The second step of the conjugation process is the reduction of the activated polysaccharide and a carrier protein to form a conjugate (reductive amination), using a reducing agent.

The activated serotype 38 polysaccharide can be conjugated to a carrier protein by a process comprising the steps of:
(c) compounding the activated serotype 38 polysaccharide with a carrier protein; and
(d) reacting the compounded activated serotype 38 polysaccharide and carrier protein with a reducing agent to form a serotype 38 polysaccharide-carrier protein conjugate.

In an embodiment, the reduction reaction is carried out in aqueous solvent. In another embodiment the reaction is carried out in aprotic solvent. In an embodiment, the reduction reaction is carried out in DMSO (dimethylsulfoxide) or in DMF (dimethylformamide)) solvent. The DMSO or DMF solvent may be used to reconstitute the activated polysaccharide and carrier protein which has been lyophilised.

The conjugation of activated serotype 38 polysaccharide with a protein carrier by reductive amination in dimethylsulfoxide (DMSO) is suitable to preserve the O-acetyl content of the polysaccharide as compared, for example, to reductive amination in aqueous phase where the level of O-acetylation of the polysaccharide may be significantly reduced. Therefore, in one or more embodiments, step (c) and step (d) are carried out in DMSO.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In an embodiment, the reducing agent is sodium cyanoborohydride.

At the end of the reduction reaction, there may be unreacted aldehyde groups remaining in the conjugates, these may be capped using a suitable capping agent. In one embodiment this capping agent is sodium borohydride (NaBH₄).

Following conjugation of serotype 38 polysaccharide to the carrier protein, the glycoconjugate can be purified (enriched with respect to the amount of polysaccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

In some embodiments, the serotype 38 glycoconjugates of the present invention comprise a saccharide having a molecular weight of between 10 kDa and 2,000 kDa. In other such embodiments, the saccharide has a molecular weight of between 50 kDa and 1,000 kDa. In other such embodiments, the saccharide has a molecular weight of between 70 kDa and 900 kDa. In other such embodiments, the saccharide has a molecular weight of between 100 kDa and 800 kDa. In other such embodiments, the saccharide has a molecular weight of between 200 kDa and 600 kDa. In further such embodiments, the saccharide has a molecular weight of 100 kDa to 1,000 kDa; 100 kDa to 900 kDa; 100 kDa to 800 kDa; 100 kDa to 700 kDa; 100 kDa to 600 kDa; 100 kDa to 500 kDa; 100 kDa to 400 kDa; 100 kDa to 300 kDa; 150 kDa to 1,000 kDa; 150 kDa to 900 kDa; 150 kDa to 800 kDa; 150 kDa to 700 kDa; 150 kDa to 600 kDa; 150 kDa to 500 kDa; 150 kDa to 400 kDa; 150 kDa to 300 kDa; 200 kDa to 1,000 kDa; 200 kDa to 900 kDa; 200 kDa to 800 kDa; 200 kDa to 700 kDa; 200 kDa to 600 kDa; 200 kDa to 500 kDa; 200 kDa to 400 kDa; 200 kDa to 300 kDa; 250 kDa to 1,000 kDa; 250 kDa to 900 kDa; 250 kDa to 800 kDa; 250 kDa to 700 kDa; 250 kDa to 600 kDa; 250 kDa to 500 kDa; 250 kDa to 400 kDa; 250 kDa to 350 kDa; 300 kDa to 1000 kDa; 300 kDa to 900 kDa; 300 kDa to 800 kDa; 300 kDa to 700 kDa; 300 kDa to 600 kDa; 300 kDa to 500 kDa; 300 kDa to 400 kDa; 400 kDa to 1,000 kDa; 400 kDa to 900 kDa; 400 kDa to 800 kDa; 400 kDa to 700 kDa; 400 kDa to 600 kDa; 500 kDa to 600 kDa. Any whole number integer within any of the above ranges is contemplated as an embodiment of the disclosure. In some such embodiments, the serotype 38 glycoconjugates are prepared using reductive amination.

In some embodiments, the serotype 38 glycoconjugate of the invention has a molecular weight of between 400 kDa and 15,000 kDa; between 500 kDa and 10,000 kDa; between 2,000 kDa and 10,000 kDa; between 3,000 kDa and 8,000 kDa; or between 3,000 kDa and 5,000 kDa. In other embodiments, the serotype 38 glycoconjugate has a molecular weight of between 500 kDa and 10,000 kDa. In other embodiments, the serotype 38 glycoconjugate has a molecular weight of between 1,000 kDa and 8,000 kDa. In still other embodiments, the serotype 38 glycoconjugate has a molecular weight of between 2,000 kDa and 8,000 kDa or between 3,000 kDa and 7,000 kDa. In further embodiments, the serotype 38 glycoconjugate of the invention has a molecular weight of between 200 kDa and 20,000 kDa; between 200 kDa and 15,000 kDa; between 200 kDa and 10,000 kDa; between 200 kDa and 7,500 kDa; between 200 kDa and 5,000 kDa; between 200 kDa and 3,000 kDa; between 200 kDa and 1,000 kDa; between 500 kDa and 20,000 kDa; between 500 kDa and 15,000 kDa; between 500 kDa and 12,500 kDa; between 500 kDa and 10,000 kDa; between 500 kDa and 7,500 kDa; between 500 kDa and 6,000 kDa; between 500 kDa and 5,000 kDa; between 500 kDa and 4,000 kDa; between 500 kDa and 3,000 kDa; between 500 kDa and 2,000 kDa; between 500 kDa and 1,500 kDa; between 500 kDa and 1,000 kDa; between 750 kDa and 20,000 kDa; between 750 kDa and 15,000 kDa; between 750 kDa and 12,500 kDa; between 750 kDa and 10,000 kDa; between 750 kDa and 7,500 kDa; between 750 kDa and 6,000 kDa; between 750 kDa and 5,000 kDa; between 750 kDa and 4,000 kDa; between 750 kDa and 3,000 kDa; between 750 kDa and 2,000 kDa; between 750 kDa and 1,500 kDa; between 1,000 kDa and 15,000 kDa; between 1,000 kDa and 12,500 kDa; between 1,000 kDa and 10,000 kDa; between 1,000 kDa and 7,500 kDa; between 1,000 kDa and 6,000 kDa; between 1,000 kDa and 5,000 kDa; between 1,000 kDa and 4,000 kDa; between 1,000 kDa and 2,500 kDa; between 2,000 kDa and 15,000 kDa; between 2,000 kDa and 12,500 kDa; between 2,000 kDa and 10,000 kDa; between 2,000 kDa and 7,500 kDa; between 2,000 kDa and 6,000 kDa; between 2,000 kDa and 5,000 kDa; between 2,000 kDa and 4,000 kDa; or between 2,000 kDa and 3,000 kDa.

In further embodiments, the serotype 38 glycoconjugate of the invention has a molecular weight of between 3,000 kDa and 20,000 kDa; between 3,000 kDa and 15,000 kDa; between 3,000 kDa and 10,000 kDa; between 3,000 kDa and 7,500 kDa; between 3,000 kDa and 5,000 kDa; between 4,000 kDa and 20,000 kDa; between 4,000 kDa and 15,000 kDa; between 4,000 kDa and 12,500 kDa; between 4,000 kDa and 10,000 kDa; between 4,000 kDa and 7,500 kDa; between 4,000 kDa and 6,000 kDa; or between 4,000 kDa and 5,000 kDa.

In further embodiments, the serotype 38 glycoconjugate of the invention has a molecular weight of between 5,000 kDa and 20,000 kDa; between 5,000 kDa and 15,000 kDa; between 5,000 kDa and 10,000 kDa; between 5,000 kDa and 7,500 kDa; between 6,000 kDa and 20,000 kDa; between 6,000 kDa and 15,000 kDa; between 6,000 kDa and 12,500 kDa; between 6,000 kDa and 10,000 kDa or between 6,000 kDa and 7,500 kDa.

The molecular weight of the glycoconjugate is measured by SEC-MALLS. Any whole number integer within any of the above ranges is contemplated as an embodiment of the disclosure.

In an embodiment, the serotype 38 glycoconjugate of the invention comprises at least 0.1, 0.2, 0.3, 0.4, 0.5, 0.6 or 0.7 or about 0.8 mM acetate per mM serotype 34 polysaccharide. In an embodiment, the glycoconjugate comprises at least 0.5, 0.6 or 0.7 mM acetate per mM serotype 38 polysaccharide. In an embodiment, the glycoconjugate comprises at least 0.6 mM acetate per mM serotype 38 polysaccharide. In another embodiment, the glycoconjugate comprises at least 0.7 mM acetate per mM serotype 34 polysaccharide.

In another embodiment, the ratio of mM acetate per mM serotype 38 polysaccharide in the glycoconjugate to mM acetate per mM serotype 38 polysaccharide in the isolated polysaccharide is at least 0.6, 0.65, 0.7, 0.75, 0.8, 0.85, 0.9, or 0.95. In an embodiment, the ratio of mM acetate per mM serotype 38 polysaccharide in the glycoconjugate to mM acetate per mM serotype 38 polysaccharide in the isolated polysaccharide is at least 0.7. In another embodiment, the ratio of mM acetate per mM serotype 38 polysaccharide in the glycoconjugate to mM acetate per mM serotype 38 polysaccharide in the isolated polysaccharide is at least 0.9.

In an embodiment, the ratio of mM acetate per mM serotype 38 polysaccharide in the glycoconjugate to mM acetate per mM serotype 38 polysaccharide in the activated polysaccharide is at least 0.6, 0.65, 0.7, 0.75, 0.8, 0.85, 0.9, or 0.95. In an embodiment, the ratio of mM acetate per mM serotype 38 polysaccharide in the glycoconjugate to mM acetate per mM serotype 38 polysaccharide in the activated polysaccharide is at least 0.7. In another embodiment, the ratio of mM acetate per mM serotype 38 polysaccharide in the glycoconjugate to mM acetate per mM serotype 38 polysaccharide in the activated polysaccharide is at least 0.9.

Another way to characterize the serotype 38 glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the CRM₁₉₇ protein starting material used to generate the conjugate materials. In an embodiment, the degree of conjugation of the serotype 34 glycoconjugate of the invention is between 2 and 15, between 2 and 13, between 2 and 10, between 2 and 8, between 2 and 6, between 2 and 5, between 2 and 4, between 3 and 15, between 3 and 13, between 3 and 10, between 3 and 8, between 3 and 6, between 3 and 5, between 3 and 4, between 5 and 15, between 5 and 10, between 8 and 15, between 8 and 12, between 10 and 15 or between 10 and 12. In an embodiment, the degree of conjugation of the serotype 34 glycoconjugate of the invention is about 2, about 3, about 4, about 5, about 6, about 7, about 8, about 9, about 10, about 11, about 12, about 13, about 14 or about 15. In an embodiment, the degree of conjugation of the serotype 38 glycoconjugate of the invention is between 4 and 7. In some such embodiments, the carrier protein is CRM₁₉₇.

The serotype 38 glycoconjugates of the invention may also be characterized by the ratio (weight/weight) of saccharide to carrier protein. In some embodiments, the ratio of serotype 38 polysaccharide to carrier protein in the glycoconjugate (w/w) is between 0.5 and 3.0 (e.g., about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0). In other embodiments, the saccharide to carrier protein ratio (w/w) is between 0.5 and 2.0, between 0.5 and 1.5, between 0.8 and 1.2, between 0.5 and 1.0, between 1.0 and 1.5 or between 1.0 and 2.0. In further embodiments, the saccharide to carrier protein ratio (w/w) is between 0.8 and 1.2. In an embodiment, the ratio of serotype 34 capsular polysaccharide to carrier protein in the conjugate is between 0.9 and 1.1. In some such embodiments, the carrier protein is CRM₁₉₇.

The serotype 34 glycoconjugates and immunogenic compositions of the invention may contain free saccharide that is not covalently conjugated to the carrier protein, but is nevertheless present in the glycoconjugate composition. The free saccharide may be noncovalently associated with (i.e., noncovalently bound to, adsorbed to, or entrapped in or with) the glycoconjugate.

In an embodiment, the serotype 38 glycoconjugate comprises less than about 50%, 45%, 40%, 35%, 30%, 25%, 20% or 15% of free serotype 38 polysaccharide compared to the total amount of serotype 38 polysaccharide. In another embodiment, the serotype 38 glycoconjugate comprises less than about 40% of free serotype 38 polysaccharide compared to the total amount of serotype 38 polysaccharide. In an embodiment, the serotype 38 glycoconjugate comprises less than about 25% of free serotype 38 polysaccharide compared to the total amount of serotype 38 polysaccharide. In an embodiment, the serotype 38 glycoconjugate comprises less than about 20% of free serotype 38 polysaccharide compared to the total amount of serotype 38 polysaccharide. In another embodiment the serotype 38 glycoconjugate comprises less than about 15% of free serotype 38 polysaccharide compared to the total amount of serotype 38 polysaccharide.

The serotype 38 glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In an embodiment, at least 30% of the serotype 38 glycoconjugate has a K_{d} below or equal to 0.3 in a CL-4B column. In another embodiment, at least 40% of the glycoconjugate has a K_{d} below or equal to 0.3 in a CL-4B column. In another embodiment, at least 45%, 50%, 55%, 60%, 65%, 70%, 75%, 80%, or 85% of the serotype 38 glycoconjugate has a K_{d} below or equal to 0.3 in a CL-4B column. In an embodiment, at least 60% of the serotype 34 glycoconjugate has a K_{d} below or equal to 0.3 in a CL-4B column. In another embodiment, between 50% and 80% of the serotype 34 glycoconjugate has a K_{d} below or equal to 0.3 in a CL-4B column. In an embodiment, between 65% and 80% of the serotype 38 glycoconjugate has a K_{d} below or equal to 0.3 in a CL-4B column.

### 2. Immunogenic Compositions of the Present Invention

In an embodiment, the number of S. pneumoniae capsular saccharides of the immunogenic composition can range from 1 serotype (or “v”, valence) to 16 different serotypes (16 v). In one embodiment, there is 1 serotype. In another embodiment, there are 2 different serotypes. In another embodiment, there are 3 different serotypes. In another embodiment, there are 4 different serotypes. In another embodiment, there are 5 different serotypes. In another embodiment, there are 6 different serotypes. In another embodiment, there are 7 different serotypes. In another embodiment, there are 8 different serotypes. In another embodiment, there are 9 different serotypes. In another embodiment, there are 10 different serotypes. In another embodiment, there are 11 different serotypes. In another embodiment, there are 12 different serotypes. In another embodiment, there are 13 different serotypes. In another embodiment, there are 14 different serotypes. In another embodiment, there are 15 different serotypes. In another embodiment, there are 16 different serotypes. The capsular saccharides are conjugated to a carrier protein to form glycoconjugates as described herein.

In an embodiment, the immunogenic composition of the invention comprises at least one glycoconjugate selected from the group consisting of a glycoconjugate from S. pneumoniae serotype 6C, 7C, 9N, 15A, 15B, 15C, 16F, 17F, 20, 23A, 23B, 31, 34, 35B, 35F, and 38. Such glyconconjugates incudes those described in sections 1.3.2 to 1.3.17, above.

In an embodiment, the immunogenic composition of the invention comprises at least one glycoconjugate of each of the two S. pneumoniae serotypes selected from the group consisting of: 6C and 7C; 6C and 9N; 6C and 15A; 6C and 15B; 6C and 150; 6C and 16F; 6C and 17F; 6C and 20; 6C and 23A; 6C and 23B; 6C and 31; 6C and 34; 6C and 35B; 6C and 35F; 6C and 38; 7C and 9N; 7C and 15A; 7C and 15B; 7C and 15C; 7C and 16F; 7C and 17F; 7C and 20; 7C and 23A; 7C and 23B; 7C and 31; 7C and 34; 7C and 35B; 7C and 35F; 7C and 38; 9N and 15A; 9N and 15B; 9N and 15C; 9N and 16F; 9N and 17F; 9N and 20; 9N and 23A; 9N and 23B; 9N and 31; 9N and 34; 9N and 35B; 9N and 35F; 9N and 38; 15A and 16F; 15A and 17F; 15A and 20; 15A and 23A; 15A and 23B; 15A and 31; 15A and 34; 15A and 35B; 15A and 35F; 15A and 38; 15B and 16F; 15B and 17F; 15B and 20; 15B and 23A; 15B and 23B; 15A and 31; 15A and 34; 15A and 35B; 15A and 35F; 15B and 38; 15C and 16F; 15C and 17F; 15C and 20; 15C and 23A; 15C and 23B; 15C and 31; 15C and 34; 15C and 35B; 15C and 35F; 15C and 38; 16F and 17F; 16F and 20; 16F and 23A; 16F and 23B; 16F and 31; 16F and 34; 16F and 35B; 16F and 35F; 16F and 38; 17F and 20; 17F and 23A; 17F and 23B; 17F and 31; 17F and 34; 17F and 35B; 17F and 35F; 17F and 38; 20 and 23A; 20 and 23B; 20 and 31; 20 and 34; 20 and 35B; 20 and 35F; 20 and 38; 23A and 31; 23A and 34; 23A and 35B; 23A and 35F; 23A and 38; 23B and 31; 23B and 34; 23B and 35B; 23B and 35F; 23B and 38; 31 and 34; 31 and 35B; 31 and 35F; 31 and 38; 34 and 35B; 34 and 35F; 34 and 38; 35B and 38; and 35F and 38.

All the glycoconjugates of the above immunogenic compositions may be individually conjugated to the carrier protein.

In an embodiment of any of the immunogenic compositions herein, the glycoconjugates from S. pneumoniae serotype 6C is conjugated to CRM₁₉₇. In an embodiment of any of the above immunogenic compositions, the glycoconjugates from S. pneumoniae serotype 7C is conjugated to CRM₁₉₇. In an embodiment of any of the above immunogenic compositions, the glycoconjugates from S. pneumoniae serotype 9N is conjugated to CRM₁₉₇. In an embodiment of any of the above immunogenic compositions, the glycoconjugates from S. pneumoniae serotype 15A is conjugated to CRM₁₉₇. In an embodiment of any of the above immunogenic compositions, the glycoconjugates from S. pneumoniae serotype 15B is conjugated to CRM₁₉₇. In an embodiment of any of the above immunogenic compositions, the glycoconjugates from S. pneumoniae serotype 15C is conjugated to CRM₁₉₇. In an embodiment of any of the above immunogenic compositions, the glycoconjugates from S. pneumoniae serotype 16F is conjugated to CRM₁₉₇. In an embodiment of any of the above immunogenic compositions, the glycoconjugates from S. pneumoniae serotype 17F is conjugated to CRM₁₉₇. In an embodiment of any of the above immunogenic compositions, the glycoconjugates from S. pneumoniae serotype 20 is conjugated to CRM₁₉₇. In an embodiment of any of the above immunogenic compositions, the glycoconjugates from S. pneumoniae serotype 23A is conjugated to CRM₁₉₇. In an embodiment of any of the above immunogenic compositions, the glycoconjugates from S. pneumoniae serotype 23B is conjugated to CRM₁₉₇. In an embodiment of any of the above immunogenic compositions, the glycoconjugates from S. pneumoniae serotype 23B is conjugated to CRM₁₉₇. In an embodiment of any of the above immunogenic compositions, the glycoconjugates from S. pneumoniae serotype 34 is conjugated to CRM₁₉₇. In an embodiment of any of the above immunogenic compositions, the glycoconjugates from S. pneumoniae serotype 35B is conjugated to CRM₁₉₇. In an embodiment of any of the above immunogenic compositions, the glycoconjugates from S. pneumoniae serotype 35F is conjugated to CRM₁₉₇. In an embodiment of any of the above immunogenic compositions, the glycoconjugates from S. pneumoniae serotype 38 is conjugated to CRM₁₉₇.

In an embodiment of any of the above immunogenic compositions, the glycoconjugates from S. pneumoniae are all individually conjugated to CRM₁₉₇.

In another embodiment of any of the immunogenic compositions herein, the glycoconjugates from S. pneumoniae are all individually conjugated to PD. In another embodiment, the glycoconjugates from S. pneumoniae are all individually conjugated to TT. In yet another embodiment, the glycoconjugates from S. pneumoniae are all individually conjugated to DT.

In another embodiment of any of the immunogenic compositions herein, the glycoconjugates from S. pneumoniae serotype 6C, 7C, 9N, 15A, 15B, 15C, 16F, 17F, 20, 23A, 23B, 31, 34, 35B, 35F, and/or 38 is/are individually conjugated to DT. In another embodiment, the glycoconjugates from S. pneumoniae serotype 6C, 7C, 9N, 15A, 15B, 15C, 16F, 17F, 20, 23A, 23B, 31, 34, 35B, 35F, and/or 38 is/are individually conjugated to TT. In another embodiment, the glycoconjugates from S. pneumoniae serotype 6C, 7C, 9N, 15A, 15B, 15C, 16F, 17F, 20, 23A, 23B, 31, 34, 35B, 35F, and/or 38 is/are individually conjugated to PD.

In another embodiment of any of the above immunogenic compositions, at least one of the glycoconjugates is individually conjugated to DT and the other glycoconjugate(s) from S. pneumoniae is/are individually conjugated to TT. In another embodiment, at least one of the glycoconjugates is individually conjugated to TT and the other glycoconjugate(s) is/are individually conjugated to DT. In another embodiment, at least one of the glycoconjugates is individually conjugated to PD and the other glycoconjugate(s) is/are individually conjugated to DT. In another embodiment, at least one of the glycoconjugates is individually conjugated to PD and the other glycoconjugate(s) is/are individually conjugated to TT. In another embodiment, at least one of the glycoconjugates is individually conjugated to TT and the other glycoconjugate(s) is/are individually conjugated to PD. In another embodiment, at least one of the glycoconjugates is individually conjugated to DT and the other glycoconjugate(s) is/are individually conjugated to PD.

In another embodiment of any of the above immunogenic compositions, at least one of the glycoconjugates is individually conjugated to CRM₁₉₇ and the other glycoconjugate(s) from S. pneumoniae is/are individually conjugated to DT. In another embodiment, at least one of the glycoconjugates is individually conjugated to CRM₁₉₇ and the other glycoconjugate(s) is/are individually conjugated to TT. In another embodiment, at least one of the glycoconjugates is individually conjugated to CRM₁₉₇ and the other glycoconjugate(s) is/are individually conjugated to PD. In another embodiment, at least one of the glycoconjugates is individually conjugated to DT and the other glycoconjugate(s) is/are individually conjugated to CRM₁₉₇. In another embodiment, at least one of the glycoconjugates is individually conjugated to TT and the other glycoconjugate(s) is/are individually conjugated to CRM₁₉₇. In another embodiment, at least one of the glycoconjugates is individually conjugated to PD and the other glycoconjugate(s) is/are individually conjugated to CRM₁₉₇.

In an embodiment the above immunogenic compositions comprise from 1 to 16 different serotypes of S. pneumoniae. In one embodiment the above immunogenic composition is a 1, 2, 3, 4, 5, 6, 7, 8, 9, 10, 11, 12, 13, 14, 15, or 16-valent pneumococcal conjugate composition.

In an embodiment, the immunogenic composition of the invention comprises conjugated S. pneumoniae saccharides from serotypes 6C, 7C, 9N, 15A, 15B, 15C, 16F, 17F, 20, 23A, 23B, 31, 34, 35B, 35F, and 38.

In an embodiment, the glycoconjugates of the immunogenic composition of the invention consists of glycoconjugates from S. pneumoniae serotypes 6C, 7C, 9N, 15A, 15B, 15C, 16F, 17F, 20, 23A, 23B, 31, 34, 35B, 35F, and 38.

In an embodiment, all the glycoconjugates of the immunogenic composition of the invention are individually conjugated to the carrier protein.

In an embodiment, the glycoconjugates of the immunogenic composition are individually conjugated to CRM₁₉₇. In an embodiment, the glycoconjugates of the immunogenic composition are individually conjugated to PD. In an embodiment, the glycoconjugates of the immunogenic composition are individually conjugated to TT. In an embodiment, the glycoconjugates of the immunogenic composition are individually conjugated to DT.

In an embodiment, at least one of the glycoconjugates of the immunogenic composition is individually conjugated to DT and the other glycoconjugate(s) from S. pneumoniae is/are individually conjugated to TT. In another embodiment, at least one of the glycoconjugates of the immunogenic composition is individually conjugated to TT and the other glycoconjugate(s) is/are individually conjugated to DT. In another embodiment, at least one of the glycoconjugates of the immunogenic composition is individually conjugated to PD and the other glycoconjugate(s) is/are individually conjugated to DT. In another embodiment, at least one of the glycoconjugates of the immunogenic composition is individually conjugated to PD and the other glycoconjugate(s) is/are individually conjugated to TT. In another embodiment, at least one of the glycoconjugates of the immunogenic composition is individually conjugated to TT and the other glycoconjugate(s) is/are individually conjugated to PD. In another embodiment, at least one of the glycoconjugates of the immunogenic composition is individually conjugated to DT and the other glycoconjugate(s) is/are individually conjugated to PD.

In another embodiment, at least one of the glycoconjugates of the immunogenic composition is individually conjugated to CRM₁₉₇ and the other glycoconjugate(s) from S. pneumoniae is/are individually conjugated to DT. In another embodiment, at least one of the glycoconjugates of the immunogenic composition is individually conjugated to CRM₁₉₇ and the other glycoconjugate(s) is/are individually conjugated to TT. In another embodiment, at least one of the glycoconjugates of the immunogenic composition is individually conjugated to CRM₁₉₇ and the other glycoconjugate(s) is/are individually conjugated to PD. In another embodiment, at least one of the glycoconjugates of the immunogenic composition is individually conjugated to DT and the other glycoconjugate(s) is/are individually conjugated to CRM₁₉₇. In another embodiment, at least one of the glycoconjugates of the immunogenic composition is individually conjugated to TT and the other glycoconjugate(s) is/are individually conjugated to CRM₁₉₇. In another embodiment, at least one of the glycoconjugates of the immunogenic composition is individually conjugated to PD and the other glycoconjugate(s) is/are individually conjugated to CRM₁₉₇.

After conjugation of the capsular polysaccharide to the carrier protein, the glycoconjugates are purified (enriched with respect to the amount of polysaccharide-protein conjugate) by a variety of techniques. These techniques include concentration/diafiltration operations, precipitation/elution, column chromatography, and depth filtration (see, for example, U.S. Patent App. Pub. No. 2007/0184072 or WO2008/079653). After the individual glycoconjugates are purified, they are compounded to formulate the immunogenic composition of the present invention. In an embodiment the above immunogenic compositions further comprise antigen(s) from other pathogens, particularly from bacteria and/or viruses as disclosed herein.

In an embodiment the above immunogenic compositions further comprise one or more adjuvants as disclosed herein.

In an embodiment the above immunogenic compositions are formulated as disclosed herein.

3. Immunogenic Compositions which May be Used in Combination with the Immunogenic Compositions of the Present Invention

In an embodiment, the immunogenic compositions of the invention are used in combination with a second immunogenic composition.

In an embodiment, the second immunogenic composition comprises at least one glycoconjugate from a Streptococcus pneumoniae serotype selected from the group consisting of serotypes 1, 3, 4, 5, 6A, 6B, 7F, 9V, 14, 18C, 19A, 19F, 23F, 22F and 33F.

In an embodiment, the second immunogenic composition comprises at least one glycoconjugate from a Streptococcus pneumoniae serotype selected from the group consisting of serotypes 1, 3, 4, 5, 6A, 6B, 7F, 9V, 14, 18C, 19A, 19F and 23F.

1. In an embodiment the second immunogenic composition comprises at least one glycoconjugate from S. pneumoniae serotypes 4, 6B, 9V, 14, 18C, 19F and 23F (such as the glycoconjugates of section 1.3.1 above).
2. In another embodiment the second immunogenic composition comprises in addition to point 1 above, at least one glycoconjugate from S. pneumoniae serotypes 1, 5 and 7F (such as the glycoconjugates of section 1.3.1 above).

In an embodiment, all the glycoconjugates of the above second immunogenic compositions are individually conjugated to the carrier protein.

In an embodiment of any of the above second immunogenic compositions, the glycoconjugates from S. pneumoniae serotypes 4, 6B, 9V, 14, 18C, 19F and 23F are conjugated to CRM₁₉₇. In an embodiment of any of the above second immunogenic compositions, the glycoconjugates from S. pneumoniae serotypes 1, 5 and 7F are conjugated to CRM₁₉₇. In an embodiment of any of the above second immunogenic compositions, the glycoconjugates from S. pneumoniae serotypes 6A and 19A are conjugated to CRM₁₉₇. In an embodiment of any of the above second immunogenic compositions, the glycoconjugates from S. pneumoniae serotype 3 is conjugated to CRM₁₉₇.

In an embodiment, the glycoconjugates from S. pneumoniae serotypes 1, 4, 5, 6B, 7F, 9V, 14 and/or 23F of any of the above second immunogenic compositions are individually conjugated to PD.

In an embodiment, the glycoconjugate from S. pneumoniae serotype 18C of any of the above second immunogenic compositions is conjugated to TT.

In an embodiment, the glycoconjugate from S. pneumoniae serotype 19F of any of the above second immunogenic compositions is conjugated to DT.

In an embodiment, the glycoconjugates from S. pneumoniae serotypes 1, 4, 5, 6B, 7F, 9V, 14 and/or 23F of any of the above second immunogenic compositions are individually conjugated to PD, the glycoconjugate from S. pneumoniae serotype 18C is conjugated to TT and the glycoconjugate from S. pneumoniae serotype 19F is conjugated to DT.

In an embodiment, the glycoconjugates from S. pneumoniae serotypes 1, 4, 5, 6B, 7F, 9V, 14 and/or 23F of any of the above second immunogenic compositions are individually conjugated to PD, the glycoconjugate from S. pneumoniae serotype 18C is conjugated to TT, the glycoconjugate from S. pneumoniae serotype 19F is conjugated to DT, the glycoconjugate from S. pneumoniae serotype 22F is conjugated to CRM₁₉₇ and the glycoconjugate from S. pneumoniae serotype 33F is conjugated to CRM₁₉₇.

In an embodiment, the above second immunogenic compositions comprise from 7 to 15 different serotypes of S. pneumoniae. In one embodiment, the above second immunogenic compositions comprise glycoconjugates from 7, 8, 9, 10, 11, 12, 13, 14 or 15 different serotypes. In one embodiment, the above second immunogenic compositions comprise glycoconjugates from 10 to 15 different serotypes. In an embodiment, the above second immunogenic composition is a 7, 8, 9, 10, 11, 12, 13, 14 or 15-valent pneumococcal conjugate composition. In an embodiment the above second immunogenic composition is a 10-valent pneumococcal conjugate composition. In an embodiment the above second immunogenic composition is an 11-valent pneumococcal conjugate composition. In an embodiment the above second immunogenic composition is a 12-valent pneumococcal conjugate composition. In an embodiment the above second immunogenic composition is a 13-valent pneumococcal conjugate composition. In an embodiment the above second immunogenic composition is a 14-valent pneumococcal conjugate composition. In an embodiment the above second immunogenic composition is a 15-valent pneumococcal conjugate composition.

In an embodiment, the above second immunogenic composition is a 7-valent pneumococcal conjugate composition wherein said 7 conjugates consists of 7 glycoconjugates from S. pneumoniae serotypes 4, 6B, 9V, 14, 18C, 19F and 23F individually conjugated to CRM₁₉₇.

In an embodiment, the above second immunogenic composition is a 10-valent pneumococcal conjugate composition wherein said 10 conjugates consists of glycoconjugates from S. pneumoniae serotypes 1, 4, 5, 6B, 7F, 9V, 14 and 23F individually conjugated to PD, glycoconjugate from S. pneumoniae serotype 18C conjugated to TT and glycoconjugate from S. pneumoniae serotype 19F conjugated to DT.

In an embodiment, the above second immunogenic composition is an 11-valent pneumococcal conjugate composition wherein said 11 conjugates consists of glycoconjugates from S. pneumoniae serotypes 1, 4, 5, 6B, 7F, 9V, 14 and 23F individually conjugated to PD, glycoconjugate from S. pneumoniae serotype 18C conjugated to TT, glycoconjugate from S. pneumoniae serotype 19F conjugated to DT and glycoconjugate from S. pneumoniae serotype 22F conjugated to CRM₁₉₇.

In an embodiment, the above second immunogenic composition is an 11-valent pneumococcal conjugate composition wherein said 11 conjugates consists of glycoconjugates from S. pneumoniae serotypes 1, 4, 5, 6B, 7F, 9V, 14 and 23F individually conjugated to PD, glycoconjugate from S. pneumoniae serotype 18C conjugated to TT, glycoconjugate from S. pneumoniae serotype 19F conjugated to DT and glycoconjugate from S. pneumoniae serotype 33F conjugated to CRM₁₉₇.

In an embodiment, the above second immunogenic composition is a 12-valent pneumococcal conjugate composition wherein said 12 conjugates consists of glycoconjugates from S. pneumoniae serotypes 1, 4, 5, 6B, 7F, 9V, 14 and 23F individually conjugated to PD, glycoconjugate from S. pneumoniae serotype 18C conjugated to TT, glycoconjugate from S. pneumoniae serotype 19F conjugated to DT, glycoconjugate from S. pneumoniae serotype 22F conjugated to CRM₁₉₇ and glycoconjugate from S. pneumoniae serotype 33F conjugated to CRM₁₉₇.

In an embodiment, the above second immunogenic composition is a 13-valent pneumococcal conjugate composition wherein said 13 conjugates consists of glycoconjugates from S. pneumoniae serotypes 1, 3, 4, 5, 6A, 6B, 7F, 9V, 14, 18C, 19A, 19F and 23F individually conjugated to CRM₁₉₇.

In an embodiment, the above second immunogenic composition is a 14-valent pneumococcal conjugate composition wherein said 14 conjugates consists of glycoconjugates from S. pneumoniae serotypes 1, 3, 4, 5, 6A, 6B, 7F, 9V, 14, 18C, 19A, 19F, 23F and 22F individually conjugated to CRM₁₉₇.

In an embodiment, the above second immunogenic composition is a 14-valent pneumococcal conjugate composition wherein said 14 conjugates consists of glycoconjugates from S. pneumoniae serotypes 1, 3, 4, 5, 6A, 6B, 7F, 9V, 14, 18C, 19A, 19F, 23F and 33F individually conjugated to CRM₁₉₇.

In an embodiment, the above second immunogenic composition is a 15-valent pneumococcal conjugate composition wherein said 15 conjugates consists of glycoconjugates from S. pneumoniae serotypes 1, 3, 4, 5, 6A, 6B, 7F, 9V, 14, 18C, 19A, 19F, 23F, 22F and 33F individually conjugated to CRM₁₉₇.

In an embodiment the dosage of the above second immunogenic is as disclosed below.

In an embodiment the above second immunogenic compositions further comprise antigen(s) from other pathogen(s), particularly from bacteria and/or viruses such as disclosed at section 6 below.

In an embodiment the above second immunogenic compositions further comprise one or more adjuvants as disclosed at section 7 below.

In an embodiment the above second immunogenic compositions are formulated as disclosed at section 8 below.

In an embodiment, the immunogenic compositions of the invention (such as any of the ones of section 2 above) are used in combination with PREVNAR® (PREVENAR® in some countries) (heptavalent vaccine), SYNFLORIX® (a decavalent vaccine) and/or PREVNAR 13® (PREVENAR 13® in some countries) (tridecavalent vaccine).

### 4. Kit of the Present Invention

In an aspect, the invention provides a kit comprising: (a) a first immunogenic composition, as defined at section 2 above; and (b) a second immunogenic composition comprising at least one glycoconjugate from a Streptococcus pneumoniae serotype selected from serotypes 1, 3, 4, 5, 6A, 6B, 7F, 9V, 14, 18C, 19A, 19F, 23F, 22F and 33F.

In an aspect, the invention provides a kit comprising: (a) a first immunogenic composition, as defined at section 2 above; and (b) a second immunogenic composition comprising at least one glycoconjugate from a Streptococcus pneumoniae serotype selected from serotypes 1, 3, 4, 5, 6A, 6B, 7F, 9V, 14, 18C, 19A, 19F and 23F.

In an aspect, the invention provides a kit comprising: (a) a first immunogenic composition, as defined at section 2 above; and (b) a second immunogenic composition as defined at section 3 above.

In an embodiment, the second immunogenic composition of the kit (part (b) of the kit) comprises glycoconjugates from S. pneumoniae serotypes 4, 6B, 9V, 14, 18C, 19F and 23F.

In an embodiment, the second immunogenic composition of the kit comprises glycoconjugates from S. pneumoniae serotypes 1, 4, 5, 6B, 7F, 9V, 14, 18C, 19F and 23F.

In an embodiment the second immunogenic composition of the kit comprises glycoconjugates from S. pneumoniae serotypes 1, 4, 5, 6A, 6B, 7F, 9V, 14, 18C, 19A, 19F and 23F (such as the glycoconjugates of section 1.3.1 above).

In an embodiment the second immunogenic composition of the kit comprises glycoconjugates from S. pneumoniae serotypes 1, 3, 4, 5, 6A, 6B, 7F, 9V, 14, 18C, 19A, 19F and 23F (such as the glycoconjugates of section 1.3.1 above).

In an embodiment the second immunogenic composition of the kit comprises glycoconjugates from S. pneumoniae serotypes 1, 4, 5, 6B, 7F, 9V, 14, 18C, 19F, 23F and 22F (such as the glycoconjugates of section 1.3.1 and 1.3.2 above).

In an embodiment the second immunogenic composition of the kit comprises glycoconjugates from S. pneumoniae serotypes 1, 4, 5, 6B, 7F, 9V, 14, 18C, 19F, 23F and 33F (such as the glycoconjugates of sections 1.3.1 and 1.3.3 above).

In an embodiment the second immunogenic composition of the kit comprises glycoconjugates from S. pneumoniae serotypes 1, 4, 5, 6B, 7F, 9V, 14, 18C, 19F, 23F, 22F and 33F (such as the glycoconjugates of section 1.3.1, 1.3.2 and 1.3.3 above).

In an embodiment the second immunogenic composition of the kit comprises glycoconjugates from S. pneumoniae serotypes 1, 3, 4, 5, 6A, 6B, 7F, 9V, 14, 18C, 19A, 19F, 23F and 22F (such as the glycoconjugates of sections 1.3.1 and 1.3.2 above).

In an embodiment the second immunogenic composition of the kit comprises glycoconjugates from S. pneumoniae serotypes 1, 3, 4, 5, 6A, 6B, 7F, 9V, 14, 18C, 19A, 19F, 23F and 33F (such as the glycoconjugates of sections 1.3.1 and 1.3.3 above).

In an embodiment the second immunogenic composition of the kit comprises glycoconjugates from S. pneumoniae serotypes 1, 3, 4, 5, 6A, 6B, 7F, 9V, 14, 18C, 19A, 19F, 23F, 22F and 33F (such as the glycoconjugates of sections 1.3.1, 1.3.2 and 1.3.3 above).

All the glycoconjugates of the second immunogenic composition of the kit may be individually conjugated to the carrier protein.

In an embodiment of any of the above kits, the glycoconjugates from S. pneumoniae serotypes 4, 6B, 9V, 14, 18C, 19F and 23F are conjugated to CRM₁₉₇. In an embodiment of any of the above kits, the glycoconjugates from S. pneumoniae serotypes 1, 5 and 7F are conjugated to CRM₁₉₇. In an embodiment of any of the above kits, the glycoconjugates from S. pneumoniae serotypes 6A and 19A are conjugated to CRM₁₉₇. In an embodiment of any of the above kits, the glycoconjugates from S. pneumoniae serotype 3 is conjugated to CRM₁₉₇.

In an embodiment, the glycoconjugates of any of the above kits are all individually conjugated to CRM₁₉₇.

In another embodiment, the glycoconjugates from S. pneumoniae serotypes 1, 4, 5, 6B, 7F, 9V, 14 and/or 23F of any of the above kits are individually conjugated to PD.

In an embodiment, the glycoconjugate from S. pneumoniae serotype 18C of any of the above kits is conjugated to TT.

In an embodiment, the glycoconjugate from S. pneumoniae serotype 19F of any of the above kits is conjugated to DT.

In an embodiment, the glycoconjugates from S. pneumoniae serotypes 1, 4, 5, 6B, 7F, 9V, 14 and/or 23F of any of the above kits are individually conjugated to PD, the glycoconjugate from S. pneumoniae serotype 18C is conjugated to TT and the glycoconjugate from S. pneumoniae serotype 19F is conjugated to DT.

In an embodiment, the glycoconjugates from S. pneumoniae serotypes 1, 4, 5, 6B, 7F, 9V, 14 and/or 23F of any of the above kits are individually conjugated to PD, the glycoconjugate from S. pneumoniae serotype 18C is conjugated to TT, the glycoconjugate from S. pneumoniae serotype 19F is conjugated to DT, the glycoconjugate from S. pneumoniae serotype 22F is conjugated to CRM₁₉₇ and the glycoconjugate from S. pneumoniae serotype 33F is conjugated to CRM₁₉₇.

In an embodiment the above second immunogenic compositions comprise from 7 to 15 different serotypes of S. pneumoniae. In one embodiment the above second immunogenic compositions comprise glycoconjugates from 7, 8, 9, 10, 11, 12, 13, 14 or 15 different serotypes. In one embodiment the above second immunogenic compositions comprise glycoconjugates from 10 to 15 different serotypes. In an embodiment the above second immunogenic composition is a 7, 8, 9, 10, 11, 12, 13, 14 or 15-valent pneumococcal conjugate composition. In an embodiment the above second immunogenic composition is a 10-valent pneumococcal conjugate composition. In an embodiment the above second immunogenic composition is an 11-valent pneumococcal conjugate composition. In an embodiment the above second immunogenic composition is a 12-valent pneumococcal conjugate composition. In an embodiment the above second immunogenic composition is a 13-valent pneumococcal conjugate composition. In an embodiment the above second immunogenic composition is a 14-valent pneumococcal conjugate composition. In an embodiment the above second immunogenic composition is a 15-valent pneumococcal conjugate composition.

In an embodiment, the above second immunogenic composition is a 7-valent pneumococcal conjugate composition wherein said 7 conjugates consists of 7 glycoconjugates from S. pneumoniae serotypes 4, 6B, 9V, 14, 18C, 19F and 23F individually conjugated to CRM₁₉₇.

In an embodiment, the above second immunogenic composition is a 10-valent pneumococcal conjugate composition wherein said 10 conjugates consists of glycoconjugates from S. pneumoniae serotypes 1, 4, 5, 6B, 7F, 9V, 14 and 23F individually conjugated to PD, glycoconjugate from S. pneumoniae serotype 18C conjugated to TT and glycoconjugate from S. pneumoniae serotype 19F conjugated to DT.

In an embodiment, the above second immunogenic composition is an 11-valent pneumococcal conjugate composition wherein said 11 conjugates consists of glycoconjugates from S. pneumoniae serotypes 1, 4, 5, 6B, 7F, 9V, 14 and 23F individually conjugated to PD, glycoconjugate from S. pneumoniae serotype 18C conjugated to TT, glycoconjugate from S. pneumoniae serotype 19F conjugated to DT and glycoconjugate from S. pneumoniae serotype 22F conjugated to CRM₁₉₇.

In an embodiment, the above second immunogenic composition is an 11-valent pneumococcal conjugate composition wherein said 11 conjugates consists of glycoconjugates from S. pneumoniae serotypes 1, 4, 5, 6B, 7F, 9V, 14 and 23F individually conjugated to PD, glycoconjugate from S. pneumoniae serotype 18C conjugated to TT, glycoconjugate from S. pneumoniae serotype 19F conjugated to DT and glycoconjugate from S. pneumoniae serotype 33F conjugated to CRM₁₉₇.

In an embodiment, the above second immunogenic composition is a 12-valent pneumococcal conjugate composition wherein said 12 conjugates consists of glycoconjugates from S. pneumoniae serotypes 1, 4, 5, 6B, 7F, 9V, 14 and 23F individually conjugated to PD, glycoconjugate from S. pneumoniae serotype 18C conjugated to TT, glycoconjugate from S. pneumoniae serotype 19F conjugated to DT, glycoconjugate from S. pneumoniae serotype 22F conjugated to CRM₁₉₇ and glycoconjugate from S. pneumoniae serotype 33F conjugated to CRM₁₉₇.

In an embodiment, the above second immunogenic composition is a 13-valent pneumococcal conjugate composition wherein said 13 conjugates consists of glycoconjugates from S. pneumoniae serotypes 1, 3, 4, 5, 6A, 6B, 7F, 9V, 14, 18C, 19A, 19F and 23F individually conjugated to CRM₁₉₇.

In an embodiment, the above second immunogenic composition is a 14-valent pneumococcal conjugate composition wherein said 14 conjugates consists of glycoconjugates from S. pneumoniae serotypes 1, 3, 4, 5, 6A, 6B, 7F, 9V, 14, 18C, 19A, 19F, 23F and 22F individually conjugated to CRM₁₉₇.

In an embodiment, the above second immunogenic composition is a 14-valent pneumococcal conjugate composition wherein said 14 conjugates consists of glycoconjugates from S. pneumoniae serotypes 1, 3, 4, 5, 6A, 6B, 7F, 9V, 14, 18C, 19A, 19F, 23F and 33F individually conjugated to CRM₁₉₇.

In an embodiment, the above second immunogenic composition is a 15-valent pneumococcal conjugate composition wherein said 15 conjugates consists of glycoconjugates from S. pneumoniae serotypes 1, 3, 4, 5, 6A, 6B, 7F, 9V, 14, 18C, 19A, 19F, 23F, 22F and 33F individually conjugated to CRM₁₉₇.

In an embodiment the above second immunogenic compositions further comprise antigens from other pathogens, particularly from bacteria and/or viruses as disclosed herein.

In an embodiment the above second immunogenic compositions further comprise one or more adjuvants as disclosed herein.

In an embodiment the above second immunogenic compositions are formulated as disclosed herein.

In an embodiment, the immunogenic compositions of the invention (such as any of the ones of section 2 above) are used in combination with PREVNAR® (PREVENAR® in some countries) (heptavalent vaccine), SYNFLORIX® (a decavalent vaccine) and/or PREVNAR 13® (PREVENAR 13® in some countries) (tridecavalent vaccine).

In an aspect of the present invention, the kit takes the form of two containers. Therefore, in one embodiment of the present invention each of the immunogenic compositions of the kit (i.e., the first immunogenic composition and the second immunogenic composition) is comprised in a separate container.

In one embodiment, the first immunogenic composition of the kit (part (a) of the kit) is comprised in a container selected from the group consisting of a vial, a syringe, a flask, a fermentor, a bioreactor, a bag, a jar, an ampoule, a cartridge and a disposable pen. In certain embodiments, the container is siliconized.

In one embodiment, the second immunogenic composition of the kit (part (b) of the kit) is comprised in a container selected from the group consisting of a vial, a syringe, a flask, a fermentor, a bioreactor, a bag, a jar, an ampoule, a cartridge and a disposable pen. In certain embodiments, the container is siliconized.

In an embodiment, the container is made of glass, metals (e.g., steel, stainless steel, aluminum, etc.) and/or polymers (e.g., thermoplastics, elastomers, thermoplastic-elastomers). In an embodiment, the container is made of glass.

In one embodiment, the first and second immunogenic compositions of the kit are comprised in a syringe or a disposable pen. In one embodiment, the first and second immunogenic compositions of the kit are comprised in a syringe. In certain embodiments, the syringes are siliconized. In certain embodiments, the siliconized syringes are made of glass.

In an embodiment, the first and second immunogenic compositions of the kit are mixed extemporaneously for simultaneous administration.

In an embodiment, the first and second immunogenic compositions are in liquid form, preferably contained in two containers. In one embodiment, the first and second containers are separate chambers in a dual-chamber syringe such that, when actuated, liquid in the first container is introduced into the second container. The resulting mixture can then exit the syringe. The two immunogenic compositions are kept separate until ready for mixing.

In an embodiment, the first and/or second immunogenic composition of the kit is/are in lyophilized form.

In an embodiment, the first immunogenic composition of the kit is in lyophilized form and the second immunogenic composition is in liquid form. In another embodiment, the second immunogenic composition of the kit is in lyophilized form and the first immunogenic composition is in liquid form. In said embodiments, the lyophilized immunogenic composition can be reconstituted extemporaneously with the liquid immunogenic composition for simultaneous administration of both immunogenic compositions.

In said embodiments, the kit contains two containers, one container includes liquid material for reconstitution and the second container includes lyophilized material.

In one embodiment the second container is hermetically sealed. In an embodiment, the liquid material is introduced into the second container via a first needle, thereby reconstituting the lyophilized material into a liquid form. The resulting mixture is then withdrawn, into a container (such as a syringe), for administration to a patient. In one embodiment the withdrawal step is via the first needle. In another embodiment, the withdrawal step is via a second needle. In an embodiment, the needle used for the withdrawal step is the same needle that is used for patient injection. In another embodiment, the needle used for the withdrawal step is different from the needle used for patient injection.

In one embodiment, the second container is a vial. In a further embodiment the first and second containers are separate chambers in a dual-chamber syringe such that, when actuated, the liquid material is introduced from the first container into the second container. The resulting mixture exits the syringe in liquid form. In an embodiment, the lyophilized and liquid materials are kept separate until ready for mixing.

In an embodiment, the kit comprises a ready-filled syringe and a vial. In one embodiment the syringe comprises a single dose of the first immunogenic composition and the vial comprises a single dose of the second immunogenic composition. In an embodiment the syringe comprises a single dose of the second immunogenic composition and the vial comprises a single dose of the first immunogenic composition. In another embodiment, the syringe and the vial comprise multiple doses.

### 5. Dosage of the Immunogenic Compositions

The amount of glycoconjugate(s) in each dose is selected as an amount which induces an immunoprotective response without significant, adverse side effects in typical vaccinees. Such amount will vary depending upon which specific immunogen is employed and how it is presented.

### 5.1 Glycoconjugate Amount

The amount of a particular glycoconjugate in an immunogenic composition can be calculated based on total polysaccharide for that conjugate (conjugated and non-conjugated). For example, a glycoconjugate with 20% free polysaccharide has about 80 μg of conjugated polysaccharide and about 20 μg of nonconjugated polysaccharide in a 100 μg polysaccharide dose. The amount of glycoconjugate can vary depending upon the pneumococcal serotype. The saccharide concentration can be determined by the uronic acid assay.

The “immunogenic amount” of the different polysaccharide components in the immunogenic composition, may diverge and each may comprise about 1 μg, about 2 μg, about 3 μg, about 4 μg, about 5 μg, about 6 μg, about 7 μg, about 8 μg, about 9 μg, about 10 μg, about 15 μg, about 20 μg, about 30 μg, about 40 μg, about 50 μg, about 60 μg, about 70 μg, about 80 μg, about 90 μg, or about 100 μg of any particular polysaccharide antigen.

Generally, each dose comprises 0.1 μg to 100 μg of polysaccharide for a given serotype, particularly 0.5 μg to 20 μg, more particularly 1.0 μg to 10 μg, and even more more particularly 2.0 μg to 5.0 μg. Any whole number integer within any of the above ranges is contemplated as an embodiment of the disclosure.

In an embodiment, each dose comprises about 1.0 μg, about 1.2 μg, about 1.4 μg, about 1.6 μg, about 1.8 μg, about 2.0 μg, about 2.2 μg, about 2.4 μg, about 2.6 μg, about 2.8 μg, about 3.0 μg, about 3.2 μg, about 3.4 μg, about 3.6 μg, about 3.8 μg, about 4.0 μg, about 4.2 μg, about 4.4 μg, about 4.6 μg, about 4.8 μg, about 5.0 μg, about 5.2 μg, about 5.4 μg, about 5.6 μg, about 5.8 μg or about 6.0 μg of polysaccharide for each particular glycoconjugate.

In an embodiment, each dose comprises about 1.1 μg, about 1.2 μg, about 1.3 μg, about 1.4 μg, about 1.5 μg, about 1.6 μg, about 1.7 μg, about 1.8 μg, about 1.9 μg, about 2.0 μg, about 2.1 μg, about 2.2 μg, about 2.3 μg, about 2.4 μg, about 2.5 μg, about 2.6 μg, about 2.7 μg, about 2.8 μg, about 2.9 μg, or about 3.0 μg μg of polysaccharide for glycoconjugates from S. pneumoniae serotype 1, 3, 4, 5, 6A, 7F, 8, 9V, 10A, 11A, 12F, 14, 15B, 18C, 19A, 19F, 22F, 23F and/or 33F.

In an embodiment, each dose will comprise about 1.1 μg, about 1.2 μg, about 1.3 μg, about 1.4 μg, about 1.5 μg, about 1.6 μg, about 1.7 μg, about 1.8 μg, about 1.9 μg, about 2.0 μg, about 2.1 μg, about 2.2 μg, about 2.3 μg, about 2.4 μg, about 2.5 μg, about 2.6 μg, about 2.7 μg, about 2.8 μg, about 2.9 μg, or about 3.0 μg μg of polysaccharide for glycoconjugates from S. pneumoniae serotype 8, 10A, 11A, 12F, 15B, 22F and 33F.

In an embodiment, each dose comprises about 2.0 μg, about 2.2 μg, about 2.4 μg, about 2.6 μg, about 2.8 μg, about 3.0 μg, about 3.2 μg, about 3.4 μg, about 3.6 μg, about 3.8 μg, about 4.0 μg, about 4.2 μg, about 4.4 μg, about 4.6 μg, about 4.8 μg, about 5.0, about 5.2 μg, about 5.4 μg, about 5.6 μg, about 5.8 μg or about 6.0 μg of polysaccharide for glycoconjugates from S. pneumoniae serotype 6B.

In an embodiment, each dose comprises about 1.5 μg to about 3.0 μg of polysaccharide for each glycoconjugate from S. pneumoniae serotype 1, 3, 4, 5, 6A, 7F, 8, 9V, 10A, 11A, 12F, 14, 15B, 18C, 19A, 19F, 22F, 23F and/or 33F, and about 3.0 μg to about 6.0 μg of polysaccharide for glycoconjugate from S. pneumoniae serotype 6B.

In an embodiment, each dose comprises about 2.0 μg to about 2.5 μg of polysaccharide for each glycoconjugate from S. pneumoniae serotype 1, 3, 4, 5, 6A, 7F, 8, 9V, 10A, 11A, 12F, 14, 15B, 18C, 19A, 19F, 22F, 23F and/or 33F, and about 4.0 μg to about 4.8 μg of polysaccharide for glycoconjugate from S. pneumoniae serotype 6B.

In an embodiment, each dose comprises about 2.2 μg of polysaccharide from each glycoconjugate from S. pneumoniae serotype 1, 3, 4, 5, 6A, 7F, 8, 9V, 10A, 11A, 12F, 14, 15B, 18C, 19A, 19F, 22F, 23F and/or 33F, and about 4.4 μg of polysaccharide for glycoconjugate from S. pneumoniae serotype 6B.

In an embodiment, each dose comprises about 1.5 μg to about 3.0 μg of polysaccharide for each glycoconjugate from S. pneumoniae serotype 8, 10A, 11A, 12F, 15B, 22F and 33F.

In an embodiment, each dose comprises about 2.0 μg to about 2.5 μg of polysaccharide for each glycoconjugate from S. pneumoniae serotype 8, 10A, 11A, 12F, 15B, 22F and 33F.

In an embodiment, each dose comprises about 2.2 μg of polysaccharide from each glycoconjugate from S. pneumoniae serotype 8, 10A, 11A, 12F, 15B, 22F and 33F.

### 5.2 Carrier Amount

Generally, each dose of an immunogenic composition of the invention comprises 1 μg to 150 μg of carrier protein, particularly 10 μg to 100 μg of carrier protein, more particularly 15 μg to 50 μg of carrier protein, and even more particularly 16 μg to 40 μg of carrier protein. In an embodiment, said carrier protein is CRM₁₉₇.

In an embodiment, each dose comprises about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg or about 75 μg of carrier protein. In an embodiment, said carrier protein is CRM₁₉₇.

In an embodiment, each dose comprises about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, or about 30 μg of carrier protein. In an embodiment, the carrier protein is CRM₁₉₇.

### 6. Further Antigens

Immunogenic compositions disclosed herein comprise conjugated S. pneumoniae saccharide antigen(s) (glycoconjugate(s)). They may also further include at least one antigen from other pathogens, particularly from bacteria and/or viruses.

In an embodiment, the immunogenic composition disclosed herein further comprises at least one antigen selected from the group consisting of a diphtheria toxoid (D), a tetanus toxoid (T), a pertussis antigen (P), an acellular pertussis antigen (Pa), a hepatitis B virus (HBV) surface antigen (HBsAg), a hepatitis A virus (HAV) antigen, a conjugated Haemophilus influenzae type b capsular saccharide (Hib), and inactivated poliovirus vaccine (IPV).

In an embodiment, the immunogenic compositions disclosed herein comprise D-T-Pa. In an embodiment, the immunogenic compositions disclosed herein comprise D-T-Pa-Hib, D-T-Pa-IPV or D-T-Pa-HBsAg. In an embodiment, the immunogenic compositions disclosed herein comprise D-T-Pa-HBsAg-IPV or D-T-Pa-HBsAg-Hib. In an embodiment, the immunogenic compositions disclosed herein comprise D-T-Pa-HBsAg-IPV-Hib.

Pertussis antigens: Bordetella pertussis causes whooping cough. Pertussis antigens in vaccines are either cellular (whole cell, in the form of inactivated B. pertussis cells) or acellular. Preparation of cellular pertussis antigens is well documented (e.g., it may be obtained by heat inactivation of phase I culture of B. pertussis). Preferably, however, the invention uses acellular antigens. Where acellular antigens are used, it is to use one, two or (preferably) three of the following antigens: (1) detoxified pertussis toxin (pertussis toxoid, or PT); (2) filamentous hemagglutinin (FHA); (3) pertactin (also known as the 69 kiloDalton outer membrane protein). FHA and pertactin may be treated with formaldehyde prior to use according to the invention. PT is preferably detoxified by treatment with formaldehyde and/or glutaraldehyde. Acellular pertussis antigens are preferably adsorbed onto one or more aluminum salt adjuvants. As an alternative, they may be added in an unadsorbed state. Where pertactin is added, it is preferably already adsorbed onto an aluminum hydroxide adjuvant. PT and FHA may be adsorbed onto an aluminum hydroxide adjuvant or an aluminum phosphate. Adsorption of all of PT, FHA and pertactin to aluminum hydroxide is most.

Inactivated poliovirus vaccine: Poliovirus causes poliomyelitis. Rather than use oral poliovirus vaccine, embodiments of the invention use IPV. Prior to administration to patients, polioviruses must be inactivated, and this can be achieved by treatment with formaldehyde. Poliomyelitis can be caused by one of three types of poliovirus. The three types are similar and cause identical symptoms, but they are antigenically different and infection by one type does not protect against infection by others. It is therefore to use three poliovirus antigens in the invention: poliovirus Type 1 (e.g., Mahoney strain), poliovirus Type 2 (e.g., MEF-1 strain), and poliovirus Type 3 (e.g., Saukett strain). The viruses are preferably grown, purified and inactivated individually, and are then combined to give a bulk trivalent mixture for use with the invention.

Diphtheria toxoid: Corynebacterium diphtheriae causes diphtheria. Diphtheria toxin can be treated (e.g., using formalin or formaldehyde) to remove toxicity while retaining the ability to induce specific anti-toxin antibodies after injection. These diphtheria toxoids are used in diphtheria vaccines. Diphtheria toxoids are those prepared by formaldehyde treatment. The diphtheria toxoid can be obtained by growing C. diphtheriae in growth medium, followed by formaldehyde treatment, ultrafiltration and precipitation. The toxoided material may then be treated by a process comprising sterile filtration and/or dialysis. The diphtheria toxoid is preferably adsorbed onto an aluminum hydroxide adjuvant.

Tetanus toxoid: Clostridium tetani causes tetanus. Tetanus toxin can be treated to give a protective toxoid. The toxoids are used in tetanus vaccines, tetanus toxoids are those prepared by formaldehyde treatment. The tetanus toxoid can be obtained by growing C. tetani in growth medium, followed by formaldehyde treatment, ultrafiltration and precipitation. The material may then be treated by a process comprising sterile filtration and/or dialysis.

Hepatitis A virus antigens: Hepatitis A virus (HAV) is one of the known agents which causes viral hepatitis. AN HAV component is based on inactivated virus, and inactivation can be achieved by formalin treatment.

Hepatitis B virus (HBV) is one of the known agents which causes viral hepatitis. The major component of the capsid is a protein known as HBV surface antigen or, more commonly, HBsAg, which is typically a 226-amino acid polypeptide with a molecular weight of ˜24 kDa. All existing hepatitis B vaccines contain HBsAg, and when this antigen is administered to a normal vaccinee, it stimulates the production of anti-HBsAg antibodies which protect against HBV infection.

For vaccine manufacture, HBsAg has been made in two ways: purification of the antigen in particulate form from the plasma of chronic hepatitis B carriers or expression of the protein by recombinant DNA methods (e.g., recombinant expression in yeast cells). Unlike native HBsAg (i.e., as in the plasma-purified product), yeast-expressed HBsAg is generally non-glycosylated, and this is the most form of HBsAg for use with the invention.

Conjugated Haemophilus influenzae type b antigens: Haemophilus influenzae type b (Hib) causes bacterial meningitis. Hib vaccines are typically based on the capsular saccharide antigen, the preparation of which is well documented. The Hib saccharide can be conjugated to a carrier protein in order to enhance its immunogenicity, especially in children. Typical carrier proteins are tetanus toxoid, diphtheria toxoid, CRM₁₉₇, H. influenzae protein D, and an outer membrane protein complex from serogroup B meningococcus. The saccharide moiety of the conjugate may comprise full-length polyribosylribitol phosphate (PRP) as prepared from Hib bacteria, and/or fragments of full-length PRP. Hib conjugates may or may not be adsorbed to an aluminum salt adjuvant.

In an embodiment the immunogenic compositions disclosed herein further include a conjugated N. meningitidis serogroup Y capsular saccharide (MenY), and/or a conjugated N. meningitidis serogroup C capsular saccharide (MenC).

In an embodiment the immunogenic compositions disclosed herein further include a conjugated N. meningitidis serogroup A capsular saccharide (MenA), a conjugated N. meningitidis serogroup W135 capsular saccharide (MenW135), a conjugated N. meningitidis serogroup Y capsular saccharide (MenY), and/or a conjugated N. meningitidis serogroup C capsular saccharide (MenC).

In an embodiment the immunogenic compositions disclosed herein further include a conjugated N. meningitidis serogroup W135 capsular saccharide (MenW135), a conjugated N. meningitidis serogroup Y capsular saccharide (MenY), and/or a conjugated N. meningitidis serogroup C capsular saccharide (MenC).

An aspect of the invention provides a kit as defined at section 4 above wherein any of the above further antigen(s) is part of the first immunogenic composition (part (a) of the kit).

An aspect of the invention provides a kit as defined at section 4 above wherein any of the above further antigen(s) is part of the second immunogenic composition (part (b) of the kit).

An aspect of the invention provides a kit as defined at section 4 above wherein any of the above further antigen(s) is part of the first immunogenic composition (part (a) of the kit) and any of the above further antigen(s) is part of the second immunogenic composition (part (b) of the kit).

### 7. Adjuvant(s)

In one or more embodiments, the immunogenic compositions disclosed herein may further comprise at least one, two or three adjuvants. The term “adjuvant” refers to a compound or mixture that enhances the immune response to an antigen. Antigens may act primarily as a delivery system, primarily as an immune modulator or have strong features of both. Suitable adjuvants include those suitable for use in mammals, including humans.

Examples of known suitable delivery-system type adjuvants that can be used in humans include, but are not limited to, alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (TWEEN® 80), 0.5% w/V sorbitan trioleate (Span 85)), water-in-oil emulsions such as MONTANIDE™, and poly(D,L-lactide-co-glycolide) (PLG) microparticles or nanoparticles.

In an embodiment, the immunogenic compositions disclosed herein comprise aluminum salts (alum) as adjuvant (e.g., aluminum phosphate, aluminum sulfate or aluminum hydroxide). In an embodiment, the immunogenic compositions disclosed herein comprise aluminum phosphate or aluminum hydroxide as adjuvant. In an embodiment, the immunogenic compositions disclosed herein comprise from 0.1 mg/mL to 1 mg/mL or from 0.2 mg/mL to 0.3 mg/ml of elemental aluminum in the form of aluminum phosphate. In an embodiment, the immunogenic compositions disclosed herein comprise about 0.25 mg/ml of elemental aluminum in the form of aluminum phosphate.

Examples of known suitable immune modulatory type adjuvants that can be used in humans include, but are not limited to, saponin extracts from the bark of the Aquilla tree (QS21, QUILA®), TLR4 agonists such as MPL (Monophosphoryl Lipid A), 3DMPL (3-O-deacylated MPL) or GLA-AQ, LT/CT mutants, cytokines such as the various interleukins (e.g., IL-2, IL-12) or GM-CSF, and the like.

Examples of known suitable immune modulatory type adjuvants with both delivery and immune modulatory features that can be used in humans include, but are not limited to, ISCOMS (see, e.g., Sjölander et al. (1998) J. Leukocyte Biol. 64:713; WO 90/03184, WO 96/11711, WO 00/48630, WO 98/36772, WO 00/41720, WO 2006/134423 and WO 2007/026190) or GLA-EM which is a combination of a TLR4 agonist and an oil-in-water emulsion.

For veterinary applications including but not limited to animal experimentation, one can use Complete Freund's Adjuvant (CFA), Freund's Incomplete Adjuvant (IFA), EMULSIGEN®, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI™, which contains three components extracted from bacteria, monophosphoryl lipid A, trehalose dimycolate and cell wall skeleton (MPL+TDM+CWS) in a 2% squalene/TWEEN® 80 emulsion.

Further exemplary adjuvants to enhance effectiveness of the pneumococcal vaccines as disclosed herein include, but are not limited to: (1) oil-in-water emulsion formulations (with or without other specific immunostimulating agents such as muramyl peptides (see below) or bacterial cell wall components), such as for example (a) SAF, containing 10% Squalane, 0.4% TWEEN® 80, 5% pluronic-blocked polymer L121, and thr-MDP either microfluidized into a submicron emulsion or vortexed to generate a larger particle size emulsion, and (b) RIBI™ adjuvant system (RAS), (Ribi Immunochem, Hamilton, MT) containing 2% Squalene, 0.2% TWEEN® 80, and one or more bacterial cell wall components such as monophosphorylipid A (MPL), trehalose dimycolate (TDM), and cell wall skeleton (CWS), preferably MPL+CWS (DETOX™); (2) saponin adjuvants, such as QS21, STIMULON™ (Cambridge Bioscience, Worcester, MA), ABISCO® (Isconova, Sweden), or ISCOMATRIX® (Commonwealth Serum Laboratories, Australia), may be used or particles generated therefrom such as ISCOMs (immunostimulating complexes), which ISCOMS may be devoid of additional detergent (e.g., WO 00/07621); (3) Complete Freund's Adjuvant (CFA) and Incomplete Freund's Adjuvant (IFA); (4) cytokines, such as interleukins (e.g., IL-1, IL-2, IL-4, IL-5, IL-6, IL-7, IL-12 (e.g., WO 99/44636)), interferons (e.g., gamma interferon), macrophage colony stimulating factor (M-CSF), tumor necrosis factor (TNF), etc.; (5) monophosphoryl lipid A (MPL) or 3-O-deacylated MPL (3dMPL) (see, e.g., GB-2220221, EP0689454), optionally in the substantial absence of alum when used with pneumococcal saccharides (see, e.g., WO 00/56358); (6) combinations of 3dMPL with, for example, QS21 and/or oil-in-water emulsions (see, e.g., EP0835318, EP0735898, EP0761231); (7) a polyoxyethylene ether or a polyoxyethylene ester (see, e.g., WO 99/52549); (8) a polyoxyethylene sorbitan ester surfactant in combination with an octoxynol (e.g., WO 01/21207) or a polyoxyethylene alkyl ether or ester surfactant in combination with at least one additional non-ionic surfactant such as an octoxynol (e.g., WO 01/21152); (9) a saponin and an immunostimulatory oligonucleotide (e.g., a CpG oligonucleotide) (e.g., WO 00/62800); (10) an immunostimulant and a particle of metal salt (see, e.g., WO 00/23105); (11) a saponin and an oil-in-water emulsion (e.g., WO 99/11241); (12) a saponin (e.g., QS21)+3dMPL+IM2 (optionally+a sterol) (e.g., WO 98/57659); (13) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc.

In an embodiment of the present invention, the immunogenic compositions as disclosed herein comprise a CpG Oligonucleotide as adjuvant. A CpG oligonucleotide as used herein refers to an immunostimulatory CpG oligodeoxynucleotide (CpG ODN), and accordingly these terms are used interchangeably unless otherwise indicated. Immunostimulatory CpG oligodeoxynucleotides contain one or more immunostimulatory CpG motifs that are unmethylated cytosine-guanine dinucleotides, optionally within certain base contexts. The methylation status of the CpG immunostimulatory motif generally refers to the cytosine residue in the dinucleotide. An immunostimulatory oligonucleotide containing at least one unmethylated CpG dinucleotide is an oligonucleotide which contains a 5′ unmethylated cytosine linked by a phosphate bond to a 3′ guanine, and which activates the immune system through binding to Toll-like receptor 9 (TLR-9). In another embodiment the immunostimulatory oligonucleotide may contain one or more methylated CpG dinucleotides, which will activate the immune system through TLR9 but not as strongly as if the CpG motif(s) was/were unmethylated. CpG immunostimulatory oligonucleotides may comprise one or more palindromes that in turn may encompass the CpG dinucleotide. CpG oligonucleotides have been described in a number of issued patents, published patent applications, and other publications, including U.S. Pat. Nos. 6,194,388; 6,207,646; 6,214,806; 6,218,371; 6,239,116; and 6,339,068.

In an embodiment of the present invention, the immunogenic compositions as disclosed herein comprise any of the CpG Oligonucleotide described at page 3, line 22, to page 12, line 36, of WO 2010/125480.

Different classes of CpG immunostimulatory oligonucleotides have been identified. These are referred to as A, B, C and P class, and are described in greater detail at page 3, line 22, to page 12, line 36, of WO 2010/125480. Methods of the invention embrace the use of these different classes of CpG immunostimulatory oligonucleotides.

In an embodiment of the present invention, the immunogenic compositions as disclosed herein comprise an A class CpG oligonucleotide. Preferably, the “A class” CpG oligonucleotide of the invention has the following nucleic acid sequence: 5′ GGGGACGACGTCGTGGGGGGG 3′ (SEQ ID NO: 1). Some non-limiting examples of A-Class oligonucleotides include: 5′ G*G*G_G_A_C_G_A_C_G_T_C_G_T_G_G*G*G*G*G*G 3′ (SEQ ID NO: 2); wherein “*” refers to a phosphorothioate bond and “_” refers to a phosphodiester bond.

In an embodiment of the present invention, the immunogenic compositions as disclosed herein comprise a B class CpG Oligonucleotide. In one embodiment, the CpG oligonucleotide for use in the present invention is a B class CpG oligonucleotide represented by at least the formula:

5′ X₁X₂CGX₃X₄ 3′, wherein X1, X2, X3, and X4 are nucleotides. In one embodiment, X2 is adenine, guanine, or thymine. In another embodiment, X3 is cytosine, adenine, or thymine.

The B class CpG oligonucleotide sequences of the invention are those broadly described above as well as disclosed in WO 96/02555, WO 98/18810 and U.S. Pat. Nos. 6,194,388; 6,207,646; 6,214,806; 6,218,371; 6,239,116 and 6,339,068. Exemplary sequences include but are not limited to those disclosed in these latter applications and patents.

In an embodiment, the “B class” CpG oligonucleotide of the invention has the following nucleic acid sequence:

(SEQ ID NO: 3)
5′ TCGTCGTTTTTCGGTGCTTTT 3′,
(SEQ ID NO: 4)
5′ TCGTCGTTTTTCGGTCGTTTT 3′,
(SEQ ID NO: 5)
5′ TCGTCGTTTTGTCGTTTTGTCGTT 3′,
(SEQ ID NO: 6)
5′ TCGTCGTTTCGTCGTTTTGTCGTT 3′,
or
(SEQ ID NO: 7)
5′ TCGTCGTTTTGTCGTTTTTTTCGA 3′.

In any of these sequences, all of the linkages may be all phosphorothioate bonds. In another embodiment, in any of these sequences, one or more of the linkages may be phosphodiester, preferably between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

Some non-limiting examples of B-Class oligonucleotides include:

(SEQ ID NO: 8)
5′ T*C*G*T*C*G*T*T*T*T*T*C*G*G*T*G*C*T*T*T*T 3′,
or
(SEQ ID NO: 9)
5′ T*C*G*T*C*G*T*T*T*T*T*C*G*G*T*C*G*T*T*T*T 3′,
or
(SEQ ID NO: 10)
5′ T*C*G*T*C*G*T*T*T*T*G*T*C*G*T*T*T*T*G*T*C*G*T*
T 3′,
or
(SEQ ID NO: 11)
5′ T*C*G*T*C*G*T*T*T*C*G*T*C*G*T*T*T*T*G*T*C*G*T*
T 3′,
or
(SEQ ID NO: 12)
5′ T*C*G*T*C*G*T*T*T*T*G*T*C*G*T*T*T*T*T*T*T*C*G*
A 3′.

wherein “*” refers to a phosphorothioate bond.

In an embodiment of the present invention, the immunogenic compositions as disclosed herein comprise a C class CpG Oligonucleotide. In an embodiment, the “C class” CpG oligonucleotides of the invention have the following nucleic acid sequence:

(SEQ ID NO: 13)
5′ TCGCGTCGTTCGGCGCGCGCCG 3′,
or
(SEQ ID NO: 14)
5′ TCGTCGACGTTCGGCGCGCGCCG 3′,
or
(SEQ ID NO: 15)
5′ TCGGACGTTCGGCGCGCGCCG 3′,
or
(SEQ ID NO: 16)
5′ TCGGACGTTCGGCGCGCCG 3′,
or
(SEQ ID NO: 17)
5′ TCGCGTCGTTCGGCGCGCCG 3′,
or
(SEQ ID NO: 18)
5′ TCGACGTTCGGCGCGCGCCG 3′,
or
(SEQ ID NO: 19)
5′ TCGACGTTCGGCGCGCCG 3′,
or
(SEQ ID NO: 20)
5′ TCGCGTCGTTCGGCGCCG 3′,
or
(SEQ ID NO: 21)
5′ TCGCGACGTTCGGCGCGCGCCG 3′,
or
(SEQ ID NO: 22)
5′ TCGTCGTTTTCGGCGCGCGCCG 3′,
or
(SEQ ID NO: 23)
5′ TCGTCGTTTTCGGCGGCCGCCG 3′,
or
(SEQ ID NO: 24)
5′ TCGTCGTTTTACGGCGCCGTGCCG 3′,
or
(SEQ ID NO: 25)
5′ TCGTCGTTTTCGGCGCGCGCCGT 3′.

In any of these sequences, all of the linkages may be all phosphorothioate bonds. In another embodiment, in any of these sequences, one or more of the linkages may be phosphodiester, preferably between the “C” and the “G” of the CpG motif making a semi-soft CpG oligonucleotide.

Some non-limiting examples of C-Class oligonucleotides include:

(SEQ ID NO: 26)
5′ T*C_G*C_G*T*C_G*T*T*C_G*G*C*G*C_G*C*G*C*C*G 3′,
or
(SEQ ID NO: 27)
5′ T*C_G*T*C_G*A*C_G*T*T*C_G*G*C*G*C_G*C*G*C*C*
G 3′,
or
(SEQ ID NO: 28)
5′ T*C_G*G*A*C_G*T*T*C_G*G*C*G*C_G*C*G*C*C*G 3′,
or
(SEQ ID NO: 29)
5′ T*C_G*G*A*C_G*T*T*C_G*G*C*G*C*G*C*C*G 3′,
or
(SEQ ID NO: 30)
5′ T*C_G*C_G*T*C_G*T*T*C_G*G*C*G*C*G*C*C*G 3′,
or
(SEQ ID NO: 31)
5′ T*C_G*A*C_G*T*T*C_G*G*C*G*C_G*C*G*C*C*G 3′,
or
(SEQ ID NO: 32)
5′ T*C_G*A*C_G*T*T*C_G*G*C*G*C*G*C*C*G 3′,
or
(SEQ ID NO: 33)
5′ T*C_G*C_G*T*C_G*T*T*C_G*G*C*G*C*C*G 3′,
or
(SEQ ID NO: 34)
5′ T*C_G*C_G*A*C_G*T*T*C_G*G*C*G*C_G*C*G*C*C*G 3′,
or
(SEQ ID NO: 35)
5′ T*C*G*T*C*G*T*T*T*T*C*G*G*C*G*C*G*C*G*C*C*G 3′,
or
(SEQ ID NO: 36)
5′ T*C*G*T*C*G*T*T*T*T*C*G*G*C*G*G*C*C*G*C*C*G 3′,
or
(SEQ ID NO: 37)
5′ T*C*G*T*C_G*T*T*T*T*A*C_G*G*C*G*C*C_G*T*G*C*C*
G 3′,
or
(SEQ ID NO: 38)
5′ T*C_G*T*C*G*T*T*T*T*C*G*G*C*G*C*G*C*G*C*C*G*T 3′

wherein “*” refers to a phosphorothioate bond and “_” refers to a phosphodiester bond.

In any of these sequences, an ethyl-uridine or a halogen may substitute for the 5′ T; examples of halogen substitutions include but are not limited to bromo-uridine or iodo-uridine substitutions.

In an embodiment of the present invention, the immunogenic compositions as disclosed herein comprise a P class CpG Oligonucleotide. In an embodiment, the CpG oligonucleotide for use in the present invention is a P class CpG oligonucleotide containing a 5′ TLR activation domain and at least two palindromic regions, one palindromic region being a 5′ palindromic region of at least 6 nucleotides in length and connected to a 3′ palindromic region of at least 8 nucleotides in length either directly or through a spacer, wherein the oligonucleotide includes at least one YpR dinucleotide. In an embodiment, said oligonucleotide is not T*C_G*T*C_G*A*C_G*T*T*C_G*G*C*G*C_G*C*G*C*C*G (SEQ ID NO: 27). In one embodiment the P class CpG oligonucleotide includes at least one unmethylated CpG dinucleotide. In another embodiment the TLR activation domain is TCG, TTCG, TTTCG, TYpR, TTYpR, TTTYpR, UCG, UUCG, UUUCG, TTT, or TTTT. In yet another embodiment the TLR activation domain is within the 5′ palindromic region. In another embodiment the TLR activation domain is immediately 5′ to the 5′ palindromic region.

In an embodiment, the “P class” CpG oligonucleotides of the invention have the following nucleic acid sequence: 5′ TCGTCGACGATCGGCGCGCGCCG 3′ (SEQ ID NO: 39).

In said sequences, all of the linkages may be all phosphorothioate bonds. In another embodiment, one or more of the linkages may be phosphodiester, preferably between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

A non-limiting example of P-Class oligonucleotides include:

(SEQ ID NO: 40)
5′ T*C_G*T*C_G*A*C_G*A*T*C_G*G*C*G*C_G*C*G*C*C*
G 3′

wherein “*” refers to a phosphorothioate bond and “_” refers to a phosphodiester bond.

In one embodiment the oligonucleotide includes at least one phosphorothioate linkage. In another embodiment all internucleotide linkages of the oligonucleotide are phosphorothioate linkages. In another embodiment the oligonucleotide includes at least one phosphodiester-like linkage. In another embodiment the phosphodiester-like linkage is a phosphodiester linkage. In another embodiment a lipophilic group is conjugated to the oligonucleotide. In one embodiment the lipophilic group is cholesterol.

In an embodiment, all the internucleotide linkages of the CpG oligonucleotides disclosed herein are phosphodiester bonds (“soft” oligonucleotides, as described in WO 2007/026190). In another embodiment, CpG oligonucleotides of the invention are rendered resistant to degradation (e.g., are stabilized). A “stabilized oligonucleotide” refers to an oligonucleotide that is relatively resistant to in vivo degradation (e.g., via an exo- or endo-nuclease). Nucleic acid stabilization can be accomplished via backbone modifications. Oligonucleotides having phosphorothioate linkages provide maximal activity and protect the oligonucleotide from degradation by intracellular exo- and endo-nucleases.

The immunostimulatory oligonucleotides may have a chimeric backbone, which have combinations of phosphodiester and phosphorothioate linkages. For purposes of the instant invention, a chimeric backbone refers to a partially stabilized backbone, wherein at least one internucleotide linkage is phosphodiester or phosphodiester-like, and wherein at least one other internucleotide linkage is a stabilized internucleotide linkage, wherein the at least one phosphodiester or phosphodiester-like linkage and the at least one stabilized linkage are different. When the phosphodiester linkage is preferentially located within the CpG motif such molecules are called “semi-soft” as described in WO 2007/026190.

Other modified oligonucleotides include combinations of phosphodiester, phosphorothioate, methylphosphonate, methylphosphorothioate, phosphorodithioate, and/or p-ethoxy linkages.

Mixed backbone modified ODN may be synthesized as described in WO 2007/026190.

The size of the CpG oligonucleotide (i.e., the number of nucleotide residues along the length of the oligonucleotide) also may contribute to the stimulatory activity of the oligonucleotide. For facilitating uptake into cells, CpG oligonucleotide of the invention preferably have a minimum length of 6 nucleotide residues. Oligonucleotides of any size greater than 6 nucleotides (even many kb long) are capable of inducing an immune response if sufficient immunostimulatory motifs are present, because larger oligonucleotides are degraded inside cells. In certain embodiments, the CpG oligonucleotides are 6 to 100 nucleotides long, preferentially 8 to 30 nucleotides long. In important embodiments, nucleic acids and oligonucleotides of the invention are not plasmids or expression vectors.

In an embodiment, the CpG oligonucleotide disclosed herein comprise substitutions or modifications, such as in the bases and/or sugars as described at paragraphs 134 to 147 of WO 2007/026190.

In an embodiment, the CpG oligonucleotide of the present invention is chemically modified. Examples of chemical modifications are known to the skilled person and are described, for example in Uhlmann et al. (1990) Chem. Rev. 90:543; S. Agrawal, Ed., Humana Press, Totowa, USA 1993; Crooke et al. (1996) Annu. Rev. Pharmacol. Toxicol. 36:107-129; and Hunziker et al. (1995) Mod. Synth. Methods 7:331-417. An oligonucleotide according to the invention may have one or more modifications, wherein each modification is located at a particular phosphodiester internucleoside bridge and/or at a particular β-D-ribose unit and/or at a particular natural nucleoside base position in comparison to an oligonucleotide of the same sequence which is composed of natural DNA or RNA.

In some embodiments of the invention, CpG-containing nucleic acids might be simply mixed with immunogenic carriers according to methods known to those skilled in the art (see, e.g., WO 03/024480).

In a particular embodiment of the present invention, any of the immunogenic compositions disclosed herein comprise from 2 μg to 100 mg of CpG oligonucleotide, preferably from 0.1 mg to 50 mg CpG oligonucleotide, preferably from 0.2 mg to 10 mg CpG oligonucleotide, preferably from 0.3 mg to 5 mg CpG oligonucleotide, preferably from 0.3 mg to 5 mg CpG oligonucleotide, even more preferably from 0.5 to 2 mg CpG oligonucleotide, even more preferably from 0.75 to 1.5 mg CpG oligonucleotide. In an embodiment, any of the immunogenic composition disclosed herein comprises about 1 mg CpG oligonucleotide.

In an embodiment, the immunogenic composition of the invention (such as defined at section 2 above), comprises an adjuvant as defined above, preferably an aluminum salt (alum) (e.g., aluminum phosphate, aluminum sulfate or aluminum hydroxide). In an embodiment, the immunogenic composition of the invention comprise aluminum phosphate or aluminum hydroxide as adjuvant.

In an embodiment, the immunogenic composition which may be used in combination with the immunogenic composition of the invention (such as defined at section 3 above), comprises an adjuvant as defined above, preferably an aluminum salt (alum) (e.g., aluminum phosphate, aluminum sulfate or aluminum hydroxide). In an embodiment, said immunogenic compostions comprise aluminum phosphate or aluminum hydroxide as adjuvant.

An aspect of the invention provides a kit as defined at section 4 above wherein only the first immunogenic composition (part (a) of the kit) comprises an adjuvant as defined above.

An aspect of the invention provides a kit as defined at section 4 above wherein only the second immunogenic composition (part (b) of the kit) comprises an adjuvant as defined above.

An aspect of the invention provides a kit as defined at section 4 above wherein both immunogenic compositions (part (a) and (b) of the kit) comprise an adjuvant as defined above.

An aspect of the invention provides a kit as defined at section 4 above wherein both immunogenic compositions (part (a) and (b) of the kit) comprise an adjuvant selected from the group consisting of aluminum phosphate, aluminum sulfate and aluminum hydroxide.

An aspect of the invention provides a kit as defined at section 4 above wherein both immunogenic compositions (part (a) and (b) of the kit) comprise aluminum phosphate as adjuvant.

An aspect of the invention provides a kit as defined at section 4 above wherein both immunogenic compositions (part (a) and (b) of the kit) comprise aluminium hydroxide as adjuvant.

An aspect of the invention provides a kit as defined at section 4 above wherein both immunogenic compositions (part (a) and (b) of the kit) comprise aluminium sulfate as adjuvant.

### 8. Formulation

The immunogenic compositions disclosed herein may be formulated in liquid form (i.e., solutions or suspensions) or in a lyophilized form. Liquid formulations may advantageously be administered directly from their packaged form and are thus ideal for injection without the need for reconstitution in aqueous medium as otherwise required for lyophilized compositions.

Formulation of the immunogenic composition disclosed herein can be accomplished using art-recognized methods. For instance, the individual pneumococcal conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

The present disclosure provides an immunogenic composition comprising any combination of glycoconjugates disclosed herein and a pharmaceutically acceptable excipient, carrier, or diluent.

In an embodiment, the immunogenic composition disclosed herein is in liquid form, preferably in aqueous liquid form.

Immunogenic compositions of the disclosure may comprise one or more of a buffer, a salt, a divalent cation, a non-ionic detergent, a cryoprotectant such as a sugar, and an anti-oxidant such as a free radical scavenger or chelating agent, or any combinations thereof.

In an embodiment, the immunogenic compositions disclosed herein comprise a buffer. In an embodiment, said buffer has a pKa of about 3.5 to about 7.5. In some embodiments, the buffer is phosphate, succinate, histidine or citrate. In certain embodiments, the buffer is succinate at a final concentration of 1 mM to 10 mM. In one particular embodiment, the final concentration of the succinate buffer is about 5 mM.

In an embodiment, the immunogenic compositions disclosed herein comprise a salt. In some embodiments, the salt is selected from the groups consisting of magnesium chloride, potassium chloride, sodium chloride and a combination thereof. In one particular embodiment, the salt is sodium chloride. In one particular embodiment, the immunogenic compositions disclosed herein comprise sodium chloride at 150 mM.

In an embodiment, the immunogenic compositions disclosed herein comprise a surfactant. In an embodiment, the surfactant is selected from the group consisting of polysorbate 20 (TWEEN™20), polysorbate 40 (TWEEN™40), polysorbate 60 (TWEEN™60), polysorbate 65 (TWEEN™65), polysorbate 80 (TWEEN™80), polysorbate 85 (TWEEN™85), TRITON™ N-101, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR® EL), soy lecithin and a poloxamer. In one particular embodiment, the surfactant is polysorbate 80. In some said embodiment, the final concentration of polysorbate 80 in the formulation is at least 0.0001% to 10% polysorbate 80 weight to weight (w/w). In some said embodiments, the final concentration of polysorbate 80 in the formulation is at least 0.001% to 1% polysorbate 80 weight to weight (w/w). In some said embodiments, the final concentration of polysorbate 80 in the formulation is at least 0.01% to 1% polysorbate 80 weight to weight (w/w). In other embodiments, the final concentration of polysorbate 80 in the formulation is 0.01%, 0.02%, 0.03%, 0.04%, 0.05%, 0.06%, 0.07%, 0.08%, 0.09% or 0.1% polysorbate 80 (w/w). In another embodiment, the final concentration of the polysorbate 80 in the formulation is 1% polysorbate 80 (w/w).

In certain embodiments, the immunogenic composition disclosed herein has a pH of 5.5 to 7.5, more preferably a pH of 5.6 to 7.0, even more preferably a pH of 5.8 to 6.0.

In one embodiment, the present invention provides a container filled with any of the immunogenic compositions disclosed herein. In one embodiment, the container is selected from the group consisting of a vial, a syringe, a flask, a fermentor, a bioreactor, a bag, a jar, an ampoule, a cartridge and a disposable pen. In certain embodiments, the container is siliconized.

In an embodiment, the container of the present invention is made of glass, metals (e.g., steel, stainless steel, aluminum, etc.) and/or polymers (e.g., thermoplastics, elastomers, thermoplastic-elastomers). In an embodiment, the container of the present invention is made of glass.

In one embodiment, the present invention provides a syringe filled with any of the immunogenic compositions disclosed herein. In certain embodiments, the syringe is siliconized and/or is made of glass.

A typical dose of the immunogenic composition disclosed herein for injection has a volume of 0.1 mL to 2 mL, more preferably 0.2 mL to 1 mL, even more preferably a volume of about 0.5 mL.

Therefore the container or syringe as defined above is filed with a volume of 0.1 mL to 2 mL, more preferably 0.2 mL to 1 mL, even more preferably a volume of about 0.5 mL of any of the immunogenic compositions defined herein.

In an embodiment, the immunogenic composition of the invention (such as defined at section 2 above) is formulated as disclosed above.

In an embodiment, the immunogenic composition which may be used in combination with the immunogenic composition of the invention (such as defined at section 3 above) is formulated as disclosed above.

An aspect of the invention provides a kit as defined at section 4 above wherein both immunogenic compositions (part (a) and (b) of the kit) are formulated as described above.

An aspect of the invention provides a kit as defined at section 4 above wherein both immunogenic compositions (part (a) and (b) of the kit) are formulated in liquid form.

An aspect of the invention provides a kit as defined at section 4 above wherein both immunogenic compositions (part (a) and (b) of the kit) are formulated in lyophilized form.

An aspect of the invention provides a kit as defined at section 4 above wherein the first immunogenic composition (part (a) of the kit) is in liquid form and the second immunogenic composition (part (b) of the kit) is in lyophilized form.

An aspect of the invention provides a kit as defined at section 4 above wherein the first immunogenic composition (part (a) of the kit) is in lyophilized form and the second immunogenic composition (part (b) of the kit) is in liquid form.

### 9. Uses of the Immunogenic Compositions and Kits of the Invention

In an embodiment, the immunogenic compositions and kits disclosed herein are for use as a medicament.

The immunogenic compositions and kits described herein may be used in various therapeutic or prophylactic methods for preventing, treating or ameliorating a bacterial infection, disease or condition in a subject. In particular, immunogenic compositions and kits described herein may be used to prevent, treat or ameliorate a S. pneumoniae infection, disease or condition in a subject.

In one or more embodiments, the invention provides a method of preventing, treating or ameliorating an infection, disease or condition associated with S. pneumoniae in a subject, comprising administering to the subject an immunologically effective amount of an immunogenic composition of described herein.

In some such embodiments, the infection, disease or condition is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess.

In an embodiment, the invention provides a method of inducing an immune response to S. pneumoniae in a subject comprising administering to the subject an immunologically effective amount of an immunogenic composition of the invention.

In an embodiment, the immunogenic compositions and kits disclosed herein are for use as a vaccine. In such embodiments the immunogenic compositions and kits described herein may be used to prevent a S. pneumoniae infection in a subject. Thus in one aspect, the invention provides a method of preventing an infection by S. pneumoniae in a subject comprising administering to the subject an immunologically effective amount of an immunogenic composition of the invention. In some such embodiments, the infection is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess. In one aspect, the subject to be vaccinated is a mammal, such as a human, cat, sheep, pig, horse, bovine or dog.

In one aspect, the immunogenic compositions and kits disclosed herein are for use in a method of preventing, treating or ameliorating an infection, disease or condition associated with S. pneumoniae in a subject. In some such embodiments, the infection, disease or condition is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess.

In an embodiment, the immunogenic compositions and kits disclosed herein are for use as a vaccine. In such embodiments the immunogenic compositions and kits described herein may be used to prevent a S. pneumoniae infection in a subject. Thus in one aspect, the immunogenic compositions and kits disclosed herein are for use in a method of preventing, an infection by S. pneumoniae in a subject. In some such embodiments, the infection is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess. In one aspect, the subject to be vaccinated is a mammal, such as a human, cat, sheep, pig, horse, bovine or dog.

The immunogenic compositions and kits of the present invention can be used to protect or treat a human susceptible to pneumococcal infection, by means of administering the immunogenic compositions via a systemic or mucosal route. In an embodiment, the immunogenic compositions disclosed herein are administered by intramuscular, intraperitoneal, intradermal or subcutaneous routes. In an embodiment, the immunogenic compositions disclosed herein are administered by intramuscular, intraperitoneal, intradermal or subcutaneous injection. In an embodiment, the immunogenic compositions disclosed herein are administered by intramuscular or subcutaneous injection.

In an embodiment, the immunogenic composition of the present disclosure comprising at least one glycoconjugate from S. pneumoniae serotype 15B (such as the glycoconjugates of section 1.3.4 above), when administered to a subject, are able to induce the formation of antibodies capable of binding to S. pneumonia serotype 15B, 15A and/or 15C as measured by a standard ELISA assay. In an embodiment, the immunogenic composition of the present disclosure comprising at least one glycoconjugate from S. pneumoniae serotype 15B (such as the glycoconjugates of section 1.3.4 above), when administered to a subject, are able to induce the formation of antibodies capable of binding to S. pneumonia serotype 15B and 15C as measured by a standard ELISA assay.

In the ELISA (Enzyme-linked Immunosorbent Assay) method, antibodies from the sera of vaccinated subjects are incubated with polysaccharides which have been adsorbed to a solid support. The bound antibodies are detected using enzyme-conjugated secondary detection antibodies.

In an embodiment said standard ELISA assay is the standardized (WHO) ELISA assay as defined by the WHO in the ‘Training manual for Enzyme linked immunosorbent assay for the quantitation of Streptococcus pneumoniae serotype specific IgG (Pn PS ELISA).’ (accessible at http://www.vaccine.uab.edu/ELISA%20protocol.pdf; last accessed on Mar. 31, 2014).

The ELISA measures type specific IgG anti-S. pneumoniae capsular polysaccharide (PS) antibodies present in human serum. When dilutions of human sera are added to type-specific capsular PS-coated microtiter plates, antibodies specific for that capsular PS bind to the microtiter plates. The antibodies bound to the plates are detected using a goat anti-human IgG alkaline phosphatase-labeled antibody followed by a p-nitrophenyl phosphate substrate. The optical density of the colored end product is proportional to the amount of anticapsular PS antibody present in the serum.

In an embodiment, the immunogenic composition of the present disclosure comprising at least one glycoconjugate from S. pneumoniae serotype 15B (such as the glycoconjugates of section 1.3.4 above) is able to elicit IgG antibodies in human which are capable of binding S. pneumoniae serotype 15B polysaccharide at a concentration of at least 0.05, 0.1, 0.2, 0.3, 0.35, 0.4 or 0.5 μg/ml as determined by ELISA assay.

In an embodiment, the immunogenic composition of the present disclosure comprising at least one glycoconjugate from S. pneumoniae serotype 15B (such as the glycoconjugates of section 1.3.4 above) is able to elicit IgG antibodies in human which are capable of binding S. pneumoniae serotype 15C polysaccharide at a concentration of at least 0.05, 0.1, 0.2, 0.3, 0.35, 0.4 or 0.5 μg/ml as determined by ELISA assay.

In an embodiment, the immunogenic composition of the present disclosure comprising at least one glycoconjugate from S. pneumoniae serotype 15B (such as the glycoconjugates of section 1.3.4 above) is able to elicit IgG antibodies in human which are capable of binding S. pneumoniae serotypes 15B and 15C polysaccharide at a concentration of at least 0.05, 0.1, 0.2, 0.3, 0.35, 0.4 or 0.5 μg/ml as determined by ELISA assay.

In an embodiment, the immunogenic composition of the present disclosure comprising at least one glycoconjugate from S. pneumoniae serotype 15B (such as the glycoconjugates of section 1.3.4 above), when administered to a subject, are able to induce the formation of antibodies capable of killing S. pneumonia serotype 15B in an opsonophagocytosis assay as disclosed herein (such as the OPA assay of Example 12).

In an embodiment, the immunogenic composition of the present disclosure comprising at least one glycoconjugate from S. pneumoniae serotype 15B (such as the glycoconjugates of section 1.3.4 above), when tested in an OPA assay as disclosed herein (such as the OPA assay of Example 12), has an OPA titer greater than the OPA titer obtained with an unconjugated native S. pneumonia serotype 15B capsular polysaccharide.

In an embodiment, the immunogenic composition of the present disclosure comprising at least one glycoconjugate from S. pneumoniae serotype 15B (such as the glycoconjugates of section 1.3.4 above), when administered to a subject, are able to induce the formation of antibodies capable of killing S. pneumonia serotype 15C in an opsonophagocytosis assay as disclosed herein (such as the OPA assay of Example 12). In an embodiment, the immunogenic composition of the present disclosure comprising at least one glycoconjugate from S. pneumoniae serotype 15B (such as the glycoconjugates of section 1.3.4 above), when tested in an OPA assay as disclosed herein (such as the OPA assay of Example 12), has an OPA titer greater than the OPA titer obtained with an unconjugated native S. pneumonia serotype 15B capsular polysaccharide.

The pneumococcal opsonophagocytic assay (OPA), which measures killing of S. pneumoniae cells by phagocytic effector cells in the presence of functional antibody and complement, is considered to be an important surrogate for evaluating the effectiveness of pneumococcal vaccines.

Opsonophagocytic assay (OPA) can be conducted by incubating together a mixture of Streptococcus pneumoniae cells, a heat inactivated human serum to be tested, differentiated HL-60 cells (phagocytes) and an exogenous complement source (e.g. baby rabbit complement). Opsonophagocytosis proceeds during incubation and bacterial cells that are coated with antibody and complement are killed upon opsonophagocytosis. Colony forming units (cfu) of surviving bacteria that escape from opsonophagocytosis are determined by plating the assay mixture. The OPA titer is defined as the reciprocal dilution that results in a 50% reduction in bacterial count over control wells without test serum. The OPA titer is interpolated from the two dilutions that encompass this 50% killing cut-off.

An endpoint titer of 1:8 or greater is considered a positive result in these killing type OPA.

In a further aspect, the present disclosure provides a method of treating or preventing a S. pneumoniae infection, disease or condition associated with S. pneumoniae serotype 15A, 15B and/or 15C in a subject, the method comprising the step of administering a therapeutically or prophylactically effective amount of any of the immunogenic compositions of the present disclosure comprising at least one glycoconjugate from S. pneumoniae serotype 15B. In an embodiment, the immunogenic composition of the present disclosure comprising at least one glycoconjugate from S. pneumoniae serotype 15B, when administered to a subject, induces the formation of antibodies capable of binding to S. pneumoniae serotype 15B, 15A and/or 15C. In an embodiment, the immunogenic composition of the present disclosure comprising at least one glycoconjugate from S. pneumoniae serotype 15B, when administered to a subject, induces the formation of antibodies capable of killing S. pneumoniae serotype 15B, 15C and/or 15A in an opsonophagocytosis assay as disclosed herein.

One embodiment of the disclosure provides a method of protecting a subject against an infection with S. pneumoniae serotype 15C, or a method of preventing infection with S. pneumoniae serotype 15C, or a method of reducing the severity of or delaying the onset of at least one symptom associated with an infection caused by S. pneumoniae serotype 15C, the methods comprising administering to a subject an immunogenic amount of any of the immunogenic composition of the present disclosure comprising at least one glycoconjugate from S. pneumoniae serotype 15B. One embodiment of the disclosure provides a method of treating or preventing a S. pneumoniae infection, disease or condition associated with S. pneumoniae serotype 15A, 15B and/or 15C in a subject, the method comprising the step of administering a therapeutically or prophylactically effective amount of any of the immunogenic composition of the present disclosure comprising at least one glycoconjugate from S. pneumoniae serotype 15B to the subject. Another embodiment provides a method of treating or preventing a S. pneumoniae infection, disease or condition associated with a S. pneumoniae serotype 15A, 15B and/or 15C in a subject, the method comprising generating a polyclonal or monoclonal antibody preparation from any of the immunogenic composition of the present disclosure comprising at least one glycoconjugate from S. pneumoniae serotype 15B, and using said antibody preparation to confer passive immunity to the subject.

In one embodiment, the disclosure relates to the use of any of the immunogenic composition of the present disclosure comprising at least one glycoconjugate from S. pneumoniae serotype 15B for the manufacture of a medicament for protecting a subject against an infection with S. pneumoniae, and/or preventing infection with S. pneumoniae, and/or reducing the severity of or delaying the onset of at least one symptom associated with an infection caused by S. pneumoniae, and/or protecting a subject against an infection with S. pneumoniae serotype 15A, 15B and/or 15C and/or preventing infection with S. pneumoniae serotype 15A, 15B and/or 15C, and/or reducing the severity of or delaying the onset of at least one symptom associated with an infection caused by S. pneumoniae serotype 15A, 15B and/or 15C.

In one embodiment, the disclosure relates to the use of any of the immunogenic compositions of the present disclosure comprising at least one glycoconjugate from S. pneumoniae serotype 15B for protecting a subject against an infection with S. pneumoniae, and/or preventing infection with S. pneumoniae, and/or reducing the severity of or delaying the onset of at least one symptom associated with an infection caused by S. pneumoniae, and/or protecting a subject against an infection with S. pneumoniae serotype 15A, 15B and/or 15C and/or preventing infection with S. pneumoniae serotype 15A, 15B and/or 15C, and/or reducing the severity of or delaying the onset of at least one symptom associated with an infection caused by S. pneumoniae serotype 15A, 15B and/or 15C.

10. Subject to be Treated with the Immunogenic Compositions and Kits of the Invention

As disclosed herein, the immunogenic compositions and kits described herein may be used in various therapeutic or prophylactic methods for preventing, treating or ameliorating a bacterial infection, disease or condition in a subject.

In an embodiment, said subject is a human. In a most embodiment, said subject is a newborn (i.e., under three months of age), an infant (i.e., from 3 months to one year of age) or a toddler (i.e., from one year to four years of age).

In an embodiment, the immunogenic compositions and kits disclosed herein are for use as a vaccine.

In such embodiment, the subject to be vaccinated may be less than 1 year of age. For example, the subject to be vaccinated can be about 1, about 2, about 3, about 4, about 5, about 6, about 7, about 8, about 9, about 10, about 11 or about 12 months of age. In an embodiment, the subject to be vaccinated is about 2, about 4 or about 6 months of age. In another embodiment, the subject to be vaccinated is less than 2 years of age. For example, the subject to be vaccinated can be about 12 to about 15 months of age. In some cases, as little as one dose of the immunogenic composition according to the invention is needed, but under some circumstances, a second, third or fourth dose may be given (see section 11 below).

In an embodiment of the present invention, the subject to be vaccinated is a human adult 50 years of age or older, more preferably a human adult 55 years of age or older. In an embodiment, the subject to be vaccinated is a human adult 65 years of age or older, 70 years of age or older, 75 years of age or older or 80 years of age or older.

In an embodiment the subject to be vaccinated is an immunocompromised individual, in particular a human. An immunocompromised individual is generally defined as a person who exhibits an attenuated or reduced ability to mount a normal humoral or cellular defense to challenge by infectious agents.

In an embodiment of the present invention, the immunocompromised subject to be vaccinated suffers from a disease or condition that impairs the immune system and results in an antibody response that is insufficient to protect against or treat pneumococcal disease.

In an embodiment, said disease is a primary immunodeficiency disorder. Preferably, said primary immunodeficiency disorder is selected from the group consisting of: combined T- and B-cell immunodeficiencies, antibody deficiencies, well-defined syndromes, immune dysregulation diseases, phagocyte disorders, innate immunity deficiencies, autoinflammatory disorders, and complement deficiencies. In an embodiment, said primary immunodeficiency disorder is selected from the one disclosed on page 24, line 11, to page 25, line 19, of WO 2010/125480.

In a particular embodiment of the present invention, the immunocompromised subject to be vaccinated suffers from a disease selected from the group consisting of: HIV-infection, acquired immunodeficiency syndrome (AIDS), cancer, chronic heart or lung disorders, congestive heart failure, diabetes mellitus, chronic liver disease, alcoholism, cirrhosis, spinal fluid leaks, cardiomyopathy, chronic bronchitis, emphysema, chronic obstructive pulmonary disease (COPD), spleen dysfunction (such as sickle cell disease), lack of spleen function (asplenia), blood malignancy, leukemia, multiple myeloma, Hodgkin's disease, lymphoma, kidney failure, nephrotic syndrome and asthma.

In an embodiment of the present invention, the immunocompromised subject to be vaccinated suffers from malnutrition.

In a particular embodiment of the present invention, the immunocompromised subject to be vaccinated is taking a drug or treatment that lowers the body's resistance to infection. In an embodiment, said drug is selected from the one disclosed on page 26, line 33, to page 26, line 4, of WO 2010/125480.

In a particular embodiment of the present invention, the immunocompromised subject to be vaccinated is a smoker.

In a particular embodiment of the present invention, the immunocompromised subject to be vaccinated has a white blood cell count (leukocyte count) below 5×10⁹ cells per liter, or below 4×10⁹ cells per liter, or below 3×10⁹ cells per liter, or below 2×10⁹ cells per liter, or below 1×10⁹ cells per liter, or below 0.5×10⁹ cells per liter, or below 0.3×10⁹ cells per liter, or below 0.1×10⁹ cells per liter.

White blood cell count (leukocyte count): The number of white blood cells (WBC) in the blood. The WBC is usually measured as part of the CBC (complete blood count). White blood cells are the infection-fighting cells in the blood and are distinct from the red (oxygen-carrying) blood cells known as erythrocytes. There are different types of white blood cells, including neutrophils (polymorphonuclear leukocytes; PMN), band cells (slightly immature neutrophils), T-type lymphocytes (T-cells), B-type lymphocytes (B-cells), monocytes, eosinophils, and basophils. All the types of white blood cells are reflected in the white blood cell count. The normal range for the white blood cell count is usually between 4,300 and 10,800 cells per cubic millimeter of blood. This can also be referred to as the leukocyte count and can be expressed in international units as 4.3-10.8×10⁹ cells per liter.

In a particular embodiment of the present invention, the immunocompromised subject to be vaccinated suffers from neutropenia. In a particular embodiment of the present invention, the immunocompromised subject to be vaccinated has a neutrophil count below 2×10⁹ cells per liter, or below 1×10⁹ cells per liter, or below 0.5×10⁹ cells per liter, or below 0.1×10⁹ cells per liter, or below 0.05×10⁹ cells per liter.

A low white blood cell count or “neutropenia” is a condition characterized by abnormally low levels of neutrophils in the circulating blood. Neutrophils are a specific kind of white blood cell that help to prevent and fight infections. The most common reason that cancer patients experience neutropenia is as a side effect of chemotherapy.

Chemotherapy-induced neutropenia increases a patient's risk of infection and disrupts cancer treatment.

In a particular embodiment of the present invention, the immunocompromised subject to be vaccinated has a CD4+ cell count below 500/mm³, or CD4+ cell count below 300/mm³, or CD4+ cell count below 200/mm³, CD4+ cell count below 100/mm³.

CD4+ cell count below 75/mm³, or CD4+ cell count below 50/mm³.

CD4 cell tests are normally reported as the number of cells in mm³. Normal CD4 counts are between 500 and 1,600, and CD8 counts are between 375 and 1,100. CD4 counts drop dramatically in people with HIV.

In an embodiment of the invention, any of the immunocompromised subjects disclosed herein is a human male or a human female.

### 11. Immunization Schedule

In some cases, as little as one dose of the immunogenic composition according to the invention is needed, but under some circumstances, such as conditions of greater immune deficiency or immune immaturity, a second, third or fourth dose may be given. Following an initial vaccination, subjects can receive one or several booster immunizations adequately spaced.

In an embodiment, the schedule of vaccination of the immunogenic composition according to the invention is a single dose. In a particular embodiment, said single dose schedule is for healthy persons being at least 2 years of age.

In an embodiment, the schedule of vaccination of the immunogenic composition according to the invention is a multiple dose schedule. A multiple dose schedule is frequently used in conditions such as immune deficiency (such as human elderly or human immunocompromised individuals) or immune immaturity (such as human newborns (i.e., under three months of age), infants (i.e., from 3 months to one year of age) or toddlers (i.e., from one year to four years of age)). In a particular embodiment, said multiple dose schedule consists of a series of 2 doses separated by an interval of about 1 month to about 12 months. In a particular embodiment, said multiple dose schedule consists of a series of 2 doses separated by an interval of about 1, 2, 3, 4, 5, 6, 7, 8, 9, 10, 11 or 12 months. In a particular embodiment, said multiple dose schedule consists of a series of 2 doses separated by an interval of about 1 month to about 6 months. In a particular embodiment, said multiple dose schedule consists of a series of 2 doses separated by an interval of about 1, 2, 3, 4, 5 or 6 months. In a particular embodiment, said multiple dose schedule consists of a series of 2 doses separated by an interval of about 1 month, or a series of 2 doses separated by an interval of about 2 months.

In another embodiment, said multiple dose schedule consists of a series of 3 doses wherein each dose is separated by an interval of about 1 month to about 12 months.

In a particular embodiment, said multiple dose schedule consists of a series of 3 doses wherein each dose is separated by an interval of about 1 month to about 6 months. In a particular embodiment, said multiple dose schedule consists of a series of 3 doses wherein each dose is separated by an interval of about 1, 2, 3, 4, 5 or 6 months. In a particular embodiment, said multiple dose schedule consists of a series of 3 doses wherein each dose is separated by an interval of about 1 month, or a series of 3 doses wherein each dose is separated by an interval of about 2 months.

In another embodiment, said multiple dose schedule consists of a series of 4 doses wherein each dose is separated by an interval of about 1 month to about 12 months.

In a particular embodiment, said multiple dose schedule consists of a series of 4 doses wherein each dose is separated by an interval of about 1 month to about 6 months. In a particular embodiment, said multiple dose schedule consists of a series of 4 doses wherein each dose is separated by an interval of about 1, 2, 3, 4, 5 or 6 months. In a particular embodiment, said multiple dose schedule consists of a series of 4 doses wherein each dose is separated by an interval of about 1 month, or a series of 4 doses wherein each dose is separated by an interval of about 2 months.

In another embodiment, said multiple dose schedule consists of a series of 3 doses wherein each dose is separated by an interval of about 1 month to about 4 months followed by a fourth dose about 10 months to about 13 months after the first dose. In another embodiment, said multiple dose schedule consists of a series of 3 doses wherein each dose is separated by an interval of about 1, 2, 3 or 4 months followed by a fourth dose about 10 months to about 13 months after the first dose. In another embodiment, said multiple dose schedule consists of a series of 3 doses wherein each dose is separated by an interval of about 1 month to about 2 months followed by a fourth dose about 10 months to about 13 months after the first dose. In another embodiment, said multiple dose schedule consists of a series of 3 doses wherein each dose is separated by an interval of about 1 month followed by a fourth dose about 10 months to about 13 months after the first dose, or a series of 3 doses wherein each dose is separated by an interval of about 2 months followed by a fourth dose about 10 months to about 13 months after the first dose.

In an embodiment, the multiple dose schedule consists of at least one dose (e.g., 1, 2 or 3 doses) in the first year of age followed by at least one toddler dose.

In an embodiment, the multiple dose schedule consists of a series of 2 or 3 doses wherein each dose is separated by an interval of about 1 month to about 2 months (for example 28-56 days between doses), starting at 2 months of age, and followed by a toddler dose at 12-18 months of age. In an embodiment, said multiple dose schedule consists of a series of 3 doses wherein each dose is separated by an interval of about 1 month to about 2 months (for example 28-56 days between doses), starting at 2 months of age, and followed by a toddler dose at 12-15 months of age. In another embodiment, said multiple dose schedule consists of a series of 2 doses separated by an interval of about 2 months, starting at 2 months of age, and followed by a toddler dose at 12-18 months of age.

In an embodiment, the multiple dose schedule consists of a 4-dose series of vaccine at 2, 4, 6, and 12-15 months of age.

In an embodiment, a prime dose is given at day 0 and one or more booster doses are given at intervals that range from about 2 to about 24 weeks between doses, preferably with a dosing interval of 4-8 weeks.

In an embodiment, a prime dose is given at day 0 and a boost is given about 3 months later.

In another embodiment, said multiple dose schedule consists of a series of 5 doses wherein each dose is separated by an interval of about 1 month to about 12 months.

In a particular embodiment, said multiple dose schedule consists of a series of 5 doses wherein each dose is separated by an interval of about 1 month to about 6 months. In a particular embodiment, said multiple dose schedule consists of a series of 5 doses wherein each dose is separated by an interval of about 1, 2, 3, 4, 5 or 6 months. In a particular embodiment, said multiple dose schedule consists of a series of 5 doses wherein each dose is separated by an interval of about 1 month, or a series of 5 doses wherein each dose is separated by an interval of about 2 months.

In another embodiment, said multiple dose schedule consists of a series of 6, 7 or 8 doses wherein each dose is separated by an interval of about 1 month to about 12 months. In a particular embodiment, said multiple dose schedule consists of a series of 6, 7 or 8 doses wherein each dose is separated by an interval of about 1 month to about 6 months. In a particular embodiment, said multiple dose schedule consists of a series of 6, 7 or 8 doses wherein each dose is separated by an interval of about 1, 2, 3, 4, 5 or 6 months. In a particular embodiment, said multiple dose schedule consists of a series of 6, 7 or 8 doses wherein each dose is separated by an interval of about 1 month. In a particular embodiment, said multiple dose schedule consists of a series of 6, 7 or 8 doses wherein each dose is separated by an interval of about 2 months.

An aspect of the invention pertains to any immunogenic composition of the invention for simultaneous, concurrent, concomitant or sequential administration with a second immunogenic composition. An aspect of the invention pertains to any kit disclosed herein for simultaneous, concurrent, concomitant or sequential administration.

By “simultaneous administration” is meant the administration of therapeutically effective doses of a first and a second immunogenic compositions in a single unit dosage form.

By “concurrent administration” is meant the administration of therapeutically effective doses of a first and a second immunogenic compositions through the same access site, but in separate unit dosage forms, within a short period of one another. Concurrent administration is essentially administering the two immunogenic compositions at about the same time but in separate dosage forms, through the same access site. The concurrent administration of the first and the second immunogenic compositions often occurs during the same physician office visit.

By “concomitant administration” is meant the administration of therapeutically effective doses of a first and a second immunogenic compositions, in separate unit dosage forms within a short period of one another at different anatomic sites. Concomitant administration is essentially administering the two immunogenic compositions at about the same time but in separate dosage forms and at different anatomic sites. The concomitant administration of the first and second immunogenic compositions often occurs during the same physician office visit.

By “sequential administration” is meant the administration of a therapeutically effective dose of a first or a second immunogenic composition alone, followed by the administration of a therapeutically effective dose of the remaining immunogenic composition after an interval of at least about 1 month. For instance in one embodiment, the first immunogenic composition is administered in a single dosage form, and then after an interval of at least about 1 month, the second immunogenic composition is administered in a separate single dosage form. In an alternative embodiment, the second immunogenic composition is administered in a single dosage form, and then after an interval of at least about 1 month, the first immunogenic composition is administered in a separate single dosage form. The sequential administration of the first and second immunogenic compositions often occurs at different physician office visits.

In an aspect of the present invention, a first immunogenic composition according to the invention (such as the ones of section 2 above) is administered simultaneously, concurrently, concomitantly or sequentially with a second immunogenic composition. In an embodiment said second immunogenic composition is any of the immunogenic compositions disclosed at section 3 above.

Therefore, an aspect of the present invention pertains to a first immunogenic composition according to the invention (such as the ones of section 2 above) for simultaneous, concurrent, concomitant or sequential use with a second immunogenic composition. In an embodiment said second immunogenic composition is any of the immunogenic compositions disclosed at section 3 above.

In some cases, as little as one dose of each of the immunogenic compositions is needed, but under some circumstances, a second, third or fourth dose of one or each of the immunogenic composition may be given. Following an initial vaccination, subjects can receive one or several booster immunizations adequately spaced.

In an embodiment, the present invention pertains to a first immunogenic composition according to the invention (such as the ones of section 2 above) for simultaneous administration with a second immunogenic composition. In an embodiment said second immunogenic composition is any of the immunogenic compositions disclosed at section 3 above.

In an embodiment, the schedule of vaccination of said simultaneous administration is a single dose. In a particular embodiment, said single dose schedule is for healthy persons being at least 2 years of age.

In an embodiment, the schedule of vaccination of said simultaneous administration is a multiple dose schedule. In a particular embodiment, said multiple dose schedule consists of a series of 2 doses separated by an interval of about 1 month to about 12 months. In a particular embodiment, said multiple dose schedule consists of a series of 2 doses separated by an interval of about 1, 2, 3, 4, 5, 6, 7, 8, 9, 10, 11 or 12 months. In a particular embodiment, said multiple dose schedule consists of a series of 2 doses separated by an interval of about 1 month to about 6 months. In a particular embodiment, said multiple dose schedule consists of a series of 2 doses separated by an interval of about 1, 2, 3, 4, 5 or 6 months. In a particular embodiment, said multiple dose schedule consists of a series of 2 doses separated by an interval of about 1 month, or a series of 2 doses separated by an interval of about 2 months.

In another embodiment, said multiple dose schedule consists of a series of 3 doses wherein each dose is separated by an interval of about 1 month to about 12 months. In a particular embodiment, said multiple dose schedule consists of a series of 3 doses wherein each dose is separated by an interval of about 1, 2, 3, 4, 5, 6, 7, 8, 9, 10, 11 or 12 months. In a particular embodiment, said multiple dose schedule consists of a series of 3 doses wherein each dose is separated by an interval of about 1 month to about 6 months. In a particular embodiment, said multiple dose schedule consists of a series of 3 doses wherein each dose is separated by an interval of about 1, 2, 3, 4, 5 or 6 months. In another embodiment, said multiple dose schedule consists of a series of 3 doses wherein each dose is separated by an interval of about 1 month, or a series of 3 doses wherein each dose is separated by an interval of about 2 months.

In a particular embodiment, said multiple dose schedule consists of a series of 4 doses separated by an interval of about 1 month to about 12 months. In a particular embodiment, said multiple dose schedule consists of a series of 4 doses separated by an interval of about 1, 2, 3, 4, 5, 6, 7, 8, 9, 10, 11 or 12 months. In a particular embodiment, said multiple dose schedule consists of a series of 4 doses separated by an interval of about 1 month to about 6 months. In a particular embodiment, said multiple dose schedule consists of a series of 4 doses separated by an interval of about 1, 2, 3, 4, 5 or 6 months. In a particular embodiment, said multiple dose schedule consists of a series of 4 doses separated by an interval of about 1 month, or a series of 4 doses separated by an interval of about 2 months.

In another embodiment, said multiple dose schedule consists of a series of 3 doses wherein each dose is separated by an interval of about 1 month to about 4 months followed by a fourth dose about 10 months to about 13 months after the first dose. In another embodiment, said multiple dose schedule consists of a series of 3 doses wherein each dose is separated by an interval of about 1, 2, 3 or 4 months followed by a fourth dose about 10 months to about 13 months after the first dose. In another embodiment, said multiple dose schedule consists of a series of 3 doses wherein each dose is separated by an interval of about 1 month to about 2 months followed by a fourth dose about 10 months to about 13 months after the first dose. In another embodiment, said multiple dose schedule consists of a series of 3 doses wherein each dose is separated by an interval of about 1 month followed by a fourth dose about 10 months to about 13 months after the first dose, or a series of 3 doses wherein each dose is separated by an interval of about 2 months followed by a fourth dose about 10 months to about 13 months after the first dose.

In an embodiment, the multiple dose schedule consists of at least one dose (e.g., 1, 2 or 3 doses) in the first year of age followed by at least one toddler dose.

In an embodiment, the multiple dose schedule consists of a series of 2 or 3 doses wherein each dose is separated by an interval of about 1 month to about 2 months (for example 28-56 days between doses), starting at 2 months of age, and followed by a toddler dose at 12-18 months of age. In an embodiment, said multiple dose schedule consists of a series of 3 doses wherein each dose is separated by an interval of about 1 month to about 2 months (for example 28-56 days between doses), starting at 2 months of age, and followed by a toddler dose at 12-15 months of age. In another embodiment, said multiple dose schedule consists of a series of 2 doses separated by an interval of about 2 months, starting at 2 months of age, and followed by a toddler dose at 12-18 months of age.

In an embodiment, the multiple dose schedule consists of a 4-dose series of vaccine administered at 2, 4, 6, and 12-15 months of age.

In an embodiment, a prime dose is given at day 0 and one or more booster doses are given at intervals that range from about 2 to about 24 weeks between doses, preferably with a dosing interval of 4-8 weeks.

In an embodiment, a prime dose is given at day 0 and a booster dose is given about 3 months later.

In a particular embodiment, said multiple dose schedule consists of a series of 5, 6, 7 or 8 doses separated by an interval of about 1 month to about 12 months. In a particular embodiment, said multiple dose schedule consists of a series of 5, 6, 7 or 8 doses separated by an interval of about 1, 2, 3, 4, 5, 6, 7, 8, 9, 10, 11 or 12 months. In a particular embodiment, said multiple dose schedule consists of a series of 5, 6, 7 or 8 doses separated by an interval of about 1 month to about 6 months. In a particular embodiment, said multiple dose schedule consists of a series of 5, 6, 7 or 8 doses separated by an interval of about 1, 2, 3, 4, 5 or 6 months. In a particular embodiment, said multiple dose schedule consists of a series of 5, 6, 7 or 8 doses separated by an interval of about 1 month, or a series of 5, 6, 7 or 8 doses separated by an interval of about 2 months.

In an embodiment, the present invention pertains to a first immunogenic composition according to the invention (such as the ones of section 2 above) for concomitant administration with a second immunogenic composition. In an embodiment said second immunogenic composition is any of the immunogenic compositions disclosed at section 3 above.

In an embodiment, the schedule of vaccination of said concomitant administration is a single dose (the administration of the first and second immunogenic composition, though in separate unit dosage forms, is considered as a single dose for purposes of defining the immunization schedule). In a particular embodiment, said single dose schedule is for healthy persons being at least 2 years of age.

In an embodiment, the schedule of vaccination of said concomitant administration is a multiple dose schedule (the administration of the first and second immunogenic composition, though in separate unit dosage forms, is considered as a single dose for purposes of defining the immunization schedule). In a particular embodiment, said multiple dose schedule consists of a series of 2 doses separated by an interval of about 1 month to about 12 months. In a particular embodiment, said schedule consists of a series of 2 doses separated by an interval of about 1, 2, 3, 4, 5, 6, 7, 8, 9, 10, 11 or 12 months. In a particular embodiment, said schedule consists of a series of 2 doses separated by an interval of about 1 month to about 6 months. In a particular embodiment, said schedule consists of a series of 2 doses separated by an interval of about 1, 2, 3, 4, 5 or 6 months. In a particular embodiment, said schedule consists of a series of 2 doses separated by an interval of about 1 month, or a series of 2 doses separated by an interval of about 2 months.

In another embodiment, said multiple dose schedule consists of a series of 3 doses wherein each dose is separated by an interval of about 1 month to about 12 months. In a particular embodiment, said schedule consists of a series of 3 doses wherein each dose is separated by an interval of about 1, 2, 3, 4, 5, 6, 7, 8, 9, 10, 11 or 12 months. In a particular embodiment, said schedule consists of a series of 3 doses wherein each dose is separated by an interval of about 1 month to about 6 months. In a particular embodiment, said schedule consists of a series of 3 doses wherein each dose is separated by an interval of about 1, 2, 3, 4, 5 or 6 months. In another embodiment, said schedule consists of a series of 3 doses wherein each dose is separated by an interval of about 1 month, or a series of 3 doses wherein each dose is separated by an interval of about 2 months.

In a particular embodiment, said multiple dose schedule consists of a series of 4 doses separated by an interval of about 1 month to about 12 months. In a particular embodiment, said multiple dose schedule consists of a series of 4 doses separated by an interval of about 1, 2, 3, 4, 5, 6, 7, 8, 9, 10, 11 or 12 months. In a particular embodiment, said multiple dose schedule consists of a series of 4 doses separated by an interval of about 1 month to about 6 months. In a particular embodiment, said multiple dose schedule consists of a series of 4 doses separated by an interval of about 1, 2, 3, 4, 5 or 6 months. In a particular embodiment, said multiple dose schedule consists of a series of 4 doses separated by an interval of about 1 month, or a series of 4 doses separated by an interval of about 2 months.

In another embodiment, said multiple dose schedule consists of a series of 3 doses wherein each dose is separated by an interval of about 1 month to about 4 months followed by a fourth dose about 10 months to about 13 months after the first dose. In another embodiment, said schedule consists of a series of 3 doses wherein each dose is separated by an interval of about 1, 2, 3 or 4 months followed by a fourth dose about 10 months to about 13 months after the first dose. In another embodiment, said schedule consists of a series of 3 doses wherein each dose is separated by an interval of about 1 month to about 2 months followed by a fourth dose about 10 months to about 13 months after the first dose. In another embodiment, said schedule consists of a series of 3 doses wherein each dose is separated by an interval of about 1 month followed by a fourth dose about 10 months to about 13 months after the first dose, or a series of 3 doses wherein each dose is separated by an interval of about 2 months followed by a fourth dose about 10 months to about 13 months after the first dose.

In an embodiment, the multiple dose schedule consists of at least one dose (e.g., 1, 2 or 3 doses) in the first year of age followed by at least one toddler dose.

In an embodiment, the multiple dose schedule consists of a series of 2 or 3 doses wherein each dose is separated by an interval of about 1 month to about 2 months (for example 28-56 days between doses), starting at 2 months of age, and followed by a toddler dose at 12-18 months of age. In an embodiment, said schedule consists of a series of 3 doses wherein each dose is separated by an interval of about 1 month to about 2 months (for example 28-56 days between doses), starting at 2 months of age, and followed by a toddler dose at 12-15 months of age. In another embodiment, said schedule consists of a series of 2 doses separated by an interval of about 2 months, starting at 2 months of age, and followed by a toddler dose at 12-18 months of age.

In an embodiment, the multiple dose schedule consists of a 4-dose series of vaccine administered at 2, 4, 6, and 12-15 months of age.

In an embodiment, a prime dose is given at day 0 and one or more booster doses are given at intervals that range from about 2 to about 24 weeks, preferably with a dosing interval of 4-8 weeks.

In an embodiment, a prime dose is given at day 0 and a boost is given about 3 months later.

In a particular embodiment, said multiple dose schedule consists of a series of 5, 6, 7 or 8 doses separated by an interval of about 1 month to about 12 months. In a particular embodiment, said multiple dose schedule consists of a series of 5, 6, 7 or 8 doses separated by an interval of about 1, 2, 3, 4, 5, 6, 7, 8, 9, 10, 11 or 12 months. In a particular embodiment, said multiple dose schedule consists of a series of 5, 6, 7 or 8 doses separated by an interval of about 1 month to about 6 months. In a particular embodiment, said multiple dose schedule consists of a series of 5, 6, 7 or 8 doses separated by an interval of about 1, 2, 3, 4, 5 or 6 months. In a particular embodiment, said multiple dose schedule consists of a series of 5, 6, 7 or 8 doses separated by an interval of about 1 month, or a series of 5, 6, 7 or 8 doses separated by an interval of about 2 months.

In another embodiment, the present invention pertains to a first immunogenic composition according to the invention (such as the ones of section 2 above) for concurrent administration with a second immunogenic composition. In an embodiment said second immunogenic composition is any of the immunogenic compositions disclosed at section 3 above.

In an embodiment, the schedule of vaccination of said concurrent administration is a single dose (the administration of the first and second immunogenic composition, though in separate unit dosage forms, is considered as a single dose for purposes of defining the immunization schedule). In a particular embodiment, said single dose schedule is for healthy persons being at least 2 years of age.

In an embodiment, the schedule of vaccination of said concurrent administration is a multiple dose schedule, in particular any of the multiple schedules disclosed above for a concomitant administration.

In an embodiment, the present invention pertains to a first immunogenic composition according to the invention (such as the ones of section 2 above) for sequential administration with a second immunogenic composition. In an embodiment said second immunogenic composition is any of the immunogenic compositions disclosed at section 3 above.

In an embodiment, the first immunogenic composition according to the invention is administered first and the second immunogenic composition is administered second. In another embodiment, the second immunogenic composition is administered first and the first immunogenic composition according to the invention is administered second.

In an embodiment, the schedule of vaccination of said sequential administration consists of a series of 2, 3, 4, 5, 6, 7 or 8 doses.

In an embodiment, the schedule of vaccination of said sequential administration consists of a series of 2, 3 or 4 doses.

In an embodiment, the schedule of vaccination of said sequential administration consists of a series of 2 doses. In an embodiment, the schedule of vaccination of said sequential administration consists of a series of 2 doses separated by an interval of about 1 month to about 12 months. In a particular embodiment, said multiple dose schedule consists of a series of 2 doses separated by an interval of about 1, 2, 3, 4, 5, 6, 7, 8, 9, 10, 11 or 12 months. In a particular embodiment, said multiple dose schedule consists of a series of 2 doses separated by an interval of about 1 month to about 6 months. In a particular embodiment, said multiple dose schedule consists of a series of 2 doses separated by an interval of about 1, 2, 3, 4, 5 or 6 months. In a particular embodiment, said multiple dose schedule consists of a series of 2 doses separated by an interval of about 1 month, or a series of 2 doses separated by an interval of about 2 months.

In an embodiment of said 2-dose schedule, the first immunogenic composition according to the invention is administered first and the second immunogenic composition is administered second. In another embodiment, the second immunogenic composition is administered first and the first immunogenic composition according to the invention is administered second.

In an embodiment of said 2-dose schedule, the first and second doses are administered in the first year of age. In an embodiment of said 2-dose schedules, the first dose is administered in the first year of age and the second dose is a toddler dose.

In an embodiment, said toddler dose is administered at 12-18 months of age. In an embodiment, said toddler dose is administered at 12-15 months of age.

In an embodiment, the schedule of vaccination of said sequential administration consists of a series of 3 doses. In a particular embodiment, said schedule consists of a series of 3 doses wherein each dose is separated by an interval of about 1 to about 12 months. In a particular embodiment, said schedule consists of a series of 3 doses wherein each dose is separated by an interval of about 1, 2, 3, 4, 5, 6, 7, 8, 9, 10, 11 or 12 months. In a particular embodiment, said schedule consists of a series of 3 doses wherein each dose is separated by an interval of about 1 month to about 6 months. In a particular embodiment, said schedule consists of a series of 3 doses wherein each dose is separated by an interval of about 1, 2, 3, 4, 5 or 6 months. In a particular embodiment, said schedule consists of a series of 3 doses wherein each dose is separated by an interval of about 1 to about 2 months. In another embodiment, said schedule consists of a series of 3 doses wherein each dose is separated by an interval of about 1 month, or a series of 3 doses wherein each dose is separated by an interval of about 2 months.

In an embodiment of said 3-dose schedule, the first and second doses are administered in the first year of age and the third dose is a toddler dose. In an embodiment, the first and second doses are separated by an interval of about 1 month to about 2 months (for example 28-56 days between doses), starting at 2 months of age, and the third dose is a toddler dose at 12-18 months of age. In an embodiment, the first and second doses are separated by an interval of about 1 month to about 2 months (for example 28-56 days between doses), starting at 2 months of age, and the third dose is a toddler dose at 12-15 months of age.

In an embodiment of said 3-dose schedule, the first immunogenic composition according to the invention is administered as the first two doses and the second immunogenic composition is administered as the third dose.

In another embodiment of said 3-dose schedule, the second immunogenic composition is administered as the first two doses and the first immunogenic composition according to the invention is administered as the third dose.

In another embodiment of said 3-dose schedule, the first immunogenic composition according to the invention is administered as the first dose, the second immunogenic composition is administered as the second dose and the first immunogenic composition according to the invention is administered as the third dose.

In yet another embodiment of said 3-dose schedule, the second immunogenic composition is administered as the first dose, the first immunogenic composition according to the invention is administered as the second dose and the second immunogenic composition is administered as the third dose.

In yet another embodiment of said 3-dose schedule, the first immunogenic composition according to the invention is administered as the first dose and the second immunogenic composition is administered as the second and third doses.

In another embodiment of said 3-dose schedule, the second immunogenic composition is administered as the first dose and the first immunogenic composition according to the invention is administered as the second and third doses.

In an embodiment, the schedule of vaccination of said sequential administration consists of a series of 4 doses.

In a particular embodiment, said schedule consists of a series of 4 doses wherein each dose is separated by an interval of about 1 to about 12 months. In a particular embodiment, said schedule consists of a series of 4 doses wherein each dose is separated by an interval of about 1, 2, 3, 4, 5, 6, 7, 8, 9, 10, 11 or 12 months. In a particular embodiment, said schedule consists of a series of 4 doses wherein each dose is separated by an interval of about 1 month to about 6 months. In a particular embodiment, said schedule consists of a series of 4 doses wherein each dose is separated by an interval of about 1, 2, 3, 4, 5 or 6 months. In a particular embodiment, said schedule consists of a series of 4 doses wherein each dose is separated by an interval of about 1 to about 2 months. In another embodiment, said schedule consists of a series of 4 doses wherein each dose is separated by an interval of about 1 month, or a series of 4 doses wherein each dose is separated by an interval of about 2 months.

In an embodiment of said 4-dose schedule, said schedule consists of a series of 3 doses wherein each dose is separated by an interval of about 1 month to about 4 months followed by a fourth dose about 10 months to about 13 months after the first dose. In another embodiment, said schedule consists of a series of 3 doses wherein each dose is separated by an interval of about 1, 2, 3 or 4 months followed by a fourth dose about 10 months to about 13 months after the first dose. In another embodiment, said schedule consists of a series of 3 doses wherein each dose is separated by an interval of about 1 month to about 2 months followed by a fourth dose about 10 months to about 13 months after the first dose. In another embodiment, said schedule consists of a series of 3 doses wherein each dose is separated by an interval of about 1 month followed by a fourth dose about 10 months to about 13 months after the first dose, or a series of 3 doses wherein each dose is separated by an interval of about 2 months followed by a fourth dose about 10 months to about 13 months after the first dose.

In an embodiment of said 4-dose schedule the first, second and third doses are administered in the first year of age and the fourth dose is a toddler dose.

In an embodiment, said 4-dose schedule consists of a series of 3 doses wherein each dose is separated by an interval of about 1 month to about 2 months (for example 28-56 days between doses), starting at 2 months of age, followed by a toddler dose at 12-18 months of age. In an embodiment, said schedule consists of a series of 3 doses wherein each dose is separated by an interval of about 1 month to about 2 months (for example 28-56 days between doses), starting at 2 months of age, followed by a toddler dose at 12-15 months of age.

In an embodiment, the multiple dose schedule consists of a 4-dose series of vaccine at 2, 4, 6, and 12-15 months of age.

In an embodiment of said 4-dose schedule, the first immunogenic composition according to the invention is administered as the first three doses and the second immunogenic composition is administered as the fourth dose.

In another embodiment of said 4-dose schedule, the second immunogenic composition is administered as the first three doses and the first immunogenic composition according to the invention is administered as the fourth dose.

In another embodiment of said 4-dose schedule, the first immunogenic composition according to the invention is administered as the first and second doses and the second immunogenic composition is administered as the third and fourth doses.

In another embodiment of said 4-dose schedule, the second immunogenic composition is administered as the first and second doses and the first immunogenic composition according to the invention is administered as the third and fourth doses.

In another embodiment of said 4-dose schedule, the first immunogenic composition according to the invention is administered as the first and second doses, the second immunogenic composition is administered as the third dose and the first immunogenic composition according to the invention is administered as the fourth dose.

In another embodiment of said 4-dose schedule, the second immunogenic composition is administered as the first and second doses, the first immunogenic composition according to the invention is administered as the third dose and the second immunogenic composition is administered as the fourth dose.

In another embodiment of said 4-dose schedule, the first immunogenic composition according to the invention is administered as the first dose and the second immunogenic composition is administered as the second, third and fourth doses. In another embodiment of said 4-dose schedule, the second immunogenic composition is administered as the first dose and the first immunogenic composition according to the invention is administered as the second, third and fourth doses.

In another embodiment of said 4-dose schedule, the first immunogenic composition according to the invention is administered as the first dose, the second immunogenic composition is administered as the second dose, the first immunogenic composition according to the invention is administered as the third dose and the second immunogenic composition is administered as the fourth dose.

In another embodiment of said 4-dose schedule, the second immunogenic composition is administered as the first dose, the first immunogenic composition according to the invention is administered as the second dose, the second immunogenic composition is administered as the third dose and the first immunogenic composition according to the invention is administered as the fourth dose.

In another embodiment of said 4-dose schedule, the first immunogenic composition according to the invention is administered as the first dose, the second immunogenic composition is administered as the second dose and the first immunogenic composition according to the invention is administered as the third and fourth doses.

In another embodiment of said 4-dose schedule, the second immunogenic composition is administered as the first dose, the first immunogenic composition according to the invention is administered as the second dose and the second immunogenic composition is administered as the third and fourth doses.

In another embodiment of said 4-dose schedule, the first immunogenic composition according to the invention is administered as the first dose, the second immunogenic composition is administered as the second and third doses and the first immunogenic composition according to the invention is administered as the fourth dose.

In another embodiment of said 4-dose schedule, the second immunogenic composition is administered as the first dose, the first immunogenic composition according to the invention is administered as the second and third doses and the second immunogenic composition is administered as the fourth dose.

In an embodiment, the schedule of vaccination of said sequential administration consists of a series of 5 doses.

In a particular embodiment, said schedule consists of a series of 5 doses wherein each dose is separated by an interval of about 1 to about 12 months. In a particular embodiment, said schedule consists of a series of 5 doses wherein each dose is separated by an interval of about 1, 2, 3, 4, 5, 6, 7, 8, 9, 10, 11 or 12 months. In a particular embodiment, said schedule consists of a series of 5 doses wherein each dose is separated by an interval of about 1 month to about 6 months. In a particular embodiment, said schedule consists of a series of 5 doses wherein each dose is separated by an interval of about 1, 2, 3, 4, 5 or 6 months. In a particular embodiment, said schedule consists of a series of 5 doses wherein each dose is separated by an interval of about 1 to about 2 months. In another embodiment, said schedule consists of a series of 5 doses wherein each dose is separated by an interval of about 1 month, or a series of 5 doses wherein each dose is separated by an interval of about 2 months.

In an embodiment said 5-dose schedule consists of a series of 4 doses wherein each dose is separated by an interval of about 1 month to about 3 months followed by a fifth dose about 10 months to about 13 months after the first dose. In another embodiment, said schedule consists of a series of 4 doses wherein each dose is separated by an interval of about 1 month to about 2 months followed by a fifth dose about 10 months to about 13 months after the first dose. In another embodiment, said schedule consists of a series of 4 doses wherein each dose is separated by an interval of about 1 month followed by a fifth dose about 10 months to about 13 months after the first dose, or a series of 4 doses wherein each dose is separated by an interval of about 2 months followed by a fifth dose about 10 months to about 13 months after the first dose.

In an embodiment of said 5-doses schedule, the first, second, third and fourth doses are administered in the first year of age and the fifth dose is a toddler dose. In an embodiment, said 5-doses schedule consists of a series of 4 doses wherein each dose is separated by an interval of about 1 month to about 2 months (for example 28-56 days between doses), starting at 2 months of age, and followed by a toddler dose at 12-18 months of age. In an embodiment, said schedule consists of a series of 4 doses wherein each dose is separated by an interval of about 1 month to about 2 months (for example 28-56 days between doses), starting at 2 months of age, and followed by a toddler dose at 12-15 months of age.

In an embodiment of said 5-doses schedule, the first immunogenic composition according to the invention (such as the ones of section 2 above, designated 1^{st} IC in the 5 below table) and the second immunogenic composition (such as disclosed at section 3 above, designated 2^{nd} IC in the below table) may be administered in the following order:

| Schedule | Dose | | | | |
|---|---|---|---|---|---|
| number | 1 | 2 | 3 | 4 | 5 |
| 1 | 2^{nd} IC | 2^{nd} IC | 2^{nd} IC | 2^{nd} IC | 1^{st} IC |
| 2 | 2^{nd} IC | 2^{nd} IC | 2^{nd} IC | 1^{st} IC | 2^{nd} IC |
| 3 | 2^{nd} IC | 2^{nd} IC | 2^{nd} IC | 1^{st} IC | 1^{st} IC |
| 4 | 2^{nd} IC | 2^{nd} IC | 1^{st} IC | 2^{nd} IC | 2^{nd} IC |
| 5 | 2^{nd} IC | 2^{nd} IC | 1^{st} IC | 1^{st} IC | 2^{nd} IC |
| 6 | 2^{nd} IC | 2^{nd} IC | 1^{st} IC | 1^{st} IC | 1^{st} IC |
| 7 | 2^{nd} IC | 2^{nd} IC | 1^{st} IC | 2^{nd} IC | 1^{st} IC |
| 8 | 2^{nd} IC | 1^{st} IC | 2^{nd} IC | 2^{nd} IC | 2^{nd} IC |
| 9 | 2^{nd} IC | 1^{st} IC | 2^{nd} IC | 2^{nd} IC | 1^{st} IC |
| 10 | 2^{nd} IC | 1^{st} IC | 2^{nd} IC | 1^{st} IC | 2^{nd} IC |
| 11 | 2^{nd} IC | 1^{st} IC | 2^{nd} IC | 1^{st} IC | 1^{st} IC |
| 12 | 2^{nd} IC | 1^{st} IC | 1^{st} IC | 2^{nd} IC | 2^{nd} IC |
| 13 | 2^{nd} IC | 1^{st} IC | 1^{st} IC | 2^{nd} IC | 1^{st} IC |
| 14 | 2^{nd} IC | 1^{st} IC | 1^{st} IC | 1^{st} IC | 2^{nd} IC |
| 15 | 2^{nd} IC | 1^{st} IC | 1^{st} IC | 1^{st} IC | 1^{st} IC |
| 16 | 1^{st} IC | 2^{nd} IC | 2^{nd} IC | 2^{nd} IC | 2^{nd} IC |
| 17 | 1^{st} IC | 2^{nd} IC | 2^{nd} IC | 2^{nd} IC | 1^{st} IC |
| 18 | 1^{st} IC | 2^{nd} IC | 2^{nd} IC | 1^{st} IC | 2^{nd} IC |
| 19 | 1^{st} IC | 2^{nd} IC | 2^{nd} IC | 1^{st} IC | 1^{st} IC |
| 20 | 1^{st} IC | 2^{nd} IC | 1^{st} IC | 2^{nd} IC | 2^{nd} IC |
| 21 | 1^{st} IC | 2^{nd} IC | 1^{st} IC | 2^{nd} IC | 1^{st} IC |
| 22 | 1^{st} IC | 2^{nd} IC | 1^{st} IC | 1^{st} IC | 2^{nd} IC |
| 23 | 1^{st} IC | 2^{nd} IC | 1^{st} IC | 1^{st} IC | 1^{st} IC |
| 24 | 1^{st} IC | 1^{st} IC | 2^{nd} IC | 2^{nd} IC | 2^{nd} IC |
| 25 | 1^{st} IC | 1^{st} IC | 2^{nd} IC | 2^{nd} IC | 1^{st} IC |
| 26 | 1^{st} IC | 1^{st} IC | 2^{nd} IC | 1^{st} IC | 2^{nd} IC |
| 27 | 1^{st} IC | 1^{st} IC | 2^{nd} IC | 1^{st} IC | 1^{st} IC |
| 28 | 1^{st} IC | 1^{st} IC | 1^{st} IC | 2^{nd} IC | 2^{nd} IC |
| 29 | 1^{st} IC | 1^{st} IC | 1^{st} IC | 2^{nd} IC | 1^{st} IC |
| 30 | 1^{st} IC | 1^{st} IC | 1^{st} IC | 1^{st} IC | 2^{nd} IC |

The above table provide the order of administration of the first and second immunogenic composition (designated 1^{st} IC and 2^{nd} IC respectively) for the different doses, for example schedule number 1 is to be read as: in embodiment of said 5-dose schedule, the second immunogenic composition is administered as the first, second, third and fourth doses and the first immunogenic composition according to the invention is administered as the fifth dose.

In an embodiment, the order of administration of the first and second immunogenic composition is according to schedule 1, 2, 3, 4, 5, 6, 7, 8, 9, 10, 11, 12, 13, 16, 17, 18, 19, 20 or 21.

In an embodiment, the schedule of vaccination of said sequential dose consists of a series of 6 doses.

In a particular embodiment, said schedule consists of a series of 6 doses wherein each dose is separated by an interval of about 1 to about 12 months. In a particular embodiment, said schedule consists of a series of 6 doses wherein each dose is separated by an interval of about 1, 2, 3, 4, 5, 6, 7, 8, 9, 10, 11 or 12 months. In a particular embodiment, said schedule consists of a series of 6 doses wherein each dose is separated by an interval of about 1 month to about 6 months. In a particular embodiment, said schedule consists of a series of 6 doses wherein each dose is separated by an interval of about 1, 2, 3, 4, 5 or 6 months. In a particular embodiment, said schedule consists of a series of 6 doses wherein each dose is separated by an interval of about 1 to about 2 months. In another embodiment, said schedule consists of a series of 6 doses wherein each dose is separated by an interval of about 1 month, or a series of 6 doses wherein each dose is separated by an interval of about 2 months.

In an embodiment said 6-dose schedule consists of a series of 5 doses wherein each dose is separated by an interval of about 1 month to about 2 months followed by a sixth dose about 10 months to about 13 months after the first dose. In another embodiment, said schedule consists of a series of 5 doses wherein each dose is separated by an interval of about 1 month followed by a sixth dose about 10 months to about 13 months after the first dose, or a series of 5 doses wherein each dose is separated by an interval of about 2 months followed by a sixth dose about 10 months to about 13 months after the first dose.

In an embodiment of said 6-doses schedule, the first, second, third, fourth and fifth doses are administered in the first year of age and the sixth dose is a toddler dose.

In an embodiment, said 6-doses schedule consists of a series of 5 doses wherein each dose is separated by an interval of about 1 month to about 2 months (for example 28-56 days between doses), starting at 2 months of age, and followed by a toddler dose at 12-18 months of age. In an embodiment, said schedule consists of a series of 5 doses wherein each dose is separated by an interval of about 1 month to about 2 months (for example 28-56 days between doses), starting at 2 months of age, and followed by a toddler dose at 12-15 months of age.

In an embodiment of said 6-doses schedule, the first immunogenic composition according to the invention (such as the ones of section 2 above) and the second immunogenic composition (such as disclosed at section 3 above) are administered in the order according to the any of the 30 schedules provided for the 5-doses schedule (see above table, schedule 1 to 30), followed by a sixth dose. In an embodiment, the first immunogenic composition according to the invention is administered as the sixth dose. In another embodiment, the second immunogenic composition is administered as the sixth dose.

In an embodiment, the schedule of vaccination of said sequential dose consists of a series of 7 doses.

In a particular embodiment, said schedule consists of a series of 7 doses wherein each dose is separated by an interval of about 1 to about 12 months. In a particular embodiment, said schedule consists of a series of 7 doses wherein each dose is separated by an interval of about 1, 2, 3, 4, 5, 6, 7, 8, 9, 10, 11 or 12 months. In a particular embodiment, said schedule consists of a series of 7 doses wherein each dose is separated by an interval of about 1 month to about 6 months. In a particular embodiment, said schedule consists of a series of 7 doses wherein each dose is separated by an interval of about 1, 2, 3, 4, 5 or 6 months. In a particular embodiment, said schedule consists of a series of 7 doses wherein each dose is separated by an interval of about 1 to about 2 months. In another embodiment, said schedule consists of a series of 7 doses wherein each dose is separated by an interval of about 1 month, or a series of 7 doses wherein each dose is separated by an interval of about 2 months.

In an embodiment said 7-dose schedule consists of a series of 6 doses wherein each dose is separated by an interval of about 1 month followed by a seventh dose about 10 months to about 13 months after the first dose.

In an embodiment of said 7-doses schedule, the first, second, third, fourth, fifth and sixth doses are administered in the first year of age and the seventh dose is a toddler dose. In an embodiment, said 7-dose schedule consists of a series of 6 doses wherein each dose is separated by an interval of about 1 month (for example 28-40 days between doses), starting at 2 months of age, and followed by a toddler dose at 12-18 months of age. In an embodiment, said schedule consists of a series of 6 doses wherein each dose is separated by an interval of about 1 month (for example 28-40 days between doses), starting at 2 months of age, and followed by a toddler dose at 12-15 months of age.

In an embodiment of said 7-doses schedule, the first immunogenic composition according to the invention (such as the ones of section 2 above) and the second immunogenic composition (such as disclosed at section 3 above) are administered in the order according to the any of the schedules provided for the 6-doses schedule (see above), followed by a seventh dose. In an embodiment, the first immunogenic composition according to the invention is administered as the seventh dose. In another embodiment, the second immunogenic composition is administered as the seventh dose.

In an embodiment, the schedule of vaccination of said sequential dose consists of a series of 8 doses.

In a particular embodiment, said schedule consists of a series of 8 doses wherein each dose is separated by an interval of about 1 to about 12 months. In a particular embodiment, said schedule consists of a series of 8 doses wherein each dose is separated by an interval of about 1, 2, 3, 4, 5, 6, 7, 8, 9, 10, 11 or 12 months. In a particular embodiment, said schedule consists of a series of 8 doses wherein each dose is separated by an interval of about 1 month to about 6 months. In a particular embodiment, said schedule consists of a series of 8 doses wherein each dose is separated by an interval of about 1, 2, 3, 4, 5 or 6 months. In a particular embodiment, said schedule consists of a series of 8 doses wherein each dose is separated by an interval of about 1 to about 2 months. In another embodiment, said schedule consists of a series of 8 doses wherein each dose is separated by an interval of about 1 month, or a series of 8 doses wherein each dose is separated by an interval of about 2 months.

In an embodiment said 8-dose schedule consists of a series of 7 doses wherein each dose is separated by an interval of about 1 month followed by an eighth dose about 10 months to about 13 months after the first dose.

In an embodiment of said 8-doses schedule, the first, second, third, fourth, fifth, sixth and seventh doses are administered in the first year of age and the eighth dose is a toddler dose. In an embodiment, said 8-dose schedule consists of a series of 7 doses wherein each dose is separated by an interval of about 1 month (for example 28-40 days between doses), starting at 2 months of age, and followed by a toddler dose at 12-18 months of age. In an embodiment, said schedule consists of a series of 7 doses wherein each dose is separated by an interval of about 1 month (for example 28-40 days between doses), starting at 2 months of age, and followed by a toddler dose at 12-15 months of age.

In an embodiment of said 8-doses schedule, the first immunogenic composition according to the invention (such as the ones of section 2 above) and the second immunogenic composition (such as disclosed at section 3 above) are administered in the order according to the any of the schedules provided for the 7-doses schedule (see above), followed by a eighth dose. In an embodiment, the first immunogenic composition according to the invention is administered as the eighth dose. In another embodiment, the second immunogenic composition is administered as the eighth dose.

In an embodiment, the present invention pertains to the sequential administration of:
(a) a first immunogenic composition according to the invention (such as the ones of section 2 above) and
(b) the concomitant administration of the first immunogenic composition according to the invention (such as the ones of section 2 above) with a second immunogenic composition.

In an embodiment said second immunogenic composition is any of the immunogenic compositions disclosed at section 3 above.

In an embodiment, the schedule of vaccination of said sequential administration consists of a series of 2 administrations. In an embodiment, the schedule of vaccination consists of a series of 2 administrations separated by an interval of about 1 month to about 12 months. In a particular embodiment, said schedule consists of a series of 2 administrations separated by an interval of about 1, 2, 3, 4, 5, 6, 7, 8, 9, 10, 11 or 12 months. In a particular embodiment, said schedule consists of a series of 2 administrations separated by an interval of about 1 month to about 6 months. In a particular embodiment, said schedule consists of a series of 2 administrations separated by an interval of about 1, 2, 3, 4, 5 or 6 months. In an embodiment, the schedule of vaccination consists of a series of 2 administrations separated by an interval of about 1 month to about 2 months. In a particular embodiment, said schedule consists of a series of 2 administrations separated by an interval of about 1 month, or a series of 2 administrations separated by an interval of about 2 months.

In an embodiment of said schedule, a first immunogenic composition according to the invention is administered first and the concomitant administration of the first immunogenic composition according to the invention with a second immunogenic composition is administered second. In another embodiment, the concomitant administration of a first immunogenic composition according to the invention with a second immunogenic composition is administered first and the first immunogenic composition according to the invention is administered second.

In an embodiment of said 2-administration schedule, the first and second administrations are administered in the first year of age. In an embodiment of said 2-administration schedule, the first administration is administered in the first year of age and the second administration is a toddler administration. In an embodiment, said toddler administration is administered at 12-18 months of age. In an embodiment, said toddler administration is administered at 12-15 months of age.

In an embodiment, the schedule of vaccination of said sequential administration consists of a series of 3 administrations. In an embodiment, said schedule consists of a series of 3 administrations separated by an interval of about 1 month to about 12 months. In a particular embodiment, said schedule consists of a series of 3 administrations wherein each administration is separated by an interval of about 1, 2, 3, 4, 5, 6, 7, 8, 9, 10, 11 or 12 months. In a particular embodiment, said schedule consists of a series of 3 administrations wherein each administration is separated by an interval of about 1 month to about 6 months. In a particular embodiment, said schedule consists of a series of 3 administrations wherein each administration is separated by an interval of about 1, 2, 3, 4, 5 or 6 months. In an embodiment, said schedule consists of a series of 3 administrations separated by an interval of about 1 month to about 2 months. In another embodiment, said schedule consists of a series of 3 administrations wherein each administration is separated by an interval of about 1 month, or a series of 3 administrations wherein each administration is separated by an interval of about 2 months.

In an embodiment of said 3-administration schedule, the first and second administrations are administered in the first year of age and the third administration is a toddler administration. In an embodiment, the first and second administrations are separated by an interval of about 1 month to about 2 months (for example 28-56 days between administrations), starting at 2 months of age, and the third administration is a toddler administration at 12-18 months of age. In an embodiment, the first and second administrations are separated by an interval of about 1 month to about 2 months (for example 28-56 days between administrations), starting at 2 months of age, and the third administration is a toddler administration at 12-15 months of age.

In an embodiment of said 3-administration schedule, the first immunogenic composition according to the invention is administered at the first and second administrations and the concomitant administration of the first immunogenic composition according to the invention with the second immunogenic composition is administered at the third administration.

In another embodiment of said 3-administration schedule, the concomitant administration of the first immunogenic composition according to the invention with the second immunogenic composition is administered at the first and second administrations and the first immunogenic composition according to the invention is administered at the third administration.

In another embodiment of said 3-administration schedule, the first immunogenic composition according to the invention is administered at the first administration, the concomitant administration of the first immunogenic composition according to the invention with the second immunogenic composition is administered at the second administration and the first immunogenic composition according to the invention is administered at the third administration.

In yet another embodiment of said 3-administration schedule, the concomitant administration of the first immunogenic composition according to the invention with the second immunogenic composition is administered at the first administration, the first immunogenic composition according to the invention is administered at the second administration and the concomitant administration of the first immunogenic composition according to the invention with the second immunogenic composition is administered at the third administration.

In yet another embodiment of said 3-administration schedule, the first immunogenic composition according to the invention is administered at the first administration and the concomitant administration of the first immunogenic composition according to the invention with the second immunogenic composition is administered at the second and third administrations.

In another embodiment of said 3-administration schedule, the concomitant administration of the first immunogenic composition according to the invention with the second immunogenic composition is administered at the first administration and the first immunogenic composition according to the invention is administered at the second and third administrations.

In an embodiment, the schedule of vaccination of said sequential administration consists of a series of 4 administrations.

In an embodiment, said schedule consists of a series of 4 administrations separated by an interval of about 1 month to about 12 months. In a particular embodiment, said schedule consists of a series of 4 administrations wherein each administration is separated by an interval of about 1, 2, 3, 4, 5, 6, 7, 8, 9, 10, 11 or 12 months. In a particular embodiment, said schedule consists of a series of 4 administrations wherein each administration is separated by an interval of about 1 month to about 6 months. In a particular embodiment, said schedule consists of a series of 4 administrations wherein each administration is separated by an interval of about 1, 2, 3, 4, 5 or 6 months. In an embodiment, said schedule consists of a series of 4 administrations separated by an interval of about 1 month to about 2 months. In another embodiment, said schedule consists of a series of 4 administrations wherein each administration is separated by an interval of about 1 month, or a series of 4 administrations wherein each administration is separated by an interval of about 2 months.

In an embodiment of said 4-administration schedule, said schedule consists of a series of 3 administrations wherein each administration is separated by an interval of about 1 month to about 4 months followed by a fourth administration about 10 months to about 13 months after the first administration. In another embodiment, said schedule consists of a series of 3 administrations wherein each administration is separated by an interval of about 1, 2, 3 or 4 months followed by a fourth administration about 10 months to about 13 months after the first administration. In another embodiment, said schedule consists of a series of 3 administrations wherein each administration is separated by an interval of about 1 month to about 2 months followed by a fourth administration about 10 months to about 13 months after the first administration. In another embodiment, said schedule consists of a series of 3 administrations wherein each administration is separated by an interval of about 1 month followed by a fourth administration about 10 months to about 13 months after the first administration, or a series of 3 administrations wherein each administration is separated by an interval of about 2 months followed by a fourth administration about 10 months to about 13 months after the first administration.

In an embodiment of said 4-administration schedule, the first, second and third administrations are administered in the first year of age and the fourth administration is a toddler administration. In an embodiment, said 4-administration schedule consists of a series of 3 administrations wherein each administration is separated by an interval of about 1 month to about 2 months (for example 28-56 days between administrations), starting at 2 months of age, and followed by a toddler administration at 12-18 months of age. In an embodiment, said schedule consists of a series of 3 administrations wherein each administration is separated by an interval of about 1 month to about 2 months (for example 28-56 days between administrations), starting at 2 months of age, and followed by a toddler administration at 12-15 months of age.

In an embodiment, said 4-administration schedule consists of a series of administrations at 2, 4, 6, and 12-15 months of age.

In an embodiment of said 4-administration schedule, the first immunogenic composition according to the invention is administered at the first, second and third administrations and the concomitant administration of the first immunogenic composition according to the invention with the second immunogenic composition is administered at the fourth administration.

In another embodiment of said 4-administration schedule, the concomitant administration of the first immunogenic composition according to the invention with the second immunogenic composition is administered at the first, second and third administrations and the first immunogenic composition according to the invention is administered at the fourth administration.

In another embodiment of said 4-administration schedule, the first immunogenic composition according to the invention is administered at the first and second administrations and the concomitant administration of the first immunogenic composition according to the invention with the second immunogenic composition is administered at the third and fourth administrations.

In another embodiment of said 4-administration schedule, the concomitant administration of the first immunogenic composition according to the invention with the second immunogenic composition is administered at the first and second administrations and the first immunogenic composition according to the invention is administered at the third and fourth administrations.

In another embodiment of said 4-administration schedule, the first immunogenic composition according to the invention is administered at the first and second administrations, the concomitant administration of the first immunogenic composition according to the invention with the second immunogenic composition is administered at the third administration and the first immunogenic composition according to the invention is administered at the fourth administration.

In another embodiment of said 4-administration schedule, the concomitant administration of the first immunogenic composition according to the invention with the second immunogenic composition is administered at the first and second administrations, the first immunogenic composition according to the invention is administered at the third administration and the concomitant administration of the first immunogenic composition according to the invention with the second immunogenic composition is administered at the fourth administration.

In another embodiment of said 4-administration schedule, the first immunogenic composition according to the invention is administered at the first administration and the concomitant administration of the first immunogenic composition according to the invention with the second immunogenic composition is administered at the second, third and fourth administrations.

In another embodiment of said 4-administration schedule, the concomitant administration of the first immunogenic composition according to the invention with the second immunogenic composition is administered at the first administration and the first immunogenic composition according to the invention is administered at the second, third and fourth administration.

In another embodiment of said 4-administration schedule, the first immunogenic composition according to the invention is administered at the first administration, the concomitant administration of the first immunogenic composition according to the invention with the second immunogenic composition is administered at the second administration, the first immunogenic composition according to the invention is administered at the third administration and the concomitant administration of the first immunogenic composition according to the invention with the second immunogenic composition is administered at the fourth administration.

In another embodiment of said 4-administration schedule, the concomitant administration of the first immunogenic composition according to the invention with the second immunogenic composition is administered at the first administration, the first immunogenic composition according to the invention is administered at the second administration, the concomitant administration of the first immunogenic composition according to the invention with the second immunogenic composition is administered at the third administration and the first immunogenic composition according to the invention is administered at the fourth administration.

In another embodiment of said 4-administration schedule, the first immunogenic composition according to the invention is administered at the first administration, the concomitant administration of the first immunogenic composition according to the invention with the second immunogenic composition is administered at the second administration and the first immunogenic composition according to the invention is administered at the third and fourth administrations.

In another embodiment of said 4-administration schedule, the concomitant administration of the first immunogenic composition according to the invention with the second immunogenic composition is administered at the first administration, the first immunogenic composition according to the invention is administered at the second administration and the concomitant administration of the first immunogenic composition according to the invention with the second immunogenic composition is administered at the third and fourth administrations.

In another embodiment of said 4-administration schedule, the first immunogenic composition according to the invention is administered at the first administration, the concomitant administration of the first immunogenic composition according to the invention with the second immunogenic composition is administered at the second and third administration and the first immunogenic composition according to the invention is administered at the fourth administration.

In another embodiment of said 4-administration schedule, the concomitant administration of the first immunogenic composition according to the invention with the second immunogenic composition is administered at the first administration, the first immunogenic composition according to the invention is administered at the second and third administrations and the concomitant administration of the first immunogenic composition according to the invention with the second immunogenic composition is administered at the fourth administration.

In an embodiment, the schedule of vaccination of said sequential administration consists of a series of 5 administrations.

In an embodiment, said schedule consists of a series of 5 administrations separated by an interval of about 1 month to about 12 months. In a particular embodiment, said schedule consists of a series of 5 administrations wherein each administration is separated by an interval of about 1, 2, 3, 4, 5, 6, 7, 8, 9, 10, 11 or 12 months. In a particular embodiment, said schedule consists of a series of 5 administrations wherein each administration is separated by an interval of about 1 month to about 6 months. In a particular embodiment, said schedule consists of a series of 5 administrations wherein each administration is separated by an interval of about 1, 2, 3, 4, 5 or 6 months. In an embodiment, said schedule consists of a series of 5 administrations separated by an interval of about 1 month to about 2 months. In another embodiment, said schedule consists of a series of 5 administrations wherein each administration is separated by an interval of about 1 month, or a series of 5 administrations wherein each administration is separated by an interval of about 2 months.

In an embodiment said schedule consists of a series of 4 administrations wherein each dose is separated by an interval of about 1 month to about 3 months followed by a fifth administration about 10 months to about 13 months after the first administration. In another embodiment, said schedule consists of a series of 4 administrations wherein each administration is separated by an interval of about 1 month to about 2 months followed by a fifth administration about 10 months to about 13 months after the first dose. In another embodiment, said schedule consists of a series of 4 administrations wherein each dose is separated by an interval of about 1 month followed by a fifth administration about 10 months to about 13 months after the first administration, or a series of 4 administrations wherein each administration is separated by an interval of about 2 months followed by a fifth administration about 10 months to about 13 months after the first administration.

In an embodiment of said 5-administration schedule, the first, second, third and fourth administrations are administered in the first year of age and the fifth administration is a toddler dose. In an embodiment, said 5-administrations schedule consists of a series of 4 administrations wherein each administration is separated by an interval of about 1 month to about 2 months (for example 28-56 days between doses), starting at 2 months of age, and followed by a toddler administration at 12-18 months of age. In an embodiment, said schedule consists of a series of 4 administrations wherein each administration is separated by an interval of about 1 month to about 2 months (for example 28-56 days between doses), starting at 2 months of age, and followed by a toddler administration at 12-15 months of age.

In an embodiment of said 5-administrations schedule, the first immunogenic composition according to the invention (designated 1^{st} IC in the below table) and the concomitant administration of the first immunogenic composition according to the invention with the second immunogenic composition (designated 1^{st} IC/2^{nd} IC in the below table) may be administered in the following order:

| Schedule | Dose | | | | |
|---|---|---|---|---|---|
| number | 1 | 2 | 3 | 4 | 5 |
| 1 | 1st IC/2nd IC | 1st IC/2nd IC | 1st IC/2nd IC | 1st IC/2nd IC | 1^{st} IC |
| 2 | 1st IC/2nd IC | 1st IC/2nd IC | 1st IC/2nd IC | 1^{st} IC | 1st IC/2nd IC |
| 3 | 1st IC/2nd IC | 1st IC/2nd IC | 1st IC/2nd IC | 1^{st} IC | 1^{st} IC |
| 4 | 1st IC/2nd IC | 1st IC/2nd IC | 1^{st} IC | 1st IC/2nd IC | 1st IC/2nd IC |
| 5 | 1st IC/2nd IC | 1st IC/2nd IC | 1^{st} IC | 1^{st} IC | 1st IC/2nd IC |
| 6 | 1st IC/2nd IC | 1st IC/2nd IC | 1^{st} IC | 1^{st} IC | 1^{st} IC |
| 7 | 1st IC/2nd IC | 1st IC/2nd IC | 1^{st} IC | 1st IC/2nd IC | 1^{st} IC |
| 8 | 1st IC/2nd IC | 1^{st} IC | 1st IC/2nd IC | 1st IC/2nd IC | 1st IC/2nd IC |
| 9 | 1st IC/2nd IC | 1^{st} IC | 1st IC/2nd IC | 1st IC/2nd IC | 1^{st} IC |
| 10 | 1st IC/2nd IC | 1^{st} IC | 1st IC/2nd IC | 1^{st} IC | 1st IC/2nd IC |
| 11 | 1st IC/2nd IC | 1^{st} IC | 1st IC/2nd IC | 1^{st} IC | 1^{st} IC |
| 12 | 1st IC/2nd IC | 1^{st} IC | 1^{st} IC | 1st IC/2nd IC | 1st IC/2nd IC |
| 13 | 1st IC/2nd IC | 1^{st} IC | 1^{st} IC | 1st IC/2nd IC | 1^{st} IC |
| 14 | 1st IC/2nd IC | 1^{st} IC | 1^{st} IC | 1^{st} IC | 1st IC/2nd IC |
| 15 | 1st IC/2nd IC | 1^{st} IC | 1^{st} IC | 1^{st} IC | 1^{st} IC |
| 16 | 1^{st} IC | 1st IC/2nd IC | 1st IC/2nd IC | 1st IC/2nd IC | 1st IC/2nd IC |
| 17 | 1^{st} IC | 1st IC/2nd IC | 1st IC/2nd IC | 1st IC/2nd IC | 1^{st} IC |
| 18 | 1^{st} IC | 1st IC/2nd IC | 1st IC/2nd IC | 1^{st} IC | 1st IC/2nd IC |
| 19 | 1^{st} IC | 1st IC/2nd IC | 1st IC/2nd IC | 1^{st} IC | 1^{st} IC |
| 20 | 1^{st} IC | 1st IC/2nd IC | 1^{st} IC | 1st IC/2nd IC | 1st IC/2nd IC |
| 21 | 1^{st} IC | 1st IC/2nd IC | 1^{st} IC | 1st IC/2nd IC | 1^{st} IC |
| 22 | 1^{st} IC | 1st IC/2nd IC | 1^{st} IC | 1^{st} IC | 1st IC/2nd IC |
| 23 | 1^{st} IC | 1st IC/2nd IC | 1^{st} IC | 1^{st} IC | 1^{st} IC |
| 24 | 1^{st} IC | 1^{st} IC | 1st IC/2nd IC | 1st IC/2nd IC | 1st IC/2nd IC |
| 25 | 1^{st} IC | 1^{st} IC | 1st IC/2nd IC | 1st IC/2nd IC | 1^{st} IC |
| 26 | 1^{st} IC | 1^{st} IC | 1st IC/2nd IC | 1^{st} IC | 1st IC/2nd IC |

The above table provides the order of administration of the first immunogenic composition according to the invention (designated 1^{st} IC in the below table) and the concomitant administration of the first immunogenic composition according to the invention with the second immunogenic composition (designated 1^{st} IC/2^{nd} IC in the below table) for the different doses, for example schedule number 1 is to be read as: in embodiment of said 5-administration schedule, the concomitant administration of the first immunogenic composition according to the invention with the second immunogenic composition is administered as the first, second, third and fourth doses and the first immunogenic composition according to the invention is administered as the fifth dose.

In an embodiment, the order of administration is according to schedule 1, 2, 3, 4, 5, 6, 7, 8, 9, 10, 11, 12, 13, 14, 16, 17, 18, 19, 20, 22 or 23.

In an embodiment, the schedule of vaccination of said sequential dose consists of a series of 6 administrations.

In an embodiment, said schedule consists of a series of 6 administrations separated by an interval of about 1 month to about 12 months. In a particular embodiment, said schedule consists of a series of 6 administrations wherein each administration is separated by an interval of about 1, 2, 3, 4, 5, 6, 7, 8, 9, 10, 11 or 12 months. In a particular embodiment, said schedule consists of a series of 6 administrations wherein each administration is separated by an interval of about 1 month to about 6 months. In a particular embodiment, said schedule consists of a series of 6 administrations wherein each administration is separated by an interval of about 1, 2, 3, 4, 5 or 6 months. In an embodiment, said schedule consists of a series of 6 administrations separated by an interval of about 1 month to about 2 months. In another embodiment, said schedule consists of a series of 6 administrations wherein each administration is separated by an interval of about 1 month, or a series of 6 administrations wherein each administration is separated by an interval of about 2 months.

In an embodiment said 6-administration schedule consists of a series of 5 administrations wherein each administration is separated by an interval of about 1 month to about 2 months followed by a sixth administration about 10 months to about 13 months after the first administration. In another embodiment, said schedule consists of a series of 5 administrations wherein each administration is separated by an interval of about 1 month followed by a sixth administration about 10 months to about 13 months after the first administration, or a series of 5 administrations wherein each administration is separated by an interval of about 2 months followed by a sixth administration about 10 months to about 13 months after the first administration.

In an embodiment of said 6-administrations schedule, the first, second, third, fourth and fifth administrations are administered in the first year of age and the sixth administration is a toddler administration. In an embodiment, said 6-administrations schedule consists of a series of 5 administrations wherein each administration is separated by an interval of about 1 month to about 2 months (for example 28-56 days between administrations), starting at 2 months of age, and followed by a toddler administration at 12-18 months of age. In an embodiment, said schedule consists of a series of 5 administrations wherein each administration is separated by an interval of about 1 month to about 2 months (for example 28-56 days between administrations), starting at 2 months of age, and followed by a toddler administration at 12-15 months of age.

In an embodiment of said 6-administrations schedule, the first immunogenic composition according to the invention and the concomitant administration of the first immunogenic composition according to the invention with the second immunogenic composition are administered in the order according to the any of the 30 schedules provided for the 5-administrations schedule (see above table, schedule 1 to 30), followed by a sixth administration. In an embodiment, the first immunogenic composition according to the invention is administered at the sixth administration. In another embodiment, the concomitant administration of the first immunogenic composition according to the invention with the second immunogenic composition is administered at the sixth administration.

In an embodiment, the schedule of vaccination of said sequential administration consists of a series of 7 administrations.

In an embodiment, said schedule consists of a series of 7 administrations separated by an interval of about 1 month to about 12 months. In a particular embodiment, said schedule consists of a series of 7 administrations wherein each administration is separated by an interval of about 1, 2, 3, 4, 5, 6, 7, 8, 9, 10, 11 or 12 months. In a particular embodiment, said schedule consists of a series of 7 administrations wherein each administration is separated by an interval of about 1 month to about 6 months. In a particular embodiment, said schedule consists of a series of 7 administrations wherein each administration is separated by an interval of about 1, 2, 3, 4, 5 or 6 months. In an embodiment, said schedule consists of a series of 7 administrations separated by an interval of about 1 month to about 2 months. In another embodiment, said schedule consists of a series of 7 administrations wherein each administration is separated by an interval of about 1 month, or a series of 7 administrations wherein each administration is separated by an interval of about 2 months.

In an embodiment said 7-administration schedule consists of a series of 6 administrations wherein each administration is separated by an interval of about 1 month followed by a seventh administration about 10 months to about 13 months after the first administration.

In an embodiment of said 7-administrations schedule, the first, second, third, fourth, fifth and sixth administrations are administered in the first year of age and the seventh administration is a toddler administration. In an embodiment, said 7-administration schedule consists of a series of 6 administrations wherein each administration is separated by an interval of about 1 month (for example 28-40 days between administrations), starting at 2 months of age, and followed by a toddler administration at 12-18 months of age. In an embodiment, said schedule consists of a series of 6 administrations wherein each administration is separated by an interval of about 1 month (for example 28-40 days between administrations), starting at 2 months of age, and followed by a toddler administration at 12-15 months of age.

In an embodiment of said 7-administrations schedule, the first immunogenic composition according to the invention (such as the ones of section 2 above) and the concomitant administration of the first immunogenic composition according to the invention with the second immunogenic composition are administered in the order according to the any of the schedules provided for the 6-administrations schedule (see above), followed by a seventh administration. In an embodiment, the first immunogenic composition according to the invention is administered at the seventh administration. In another embodiment, the concomitant administration of the first immunogenic composition according to the invention with the second immunogenic composition is administered at the seventh administration.

In an embodiment, the schedule of vaccination of said sequential administration consists of a series of 8 administrations.

In an embodiment, said schedule consists of a series of 8 administrations separated by an interval of about 1 month to about 12 months. In a particular embodiment, said schedule consists of a series of 8 administrations wherein each administration is separated by an interval of about 1, 2, 3, 4, 5, 6, 7, 8, 9, 10, 11 or 12 months. In a particular embodiment, said schedule consists of a series of 8 administrations wherein each administration is separated by an interval of about 1 month to about 6 months. In a particular embodiment, said schedule consists of a series of 8 administrations wherein each administration is separated by an interval of about 1, 2, 3, 4, 5 or 6 months. In an embodiment, said schedule consists of a series of 8 administrations separated by an interval of about 1 month to about 2 months. In another embodiment, said schedule consists of a series of 8 administrations wherein each administration is separated by an interval of about 1 month, or a series of 8 administrations wherein each administration is separated by an interval of about 2 months.

In an embodiment said 8-administration schedule consists of a series of 7 administrations wherein each administration is separated by an interval of about 1 month followed by an eight administration about 10 months to about 13 months after the first administration.

In an embodiment of said 8-administrations schedule, the first, second, third, fourth, fifth, sixth and seventh administrations are administered in the first year of age and the eighth administration is a toddler administration. In an embodiment, said 8-administration schedule consists of a series of 7 administrations wherein each administration is separated by an interval of about 1 month (for example 28-40 days between administrations), starting at 2 months of age, and followed by a toddler administration at 12-18 months of age. In an embodiment, said schedule consists of a series of 7 administrations wherein each administration is separated by an interval of about 1 month (for example 28-40 days between administrations), starting at 2 months of age, and followed by a toddler administration at 12-15 months of age.

In an embodiment of said 8-administrations schedule, the first immunogenic composition according to the invention (such as the ones of section 2 above) and the concomitant administration of the first immunogenic composition according to the invention with the second immunogenic composition are administered in the order according to the any of the schedules provided for the 7-administrations schedule (see above), followed by a eighth dose. In an embodiment, the first immunogenic composition according to the invention is administered at the eighth dose. In another embodiment, the concomitant administration of the first immunogenic composition according to the invention with the second immunogenic composition is administered at the eighth dose.

In an embodiment, in the administration schedules disclosed above the concomitant administration(s) is/are replaced by a concurrent administration.

In an embodiment, the present invention pertains to the sequential administration of:
(a) the second immunogenic composition (such as the ones of section 3 above) and
(b) the concomitant administration of the first immunogenic composition according to the invention (such as the ones of section 2 above) with said second immunogenic composition.

In an embodiment said second immunogenic composition is any of the immunogenic compositions disclosed at section 3 above.

In an embodiment, the schedule of administration is any one of the schedules disclosed above for the sequential administration of a first immunogenic composition according to the invention and the concomitant administration of the first immunogenic composition according to the invention with a second immunogenic composition (bottom of page 151- to top of 164), wherein administration of said second immunogenic composition of (a) replace administration of the first immunogenic composition of (a) in said schedules.

In an embodiment, in any of the administration schedules disclosed above a concomitant administration(s) is/are replaced by a concurrent administration.

In an embodiment, the immunogenic compositions disclosed herein are administered by intramuscular or subcutaneous injection.

In an embodiment, the immunogenic compositions are administered by intramuscular injection in a thigh or arm. In an embodiment, the injection site is the anterolateral thigh muscle or the deltoid muscle.

In an embodiment, the immunogenic compositions are administered by subcutaneous injection in a thigh or an arm. In an embodiment, the injection site is the fatty tissue over the anterolateral thigh muscle or the fatty tissue over triceps.

In case of concomitant administration, the first injection can be made in one thigh and the second in the other thigh (preferably in the anterolateral thigh muscles). Alternatively, the first injection can be made in one arm and the second in the other arm (preferably in the deltoid muscles). The first injection can also be made in a thigh and the second in an arm or the first injection in an arm and the second in a thigh.

In an aspect the invention pertains to the kit of the present invention (such as the ones of section 4 above) for use in any of the immunization schedules disclosed above.

As used herein, the term “about” means within a statistically meaningful range of a value, such as a stated concentration range, time frame, molecular weight, temperature or pH. Such a range can be within an order of magnitude, typically within 20%, more typically within 10%, and even more typically within 5% or within 1% of a given value or range. Sometimes, such a range can be within the experimental error typical of standard methods used for the measurement and/or determination of a given value or range. The allowable variation encompassed by the term “about” will depend upon the particular system under study, and can be readily appreciated by one of ordinary skill in the art. Whenever a range is recited within this application, every whole number integer within the range is also contemplated as an embodiment of the disclosure.

The terms “comprising”, “comprise” and “comprises” herein are intended by the inventors to be optionally substitutable with the terms “consisting essentially of”, “consist essentially of”, “consists essentially of”, “consisting of”, “consist of” and “consists of”, respectively, in every instance.

An “immunogenic amount”, an “immunologically effective amount”, a “therapeutically effective amount”, a “prophylactically effective amount”, or “dose”, each of which is used interchangeably herein, generally refers to the amount of antigen or immunogenic composition sufficient to elicit an immune response, either a cellular (T cell) or humoral (B cell or antibody) response, or both, as measured by standard assays known to one skilled in the art.

All references or patent applications cited within this patent specification are incorporated by reference herein.

The invention is illustrated in the accompanying examples. The examples below are carried out using standard techniques, which are well known and routine to those of skill in the art, except where otherwise described in detail. The examples are illustrative, but do not limit the invention.

SEC-MALLS is used for the determination of the molecular weight of polysaccharides and polysaccharide-protein conjugates. SEC is used to separate the polysaccharides by hydrodynamic volume. Refractive index (RI) and multi-angle laser light scattering (MALLS) detectors are used for the determination of the molecular weight. When light interacts with matter, it scatters and the amount of scattered light is related to the concentration, the square of the dn/dc (the specific refractive index increments), and the molar mass of the matter. The molecular weight measurement is calculated based on the readings from the scattered light signal from the MALLS detector and the concentration signal from the RI detector.

Opsonophagocytic activity (OPA) assays are used to measure functional antibodies in murine sera specific for S. pneumonia serotypes disclosed herein. Test serum is set up in assay reactions that measure the ability of capsular polysaccharide specific immunoglobulin to opsonize bacteria, trigger complement deposition, thereby facilitating phagocytosis and killing of bacteria by phagocytes. The OPA titer is defined as the reciprocal dilution that results in a 50% reduction in bacterial count over control wells without test serum. The OPA titer is interpolated from the two dilutions that encompass this 50% killing cut-off.

OPA procedures are based on methods described in Hu et al. (2005) Clin Diagn Lab Immunol 12 (2): 287-295 with the following modifications. Test serum is serially diluted 2.5-fold and added to microtiter assay plates. Live serotype target bacterial strains are added to the wells and the plates are shaken at 25° C. for 30 minutes. Differentiated HL-60 cells (phagocytes) and baby rabbit serum (3- to 4-week old, PEL-FREEZ®, 12.5% final concentration) are then added to the wells, and the plates are shaken at 37° C. for 45 minutes. To terminate the reaction, 80 μL of 0.9% NaCl is added to all wells, mixed, and a 10 μL aliquot is transferred to the wells of MULTISCREEN® HTS HV filter plates (MILLIPORE®) containing 200 μL of water. Liquid is filtered through the plates under vacuum, and 150 μL of HYSOY® medium is added to each well and filtered through. The filter plates are then incubated at 37° C., 5% CO₂ overnight and are then fixed with Destain Solution (Bio-Rad Laboratories, Inc., Hercules, CA). The plates are then stained with Coomassie Blue and destained once. Colonies are imaged and enumerated on a Cellular Technology Limited (CTL) (Shaker Heights, OH) IMMUNOSPOT® Analyzer. Raw colony counts are used to plot kill curves and calculate OPA titers.

All publications and patent applications mentioned in the specification are indicative of the level of those skilled in the art to which this invention pertains. All publications and patent applications are hereby incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

1. An immunogenic composition comprising at least one glycoconjugate comprising a Streptococcus pneumoniae (S. pneumoniae) capsular polysaccharide conjugated to a carrier protein, wherein said capsular polysaccharide is from a S. pneumoniae serotype selected from the group consisting of S. pneumoniae serotypes 6C, 7C, 9N, 15A, 15B, 15C, 16F, 17F, 20, 23A, 23B, 31, 34, 35B, 35F, and 38.

2. The immunogenic composition of claim 1, wherein said composition comprises a glycoconjugate comprising capsular polysaccharide from S. pneumoniae serotype 7C and a glycoconjugate comprising capsular polysaccharide from S. pneumoniae serotype 38.

3. The immunogenic composition of claim 1, wherein the capsular polysaccharide of said glycoconjugates are individually conjugated to CRM₁₉₇.

4. The immunogenic composition of claim 1, wherein at least one of said serotype 6C, 7C, 9N, 15A, 15B, 15C, 16F, 17F, 20, 23A, 23B, 31, 34, 35B, 35F, or 38 glycoconjugate has a molecular weight of between 1,000 kDa and 20,000 kDa.

5. The immunogenic composition of claim 1, wherein at least one of said serotype 6C, 7C, 9N, 15A, 15B, 15C, 16F, 17F, 20, 23A, 23B, 31, 34, 35B, 35F, or 38 glycoconjugate comprises less than about 50% of free serotype 6C, 7C, 9N, 15A, 15B, 15C, 16F, 17F, 20, 23A, 23B, 31, 34, 35B, 35F, or 38 capsular polysaccharide compared to the total amount of serotype 6C, 7C, 9N, 15A, 15B, 15C, 16F, 17F, 20, 23A, 23B, 31, 34, 35B, 35F, or 38 capsular polysaccharide.

6. The immunogenic composition of claim 1, wherein the degree of conjugation of at least one of said serotype 6C, 7C, 9N, 15A, 15B, 15C, 16F, 17F, 20, 23A, 23B, 31, 34, 35B, 35F, or 38 glycoconjugate is between 2 and 15.

7. The immunogenic composition of claim 1, wherein at least one of said serotype 6C, 7C, 9N, 15A, 15B, 15C, 16F, 17F, 20, 23A, 23B, 31, 34, 35B, 35F, or 38 glycoconjugate is prepared using reductive amination.

8. The immunogenic composition of claim 1, wherein at least one of said serotype 6C, 7C, 9N, 15A, 15B, 15C, 16F, 17F, 20, 23A, 23B, 31, 34, 35B, 35F, or 38 glycoconjugate has a molecular weight of between 400 kDa and 15,000 kDa.

9. The immunogenic composition of claim 1, wherein at least one of said serotype 6C, 7C, 9N, 15A, 15B, 15C, 16F, 17F, 20, 23A, 23B, 31, 34, 35B, 35F, or 38 glycoconjugate has a molecular weight of between 1,000 kDa and 8,000 kDa.

10. The immunogenic composition of claim 1, wherein at least one of said serotype 6C, 7C, 9N, 15A, 15B, 15C, 16F, 17F, 20, 23A, 23B, 31, 34, 35B, 35F, or 38 glycoconjugate is prepared using reductive amination.

11. The immunogenic composition of claim 1, wherein each dose of said immunogenic composition comprises 0.1 μg to 100 μg of polysaccharide of each serotype.

12. The immunogenic composition of claim 1, wherein each dose of said immunogenic composition comprises 1.0 μg to 10 μg of polysaccharide of each serotype.

13. The immunogenic composition of claim 1, wherein each dose of said immunogenic composition comprises about 1.0 μg, about 1.2 μg, about 1.4 μg, about 1.6 μg, about 1.8 μg, 2.0 μg, about 2.2 μg, about 2.4 μg, about 2.6 μg, about 2.8 μg, about 3.0 μg, about 3.2 μg, about 3.4 μg, about 3.6μ, about 3.8μ, about 4.0μ, about 4.2μ, about 4.4μ, about 4.6μ, about 4.8μ, about 5.0 μg, about 5.2 μg, about 5.4 μg, about 5.6 μg, about 5.8 μg or about 6.0 μg of polysaccharide for each serotype glycoconjugate.

14. The immunogenic composition of claim 1, wherein said immunogenic composition further comprises at least one antigen selected from the group consisting of a diphtheria toxoid (D), a tetanus toxoid (T), a pertussis antigen (P), an acellular pertussis antigen (Pa), a hepatitis B virus (HBV) surface antigen (HBsAg), a hepatitis A virus (HAV) antigen, a conjugated Haemophilus influenzae type b capsular saccharide (Hib), and inactivated poliovirus vaccine (IPV).

15. The immunogenic composition of claim 1, wherein said immunogenic composition further comprises at least one adjuvant selected from the group consisting of aluminum phosphate, aluminum sulfate or aluminum hydroxide, calcium phosphate, liposomes, an oil-in-water emulsion, MF59 (4.3% w/v squalene, 0.5% w/v polysorbate 80, 0.5% w/v sorbitan trioleate), a water-in-oil emulsion, MONTANIDE™, poly(D,L-lactide-co-glycolide) (PLG) microparticles and poly(D,L-lactide-co-glycolide) (PLG) nanoparticles.

16. The immunogenic composition of claim 1, wherein said immunogenic composition further comprises a CpG Oligonucleotide.

17. The immunogenic composition of claim 1, wherein said immunogenic composition has a pH of 5.5 to 7.5.

18. The immunogenic composition of claim 1, wherein said immunogenic composition is simultaneously, concurrently, concomitantly or sequentially administered with a second immunogenic composition comprising at least one glycoconjugate comprising capsular polysaccharide from a Streptococcus pneumoniae serotype selected from the group consisting of serotypes 1, 3, 4, 5, 6A, 6B, 7F, 9V, 14, 18C, 19A, 19F, 23F, 22F and 33F.

19. (canceled)

20. (canceled)

21. The immunogenic composition of claim 1, further comprising at least 13 glycoconjugates, each comprising capsular polysaccharide from a different S. pneumoniae serotype selected from the group consisting of serotypes 6C, 9N, 15A, 15B, 16F, 20, 23A, 23B, 31, 35B, and 35F.

22. A method of preventing, treating or ameliorating an infection, disease, or condition associated with S. pneumoniae in a subject, comprising administering to a subject an immunologically effective amount of the immunogenic composition of claim 1.


SEQUENCE LISTING (40 sequences)

SEQ ID NO: 1 - DNA, 21 bp, synthetic construct
  misc_feature 1..21: note = Synthetic nucleotide sequence
  source 1..21: mol_type = other DNA
ggggacgacgtcgtggggggg

SEQ ID NO: 2 - DNA, 21 bp, synthetic construct
  misc_feature 1..21: note = Synthetic nucleotide sequence
  source 1..21: mol_type = other DNA
ggggacgacgtcgtggggggg

SEQ ID NO: 3 - DNA, 21 bp, synthetic construct
  misc_feature 1..21: note = Synthetic nucleotide sequence
  source 1..21: mol_type = other DNA
tcgtcgtttttcggtgctttt

SEQ ID NO: 4 - DNA, 21 bp, synthetic construct
  misc_feature 1..21: note = Synthetic nucleotide sequence
  source 1..21: mol_type = other DNA
tcgtcgtttttcggtcgtttt

SEQ ID NO: 5 - DNA, 24 bp, synthetic construct
  misc_feature 1..24: note = Synthetic nucleotide sequence
  source 1..24: mol_type = other DNA
tcgtcgttttgtcgttttgtcgtt

SEQ ID NO: 6 - DNA, 24 bp, synthetic construct
  misc_feature 1..24: note = Synthetic nucleotide sequence
  source 1..24: mol_type = other DNA
tcgtcgtttcgtcgttttgtcgtt

SEQ ID NO: 7 - DNA, 24 bp, synthetic construct
  misc_feature 1..24: note = Synthetic nucleotide sequence
  source 1..24: mol_type = other DNA
tcgtcgttttgtcgtttttttcga

SEQ ID NO: 8 - DNA, 21 bp, synthetic construct
  misc_feature 1..21: note = Synthetic nucleotide sequence
  source 1..21: mol_type = other DNA
tcgtcgtttttcggtgctttt

SEQ ID NO: 9 - DNA, 21 bp, synthetic construct
  misc_feature 1..21: note = Synthetic nucleotide sequence
  source 1..21: mol_type = other DNA
tcgtcgtttttcggtcgtttt

SEQ ID NO: 10 - DNA, 24 bp, synthetic construct
  misc_feature 1..24: note = Synthetic nucleotide sequence
  source 1..24: mol_type = other DNA
tcgtcgttttgtcgttttgtcgtt

SEQ ID NO: 11 - DNA, 24 bp, synthetic construct
  misc_feature 1..24: note = Synthetic nucleotide sequence
  source 1..24: mol_type = other DNA
tcgtcgtttcgtcgttttgtcgtt

SEQ ID NO: 12 - DNA, 24 bp, synthetic construct
  misc_feature 1..24: note = Synthetic nucleotide sequence
  source 1..24: mol_type = other DNA
tcgtcgttttgtcgtttttttcga

SEQ ID NO: 13 - DNA, 22 bp, synthetic construct
  misc_feature 1..22: note = Synthetic nucleotide sequence
  source 1..22: mol_type = other DNA
tcgcgtcgttcggcgcgcgccg

SEQ ID NO: 14 - DNA, 23 bp, synthetic construct
  misc_feature 1..23: note = Synthetic nucleotide sequence
  source 1..23: mol_type = other DNA
tcgtcgacgttcggcgcgcgccg

SEQ ID NO: 15 - DNA, 21 bp, synthetic construct
  misc_feature 1..21: note = Synthetic nucleotide sequence
  source 1..21: mol_type = other DNA
tcggacgttcggcgcgcgccg

SEQ ID NO: 16 - DNA, 19 bp, synthetic construct
  misc_feature 1..19: note = Synthetic nucleotide sequence
  source 1..19: mol_type = other DNA
tcggacgttcggcgcgccg

SEQ ID NO: 17 - DNA, 20 bp, synthetic construct
  misc_feature 1..20: note = Synthetic nucleotide sequence
  source 1..20: mol_type = other DNA
tcgcgtcgttcggcgcgccg

SEQ ID NO: 18 - DNA, 20 bp, synthetic construct
  misc_feature 1..20: note = Synthetic nucleotide sequence
  source 1..20: mol_type = other DNA
tcgacgttcggcgcgcgccg

SEQ ID NO: 19 - DNA, 18 bp, synthetic construct
  misc_feature 1..18: note = Synthetic nucleotide sequence
  source 1..18: mol_type = other DNA
tcgacgttcggcgcgccg

SEQ ID NO: 20 - DNA, 18 bp, synthetic construct
  misc_feature 1..18: note = Synthetic nucleotide sequence
  source 1..18: mol_type = other DNA
tcgcgtcgttcggcgccg

SEQ ID NO: 21 - DNA, 22 bp, synthetic construct
  misc_feature 1..22: note = Synthetic nucleotide sequence
  source 1..22: mol_type = other DNA
tcgcgacgttcggcgcgcgccg

SEQ ID NO: 22 - DNA, 22 bp, synthetic construct
  misc_feature 1..22: note = Synthetic nucleotide sequence
  source 1..22: mol_type = other DNA
tcgtcgttttcggcgcgcgccg

SEQ ID NO: 23 - DNA, 22 bp, synthetic construct
  misc_feature 1..22: note = Synthetic nucleotide sequence
  source 1..22: mol_type = other DNA
tcgtcgttttcggcggccgccg

SEQ ID NO: 24 - DNA, 24 bp, synthetic construct
  misc_feature 1..24: note = Synthetic nucleotide sequence
  source 1..24: mol_type = other DNA
tcgtcgttttacggcgccgtgccg

SEQ ID NO: 25 - DNA, 23 bp, synthetic construct
  misc_feature 1..23: note = Synthetic nucleotide sequence
  source 1..23: mol_type = other DNA
tcgtcgttttcggcgcgcgccgt

SEQ ID NO: 26 - DNA, 22 bp, synthetic construct
  misc_feature 1..22: note = Synthetic nucleotide sequence
  source 1..22: mol_type = other DNA
tcgcgtcgttcggcgcgcgccg

SEQ ID NO: 27 - DNA, 23 bp, synthetic construct
  misc_feature 1..23: note = Synthetic nucleotide sequence
  source 1..23: mol_type = other DNA
tcgtcgacgttcggcgcgcgccg

SEQ ID NO: 28 - DNA, 21 bp, synthetic construct
  misc_feature 1..21: note = Synthetic nucleotide sequence
  source 1..21: mol_type = other DNA
tcggacgttcggcgcgcgccg

SEQ ID NO: 29 - DNA, 19 bp, synthetic construct
  misc_feature 1..19: note = Synthetic nucleotide sequence
  source 1..19: mol_type = other DNA
tcggacgttcggcgcgccg

SEQ ID NO: 30 - DNA, 20 bp, synthetic construct
  misc_feature 1..20: note = Synthetic nucleotide sequence
  source 1..20: mol_type = other DNA
tcgcgtcgttcggcgcgccg

SEQ ID NO: 31 - DNA, 20 bp, synthetic construct
  misc_feature 1..20: note = Synthetic nucleotide sequence
  source 1..20: mol_type = other DNA
tcgacgttcggcgcgcgccg

SEQ ID NO: 32 - DNA, 18 bp, synthetic construct
  misc_feature 1..18: note = Synthetic nucleotide sequence
  source 1..18: mol_type = other DNA
tcgacgttcggcgcgccg

SEQ ID NO: 33 - DNA, 18 bp, synthetic construct
  misc_feature 1..18: note = Synthetic nucleotide sequence
  source 1..18: mol_type = other DNA
tcgcgtcgttcggcgccg

SEQ ID NO: 34 - DNA, 22 bp, synthetic construct
  misc_feature 1..22: note = Synthetic nucleotide sequence
  source 1..22: mol_type = other DNA
tcgcgacgttcggcgcgcgccg

SEQ ID NO: 35 - DNA, 22 bp, synthetic construct
  misc_feature 1..22: note = Synthetic nucleotide sequence
  source 1..22: mol_type = other DNA
tcgtcgttttcggcgcgcgccg

SEQ ID NO: 36 - DNA, 22 bp, synthetic construct
  misc_feature 1..22: note = Synthetic nucleotide sequence
  source 1..22: mol_type = other DNA
tcgtcgttttcggcggccgccg

SEQ ID NO: 37 - DNA, 24 bp, synthetic construct
  misc_feature 1..24: note = Synthetic nucleotide sequence
  source 1..24: mol_type = other DNA
tcgtcgttttacggcgccgtgccg

SEQ ID NO: 38 - DNA, 23 bp, synthetic construct
  misc_feature 1..23: note = Synthetic nucleotide sequence
  source 1..23: mol_type = other DNA
tcgtcgttttcggcgcgcgccgt

SEQ ID NO: 39 - DNA, 23 bp, synthetic construct
  misc_feature 1..23: note = Synthetic nucleotide sequence
  source 1..23: mol_type = other DNA
tcgtcgacgatcggcgcgcgccg

SEQ ID NO: 40 - DNA, 23 bp, synthetic construct
  misc_feature 1..23: note = Synthetic nucleotide sequence
  source 1..23: mol_type = other DNA
tcgtcgacgatcggcgcgcgccg